(19) United States
(12) Patent Application Publication
(10) Pub. No.: US 2023/0102191 A1
(43) Pub. Date: Mar. 30, 2023

(54) NONIONIC POLYMERIC FATTY ACID COMPOUNDS FOR THE TREATMENT OF FIBROUS AMINO ACID-BASED SUBSTRATES, ESPECIALLY HAIR

(71) Applicant: MOMENTIVE PERFORMANCE MATERIALS GMBH, Leverkusen (DE)

(72) Inventors: Roland WAGNER, Bonn (DE); Katharina STREICHER, Leverkusen (DE); Laura ROHMANN, Düsseldorf (DE); Christian WENSKE, Solingen (DE); Shrutisagar Dattatraya HAVELI, White Plains, NY (US)

(21) Appl. No.: 17/786,128

(22) PCT Filed: Dec. 15, 2020
(86) PCT No.: PCT/IB2020/001058

§ 371 Date: Jun. 16, 2022

Related U.S. Application Data
(60) Provisional application No. 62/948,982, filed on Dec. 17, 2019.

(51) Int. Cl. A61K 8/85; A61Q 5/12; A61K 8/37

(52) U.S. Cl. CPC A61K 8/85; A61Q 5/12; A61K 8/37; A61K 8/375

(57) ABSTRACT

The present invention is directed at hair care formulations comprising at least one non-ionic compound containing at least one terminal estolide moiety, at non-ionic compounds containing at least one terminal estolide moiety, the use of non-ionic compounds containing at least one terminal estolide moiety in cosmetic formulations for skin and/or hair care, the use of non-ionic compounds containing at least one terminal estolide moiety for the treatment of fibers, and at compositions containing at least one such compound for the treatment of hair.

### FIELD OF THE INVENTION

This invention relates to non-ionic polymeric fatty acid compounds, the use of nonionic polymeric fatty acid compounds, aqueous compositions comprising the same, cosmetic compositions comprising the same, in particular, hair care compositions, and their use for the treatment of hair.

### BACKGROUND OF THE INVENTION

Hair generally can be straight, wavy, curly, kinky or twisted. A human hair includes three main morphological components, the cuticle (a thin, outer-most shell of several concentric layers), the cortex (the main body of the hair), and, in case of higher diameter hair, the medulla (a thin, central core). The cuticle and cortex provide the hair strand's mechanical properties, that is, its tendency to have a wave, curl, or kink. A straight hair strand can resemble a rod with a circular cross-section, a wavy hair strand can appear compressed into an oval cross-section, a curly strand can appear further compressed into an elongated ellipse cross-section, and a kinky hair strand cross-section can be flatter still.

The primary component of hair is the cross-linked, alpha-helix protein keratin. Keratins are intermediate filament proteins found specifically in epithelial cells, e.g. human skin and hair, wool, feathers, and nails. The α-helical type I and II keratin intermediate filament proteins (KIFs) with molecular weights around 45-60 kDa are embedded in an amorphous matrix of keratin-associated proteins (KAPs) with molecular weights between 20 to 30 kDa (M. A. Rogers, L. Langbein, S. Praetzel-Wunder, H. Winter, J. Schweizer, J. Int Rev Cytol. 2006; 251:209-6); both intra- and intermolecular disulfide bonds provided by cystines contribute to the cytoskeletal protein network maintaining the cellular scaffolding. In addition to the disulfide cross-links ionic bonding or salt bridges which pair various amino acids found in the hair proteins contribute to the hair strand's outward shape.

It is known in the art that hair can be treated with functionalized silicones and hydrocarbons which deliver one or more cosmetic benefits, such as conditioning, shine and UV protection as well as color retention. Typically, these silicone and hydrocarbon based derivatives are physically deposited on the fiber surface (cuticle) and therefore responsible for the outward appearance of the hair, i.e. smoothness, silkiness, friction, alignment and combability. Advanced silicone derivatives are generally regarded as high performing materials with respect to attributes such as smooth and silky hair feel, friction reduction, eased combability and hair color protection.

Quaternized silicones are described in prior art disclosures, i.e. in U.S. Pat. No. 4,891,166, EP 282720, US 2008027202, U.S. Pat. Nos. 6,730,766, 6,240,929, WO 02/10257, WO 02/10259, WO 2004/069137, WO 2013/148629, WO 2013/148635, WO 2013/148935.

Typical hydrocarbon based mono quaternary ammonium compounds are saturated or unsaturated fatty acid-based mono-fatty ester and di-fatty ester quats as well as fatty amido quats having 10 to 24 carbon atoms in the alkyl chain(s). Details on these materials containing quaternary ammonium groups are disclosed for example in US 2009/0000638, WO 2012/027369, US 2013/259820 and U.S. Pat. Nos. 5,880,086, 6,465,419, 6,462,014, 6,323,167, 6,037,315, 5,854,201, 5,750,490, 5,463,094, US 2003/013627. Certain mono quats containing polymerized fatty acids as hydrophobic tails were proposed for personal care applications in WO 2004/093834.

Nonionic ester derivatives containing polymerized fatty acids were proposed as lubricants (US 2011/0282084).

C3 to C20 alcohol esters of polymeric fatty acids were proposed as conditioning agents (WO 2016/174256). WO 2013/009471 describes C1 to C40 alcohol esters of polymeric fatty acids for the same application.

Esters of Guerbet alcohols with ricinoleic acid improve the gloss of skin (U.S. Pat. No. 5,786,389). Polyesters of Guerbet alcohols with dimer acid and dimer alcohols were proposed for the same application (U.S. Pat. No. 7,259,226). Polyesters of polyalkylene oxides with dimer acid and a monofunctional acid are skin conditioners (U.S. Pat. No. 6,800,275).

Esterified monomeric and polymeric castor oil derivatives are also known and were i.e. proposed as lubricants (U.S. Pat. No. 2,049,072; A. Erdem-Senatalar et. al., J. Am. Oil Chem. Soc. 1994, 71(9), 1035-1037; A. T. Erciyes et. al., J. Am. Oil Chem. Soc. 1991, 68(9), 639-641; K. D. Pathak, J. Scientific & Industrial Res. 1955, 14B, 637-639). EP 665286 describes estolide moieties containing triglycerides as lubricants and thickening agents.

The partial transesterification of polymeric fatty acids with plant-based oils yields products useful as emollients and humectants (FR 2858616).

The esterification of castor oil with succinic acid or dimer acid and a monofunctional C6 to C34 acid yields polyesters which provide gloss on skin ((U.S. Pat. Nos. 6,787,129, 6,670,441). There has been a need for efficient compounds for the treatment of fibrous amino acid based substrates, especially hair which can be synthesized in a straight forward, cost efficient and flexible way, largely based on sustainable raw materials, which are easy to formulate and easy to use, yielding long term stable formulations even in the presence of other performance ingredients and which are useful for the conditioning of hair, for an improved dry and wet combability of hair, the smoothness and a pleasant alignment of hair. In particular, benefits regarding an improved wet and dry combability close to silicone-based conditioning agents should be achieved.

The present inventors found that non-ionic polymeric fatty acid based estolide compounds and aqueous compositions comprising the same are suitable to satisfy the above need. The present invention accordingly provides nonionic polymeric fatty acid-based estolide compounds, aqueous compositions comprising the same, cosmetic compositions comprising the same, in particular, hair care compositions, and their use for the treatment of hair, which nonionic polymeric fatty acid based estolide compounds can be synthesized in a straightforward, cost-efficient and flexible way, largely based on sustainable raw materials, are easy to formulate and to use, and are useful for the conditioning of hair, for an improved dry and wet combability of hair, the smoothness and a pleasant alignment of hair.

### SUMMARY OF THE INVENTION

The present invention relates to nonionic polymeric fatty acid-based estolide compounds, aqueous compositions comprising the same, cosmetic compositions comprising the same, in particular, hair care compositions, and their use for the treatment of hair.

According to the present invention, the present invention, a hair care formulation is provided containing at least one compound of the formula (I):

R¹(—X—C(O)—F)ₚ (I)

wherein

R¹ in formula (I) is selected from a p-valent, optionally substituted hydrocarbon radical and may contain optionally one or more groups selected from —O—, —NH—, —C(O)—, —C(S)—, tertiary amino groups

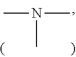

quaternary ammonium groups, and can be optionally substituted by one or more selected from carboxyl groups or hydroxyl groups,

p 2, more preferably 2-811,

X can be the same or different and is selected from —O—, or —NR¹⁰—, wherein R¹⁰ is selected from the group consisting of hydrogen, or optionally substituted straight-chain, cyclic or branched, saturated, unsaturated or aromatic hydrocarbon radicals which have up to 100 carbon atoms which optionally contain one or more groups selected from —O—, —NH—, —C(O)—, —C(S)—, tertiary amino groups

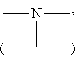

or in formula (I) R¹⁰ may form a bond to R¹ to form a cyclic structure,

F can be the same or different and is selected from optionally substituted straight-chain, cyclic or branched, saturated, unsaturated or aromatic hydrocarbon radicals which have up to 1005 carbon atoms, which optionally contain one or more groups selected from —O—, —NH—, —C(O)—, —C(S)—, tertiary amino groups

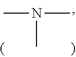

and can be optionally substituted by one or more selected from carboxyl, hydroxyl or halide groups,

with the proviso that at least one of the radicals F contains at least one moiety of the formula (II) or formula (II*):

—R⁶(—X—C(O)—R⁶)ₘ—X—C(O)—R⁷ (II)

—R⁶(—X—C(O)—R⁶)ₘ—X—C(O)—R⁷* (II*)

wherein

X is as defined above,

m=0 to 20, preferably 1 to 20,

R⁶ is independently selected from a divalent optionally substituted straight-chain, cyclic or branched, saturated or unsaturated hydrocarbon radical which have up to 36 carbon atoms,

R⁷ is independently selected from optionally substituted straight-chain, cyclic or branched, saturated or unsaturated hydrocarbon radicals which have 1 to 1000 carbon atoms, optionally containing one or more groups selected from —O—, —NH—, —C(O)—, —C(S)—, tertiary amino groups

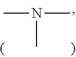

quaternary ammonium groups

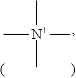

and which can be substituted with carboxyl, hydroxyl, or halide groups, wherein the radical R⁷ cannot contain an internal carboxy group or amide, i.e. R⁷ cannot contain a combination of a —C(O)— group and a —O— group or a combination of a —C(O)— group and a —NH— or tertiary amino group, with the proviso that in at least one moiety of the formula (II) R⁷ has at least 2, preferably at least 6 carbon atoms, and in the same moiety of the formula (II) at least one R⁶ has at least 6, preferably at least 8 carbon atoms,

R⁷* is independently selected from optionally substituted branched or dendrimeric hydrocarbon radicals which have 1 to 1000 carbon atoms, optionally containing one or more groups selected from —O—, —NH—, —C(O)—, —C(S)—, tertiary amino groups

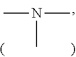

quaternary ammonium groups

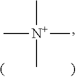

and which can be substituted with carboxyl, hydroxyl, or halide groups, wherein the radical R⁷* is terminated by two or more groups of the general structure

—X—C(O)—T

wherein X is as defined above, and

T is a monovalent straight-chain, cyclic or branched, saturated or unsaturated hydrocarbon radical optionally substituted with carboxyl, hydroxyl, or halide groups with up to 36 carbon atoms,

with the proviso that in at least one moiety of the formula (II*) R⁷* is terminated by one or more groups T having at least 2, preferably at least 6 carbon atoms, and in the same moiety of the formula (II*) at least one R⁶ has at least 6, preferably at least 8 carbon atoms,

or

containing at least one compound of the general formula (IV)

R¹(—C(O)—X—G)_{q} (IV),

wherein

X is as defined above,

R¹ in formula (IV) is selected from q-valent, optionally substituted hydrocarbon radicals which preferably have up to 1000 carbon atoms, and may contain optionally one or more groups selected from —O—, —NH—, —C(O)—, —C(S)—, tertiary amino groups

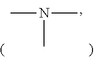

quaternary ammonium groups, and can be optionally substituted by one or more selected from carboxyl groups or hydroxyl groups,

q=2 to 55, preferably 2 to 40, more preferably 2 to 4, and

G can be the same or different and is selected from optionally substituted straight-chain, cyclic or branched, saturated, unsaturated or aromatic hydrocarbon radicals which have up to 1005 carbon atoms, which optionally contain one or more groups selected from —O—, —NH—, —C(O)—, —C(S)—, and tertiary amino groups

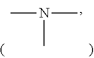

and can be substituted by one or more selected from carboxyl, hydroxyl or halide groups,

with the proviso that at least one of the radicals G contains at least one moiety of the formula (V):

—R⁶(—C(O)—X—R⁶)ₘ—C(O)—X—R¹¹ (V)

wherein

X is as defined above

m=0 to 20, preferably 1 to 20,

R⁶ in formula (V) is as defined above for formula (I),

R¹¹ is independently selected from optionally substituted straight-chain, cyclic or branched, saturated or unsaturated hydrocarbon radicals which have 1 to 1000 carbon atoms, optionally containing one or more groups selected from —O—, —NH—, —C(O)—, —C(S)—, tertiary amino groups

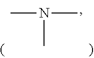

quaternary ammonium groups

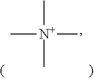

and which can be substituted with carboxyl, hydroxyl, or halide groups, wherein the radical R¹¹ cannot contain an internal carboxy group or amide, i.e. R¹¹ cannot contain a combination of a —C(O)— group and a —O— group or a combination of a —C(O)— group and a —NH— or tertiary amino group, with the proviso that in at least one moiety of the formula (V) R¹¹ has at least 2, preferably at least 6 carbon atoms, and in the same moiety of the formula (V) at least one R⁶ has at least 6, preferably at least 8 carbon atoms, with the general proviso that the compound of the formula (I) is not exclusively composed of glycerol and ricinoleic acid moieties, i.e. polyglycerol polyricinoleate (PGPR).

### DETAILED DESCRIPTION OF THE INVENTION

According to the invention, estolides are natural and synthetic compounds, in particular derived from fats and oils, more specifically from the fatty acid compounds typically obtainable by hydrolysis of oils and fats.

The estolide structure is identified by the secondary ester linkage of one fatty acyl molecule to the alkyl backbone of another fatty acid fragment. The terms “fatty acid” and “fatty acyl molecule” seem to imply that the individual residue needs to be derived from a component of a fat, which is not the case. The term “fatty acid” herein refers to carboxylic acids with chain-shaped organyl groups, in particular unbranched aliphatic monocarboxylic acids. Fatty acids differ from each other by their number of carbon atoms (chain length) and, when referring to unsaturated fatty acids, the number and position of double bonds. Fatty acids may be classified as short chain fatty acids with up to 7 carbons atoms, middle chain fatty acids with 8 to 12 carbon atoms, long chain fatty acids with 13 to 21 carbon atoms, and very long chain fatty acids with more than 22 carbon atoms.

According to the invention, the residue R′ in formula (I) is selected from a p-valent, optionally substituted hydrocarbon radical and may contain optionally one or more groups selected from —O—, —NH—, —C(O)—, —C(S)—, tertiary amino groups

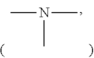

quaternary ammonium groups, and can be optionally substituted by one or more selected from carboxyl groups or hydroxyl groups.

According to the invention, the residue R¹ is p-valent, wherein p is 2 to 811, preferably 2 to 100, more preferably 2-50, even more preferably p is 2 to 30, which indicates that the residue R¹ bears p residues of the structure (—X—C(O)—F), with F as defined below. Accordingly, the term “p-valent” does not refer to or restrict the number of optional further substituents other than (—X—C(O)—F) of the residue R¹, which can be carboxylic groups or hydroxyl groups.

According to the invention the wording “optionally substituted hydrocarbon radical” that may contain optionally one or more specific groups and can be substituted by one or more specific groups refers to an organyl radical which is linked to one or more further groups via at least one of its carbon atoms, wherein the hydrocarbyl structure of the radical may be interrupted by the specific groups as defined to be contained, and one or more hydrogen atoms of the hydrocarbyl group can be substituted by the substituent groups as indicated.

In case of R¹, for example, one or more hydrogen atoms may be substituted by a hydroxyl group or by a carboxylic group.

Further, as the optionally substituted hydrocarbon radical R¹ specifically may contain one or more groups selected from —O—, —NH—, —C(O)—, —C(S)— and tertiary amino groups

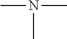

the hydrocarbyl structure of a R¹ group may be interrupted by these groups or combinations thereof. Accordingly, the residue may contain ester groups, carboxyl groups, amide groups, ether groups, amino groups, carbonyl groups, thione groups, thio carboxylate groups, thio ester groups, carbamate groups, urethane groups, epoxide groups and all other groups as specified for this radical, and combinations thereof. The same principle applies to the optionally substituted hydrocarbon radicals R², R³, R⁴, R⁵, R⁶, R⁷, R⁷* and R¹⁰.

The hydrocarbyl structure of R¹, which is p-valent regarding the residues (—X—C(O)—F), is preferably selected from the group consisting of linear, branched or cyclic alkylene groups, linear, branched or cyclic alkenylene groups, linear, branched or cyclic alkynylene groups, linear, branched or cyclic alkarylene groups, linear, branched or cyclic aralkylene groups and linear, branched or cyclic arylene groups, for instance phenylene, benzylene or tolylene groups, in particular from such groups having 1 to 1000 carbon atoms, more particular 1 to 150 carbon atoms.

Preferably, the hydrocarbon structures are linear or branched alkylene groups, or linear or branched alkylene groups interrupted by ether groups, ester groups or both ether and ester groups in particular branched structures derived from products as obtained by esterification of polyols with mono- or polyhydroxycarboxylic acids with up to 150 carbon atoms, or linear alkylene groups with up to 22 carbon atoms.

More preferably, the p-valent R¹ radical is selected from alkylene groups, which may be selected from the group consisting of linear, branched and cyclic alkylene groups, in particular from linear C1-C22 alkyl groups such as methylene, ethylene, n-propylene, n-butylene, n-pentylene, n-hexylene, n-heptylene or n-octylene groups, branched C1-C22 alkylene groups iso-propylene, iso-butylene, tert-butylene, iso-butylene, tert-pentylene, neo-pentylene, and 2-ethylhexylene groups.

There is no limitation regarding which C-atoms of the hydrocarbyl radicals bear the (—X—C(O)—F) groups attached to R¹.

Regarding the presence of functional groups optionally contained in R¹ and optional substituents, it is preferred that R¹ is derived from glycidyl compounds, glycerol and glycerol derivatives, in particular glycidol, glycerol diglycidyl ether, diglycidyl ether and polyglycerol compounds, or when R¹ is a linear alkylene group, in particular an alkylene group not bearing further substituents in addition to the (—X—C(O)—F) groups and even more preferred when R¹ is derived from the condensation product of glycidol, glycerol, glycerol diglycidyl ether, diglycidyl ether and polyglycerol compounds and C8-C24 monohydroxy fatty acids, in particular ricinoleic acid, lesquerolic acid or 12-hydroxyl stearic acid.

It is clear to the skilled person that usually the groups (—X—C(O)—F) are attached to the radical R¹ via —X—C(O)— units, in particular —O—C(O)— units, at positions which are substituted by —OH or —NHR¹⁰ groups in a parent compound from which R¹ is derived.

For example, the R¹ group derived from glycerol is a 1,2,3-propylene radical, wherein “1,2,3” indicates the positions at which the radical is substituted by the (—X—C(O)—F)-groups.

According to the invention, the term “optionally substituted hydrocarbon residue” does not impose any further restrictions on the radicals, and accordingly they are limited by the groups which can be optionally contained or present as substituents, the number of carbon atoms of the residues as specified, and the way they are bonded to other structural moieties of the compound according to the invention as defined by formula (I), formula (II), formula (II*), formula (III), formula (IV), formula (V) or any further formula used to define an embodiment according to the invention.

According to the invention, the group X can be the same or different and is selected from —O—, or —NR¹⁰—, wherein R¹⁰ is selected from the group consisting of hydrogen, or optionally substituted straight-chain, cyclic or branched, saturated, unsaturated or aromatic hydrocarbon radicals which have up to 100 carbon atoms which optionally contain one or more groups selected from —O—, —NH—, —C(O)—, —C(S)—, tertiary amino groups

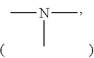

or in formula (I) R¹⁰ may form a bond to R¹ to form a cyclic structure.

Preferred examples for R¹⁰ are C1-C10 alkyl groups, in particular methyl, ethyl, n-propyl, iso-propyl, n-butyl, iso-butyl, tert-butyl, n-pentane and n-hexane groups, cyclopentyl groups and cyclohexane groups, C2-C10 alkenyl groups, in particular vinyl groups and allyl groups, and C6-C12 aromatic groups, in particular phenyl groups, tolyl groups, and benzyl groups, wherein each of the named groups may be substituted by hydroxyl groups or halide groups.

According to the invention, the residue F can be the same or different and is selected from optionally substituted straight-chain, cyclic or branched, saturated, unsaturated or aromatic hydrocarbon radicals which have up to 1005 carbon atoms, which optionally contain one or more groups selected from —O—, —NH—, —C(O)—, —C(S)—, tertiary amino groups

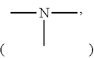

and can be optionally substituted by one or more selected from carboxyl, hydroxyl or halide groups, with the proviso that at least one compound of the hair care formulation contains at least one moiety of the formula (II) or of the formula (II*)

—R⁶(X—C(O)—R⁶)ₘ—X—C(O)—R⁷ (II)

—R⁶(X—C(O)—R⁶)ₘ—X—C(O)—R^{7*} (II*).

Preferably, the group F only consists of a group of the formula (II), or

The group F only consists of a group of the formula (II*).

According to the invention, R⁶ is independently selected from an optionally substituted straight-chain, cyclic or branched, saturated or unsaturated hydrocarbon radical which have up to 36 carbon atoms, and thus R⁶ can be a divalent, optionally substituted straight-chain, cyclic or branched, saturated, unsaturated or aromatic hydrocarbon radical, i.e. R⁶ can represent a hydrocarbyl group selected from the group consisting of linear, branched or cyclic alkylene groups, linear, branched or cyclic alkenylene groups, linear, branched or cyclic alkynylene groups, linear, branched or cyclic alkarylene groups, linear, branched or cyclic aralkylene groups and linear, branched or cyclic arylene groups, for instance phenylene, benzylene or tolylene groups, in particular from such groups having 1 to 100 carbon atoms, each optionally containing one or more functional groups as indicated above.

More preferably, the R⁶ radical is selected from linear alkylene groups and linear alkenylene groups, in particular from linear C6-C24 alkylene such as hexylene, heptylene, octylene, nonylene, decylene, undecylene, dodecylene, tridecylene, tetradecylene, pentadecylene, hexadecylene, heptadecylene, octadecylene, nonadecylene, eicosylene, henicosylene, doicosylene, tricosylene, and tetraicosylene, or linear C6-C24 alkenylene groups such as hexenylene, heptenylene, octenylene, nonenylene, decenylene, undecenylene, dodecenylene, tridecenylene, tetradecenylene, pentadecenylene, hexadecenylene, heptadecenylene, octadecenylene, nonadecenylene, eicosenylene, henicosenylene, doicosenylene, tricosenylene, and tetraicosenylene, wherein the groups are most preferably bonded to the adjacent C(O) group by a terminal C-atom.

There is no limitation regarding at which C-atoms of the hydrocarbyl radicals the adjacent group C(O) group and X group are attached to R⁶.

However, R⁶ is preferably derived from a hydroxycarboxylic acid bearing one or more hydroxylic groups, more preferably from a monohydroxy carboxylic acid, most preferably from C7-C25 fatty acids bearing one hydroxyl group as substituent. Accordingly, R⁶ preferably represents the alkylene or alkenylene chain of such carboxylic acids. For instance, if R⁶ is derived from ricinoleic acid

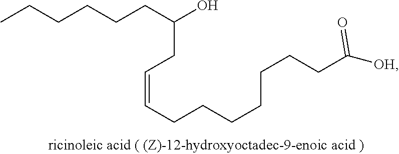 [SMILES: CCCCCCC(O)C/C=C\CCCCCCCC(=O)O]

then R⁶ represents a 1,11-heptadec-8-enyl radical

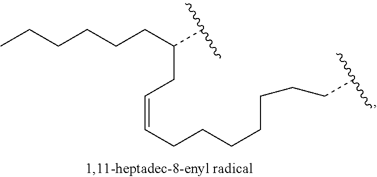

wherein “1,11” indicates the positions in which the radical is attached to the adjacent groups X and C(O).

Preferred examples for R⁶ are the structures derived from a corresponding hydroxyl carboxylic acid by abstraction of the carboxylate group and one OH group, wherein the hydroxyl carboxylic acid is preferably selected from ricinoleic acid, lesquerolic acid, 10-hydroxy octadecanoic acid, 12-hydroxy octadecanoic acid, 14-hydroxy tetradecanoic acid, 10-hydroxy stearic acid, 12-hydroxy stearic acid, or dihydroxy carboxylic acids, in particular 2,2′-di-hydroxymethyl propanoic acid, 9,10-dihydroxy stearic acid, or polyhydroxy carboxylic acids, in particular gluconic acid. Most preferably, R⁶ is derived in the above-stated manner from lesquerolic acid or ricinoleic acid. In both cases the naturally occurring enantiomers of the compounds, i.e. (9Z,12R)-12-hydroxyoctadec-9-enoic acid obtained by saponification or fractional distillation of hydrolysed castor oil, which is the seed oil of the castor plant, and (11Z, 14R)-14-hydroxyicos-11-enoic acid as isolated from Paysonia and Physaria species, are particularly preferred. However, the racemates, the S enantiomers as well as the E-configured isomers of the compounds, the racemates, the enantiomers and any possible mixture thereof are also preferred according to the invention.

The number m of the R⁶-containing repeating units (—X—C(O)—R⁶) of the at least one moiety present in a group F of the compound of the general formula (I) is from 0 to 20, preferably from 0 to 15, 0 to 12, 0 to 10, 0 to 8, or from 1 to 20, from 2 to 20, from 3 to 20, from 4 to 20, from 5 to 20, specifically 0, 1, 2, 3, 4, 5, 6, 7, 8, 9, or 10.

R⁷ is independently selected from optionally substituted straight-chain, cyclic or branched, saturated or unsaturated hydrocarbon radicals which have 1 to 1000 carbon atoms, optionally containing one or more groups selected from —O—, —NH—, —C(O)—, —C(S)—, tertiary amino

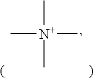

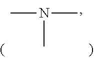

groups quaternary ammonium groups and which can be substituted with carboxyl, hydroxyl, or halide groups, wherein the radical R⁷ cannot contain an internal carboxy group or amide, i.e. R⁷ cannot contain a combination of a —C(O)— group and a —O— group or a combination of a —C(O)— group and a —NH— or tertiary amino group,

According to the invention, the radicals R⁷ can be the same or different selected from optionally substituted straight-chain, cyclic or branched, saturated or unsaturated hydrocarbon radicals which have 1 to 36 carbon atoms, and can thus represent a hydrocarbyl group selected from the group consisting of linear, branched or cyclic alkyl groups, linear, branched or cyclic alkenyl groups, linear, branched or cyclic alkynyl groups, linear, branched or cyclic alkaryl groups, linear, branched or cyclic aralkyl groups and linear, branched or cyclic aryl groups, for instance phenyl, benzyl or tolyl, in particular from such groups having 6 to 24 carbon atoms, each optionally containing one or more functional groups as indicated above.

More preferably, the R⁷ radical is selected from linear alkyl groups and linear alkenyl groups, in particular from linear C6-C24 alkyl groups such as hexyl, heptyl, octyl, nonyl, decyl, undecyl, dodecyl, tridecyl, tetradecyl, pentadecyl, hexadecyl, heptadecyl, octadecylene, nonadecyl, eicosyl, henicosyl, doicosyl, tricosyl, and tetraicosyl, or linear C6-C24 alkenyl groups such as hexenyl, heptenyl, octenyl, nonenyl, decenyl, undecenyl, dodecenyl, tridecenyl, tetradecenyl, pentadecenyl, hexadecenyl, heptadecenyl, octadecenyl, nonadecenyl, eicosenyl, henicosenyl, doicosenyl, tricosenyl, and tetraicosenyl, wherein the groups are most preferably bonded to the adjacent C(O) group or X group by a terminal C-atom.

There is no limitation regarding at which C-atom of the hydrocarbyl radicals the adjacent group C(O) group is attached to R⁷.

However, R⁷ is preferably derived from a carboxylic acid or a hydroxycarboxylic acid bearing one or more hydroxylic groups, more preferably from a carboxylic acid or monohydroxy carboxylic acid, most preferably from C7-C25 fatty acid bearing no hydroxyl group as substituent. Accordingly, R⁷ preferably represents the alkyl or alkenyl chain of such carboxylic acids. For instance, if R⁷ is derived from ricinoleic acid

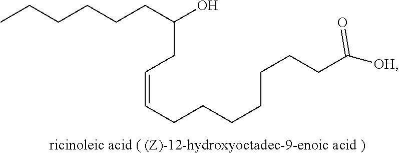 [SMILES: CCCCCCC(O)C/C=C\CCCCCCCC(=O)O]

then R⁷ represents an 11-hydroxy heptadec-8-enyl radical

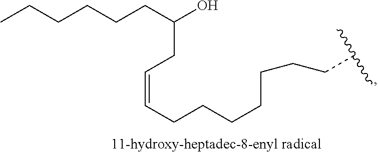

or if R⁷ is derived from oleic acid,

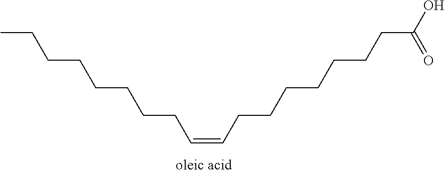 [SMILES: CCCCCCCC/C=C\CCCCCCCC(=O)O]

then R⁷ represents an heptadec-8-enyl radical

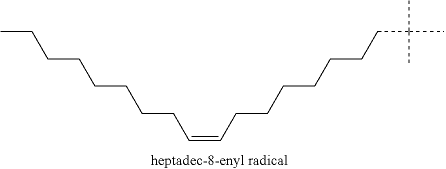

Preferred examples for R⁷ are the structures derived from a corresponding carboxylic acid or hydroxyl carboxylic acid by abstraction of the carboxylate group, wherein the carboxylic acid may be selected from acetic acid, propionic acid, butyric acid, valeric acid, caproic acid, enanthic acid, caprylic acid, pelargonic acid, capric acid, undecanoic acid, lauric acid, tridecanoic acid, myristic acid, pentadecanoic acid, palmitic acid, margaric acid, stearic acid, linoleic acid, α-linolenic acid, γ-linolenic acid, nonadecylic acid, arachidic acid, mead's acid, arachidonic acid, heneicosanoic acid, docosanoic acid, tricosylic acid and lignoceric acid, from hydroxyl carboxylic acid such as lesquerolic acid, ricinoleic acid, 10-hydroxy octadecanoic acid, 12-hydroxy octadecanoic acid, 14-hydroxy tetradecanoic acid, 10-hydroxy stearic acid, 12-hydroxy stearic acid, or from dihydroxy carboxylic acids, in particular 2,2′-di-hydroxymethyl propanoic acid, 9,10-dihydroxy stearic acid, or polyhydroxy carboxylic acids, in particular gluconic acid.

Although the radical R⁷ can optionally contain one or more groups selected from —O—, —NH—, —C(O)—, —C(S)—, tertiary amino groups

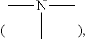

quaternary ammonium groups

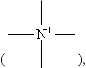

and may be substituted with OH groups or halide groups, the radical R⁷ cannot contain a combination of a —C(O)— group and a —O— group or a combination of a —C(O)— group and a —NH— or tertiary amino group forming an internal carboxylate group, i.e. an internal ester group, or an internal amide group.

According to the invention, it is mandatory that in at least one compound of the hair care formulation at least one moiety of the formula (II) R⁷ has at least 2, preferably at least 6 carbon atoms, and in the same moiety of the formula (II) at least one R⁶ has at least 6, preferably at least 8 carbon atoms.

According to the invention, the group R⁷* of the formula (II*) is independently selected from optionally substituted branched or dendrimeric hydrocarbon radicals which have 1 to 1000 carbon atoms, optionally containing one or more groups selected from —O—, —NH—, —C(O)—, —C(S)—, tertiary amino groups

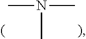

quaternary ammonium groups

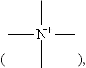

and which can be substituted with carboxyl, hydroxyl, or halide groups, wherein the radical R⁷* is terminated by two or more groups of the general structure

—X—C(O)-T

wherein X is as defined above, and

T is a monovalent straight-chain, cyclic or branched, saturated or unsaturated hydrocarbon radical optionally substituted with carboxyl, hydroxyl, or halide groups with up to 36 carbon atoms,

with the proviso that in at least one moiety of the formula (II*) R⁷* is terminated by one or more groups T having at least 2, preferably at least 6 carbon atoms, and in the same moiety of the formula (II*) at least one R⁶ has at least 6, preferably at least 8 carbon atoms.

As R⁷* is defined as a monovalent group, in order to provide the structural feature of being terminated by at least two groups of the general structure

—X—C(O)-T,

the presence of at least one branching structure is required in the residue R⁷*.

In the case R⁷* is a branched hydrocarbon radical, as the branching structure, the group comprises at least one moiety of the general formula

—B(—O—)_{b},

wherein B is a linear or branched hydrocarbon group having 3-20 carbon atoms, 3-10 and b is 2 or more, preferably 2-6, more preferably 2-4, and wherein

the groups (—O—) linked to the group B on the one side are linked to a C atom on the other side.

Therein, the C atom may be of a CH2 group or of a carbonyl group.

In the case R⁷* is a dendrimeric hydrocarbon radical, the group comprises at least one moiety of the general formula

—B(—O—)_{b},

wherein B and b are as defined above, and the groups (—O—) linked to the group B on the one side are linked to a C atom on the other side, and

at least one further moiety acting as branching structure of the general formula

—C(O)—B(—O—)_{b},

wherein B and b are as defined above, and the groups (—O—) linked to the group B on the one side are linked to a C atom on the other side, wherein the C atom may be of a CH₂ group or of a carbonyl group. The term dendrimeric hydrocarbon structure thus refers to a branched structure containing at least two consecutive branching structures.

Each group T constitutes one of at least two terminal groups of a R⁷* group and is typically derived from a fatty acid. Accordingly, the group T is preferably a linear saturated or monounsaturated hydrocarbon radical having 2 to 24 carbon atoms.

The group T is preferably linked to an (—O—) group of a branching structure of the general formula —B(—O—)_{b}, or —C(O)—B(—O—)_{b} via a carbonyl group or via an estolide chain. By the presence of the above-described branching structures, R⁷* adopts a branched or even dendrimeric structure.

The following structure is an example of R⁷* being a branched hydrocarbon radical as defined above:

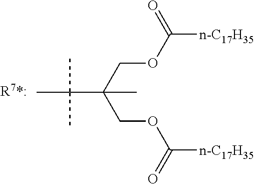

Therein, the branching structure of the general formula B(—O—)_{b} is derived from 2,2′-dihydroxymethylpropionic acid, and the group T is a n-heptadecanyl group linked to the branching structure. It is derived from stearic acid and linked to the group B by a —C(O)—O-unit. Accordingly, the structure is terminated by two groups of the general structure —X—(CO)-T and contains a branching structure of the formula —B(—O—)₂.

An example of a group from which a branched group R⁷* may be derived is displayed below:

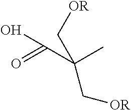

with R

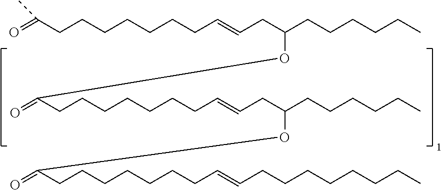

In the corresponding group R⁷*, the branching structure is as in the previous structure, however, the two terminal groups T, which are n-heptadec-9-enyl groups derived from oleic acid, are attached to the branching structure via a ricinoleic-acid derived estolide chain structure.

Another example of a group R⁷* according to the invention being a dendrimeric hydrocarbon is displayed below:

Therein, the branching structure —B(—O—)_{b} is directly followed by two further branching structures —(C(O)—B(—O—)_{b}, resulting in a further increase of terminating groups of the general structure —X—C(O)-T:

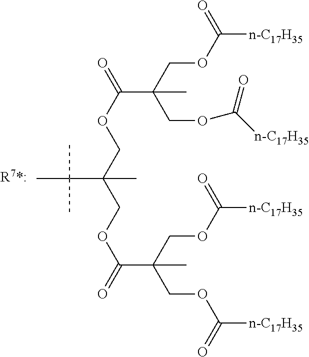

Therein, the branching structures are derived from 2,2′-dihydroxymethylpropionic acid, and the terminal groups are based on stearic acid.

It is also within the scope of the invention as defined above that in the branched or dendrimeric group R7*, the terminal groups —X—C(O)-T are not linked directly to the groups B of a branching structure, but are linked to the (—O—) groups of the branching structures by hydrocarbon groups such as optionally substituted or heteroatom-group-containing alkylenes or alkenylenes, preferably n-alkylenes having 2 to 10 carbon atoms, poly(alkylene oxide) groups such as poly (ethylene oxide) or poly (propylene oxide) groups, or in particular by oligo- or polyester groups, i.e. by estolide chains.

In the following example, the stearic acid-based groups —X—C(O)-T are linked to the branching structures by an estolide chain:

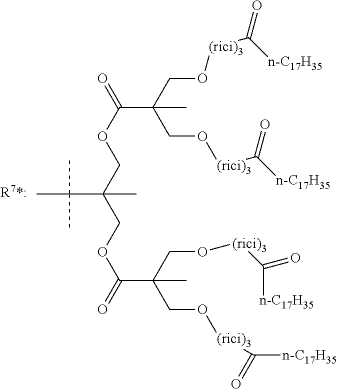

with

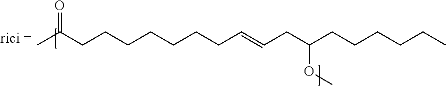

In the same manner, it is also within the scope of the invention as defined above that in the dendrimeric group R⁷*, the one or more branching elements of the structure —(C(O)—B(—O—)_{b} are not directly attached to a branching element of the structure —B(—O—)_{b} or —(C(O)—B(—O—)_{b}, but via hydrocarbon groups such as optionally substituted or heteroatom-group-containing alkylenes or alkenylenes, preferably n-alkylenes having 2 to 10 carbon atoms, poly(alkylene oxide) groups such as poly (ethylene oxide) or poly (propylene oxide) groups, or in particular by oligo- or polyester groups, i.e. by estolide chains.

In the following example, the branching structures are linked by estolide chains:

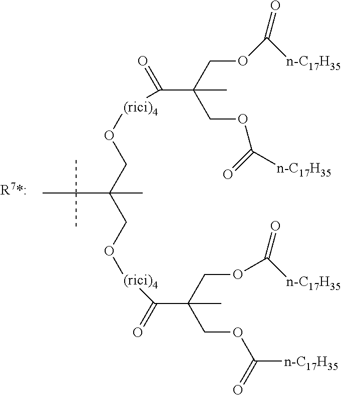

with

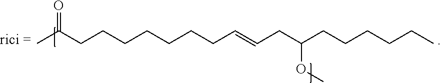

In another preferred embodiment of the invention R⁷* is derived from carboxyclic acids bearing additional ester moieties, preferably monocarboxylic acids bearing one ester moiety. Preferred examples are derived from the reaction of C2 to C24 alcohols, in particular lauryl alcohol, oleyl alcohol, stearyl alcohol, with carboxylic acid anhydrides, in particular succinic anhydride, maleic anhydride, itaconic anhydride, optionally in the presence of an esterification catalyst, yielding the corresponding ester moieties bearing carboxylic acids, exemplified by

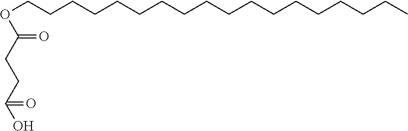 [SMILES: CCCCCCCCCCCCCCCCCCOC(=O)CCC(=O)O]

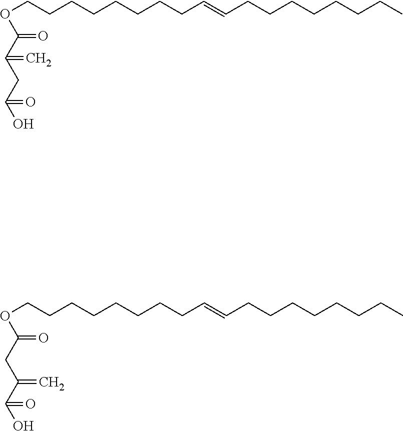 [SMILES: C=C(CC(=O)O)C(=O)OCCCCCCCC/C=C/CCCCCCCC ; C=C(CC(=O)OCCCCCCCC/C=C/CCCCCCCC)C(=O)O]

Alternatively, these groups R⁷*, derived from carboxylic acids bearing additional ester groups, can be introduced by sequential reaction of an OH terminated intermediate with in particular. a carboxylic acid anhydride and subsequent esterification with an appropriate alcohol, as exemplified by example 28.

According to the invention, the residue R¹ in formula (IV) is defined in the same manner as for formula (I), except for it is a q-valent residue, and q is 2 to 55, preferably 2 to 40, more preferably 2 to 4, which indicates that the residue R¹ in formula (IV) bears q residues of the structure (—C(O)—X-G), with G as defined below. Accordingly, the term “q-valent” does not refer to or restrict the number of optional further substituents other than (—C(O)—X-G) of the residue R¹ in formula (IV), which can be carboxylic groups or hydroxyl groups.

The hydrocarbyl structure of R¹ of formula (IV), which is q-valent regarding the residues (—C(O)—X-G), is preferably selected from the group consisting of linear, branched or cyclic alkylene groups, linear, branched or cyclic alkenylene groups, linear, branched or cyclic alkynylene groups, linear, branched or cyclic alkarylene groups, linear, branched or cyclic aralkylene groups and linear, branched or cyclic arylene groups, for instance phenylene, benzylene or tolylene groups, in particular from such groups having 1 to 1000 carbon atoms, more particular 1 to 150 carbon atoms.

Preferably, the hydrocarbon structures are linear or branched alkylene groups, or linear or branched alkylene groups interrupted by ether groups, ester groups or both ether and ester groups in particular branched structures derived from products as obtained by esterification of polyols with mono- or polyhydroxycarboxylic acids with up to 150 carbon atoms, or linear alkylene groups with up to 22 carbon atoms. Also preferred are C2-C6 linear alkenylene groups, in particular 1,2 ethenylene radicals derived from maleic acid or fumaric acid.

More preferably, the q-valent R¹ radical of formula (IV) is selected from alkylene groups, which may be selected from the group consisting of linear, branched and cyclic alkylene groups, in particular from linear C1-C22 alkyl groups such as methylene, ethylene, n-propylene, n-butylene, n-pentylene, n-hexylene, n-heptylene or n-octylene groups, branched C1-C22 alkylene groups iso-propylene, iso-butylene, tert-butylene, iso-butylene, tert-pentylene, neo-pentylene, and 2-ethylhexylene groups. Particularly preferred are 1,1-methylene, 1,2-ethylene, 1,3-propylene, 1,2,3-propylene and 1,4-butylene radicals.

There is no limitation regarding which C-atoms of the hydrocarbyl radicals bear the (—C(O)—X-G) groups attached to R¹ in formula (IV).

Regarding the presence of functional groups optionally contained in R¹ of formula (IV) and optional substituents, it is preferred that R¹ of the formula (IV) is derived from glycidyl compounds, glycerol and glycerol derivatives, in particular glycidol, glycerol diglycidyl ether, diglycidyl ether and polyglycerol compounds, more particular from compounds derived from the condensation product of glycidol, glycerol, glycerol diglycidyl ether, diglycidyl ether and polyglycerol compounds and C8-C24 monohydroxy fatty acids, in particular ricinoleic acid, lesquerolic acid or 12-hydroxyl stearic acid, or when R¹ is a linear alkylene group, in particular an alkylene group not bearing further substituents in addition to the (—C(O)—X-G) groups.

It is clear to the skilled person that the groups (—C(O)—X-G) are usually attached to the radical R¹ via —C(O)—X— units, in particular —C(O)—O— units, at positions which are substituted by —C(O)OH groups in a parent compound from which R¹ is derived.

For example, the R¹ group derived from succinic acid is a 1,2-ethylene radical, wherein “1,2” indicates the positions at which the radical is substituted by the (—C(O)—X-G)-groups.

According to the invention, X, R¹⁰, R⁶ and m are defined in the same manner for formula (IV) and (V) as for formula (I) and (II).

As defined above, R¹¹ is independently selected from optionally substituted straight-chain, cyclic or branched, saturated or unsaturated hydrocarbon radicals which have 1 to 1000 carbon atoms, optionally containing one or more groups selected from —O—, —NH—, —C(O)—, — C(S)—, tertiary amino groups

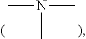

quaternary ammonium groups

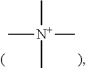

and which can be substituted with carboxyl, hydroxyl, or halide groups.

Thus R¹¹ may represent a hydrocarbyl group selected from the group consisting of linear, branched or cyclic alkyl groups, linear, branched or cyclic alkenyl groups, linear, branched or cyclic alkynyl groups, linear, branched or cyclic alkaryl groups, linear, branched or cyclic aralkyl groups and linear, branched or cyclic aryl groups, for instance phenyl, benzyl or tolyl, in particular from such groups having 6 to 24 carbon atoms, each optionally containing one or more functional groups as indicated above.

More preferably, the R¹¹ radical is selected from linear alkyl groups and linear alkenyl groups, in particular from linear C6-C24 alkyl groups such as hexyl, heptyl, octyl, nonyl, decyl, undecyl, dodecyl, tridecyl, tetradecyl, pentadecyl, hexadecyl, heptadecyl, octadecylene, nonadecyl, eicosyl, henicosyl, doicosyl, tricosyl, and tetraicosyl, or linear C6-C24 alkenyl groups such as hexenyl, heptenyl, octenyl, nonenyl, decenyl, undecenyl, dodecenyl, tridecenyl, tetradecenyl, pentadecenyl, hexadecenyl, heptadecenyl, octadecenyl, nonadecenyl, eicosenyl, henicosenyl, doicosenyl, tricosenyl, and tetraicosenyl, wherein the residues may bear one or more hydroxyl or carboxyl substituents, and wherein the groups are most preferably bonded to the adjacent C(O) group or X group by a terminal C-atom.

There is no limitation regarding at which C-atoms of the hydrocarbyl radicals the adjacent groups X group are attached to R¹¹.

However, R¹¹ is preferably derived from monoalcohols, diols or polyols bearing more than two OH-groups, or from monohydroxy carboxylic acid or carboxylic acids bearing more than one hydroxylic groups, more preferably from a monoalcohol or a monohydroxy carboxylic acid, most preferably from C6-C24 fatty acids with one hydroxyl group as substituent. Accordingly, R¹¹ preferably represents the alkyl or alkenyl chain of such carboxylic acids. For instance, if R¹¹ is derived from ricinoleic acid

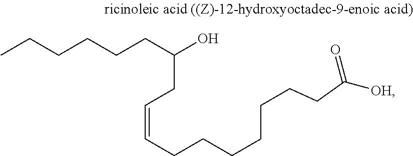 [SMILES: CCCCCCC(O)C/C=C\CCCCCCCC(=O)O]

then R¹¹ represents a 12-(octadec-9-enoic acid) radical

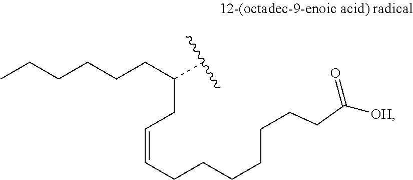 [SMILES: *[C@H](C/C=C\CCCCCCCC(=O)O)CCCCCC]

or if R¹¹ is derived from dodecanol,

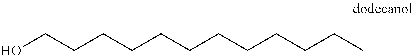

then R⁷ represents a 1-dodecyl radical

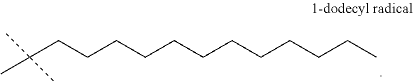

Preferred examples of R¹¹ are the structures formally derived from a corresponding hydroxyl carboxylic acid by abstraction of an hydroxyl group, wherein the hydroxyl carboxylic acid may be selected from monohydroxy carboxylic acids, such as lesquerolic acid, ricinoleic acid, 10-hydroxy octadecanoic acid, 12-hydroxy octadecanoic acid, 14-hydroxy tetradecanoic acid, 10-hydroxy stearic acid, 12-hydroxy stearic acid, or from dihydroxy carboxylic acids, in particular 2,2′-di-hydroxymethyl propanoic acid, 9,10-dihydroxy stearic acid, or from polyhydroxy carboxylic acids, in particular gluconic acid.

Likewise, preferred examples of R¹¹ are the structures formally derived from the corresponding mono- and diols by formal abstraction of one hydroxyl group, wherein the alcohols may be selected from the group consisting of methanol, ethanol, 2-propanol, 1-butanol, t-butanol, undec-10-en-ol, oleyl alcohol, stearyl alcohol, 1,2,-propanediol, 1,3-propanediol, 1,3-butanediol, 1,4-butanediol, 1,2 hexanediol, 1,6-hexanediol.

Further preferred examples of R¹¹ are compounds formally derived as described above from glycerol, diglycerol, triglycerol, and linear or branched oligoglycerols formally comprising from 4 to 6 glycerol units, trimethylol propane, castor oil (ricinoleic acid triglyceride), lesquerella oil (lesquerolic acid triglyceride), pentaerythritol, sorbitol, polyalkylene oxides, such as ethylene oxide-, propylene oxide- and/or butylene oxide-based polyethers.

Although the radical R¹¹ can optionally contain one or more groups selected from —O—, —NH—, —C(O)—, —C(S)—, tertiary amino groups

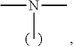

quaternary ammonium groups

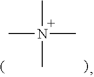

and may be substituted with OH groups or carboxyl groups, the radical R⁷ cannot contain a combination of a —C(O)— group and a —O— group or a combination of a —C(O)— group and a —NH— or tertiary amino group forming an internal carboxylate group, i.e. an internal ester group, or an internal amide group.

According to the invention, it is mandatory that in at least one compound of the formula (IV) of the hair care formulation at least one moiety of the formula (V) R¹¹ has at least 2, preferably at least 6 carbon atoms, and in the same moiety of the formula (V) at least one R⁶ has at least 6, preferably at least 8 carbon atoms.

According to the invention, it is also mandatory that the compound of the formula (I) is not exclusively composed of glycerol and ricinoleic acid moieties, i.e. it is not a polyglycerol polyricinolate (PGPR).

According to the invention, the term “polyglycerol polyricinoleate” or “PGPR” comprises structures of oligomeric glycerol which is esterified or partially esterified by polyricinoleic acid and/or ricinoleic acid.

Therein, the oligomeric glycerol is i.e. obtained in a base-catalyzed oligomerization reaction of glycerol or reaction of glycerol with 2,3-epoxy 1-propanol (glycidol).

The polyricinoleic acid structure is obtained by condensation of two or more ricinoleic acid molecules via intermolecular esterification.

Accordingly, polyglycerol polyricinoleate is displayed by the following structure:

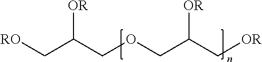

wherein n is an integer of typically 1-4, but may also be more, and

R is independently selected from H and/or ricinoleic acid and/or polyricinoleic acid residue-Both the ricinoleic acid residues and polyricinoleic acid residues are bonded to the polyglycerol structure via the carboxylic acid group.

The ricinoleic acid residue thus has the following structure,

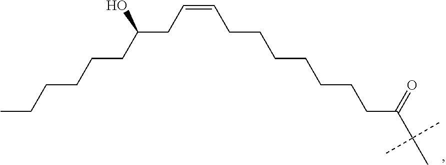

and the polyricinoleic acid residue is represented by the following structure:

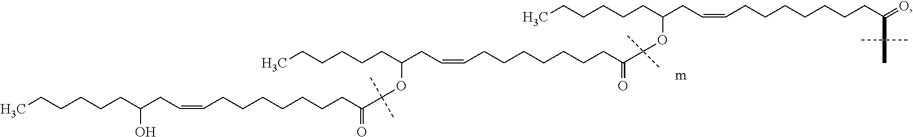

wherein m is typically 2 or 3, but may also be more.

In a preferred embodiment of the present invention, a hair care formulation is provided containing at least one compound of the formula (I) or (IV), wherein in the compound of formula (I) or (IV) R¹ is as defined above with up to 10000 carbon atoms, preferably up to 1000, more preferably up to 300, even more preferably up to 100, most preferably up to 50 carbon atoms.

According to this embodiment, R¹ of the formula (I) is preferably selected from linear or branched alkylene groups, or linear or branched alkylene groups interrupted by ether groups, ester groups or both ether and ester groups, in particular branched structures derived from products as obtained by esterification of polyols with mono- or polyhydroxycarboxylic acids, or linear alkylene groups.

More preferably, the R¹ radical of the formula (I) according to this embodiment is selected from alkylene groups selected from the group consisting of linear and branched alkylene groups, in particular from linear C1-C22 alkyl groups such as methylene, ethylene, n-propylene, n-butylene, n-pentylene, n-hexylene, n-heptylene or n-octylene groups, branched C1-C22 alkylene groups iso-propylene, iso-butylene, tert-butylene, iso-butylene, tert-pentylene, neo-pentylene, and 2-ethylhexylene groups.

Regarding the presence of functional groups optionally contained in the R¹ radical of the formula (I) according to this embodiment and its optional substituents, it is preferred that R¹ is derived from glycidyl compounds, glycerol and glycerol derivatives, in particular glycidol, glycerol, glycerol diglycidyl ether, diglycidyl ether and polyglycerol compounds, or when R¹ is a linear alkylene group, in particular an alkylene group not bearing further substituents in addition to the (—X—C(O)—F) groups is preferred, and it is even more preferred when R¹ is derived from the condensation product of glycidol, glycerol, glycerol diglycidyl ether, diglycidyl ether and polyglycerol compounds and C8-C24 monohydroxy fatty acids, in particular ricinoleic acid, lesquerolic acid or 12-hydroxyl stearic acid.

Preferably, R¹ of the formula (IV) according to this embodiment is selected from linear or branched alkylene groups, or linear or branched alkylene groups interrupted by ether groups, ester groups or both ether and ester groups in particular branched structures derived from products as obtained by esterification of polyols with mono- or polyhydroxycarboxylic acids, or linear alkylene groups. Also preferred are C2-C₆ linear alkenylene groups, in particular 1,2 ethenylene radicals derived from maleic acid or fumaric acid.

More preferably, the R¹ radical of formula (IV) is selected from alkylene groups, which may be selected from the group consisting of linear or branched alkylene groups, in particular from linear C1-C22 alkyl groups such as methylene, ethylene, n-propylene, n-butylene, n-pentylene, n-hexylene, n-heptylene or n-octylene groups, branched C1-C22 alkylene groups iso-propylene, iso-butylene, tert-butylene, iso-butylene, tert-pentylene, neo-pentylene, and 2-ethylhexylene groups. Particularly preferred are 1,1-methylene, 1,2-ethylene, 1,3-propylene, 1,2,3-propylene and 1,4-butylene radicals.

Regarding the presence of functional groups optionally contained in R¹ of formula (IV) and optional substituents, it is preferred that R¹ of the formula (IV) is derived from glycidyl compounds, glycerol and glycerol derivatives, in particular glycidol, glycerol diglycidyl ether, diglycidyl ether and polyglycerol compounds, more particular from compounds derived from the condensation product of glycidol, glycerol, glycerol diglycidyl ether, diglycidyl ether and polyglycerol compounds and C8-C24 monohydroxy fatty acids, in particular ricinoleic acid, lesquerolic acid or 12-hydroxyl stearic acid, or when R¹ is a linear alkylene group, in particular an alkylene group not bearing further substituents in addition to the (—C(O)—X-G) groups.

In another preferred embodiment of the present invention, a hair care formulation is provided containing at least one compound of the formula (I) or (IV), wherein in the compound of formula (I) or (IV) the number of carbon atoms in any R⁷ or R¹¹ of the compound is from 3 to 300, preferably 3 to 100, more preferably 3 to 50, even more preferably 3 to 36, further preferably 3 to 24, and most preferably 11 to 24.

According to this embodiment, R⁷ is preferably independently selected from optionally substituted linear, branched or cyclic alkyl groups, linear, branched or cyclic alkenyl groups, linear, branched or cyclic alkynyl groups, linear, branched or cyclic alkaryl groups, linear, branched or cyclic aralkyl groups and linear, branched or cyclic aryl groups, for instance phenyl, benzyl or tolyl, in particular from such groups having 6 to 24 carbon atoms, each optionally containing one or more functional groups as indicated above.

More preferably, the R⁷ radical is selected from linear alkyl groups and linear alkenyl groups, in particular from linear C6-C24 alkyl groups such as hexyl, heptyl, octyl, nonyl, decyl, undecyl, dodecyl, tridecyl, tetradecyl, pentadecyl, hexadecyl, heptadecyl, octadecylene, nonadecyl, eicosyl, henicosyl, doicosyl, tricosyl, and tetraicosyl, or linear C6-C24 alkenyl groups such as hexenyl, heptenyl, octenyl, nonenyl, decenyl, undecenyl, dodecenyl, tridecenyl, tetradecenyl, pentadecenyl, hexadecenyl, heptadecenyl, octadecenyl, nonadecenyl, eicosenyl, henicosenyl, doicosenyl, tricosenyl, and tetraicosenyl, wherein the groups are most preferably bonded to the adjacent C(O) group or X group by a terminal C-atom.

According to this embodiment, R¹¹ is preferably independently selected from optionally substituted linear, branched or cyclic alkyl groups, linear, branched or cyclic alkenyl groups, linear, branched or cyclic alkynyl groups, linear, branched or cyclic alkaryl groups, linear, branched or cyclic aralkyl groups and linear, branched or cyclic aryl groups, for instance phenyl, benzyl or tolyl, in particular from such groups having 6 to 24 carbon atoms, each optionally containing one or more functional groups as indicated above.

More preferably, the R¹¹ radical is selected from linear alkyl groups and linear alkenyl groups, in particular from linear C6-C24 alkyl groups such as hexyl, heptyl, octyl, nonyl, decyl, undecyl, dodecyl, tridecyl, tetradecyl, pentadecyl, hexadecyl, heptadecyl, octadecylene, nonadecyl, eicosyl, henicosyl, doicosyl, tricosyl, and tetraicosyl, or linear C6-C24 alkenyl groups such as hexenyl, heptenyl, octenyl, nonenyl, decenyl, undecenyl, dodecenyl, tridecenyl, tetradecenyl, pentadecenyl, hexadecenyl, heptadecenyl, octadecenyl, nonadecenyl, eicosenyl, henicosenyl, doicosenyl, tricosenyl, and tetraicosenyl, wherein the residues may bear one or more hydroxyl or carboxyl substituents, and wherein the groups are most preferably bonded to the adjacent C(O) group or X group by a terminal C-atom.

In yet another preferred embodiment of the present invention, a hair care formulation is provided containing at least one compound of the formula (I) or (IV), wherein the compound of formula (I) or (IV) has a molecular weight in the range of from 1500 to 200000 g/mol, preferably 1500 to 100000 g/mol, more preferably 1500 to 30000 g/mol, even more preferably 1500 to 10000 g/mol, further preferably 1500 to 5000 g/mol, and most preferably 1500 to 3000 g/mol. In a further preferred embodiment of the present invention, a hair care formulation is provided containing at least one compound of the formula (I) or (IV), wherein the compound of formula (I) contains 2 to 100 moieties of the formula (II), more preferably 2 to 50 moieties of the formula (II), even more preferably 2 to 20, further preferably 2 to 10, and even further preferably 2 to 6, and most preferably 2 to 4 moieties of the formula (II), or wherein the compound of the formula (IV) contains 2 to 100 moieties of the formula (V), more preferably 2 to 50 moieties of the formula (V), even more preferably 2 to 20, further preferably 2 to 10, and even further preferably 2 to 6, and most preferably 2 to 4 moieties of the formula (V), or wherein the compound of the formula (I) contains 2 to 100 moieties of the formula (II*), more preferably 2 to 50 moieties of the formula (II*), even more preferably 2 to 20, further preferably 2 to 10, and even further preferably 2 to 6, and most preferably 2 to 4 moieties of the formula (II*).

In the present invention, the presence of the moieties of the formula (II) or (V) or (II*), i.e. the estolide moieties of the compounds, is decisive for obtaining and modulating the desired properties of the preferably fatty-acid based compounds and thus of the hair care formulations containing these compounds.

In a preferred embodiment of the present invention, a hair care formulation is provided containing at least one compound of the formula (I), wherein at least one compound of the general formula (I) is represented by the general formula (III)

{[(F—C(O)—X—)₁₋₅R³(—X—C(O)—)]₁₋₃R⁴—C(O)—X—}ᵣ—R²(—X—C(O)—F)ₛ (III)

wherein X is as defined above,

R² in formula (III) is selected from (r+s)-valent, optionally substituted hydrocarbon radicals which have up to 1000 carbon atoms, and may contain optionally one or more groups selected from —O—, —NH—, —C(O)—, —C(S)—, tertiary amino groups

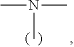

and quaternary ammonium groups, and can be optionally substituted by one or more carboxylic groups or hydroxyl groups, and optionally forms a bond to the nitrogen atom in the group —NR¹⁰—, in case R¹⁰ is a bond to R²,

R³ is selected from di- to hexavalent, optionally substituted hydrocarbon radicals which have up to 1000 carbon atoms, and may contain optionally one or more groups selected from —O—, —NH—, —C(O)—, —C(S)—, tertiary amino groups

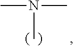

wherein if a plurality of R³ is present in formula (III), they can be the same or different,

R⁴ is selected from divalent to tetravalent optionally substituted straight-chain, cyclic or branched, saturated, unsaturated or aromatic hydrocarbon radicals which have up to 300 carbon atoms, which optionally contain one or more groups selected from —O—, —NH—, —C(O)—, —C(S)—, tertiary amino groups

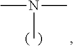

wherein if a plurality of R⁴ is present in formula (III), they can be the same or different,

and wherein

r+s=2 to 55,

r=0 to 54,

s≥1,

and in formula (III) F is as defined above with the proviso that at least one of the radicals F contains at least one moiety of the formula (II) or formula (II*):

—R⁶(—X—C(O)—R⁶)ₘ—X—C(O)—R⁷ (II)

—R⁶(—X—C(O)—R⁶)ₘ—X—C(O)—R⁷* (II*)

wherein

X, R⁶, R⁷ and R⁷* are as defined above,

m=0 to 20, preferably 1 to 20,

and with the proviso that in at least one moiety of the formula (II) R⁷ has at least 2, preferably at least 6 carbon atoms, and in the same moiety of the formula (II) at least one R⁶ has at least 6, preferably at least 8 carbon atoms, or that that in at least one moiety of the formula (II*) R⁷* is terminated by one or more groups T having at least 2, preferably at least 6 carbon atoms, and in the same moiety of the formula (II*) at least one R⁶ has at least 6, preferably at least 8 carbon atoms.

According to the invention, R² in formula (III) is defined as being selected from (r+s)-valent, optionally substituted hydrocarbon radicals which have up to 1000 carbon atoms, and may contain optionally one or more groups selected from —O—, —NH—, —C(O)—, —C(S)—, tertiary amino groups

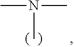

and quaternary ammonium groups, and can be optionally substituted by one or more carboxylic groups or hydroxyl groups, and optionally forms a bond to the nitrogen atom in the group —NR¹⁰—, in case R¹⁰ is a bond to R².

R² is (r+s)-valent, wherein (r+s) is 2 to 55, preferably 2 to 40, more preferably 2-30, and r indicates the number of groups of the structure

—{[(F—C(O)—X—)₁₋₅R³(—X—C(O)—)]₁₋₃R⁴—C(O)—X—}

bonded to R² and is r=0 to 54, preferably 0 to 20, more preferably 0 to 10, most preferably selected from 0, 1, 2, 3, 4, 5, 6, 7, or 8, while s indicates the number of groups of the structure —(—X—C(O)—F) bonded to R² and is s≥1, preferably 1 to 50, more preferably 1 to 30, even more preferably 1 to 20, most preferably selected from 1, 2, 3, 4, 5, 6, 7 or 8. It is also particularly preferred that r=0 and s=1 to 39, specifically if r=0 and s=1, 2, 3, 4, 5, 6, 7, 8, 9 or 10.

The term “(r+s)-valent” does not refer to or restrict the number of optional further substituents other than (—X—C(O)—F) of the residue R², which can be carboxylic groups or hydroxyl groups.

The hydrocarbyl structure of R², which is s-valent regarding the residues (—X—C(O)—F) and r-valent regarding the residues —{[(F—C(O)—X—)₁₋₅R³(—X—C(O)—)]₁₋₃R⁴—C(O)—X—}, is preferably selected from the group consisting of linear, branched or cyclic alkylene groups, linear, branched or cyclic alkenylene groups, linear, branched or cyclic alkynylene groups, linear, branched or cyclic alkarylene groups, linear, branched or cyclic aralkylene groups and linear, branched or cyclic arylene groups, for instance phenylene, benzylene or tolylene groups, in particular from such groups having 1 to 1000 carbon atoms, more particular 1 to 150 carbon atoms.

Preferably, the hydrocarbon structures are linear or branched alkylene groups, or linear or branched alkylene groups interrupted by ether groups, ester groups or both ether and ester groups, in particular branched structures derived from products as obtained by esterification of polyols with mono- or polyhydroxycarboxylic acids with up to 150 carbon atoms, or linear alkylene groups with up to 22 carbon atoms.

More preferably, the (r+s)-valent R² radical is selected from alkylene groups, which may be selected from the group consisting of linear, branched and cyclic alkylene groups, in particular from linear C1-C22 alkyl groups such as methylene, ethylene, n-propylene, n-butylene, n-pentylene, n-hexylene, n-heptylene or n-octylene groups, branched C1-C22 alkylene groups iso-propylene, iso-butylene, tert-butylene, iso-butylene, tert-pentylene, neo-pentylene, and 2-ethylhexylene groups.

There is no limitation regarding which C-atoms of the hydrocarbyl radicals bear the (—X—C(O)—F) groups and {[(F—C(O)—X—)₁₋₅R³(—X—C(O)—)]₁₋₃R⁴—C(O)—X—}—groups attached to R².

It is, however, preferred that each carbon atom of R² does not bear more than one (—X—C(O)—F) group or {[(F—C(O)—X—)₁₋₅R³(—X—C(O)—)]₁₋₃R⁴—C(O)—X—}—group.

Regarding the presence of functional groups optionally contained in R² and optional substituents, it is preferred that R² is derived from glycidyl compounds, glycerol and glycerol derivatives, in particular glycidol, glycerol diglycidyl ether, diglycidyl ether and polyglycerol compounds, or when R² is a linear alkylene group, in particular an alkylene group not bearing further substituents in addition to the —(—X—C(O)—F) groups and {[(F—C(O)—X—)₁₋₅R³(—X—C(O)—)]₁₋₃R⁴—C(O)—X—}—groups, and even more preferred when R² is derived from the condensation product of glycidol, glycerol, glycerol diglycidyl ether, diglycidyl ether and polyglycerol compounds and C8-C24 monohydroxy fatty acids, in particular ricinoleic acid, lesquerolic acid or 12-hydroxyl stearic acid.

It is clear to the skilled person that usually the groups —(—X—C(O)—F) and {[(F—C(O)—X—)₁₋₅R³(—X—C(O)—)]₁₋₃R⁴—C(O)—X—}—are attached to the radical R² via —X—C(O)— units, in particular —O—C(O)— units, at positions which are substituted by —OH or —NHR¹⁰ groups in a parent compound from which R² is derived.

For example, the R² group derived from glycerol is a 1,2,3-propylene radical, wherein “1,2,3” indicates the positions at which the radical is substituted by the (—X—C(O)—F)-groups or (—X—C(O)—F) and {[(F—C(O)—X—)₁₋₅R³(—X—C(O)—)]₁₋₃R⁴—C(O)—X—}—groups.

According to the invention, R³ is selected from di- to hexavalent, optionally substituted hydrocarbon radicals which have up to 1000 carbon atoms, and may contain optionally one or more groups selected from −O—, —NH—, —C(O)—, —C(S)—, tertiary groups

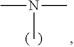

wherein if a plurality of R³ is present in formula (III), they can be the same or different. When R³ is defined as being di- to hexavalent, this indicates the number of groups of the formula (F—C(O)—X—)— plus one, as one valence is used for bonding to the X group of the moiety —(X—C(O))— which links R³ to R⁴, while the other valences are used for bonding to groups of the structure (F—C(O)—X—)—. It is clear to the person skilled in the art that the groups X, in particular with X being 0, by which R³ is bonded to the moiety or moieties (F—C(O)—X—)—or to the moiety —(X—C(O))— linking R³ to R⁴, are stemming from the OH or NHR¹⁰ groups of a compound from which R³ is derived, and accordingly the radical constituting R³ is the hydrocarbyl backbone of these compounds. For instance, R³ being an 1,2,3-propylene radical is derived from glycerol by abstraction of the OH groups. In the preparation of a compound containing such R³, it is expected that a step of esterification of glycerol is involved to obtain such R³ group.

The hydrocarbyl structure of the di- to hexavalent R³ radicals is preferably selected from the group consisting of linear, branched or cyclic alkylene groups, linear, branched or cyclic alkenylene groups, linear, branched or cyclic alkynylene groups, linear, branched or cyclic alkarylene groups, linear, branched or cyclic aralkylene groups and linear, branched or cyclic arylene groups, for instance phenylene, benzylene or tolylene groups, in particular from such groups having 1 to 1000 carbon atoms, more particular 1 to 150 carbon atoms.

Preferably, the hydrocarbon structures are linear or branched alkylene groups, or linear or branched alkylene groups interrupted by ether groups, ester groups or both ether and ester groups, in particular branched structures derived from products as obtained by esterification of polyols with mono- or polyhydroxycarboxylic acids with up to 150 carbon atoms, or linear alkylene groups with up to 22 carbon atoms.

More preferably, the R³ radicals are selected from alkylene groups, which may be selected from the group consisting of linear, branched and cyclic alkylene groups, in particular from linear C1-C22 alkyl groups such as methylene, ethylene, n-propylene, n-butylene, n-pentylene, n-hexylene, n-heptylene or n-octylene groups, branched C1-C22 alkylene groups such as iso-propylene, iso-butylene, tert-butylene, iso-butylene, tert-pentylene, neo-pentylene, and 2-ethylhexylene groups.

There is no limitation regarding which C-atoms of the hydrocarbyl radicals R³ bear the (—X—C(O)—F)— groups and which C-atom bonds to the —(X—C(O)—) group via which R³ is linked to R⁴.

It is, however, preferred that each carbon atom of R³ does not bear more than one (—X—C(O)—F) group or {[(F—C(O)—X—)₁₋₅R³(—X—C(O)—)]₁₋₃R⁴—C(O)—X—}—group.

Regarding the presence of functional groups optionally contained in R³, it is preferred that R⁴ contains ether groups, ester groups or both of them, or when R³ is derived from glycidyl compounds, glycerol and glycerol derivatives, in particular glycidol, glycerol diglycidyl ether, diglycidyl ether and polyglycerol compounds, or when R⁴ is a linear alkylene group, in particular an alkylene group not bearing further substituents in addition to the —(—X—C(O)—F) groups and —(X—C(O)—) group via which R³ is linked to R⁴ and not containing any functional groups,

and even more preferred when R³ is a 1,2-ethylene radical, a 1,3-propylene radical, a 1,4-butylene radical, a 1,5-pentylene radical, a 1,12-octadecylene radical, a 1,14-octadecylene radical, a 1,2,3-propylene radical, a 1,2,4-butylene radical, a 1,2,5-pentylene radical, a 1,3,5-pentylene radical, a 1,2,3,4-butylene radical, a 1,2,3,4-pentylene radical, a 1,2,4,5-pentylene radical, a 1,2,3,4,5-pentylene group, or a 1,2,3,4,5,6-hexylene radical.

According to the invention, R⁴ is selected from divalent to tetravalent optionally substituted straight-chain, cyclic or branched, saturated, unsaturated or aromatic hydrocarbon radicals which have up to 300 carbon atoms, which optionally contain one or more groups selected from —O—, —NH—, —C(O)—, —C(S)—, tertiary amino groups

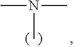

wherein if a plurality of R⁴ is present in formula (III), they can be the same or different.

As R⁴ is linked to R² and R³ via —(C(O)—X)— moieties, wherein the 2 to 4 terminal C-atoms of the R⁴ radical are bonded to the carbonyl group of the —(C(O)—X)— linkage groups, R⁴ is structurally derived from di- to tetracarboxylic acids. In case R⁴ is derived from hydrocarbon carboxylic acids not containing further functional groups or substituents, the R⁴ radical is a hydrocarbyl radical, as for instance an ethylene or propylene radical. In most cases, however, the structure from which R⁴ is derived is obtained by condensation of di- to tetracarboxylic acids with monohydroxy carboxylic acids, or of di- to tetracarboxylic acids, in particular of dicarboxylic acids, with diols, triols or polyols containing more than three hydroxy groups. In these cases, the R⁴ radicals contain internal ester groups. For example, if R⁴ is derived from the cross-condensation product of one equivalent of succinic acid and one equivalent of ricinoleic acid, the ester group obtained by condensation of one carboxylic group of the succinic acid and the hydroxy group of ricinoleic acid forms is present in the R⁴ radical, while the other carboxylate group of the succinic acid and the carboxylate group of the ricinoleic acid provide the carbonyl groups adjacent to the R⁴ radical in the structure of formula (III).

Considering the structure of the formula (III) and the definitions of F, R², R³ and R⁴, it is clear in a given structure at which point, when starting from F moving to the center of the structure, F stops and R³ or R² start, and at which point, if present, R³ ends and R⁴ starts. Regarding the question where R⁴ stops and R² starts, there may be several options which are allowable in a specific structure with regards to the definitions of R² and R⁴. For the sake of clarity, it is defined that according to the present invention, as much of the structure of a compound according to the formula (III) as possible is subsumed as belonging to R², thus rendering the number of C-atoms of R⁴ as low as possible.

In a further preferred embodiment of the present invention, a hair care formulation is provided containing at least one compound of the formula (I) represented by the general formula (III), or of the formula (IV) as defined above,

wherein R¹ in the compound of formula (IV) or R² in the compound of the formula (III) is selected from optionally substituted hydrocarbon radicals which have 2 to 300 carbon atoms, more preferred 3 to 200 carbon atoms, even more preferred 3 to and 150 carbon atoms, specifically 3 to 50 carbon atoms, more specifically 3 to 20 carbon atoms may contain optionally one or more groups selected from —O—, —NH—, —C(O)—, —C(S)—, and tertiary amino groups

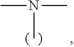

and can be optionally substituted with carboxy groups or hydroxyl groups.

According to this embodiment, R¹ in formula (IV) or R² in the compound of the formula (III) is preferably selected from the group consisting of optionally substituted linear or branched alkylene groups, or linear or branched alkylene groups interrupted by ether groups, ester groups or both ether and ester groups in particular branched structures derived from products as obtained by esterification of polyols with mono- or polyhydroxycarboxylic acids with up to 150 carbon atoms, or linear alkylene groups with up to 22 carbon atoms.

More preferably, the R¹ radical of formula (IV) or the R² radical of formula (III) is selected from alkylene groups, which may be selected from the group consisting of linear, branched alkylene groups, in particular from linear alkylene groups such as methylene, ethylene, n-propylene, n-butylene, n-pentylene, n-hexylene, n-heptylene or n-octylene groups, branched alkylene groups iso-propylene, iso-butylene, tert-butylene, iso-butylene, tert-pentylene, neo-pentylene, and 2-ethylhexylene groups.

Regarding the presence of functional groups optionally contained in R¹ of formula (IV) or R² of the formula (III) and optional substituents, it is preferred that the R¹ and R² radicals are derived from glycidyl compounds, glycerol and glycerol derivatives, in particular glycidol, glycerol, glycerol diglycidyl ether, diglycidyl ether and polyglycerol compounds, or when R¹ of formula (IV) or R² of the formula (III) is a linear alkylene group, in particular an alkylene group not bearing further substituents in addition to the (—X—C(O)—F) groups and even more preferred when R¹ is derived from the condensation product of glycidol, glycerol, glycerol diglycidyl ether, diglycidyl ether and polyglycerol compounds and C8-C24 monohydroxy fatty acids, in particular ricinoleic acid, lesquerolic acid or 12-hydroxyl stearic acid.

It is clear to the skilled person that usually the groups (—X—C(O)—F) are attached to the radical R¹ via —X—C(O)— units, in particular —O—C(O)— units, at positions which are substituted by —OH or —NHR¹⁰ groups in a parent compound from which R¹ is derived.

For example, the R¹ group derived from glycerol is a 1,2,3-propylene radical, wherein “1,2,3” indicates the positions at which the radical is substituted by the (—X—C(O)—F)-groups.

In a further preferred embodiment of the present invention, a hair care formulation is provided containing at least one compound of the formula (I) represented by the general formula (III) as defined above, or of the formula (IV), wherein R³ is selected from di- to tetravalent residues, specifically divalent residues, trivalent residues, or tetravalent residues.

In another preferred embodiment according to the invention, a hair care formulation is provided containing at least one compound of the formula (I) represented by the general formula (III) as defined above, wherein R³ is selected from optionally substituted hydrocarbon radicals which have up to 300 carbon atoms, more preferred 3 to 200 carbon atoms, even more preferred 3 to and 150 carbon atoms, specifically 3 to 50 carbon atoms, more specifically 3 to 20 carbon atoms may contain optionally one or more groups selected from —O—, —NH—, —C(O)—, —C(S)—, tertiary amino groups

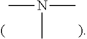

Preferred groups R³ according to this embodiment are linear C1-C22 alkylene groups such as methylene, ethylene, n-propylene, n-butylene, n-pentylene, n-hexylene, n-heptylene or n-octylene groups, branched C1-C22 alkylene groups such as iso-propylene, iso-butylene, tert-butylene, iso-butylene, tert-pentylene, neo-pentylene, and 2-ethylhexylene groups, it is more preferred when R³ when R³ is a 1,2-ethylene radical, a 1,3-propylene radical, a 1,4-butylene radical, a 1,5-pentylene radical, a 1,12-octadecylene radical, a 1,14-octadecylene radical, 1,2,3-propylene radical, a 1,2,4-butylene radical, a 1,2,5-pentylene radical, a 1,3,5-pentylene radical, a 1,2,3,4-butylene radical, a 1,2,3,4-pentylene radical, a 1,2,4,5-pentylene radical, a 1,2,3,4,5-pentylene group, or a 1,2,3,4,5,6-hexylene radical.

In still another preferred embodiment according to the invention, a hair care formulation is provided containing at least one compound of the formula (I), which may be represented by the general formula (III), or of the formula (IV) as defined above, wherein F and G are selected from optionally substituted straight-chain, cyclic or branched, saturated, unsaturated or aromatic hydrocarbon radicals which have up to 200 carbon atoms, preferred 10 to 200 carbon atoms, more preferred 10 to 150, even more preferred 10 to 100 carbon atoms, which optionally contain one or more groups selected from —O—, —NH—, —C(O)—, —C(S)—, and tertiary amino groups

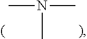

and can be optionally substituted by one or more carboxyl groups, hydroxyl groups or halide groups.

The halide groups are independently selected from fluoro, chloro, bromo or iodo groups, wherein chloro groups are preferred.

According to this embodiment, preferably the group F consists exclusively from a moiety of the formula (II), wherein R⁶ and R⁷ are hydrocarbyl groups not containing further functional groups optionally substituted with hydroxy groups, and also preferably the group G consists exclusively from a moiety of the formula (V), wherein R⁶ and R¹¹ are hydrocarbyl groups not containing further functional groups optionally substituted with hydroxy groups, or preferably the group F consists exclusively of a moiety of the formula (II*) as defined above, and more preferably of a moiety of the formula (II*) wherein R⁷ is hydrocarbyl groups not containing functional groups except —C(O)— and —O— groups.

In an also preferred embodiment according to the invention, a hair care formulation is provided containing at least one compound of the formula (III), wherein R⁴ is selected from a divalent to tetravalent, preferred divalent, trivalent, tetravalent optionally substituted straight-chain, cyclic or branched, saturated, unsaturated or aromatic hydrocarbon radicals which have 2 to 300 carbon atoms, preferred 5 to 200 carbon atoms, more preferred 8 to 150 carbon atoms, even more preferred 10 to 120 carbon atoms, which optionally contain one or more groups selected from —O—, —NH—, —C(O)—, —C(S)—, and tertiary amino groups

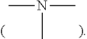

In another preferred embodiment according to the invention, a hair care formulation is provided containing at least one compound of the formula (III), wherein r=0 to 50 or 1 to 50, preferred 0 to 20, more preferred 0 to 10, even more preferred 1 to 10, specifically 1 to 5, more specifically 0, 1, 2, 3, 4, 5, even more specifically 2.

In a further preferred embodiment according to the invention, a hair care formulation is provided containing at least one compound of the formula (I), which may be specifically represented by the general formula (III), wherein at least one of the radicals F contains at least one moiety selected from the moieties

—R⁶(—O—C(O)—R⁶)ₘ—O—C(O)—R⁷,

—R⁶(—NR¹⁰—C(O)—R⁶)ₘ—O—C(O)—R⁷,

—R⁶(—NR¹⁰—C(O)—R⁶)ₘ—NR¹⁰—C(O)—R⁷,

preferably —R⁶(—O—C(O)—R⁶)ₘ—O—C(O)—R⁷,

wherein R¹⁰, R⁶, R⁷, and m are as defined above,

or wherein in the compound of formula (I) or (III):

at least one of the radicals F contains at least one moiety selected from the moieties

—R⁶(—O—C(O)—R⁶)ₘ—O—C(O)—R⁷*,

—R⁶(—NR¹⁰—C(O)—R⁶)ₘ—O—C(O)—R⁷*,

—R⁶(—NR¹⁰—C(O)—R⁶)ₘ—NR¹⁰—C(O)—R⁷*,

preferably —R⁶(—O—C(O)—R⁶)ₘ—O—C(O)—R⁷*, wherein R¹⁰, R⁶, R⁷*, and m are as defined above.

According to this embodiment, in the above structures R⁶ is preferably independently selected from optionally hydroxyl-substituted hexylene, heptylene, octylene, nonylene, decylene, undecylene, dodecylene, tridecylene, tetradecylene, pentadecylene, hexadecylene, heptadecylene, octadecylene, nonadecylene, eicosylene, henicosylene, doicosylene, tricosylene, and tetraicosylene, or hexenylene, heptenylene, octenylene, nonenylene, decenylene, undecenylene, dodecenylene, tridecenylene, tetradecenylene, pentadecenylene, hexadecenylene, heptadecenylene, octadecenylene, nonadecenylene, eicosenylene, henicosenylene, doicosenylene, tricosenylene, and tetraicosenylene, wherein the groups are most preferably bonded to the adjacent C(O) group or X group by a terminal C-atom,

R⁷ is independently selected from optionally hydroxyl-substituted hexyl, heptyl, octyl, nonyl, decyl, undecyl, dodecyl, tridecyl, tetradecyl, pentadecyl, hexadecyl, heptadecyl, octadecylene, nonadecyl, eicosyl, henicosyl, doicosyl, tricosyl, and tetraicosyl, or hexenyl, heptenyl, octenyl, nonenyl, decenyl, undecenyl, dodecenyl, tridecenyl, tetradecenyl, pentadecenyl, hexadecenyl, heptadecenyl, octadecenyl, nonadecenyl, eicosenyl, henicosenyl, doicosenyl, tricosenyl, and tetraicosenyl, wherein the groups are most preferably bonded to the adjacent C(O) group by a terminal C-atom, and

R⁷* is as defined in any of the embodiments according to the invention,

m is 0-10, preferably 1, 2, 3, 4 or 5.

In an also preferred embodiment according to the invention, a hair care formulation is provided containing at least one compound of the formula (IV), wherein at least one of the radicals G contains at least one moiety

—R⁶(—C(O)—O—R⁶)ₘ—C(O)—O—R¹¹,

—R⁶(—C(O)—NR¹⁰—R⁶)ₘ—C(O)—O—R¹¹,

—R⁶(—C(O)—NR¹⁰—R⁶)ₘ—C(O)—NR¹⁰—R¹¹,

preferably —R⁶(—C(O)—O—R⁶)ₘ—C(O)—O—R¹¹, wherein R¹⁰, R⁶, R¹¹, and m are as defined above.

Preferably, in the above structures according to this embodiment,

R⁶ is selected from independently selected from optionally hydroxyl-substituted hexylene, heptylene, octylene, nonylene, decylene, undecylene, dodecylene, tridecylene, tetradecylene, pentadecylene, hexadecylene, heptadecylene, octadecylene, nonadecylene, eicosylene, henicosylene, doicosylene, tricosylene, and tetraicosylene, or hexenylene, heptenylene, octenylene, nonenylene, decenylene, undecenylene, dodecenylene, tridecenylene, tetradecenylene, pentadecenylene, hexadecenylene, heptadecenylene, octadecenylene, nonadecenylene, eicosenylene, henicosenylene, doicosenylene, tricosenylene, and tetraicosenylene, wherein the groups are most preferably bonded to the adjacent C(O) group or X group by a terminal C-atom,

R¹¹ is independently selected from optionally hydroxyl-substituted hexyl, heptyl, octyl, nonyl, decyl, undecyl, dodecyl, tridecyl, tetradecyl, pentadecyl, hexadecyl, heptadecyl, octadecylene, nonadecyl, eicosyl, henicosyl, doicosyl, tricosyl, and tetraicosyl, or hexenyl, heptenyl, octenyl, nonenyl, decenyl, undecenyl, dodecenyl, tridecenyl, tetradecenyl, pentadecenyl, hexadecenyl, heptadecenyl, octadecenyl, nonadecenyl, eicosenyl, henicosenyl, doicosenyl, tricosenyl, and tetraicosenyl, wherein the residues may bear one or more hydroxyl or carboxyl substituents, and wherein the groups are most preferably bonded to the adjacent C(O) group by a terminal C-atom, and

m is 0-10, preferably 1, 2, 3, 4 or 5.

In a preferred embodiment according to the invention, a hair care formulation is provided containing at least one compound of the formula (I), which may be specifically represented by the formula (III), or of the formula (IV), wherein in the formula (II) or (V) or (II*) m=0 to 10, preferred 1 to 10, more preferred 0 to 6, even more preferred 1 to 6, specifically 0, 1, 2, 3, 4, 5, 6, more specifically 0 or 1.

In another preferred embodiment according to the invention, a hair care formulation is provided containing at least one compound of the formula (I), which may be specifically represented by the formulas (III), or of the formula (IV), wherein R¹⁰ is selected from the group consisting of hydrogen, n-, iso-, or tert.-C₁-C₂₂-alkyl, C₂-C₂₂-alkoxyalkyl, C₅-C₃₀-cycloalkyl, C₆-C₃₀-aryl, C₆-C₃₀-aryl(C₁-C₆)alkyl, C₆-C₃₀-alkylaryl, C₂-C₂₂-alkenyl, C₂-C₂₂-alkenyloxyalkyl, which optionally can be each substituted by hydroxyl and halogen, and which optionally can contain one or more ether groups (—O—), preferably R¹⁶ is selected from hydrogen or n-, iso-, and tert.-C₁-C₂₂-alkyl.

Therein, the most preferred C1-C22-alkyl groups according to this embodiment are methyl, ethyl, n-propyl, iso-propyl, n-butyl, iso-butyl, tert-butyl, n-pentane and n-hexane groups, cyclopentyl groups and cyclohexane groups,

In yet another preferred embodiment according to the invention, a hair care formulation is provided containing at least one compound of the formula (I), which may be specifically represented by the formula (III), or of the formula (IV), wherein R⁶ is independently selected from optionally substituted straight-chain, cyclic or branched, saturated or unsaturated radicals which have up to 24 carbon atoms, preferred 1 to 24, more preferred 2 to 20 carbon atoms, even more preferred 8 to 18 carbon atoms.

Most preferably, according to this embodiment R⁶ is selected from radicals derived from lesquerolic acid, ricinoleic acid, 12-hydroxy stearic acid or 14-hydroxy stearic acid, specifically from ricinoleic acid.

In a further preferred embodiment according to the invention, a hair care formulation is provided containing at least one compound of the formula (I), which may be specifically represented by the formula (III), wherein R⁷ is independently selected from optionally substituted straight-chain, cyclic or branched, saturated or unsaturated hydrocarbon radicals which have up to 24 carbon atoms, preferred 1 to 24, more preferred 2 to 20 carbon atoms, even more preferred 8 to 18 carbon atoms, which can be optionally substituted by one or more groups selected from —C(O)O—, carboxyl, hydroxyl, or halide groups.

According to this embodiment, the halide groups are independently selected from fluoro, chloro, bromo or iodo groups, wherein chloro groups are preferred.

Most preferably, according to this embodiment the R⁷ radical is derived from oleic acid or stearic acid, specifically from oleic acid.

In another preferred embodiment according to the invention, a hair care formulation is provided containing at least one compound of the formula (IV), wherein R¹¹ is independently selected from optionally substituted straight-chain, cyclic or branched, saturated or unsaturated hydrocarbon radicals which have up to 24 carbon atoms, preferred 1 to 24, more preferred 2 to 20 carbon atoms, even more preferred 8 to 18 carbon atoms.

Most preferably, according to this embodiment the R¹¹ radical is derived from monohydroxy carboxylic acids, lesquerolic acid, ricinoleic acid, 10-hydroxy octadecanoic acid, 12-hydroxy octadecanoic acid, 14-hydroxy tetradecanoic acid, 10-hydroxy stearic acid, 12-hydroxy stearic acid, or from dihydroxy carboxylic acids, in particular 2,2′-di-hydroxymethyl propanoic acid, 9,10-dihydroxy stearic acid, or from mono- and diols, in particular from methanol, ethanol, 2-propanol, 1-butanol, t-butanol, undec-10-en-ol, oleyl alcohol, stearyl alcohol, 1,2,-propanediol, 1,3-propanediol, 1,3-butanediol, 1,4-butanediol, 1,2 hexanediol, 1,6-hexanediol, specifically from ricinoleic acid.

In a further preferred embodiment according to the invention, a hair care formulation is provided containing at least one compound of the formula (I), which may be specifically represented by the formula (III), wherein the total number of carbon atoms in R⁶+R⁷ (Σcarbon atoms R⁶, R⁷) in each single moiety of the general formula (II) composed of R⁶ and R⁷ is 10 to 300, preferred 15 to 200, more preferred 20 to 150, even more preferred 30 to 100, or wherein in the compound of formula (I) or (III) the total number of carbon atoms in R⁶+R⁷* (Σcarbon atoms R⁶, R⁷*) in each single moiety of the general formula (II*) composed of R⁶ and R⁷* is 10 to 300, preferred 15 to 200, more preferred 20 to 150, even more preferred 30 to 100.

In another preferred embodiment according to the invention, a hair care formulation is provided containing at least one compound represented by the formula (IV), wherein the total number of carbon atoms in R⁶+R¹¹ (Σcarbon atoms R⁶, R¹¹) in each single moiety of the general formula (V) composed of R⁶ and R¹¹ is 10 to 300, preferred 15 to 200, more preferred 20 to 150, even more preferred 30 to 100.

In yet another preferred embodiment according to the invention, a hair care formulation is provided containing at least one compound of the formula (I), which may be specifically represented by the formula (III), or of the formula (IV), wherein R² in the formula (III) or R¹ in the compound of formula (IV) is selected from divalent to hexavalent, preferred divalent to tetravalent, more preferred divalent to trivalent, in particular divalent, trivalent, tetravalent, pentavalent, even more preferably divalent, trivalent and tetravalent or hexavalent optionally substituted hydrocarbon radicals, preferably optionally hydroxyl, amino or amido substituted straight-chain, cyclic or branched, saturated, unsaturated or aromatic, preferably saturated radicals, preferably derived from polyols, from dihydroxy carboxylic acids with up to 25 carbon atoms, preferably selected from the groups consisting of 2,2′-di-hydroxymethyl propanoic acid, 9,10-dihydroxy stearic acid, or from polyhydroxy carboxylic acids with up to 25 carbon atoms, preferably selected from the groups consisting of 3,5-dihydroxy-3-methylpentanoic acid, and sugar acids, such as gluconic acid, glyceric acid, xylonic acid, and ascorbic acid.

As already described above in a general way, the term “derived” according to the invention describes that the structures of the radicals R² or R¹ are formally obtained by abstraction of hydroxyl groups, amino groups and carboxylic groups, respectively, which in the formulas (I), (III) or (IV) form the groups adjacent to the R¹ or R², respectively, such as —C(O)—X—, wherein X is O or NR¹⁰ as described above. Following this definition, it is clear to the person skilled in the art that for instance in a compound of the formula (IV), in which R¹ is a divalent 1,2-ethylene radical, the group R¹ can be defined as being derived from succinic acid.

In a further preferred embodiment according to the invention, a hair care formulation is provided containing at least one compound of the formula (I), which may be specifically represented by the formula (III), or of the formula (IV), wherein R¹⁰ is selected from the group consisting of hydrogen, n-, iso-, or tert.-C₁-C₂₂-alkyl, more preferred hydrogen.

In still a further preferred embodiment according to the invention, a hair care formulation is provided containing at least one compound of the formula (I), which may be specifically represented by the formula (III), or of the formula (IV), wherein R⁶ is derived from monohydroxy carboxylic acids with up to 25 carbon atoms, preferably independently selected from the group consisting of glycolic acid, lactic acid, 2-hydroxy butyric acid, 3-hydroxy-butyric acid, 4-hydroxy butyric acid, 14-hydroxy tetradecanoic acid, 10-hydroxy stearic acid, 12-hydroxy stearic acid, ricinoleic acid, and lesquerolic acid.

Therein, in case R⁶ is derived from carboxylic acids bearing more than one hydroxyl group, only one of the hydroxyl groups is utilized to constitute the structural element X in an ester group of a moiety of the general formula (II) or (V) linking the group R⁶ to R⁷, R¹¹ or another R⁶ group, or linking a group R⁶ to a R⁷* group.

In a preferred embodiment according to the invention, a hair care formulation is provided containing at least one compound of the formula (I), which may be specifically represented by the formula (III), wherein R⁷ is derived from carboxylic acids with up to 25 carbon atoms which do not have hydroxyl substituents, preferably independently selected from the group consisting of acetic acid, propanoic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, docosanoic acid, 2-ethyl hexanoic acid, 2,2-dimethyl propionic acid, 2,2-dimethyl heptanoic acid, 2,2-dimethyl octanoic acid, neodecanoic acid, undecyl-10-en-ic acid, oleic acid, linoleic acid, linolenic acid, and erucic acid.

Most preferably, according to this embodiment R⁷ is derived from oleic acid, linoleic acid or linolenic acid, in particular when R⁷ is combined in a moiety of the formula (II) with one or more R⁶ groups derived from ricinoleic acid or lesquerolic acid.

In another preferred embodiment according to the invention, a hair care formulation is provided containing at least one compound of the formula (I) represented by the formula (IV), wherein in the compound of formula (IV) R¹¹ is derived from linear or branched alcohols with up to 26 carbon atoms, preferably independently selected from the group consisting of methanol, ethanol, 2-propanol, 1-butanol, t-butanol, undec-10-en-ol, oleyl alcohol, stearyl alcohol, 1,2,-propanediol, 1,3-propanediol, 1,3-butanediol, 1,4-butanediol, 1,2 hexanediol, 1,6-hexanediol, glycerol, diglycerol, triglycerol, and linear or branched oligoglycerols formally comprising from 4 to 6 glycerol units, trimethylol propane, castor oil (ricinoleic acid triglyceride), lesquerella oil (lesquerolic acid triglyceride), pentaerythritol, sorbitol, polyalkylene oxides, such as ethylene oxide-, propylene oxide- and/or butylene oxide-based polyethers, 1,2,-propanediol, 1,3-propanediol, 1,3-butanediol, 1,4-butanediol, 1,2 hexanediol, and 1,6-hexanediol.

Most preferably, according to this embodiment R¹¹ is derived from glycerol or oligoglycerols, castor oil or poly-(C2-C4)-alkylene oxides, in particular when R¹¹ is combined in a moiety of the formula (V) with one or more R⁶ groups derived from ricinoleic acid or lesquerolic acid.

In yet another preferred embodiment according to the invention, a hair care formulation is provided containing at least one compound of the formula (I), which may be specifically represented by the formula (III), wherein at least one, preferably both of R⁶ and R⁷ of the moieties of the general formula (II) are derived from unsaturated carboxylic acids.

More preferably, according to this embodiment R⁶ is derived from ricinoleic acid, most preferably R⁶ is derived from ricinoleic acid and R⁷ is derived from oleic acid.

In another preferred embodiment according to the invention, a hair care formulation is provided containing at least one compound of the formula (I), which is specifically represented by the formulas (III), or of the formula (IV), wherein R¹ in the compound of the formula (IV) or R² in the compound of the formula (III) is selected from the group consisting of:
residues derived from polyols, preferably derived from the group consisting of 1,2-propanediol, 1,3-propanediol, 1,3-butanediol, 1,4-butanediol, 1,2-hexanediol, 1,6-hexanediol, glycerol, diglycerol, triglycerol and linear or branched oligoglycerols such as oligoglycerols formally comprising from 4 to 6 glycerol units, trimethylol propane, pentaerythritol, sorbitol,
residues derived from polyalkylene oxides, such as ethylene oxide-, propylene oxide- and/or butylene oxide-based polyethers, in particular derived from polyethylene glycols, such as diethylene glycol, triethylene glycol, tetraethylene glycol, and pentaethylene glycol, or derived from polypropylene glycols, such as dipropylene glycol, (in particular derived from 2,2′-oxydi-1-propanol, 1,1′-oxydi-2-propanol, and 2-(2-hydroxypropoxy)-1-propanol), tripropylene glycol, tetrapropylene glycol, pentapropylene glycol, derived from mixed ethylene oxide and butylene oxide based copolyethers, derived from mixed propylene oxide and butylene oxide based copolyethers, and derived from mixed ethylene oxide and propylene oxide and butylene oxide based copolyethers, oligomeric or polymeric polyols, such as hydroxy-functional polyacrylates, hydroxy-functional polyesters, hydroxy-functional polyurethanes,
residues derived from dihydroxycarboxylic acids, preferably derived from the group consisting of 2,2′-dihydroxymethyl propanoic acid and 9,10-dihydroxy stearic acid,
residues derived from polyhydroxy carboxylic acids with up to 25 carbon atoms, preferably selected from the groups consisting of 3,5-dihydroxy-3-methylpentanoic acid, and sugar acids, such as gluconic acid, glyceric acid, xylonic acid, and ascorbic acid,
residues derived from epoxy compounds, preferably derived from ether epoxy compounds, in total having more than one, preferred more than two carbon atoms, preferred selected from ethylene oxide, propylene oxide, butylene oxide, glycidyl ethers such as prepared in particular from the reaction of epichlorhydrin with alcohols, such as methanol, ethanol, 2-propanol, 1-butanol, t-butanol, undec-10-en-ol, oleyl alcohol, stearyl alcohol, 1,2,-propanediol, 1,3-propanediol, 1,3-butanediol, 1,4-butanediol, 1,2 hexanediol, 1,6-hexanediol, glycerol, diglycerol, triglycerol, and linear or branched oligoglycerols, in particular formally comprising from 4 to 6 glycerol units, trimethylol propane, castor oil (ricinoleic acid triglyceride), lesquerella oil (lesquerolic acid triglyceride), pentaerythritol, sorbitol, polyalkylene oxides, such as ethylene oxide-, propylene oxide- and/or butylene oxide-based polyethers, in particular derived from polyethylene glycols, such as diethylene glycol, triethylene glycol, tetraethylene glycol, and pentaethylene glycol, or derived from polypropylene glycols, like dipropylene glycol (in particular derived from 2,2′-oxydi-1-propanol, 1,1′-oxydi-2-propanol, and 2-(2-hydroxypropoxy)-1-propanol), tripropylene glycol, tetrapropylene glycol, pentapropylene glycol, derived from mixed ethylene oxide and butylene oxide based copolyethers, derived from mixed propylene oxide and butylene oxide based copolyethers, and derived from mixed ethylene oxide and propylene oxide and butylene oxide based copolyethers, or preferred glycidyl esters, with acids, in particular neodecanoic acid, oligomeric or polymeric polyols, such as hydroxy-functional polyacrylates, hydroxy-functional polyesters, hydroxy-functional polyurethanes,
residues derived from ester compounds obtained from the reaction of polyols or epoxides with hydroxy carboxylic acids, ester compounds obtained from the reaction of alcohols,

in particular alcohols selected from 1,2,-propanediol, 1,3-propanediol, 1,3-butanediol, 1,4-butanediol, 1,2 hexanediol, 1,6-hexanediol, glycerol, diglycerol, triglycerol and linear or branched oligoglycerols such as formally comprising from 4 to 6 glycerol units, trimethylol propane, castor oil (ricinoleic acid triglyceride), lesquerella oil pentaerythritol, sorbitol, polyalkylene oxides, such as ethylene oxide-, propylene oxide- and/or butylene oxide-based polyethers, in particular derived from polyethylene glycols, such as diethylene glycol, triethylene glycol, tetraethylene glycol, and pentaethylene glycol, or derived from polypropylene glycols, such as dipropylene glycol (in particular derived from 2,2′-oxydi-1-propanol, 1,1′-oxydi-2-propanol, and 2-(2-hydroxypropoxy)-1-propanol), tripropylene glycol, tetrapropylene glycol, pentapropylene glycol, derived from mixed ethylene oxide and butylene oxide based copolyethers, derived from mixed propylene oxide and butylene oxide based copolyethers, and derived from mixed ethylene oxide and propylene oxide and butylene oxide based copolyethers,

or epoxy compounds, in particular selected from ethylene oxide, propylene oxide, butylene oxide, glycidyl ethers, with alcohols, in particular. methanol, ethanol, 2-propanol, 1-butanol, t-butanol, undec-10-en-ol, oleyl alcohol, stearyl alcohol, 1,2,-propanediol, 1,3-propanediol, 1,3-butanediol, 1,4-butanediol, 1,2 hexanediol, 1,6-hexanediol, glycerol, diglycerol, triglycerol and higher linear or branched oligoglycerols, trimethylol propane, castor oil (ricinoleic acid triglyceride), lesquerella oil (lesquerolic acid triglyceride), pentaerythritol, sorbitol, polyalkylene oxides, such as ethylene oxide-, propylene oxide- and/or butylene oxide-based polyethers, in particular derived from polyethylene glycols, such as diethylene glycol, triethylene glycol, tetraethylene glycol, and pentaethylene glycol., or derived from polypropylene glycols, such as dipropylene glycol (in particular derived from 2,2′-oxydi-1-propanol, 1,1′-oxydi-2-propanol, and 2-(2-hydroxypropoxy)-1-propanol), tripropylene glycol, tetrapropylene glycol, pentapropylene glycol, derived from mixed ethylene oxide and butylene oxide based copolyethers, derived from mixed propylene oxide and butylene oxide based copolyethers, and derived from mixed ethylene oxide and propylene oxide and butylene oxide based copolyethers, or preferred glycidyl esters,

with hydroxyl functionalized carboxylic acids, in particular lactic acid, 4-hydroxy butanoic acid, ricinoleic acid, lesquerolic acid, 2,2-bis(hydroxymethyl) propionic acid, malic acid, tartaric acid, gluconic acid, especially preferred are the esters of glycerol with ricinoleic acid, i.e. castor oil, and lesquerolic acid, i.e. lesquerella oil,
residues derived from ester compounds obtained from the reaction of alcohols or epoxides with hydroxyl-free carboxylic acids,

such as divalent to hexavalent, preferably divalent to tetravalent, more preferably divalent to trivalent, even more preferably divalent, trivalent and tetravalent optionally amino or amido substituted straight-chain, cyclic or branched, saturated, unsaturated or aromatic radicals, derived from the reaction of alcohols, i.e. 1,2,-propanediol, 1,3-propanediol, 1,3-butanediol, 1,4-butanediol, 1,2 hexanediol, 1,6-hexanediol, glycerol, diglycerol, triglycerol and higher linear or branched oligoglycerols, trimethylol propane, castor oil (ricinoleic acid triglyceride), lesquerella oil pentaerythritol, sorbitol, polyalkylene oxides, such as ethylene oxide-, propylene oxide- and/or butylene oxide-based polyethers, in particular derived from polyethylene glycols, like diethylene glycol, triethylene glycol, tetraethylene glycol, and pentaethylene glycol, or derived from polypropylene glycols, such as dipropylene glycol (in particular derived from 2,2′-oxydi-1-propanol, 1,1′-oxydi-2-propanol, and 2-(2-hydroxypropoxy)-1-propanol), tripropylene glycol, tetrapropylene glycol, pentapropylene glycol, derived from mixed ethylene oxide and butylene oxide based copolyethers, derived from mixed propylene oxide and butylene oxide based copolyethers, and derived from mixed ethylene oxide and propylene oxide and butylene oxide based copolyethers, or

epoxy compounds, in particular ethylene oxide, propylene oxide, butylene oxide, glycidyl ethers with alcohols, in particular methanol, ethanol, 2-propanol, 1-butanol, t-butanol, undec-10-en-ol, oleyl alcohol, stearyl alcohol, 1,2,-propanediol, 1,3-propanediol, 1,3-butanediol, 1,4-butanediol, 1,2-hexanediol, 1,6-hexanediol, glycerol, diglycerol, triglycerol and higher linear or branched oligoglycerols, trimethylol propane, castor oil (ricinoleic acid triglyceride), lesquerella oil (lesquerolic acid triglyceride), pentaerythritol, sorbitol, polyalkylene oxides, such as ethylene oxide-, propylene oxide- and/or butylene oxide-based polyethers, in particular derived from polyethylene glycols, such as diethylene glycol, triethylene glycol, tetraethylene glycol, and pentaethylene glycol, or derived from polypropylene glycols, such as dipropylene glycol (in particular derived from 2,2′-oxydi-1-propanol, 1,1′-oxydi-2-propanol, and 2-(2-hydroxypropoxy)-1-propanol), tripropylene glycol, tetrapropylene glycol, pentapropylene glycol, derived from mixed ethylene oxide and butylene oxide based copolyethers, derived from mixed propylene oxide and butylene oxide based copolyethers, and derived from mixed ethylene oxide and propylene oxide and butylene oxide based copolyethers, or

preferably glycidyl esters,

with acids, in particular neodecanoic acid, with carboxylic acids, in particular acetic acid, propionic acid, butyric acid, dodecanoic acid, stearic acid, oleic acid, oxalic acid, malonic acid, succinic acid, maleic acid, itaconic acid, phthalic acid, terephthalic acid, trimellitic acid, pyromellitic acid, dimer fatty acids, preferably derived from a reaction of anhydrides or acid chlorides with alcohols,
residues derived from monocarboxylic acids, as mentioned above, or polycarboxylic acids such as succinic acid, maleic acid, itaconic acid, phthalic acid, terephthalic acid, trimellitic acid, pyromellitic acid, dimer fatty acids, preferably their anhydrides or acid chlorides, and polyacrylic acids.

In a further preferred embodiment according to the invention, a hair care formulation is provided containing at least one compound of the formula (I) represented by the formula (III), or of the formula (IV), wherein R¹ in the formula (IV) or at least one of R² and R³ in the formula (III) is a divalent hydrocarbon radical, derived from the polyalkylene oxy groups as mentioned above of the general formula:

—[CH₂CH₂O]_{q1}—[CH₂CH(CH₃)O]ᵣ₁—[CH₂CH(C₂H₅)O]ₛ₁—{[CH₂CH₂]_{g2}—[CH₂CH(CH₃)]ᵣ₂—[CH₂CH(C₂H₅)]ₛ₂}—

with

q1=0 to 49, preferred 0 to 10, more preferred 1 to 10, even more preferred 1 to 5,

r1=0 to 32, preferred 0 to 10, more preferred 1 to 10, even more preferred 1 to 5,

s1=0 to 24, preferred 0 to 10, more preferred 1 to 10, even more preferred 1 to 5,

q2=0 or 1,

r2=0 or 1,

s2=0 or 1, and

Σ(q2+r2+s2)=1,

with the proviso that the sum of the carbon atoms in such polyalkylene oxide groups is 2 to 100, preferred 2 to 50, more preferred 2 to 30, even more preferred 2 to 20, specifically 2 to 15.

According to this embodiment, preferably, q2=0, and one or two of q1, r1 and s1 are 0,

and more preferably

q2=0, r1 and s1 are 0, or

q2=0, q1 and s1 are 0.

In still a further preferred embodiment according to the invention, a hair care formulation is provided containing at least one compound of the formula (I) represented by the formula (III) or of the formula (IV), wherein R¹ in the formula (IV) or at least one of R² and R³ in the formula (III) is

a divalent hydrocarbon radical, derived from oligoglycerols as mentioned above, of the general formula:

—[CH₂CH(R⁸)CH₂O]ₜ₁—[CH₂CH(R⁸)CH₂)]ₜ₂—

with

t1=0 to 32, preferred 0 to 10, more preferred 1 to 10, even more preferred 1 to 5, specifically 1 and 2,

t2=1,

R⁸═OH or —O—C(O)—R⁶—N⁺(R¹⁰)₃, wherein R¹⁰ and R⁶ are as defined above,

with the proviso that the sum of the carbon atoms of R⁸ is 2 to 100, preferred 2 to 50, more preferred 2 to 30, even more preferred 2 to 20, specifically 2 to 15.

In another preferred embodiment according to the invention, a hair care formulation is provided containing at least one compound of the formula (I) represented by the formula (III), or of the formula (IV), wherein R¹ in the formula (IV) or at least one of R² and R³ in the formula (III) is a divalent hydrocarbon radical comprising at least one ester group of the general formulas:

—[CH₂CH₂O]_{q1}—R⁹—[CH₂CH₂O]_{q1}—[CH₂CH₂]_{q2}—

with

q1 can be the same or different and are as defined above and q2=1

and

—[CH₂CH(R⁸)CH₂O]ₜ₁—R⁹—[CH₂CH(R⁹)CH₂O]ₜ₁—[CH₂CH(R⁸)CH₂)]ₜ₂—

with

t1=0 to 32, preferred 0 to 10, more preferred 1 to 10, even more preferred 1 to 5, specifically 1 and 2,

t2=1,

R⁸═OH or —O—C(O)—R⁶-N+(R¹⁰)₃, wherein R¹⁰ and R⁶ are as defined above,

with the proviso that the sum of the carbon atoms of R⁸ is 2 to 100, preferred 2 to 50, more preferred 2 to 30, even more preferred 2 to 20, specifically 2 to 15 and

R⁹ is selected from —C(O)C(O)O—, —C(O)(CH₂)₁₋₈C(O)O—, such as derived from succinic acid, adipic acid, sebacic acid, or —C(O)(C₆H₄)C(O)O—, i.e. derived from phthalic and terephthalic acid, —C(O)CH═CHC(O)O—, —C(O)C(═CH₂)—CH₂C(O)O—, —C(O)CH(OH)CH(OH)C(O)O—,

with the proviso that the sum of the carbon atoms in R⁹ is 2 to 100, preferred 2 to 50, more preferred 2 to 30, even more preferred 2 to 20, specifically 2 to 15.

In a further preferred embodiment according to the invention, a hair care formulation is provided containing at least one compound of the formula (I) represented by the formula (III), or of the formula (IV), wherein R¹ in the formula (IV) or R² in the formula (III) is a hydrocarbon residue which does not contain a heteroatom or contains one or more groups —O—, preferably one to five —O— groups, and wherein the groups —O— are preferably ether groups, but can also form an ester group together with a carbonyl group, and wherein the groups R² can be optionally substituted by one or more hydroxyl groups, but are preferably not substituted by hydroxyl groups.

According to this embodiment, it is more preferred that when the compound contains ether or ester groups, it does not contain any further heteroatoms other than oxygen, most preferably the compound contains one to five —O— groups and no further heteroatoms except for optional carbonyl O atoms.

In another preferred embodiment according to the invention, a hair care formulation is provided containing at least one compound of the formula (I), which may be specifically represented by the formula (III), wherein in the one or more groups F in at least one moiety of the formula:

—R⁶(—X—C(O)—R⁶)ₘ—X—C(O)—R⁷ (II)

X and m are as defined above, and

R⁶ is independently derived from lactic acid, ricinoleic acid, lesquerolic acid, 10-hydroxy stearic acid, 12-hydroxy stearic acid, 14-hydroxy tetradecanoic acid, and

R⁷ is derived from octadecanoic acid, eicosanoic acid, docosanoic acid, 2-ethyl hexanoic acid, 2,2-dimethyl propionic acid, neodecanoic acid, or oleic acid.

According to this embodiment, it is preferred when R⁶ is independently derived from 10-hydroxy stearic acid and ricinoleic acid, it is more preferred when R⁶ is independently derived from 10-hydroxy stearic acid and ricinoleic acid and R⁷ is derived from oleic acid.

In yet another preferred embodiment according to the invention, a hair care formulation is provided containing at least one compound of the formula (I), which may be specifically represented by the formula (III), or of the formula (IV), wherein R¹⁰ is preferably selected from the group consisting of hydrogen, or a bond to R¹ or R², thereby forming a cyclic structure of the schematic formulas:

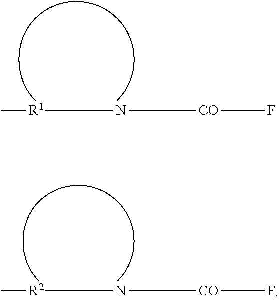

In a further preferred embodiment according to the invention, a hair care formulation is provided containing at least one compound of the formula (I), which may be represented by the general formula (III), wherein the groups F contain at least one moiety of the formula (II):

—R⁶(—X—C(O)—R⁶)ₘ—X—C(O)—R⁷ (II)

wherein R⁶, R⁷, X and m are as defined above, and wherein each moiety of the formula (II) comprises at least two different groups R⁶, wherein said groups R⁶ are arranged either random or blockwise, or wherein

the groups F contain at least one moiety of the formula (II*):

—R⁶(—X—C(O)—R⁶)ₘ—X—C(O)—R⁷* (II*)

wherein R⁶, R⁷*, X and m are as defined above, and wherein each moiety of the formula (II*) comprises at least two different groups R⁶, wherein said groups R⁶ are arranged either random or blockwise.

Preferably, according to this embodiment said R⁶ groups are arranged blockwise. According to the invention, blockwise arrangement refers to a structure of the formula (II) wherein two or more subunits each consisting of two or more R⁶ radicals of the same type are linked by covalent bonds or by single R⁶ groups being different from the R⁶ in the above-mentioned subunits acting as junction groups.

In yet another preferred embodiment according to the invention, a hair care formulation is provided containing at least one compound of the formula (I), which may be represented by the general formula (III), wherein the groups F contain at least one moiety of the formula (VI):

—R⁶(—X—C(O)—R⁶¹)ₘ₁(—X—C(O)—R⁶²)ₘ₂—X—C(O)—R⁷ (VI):

wherein R⁶ in formula (VI) is selected from R⁶¹ and R⁶², X and R⁷ are as defined above, and R⁶¹ and R⁶² represent two different groups R⁶ as defined in the previous embodiments, and wherein

m1=0 to 20, preferred 0 to 10, more preferred 0 to 6, even more preferred 1 to 6, specifically 0, 1, 2, 3, 4, 5, or 6,

m2=0 to 20, preferred 0 to 10, more preferred 0 to 6, even more preferred 1 to 6, specifically 0, 1, 2, 3, 4, 5, or 6,

m=m1+m2=0 to 20, preferred 0 to 10, more preferred 0 to 6, even more preferred 1 to 6, specifically 0, 1, 2, 3, 4, 5, or 6.

In a further preferred embodiment of the present invention, a hair care formulation is provided containing at least one compound of the formula (I), which may be represented by the general formula (III), wherein in the compound of formula (I) or (III):

the groups F contain at least one moiety of the formula (VI):

—R⁶(—X—C(O)—R⁶¹)ₘ₁(—X—C(O)—R⁶²)ₘ₂—X—C(O)—R⁷ (VI)

wherein X, m1 and m2 are as defined above, and R⁶, R⁶¹, R⁶² and R⁷ are as defined in the following table:

| R⁶ derived from | R⁶¹ derived from | R⁶² derived from | R⁷ derived from |
|---|---|---|---|
| unsaturated acids, in | unsaturated acids, in | unsaturated acids, in | unsaturated acids, in |
| particular ricinoleic | particular ricinoleic | particular ricinoleic | particular oleic acid |
| acid or lesquerolic acid | acid or lesquerolic acid | acid or lesquerolic acid | |
| unsaturated acids, in | unsaturated acids, in | unsaturated acids, in | saturated acids, in |
| particular ricinoleic | particular ricinoleic | particular ricinoleic | particular |
| acid or lesquerolic acid | acid or lesquerolic acid | acid or lesquerolic acid | octadecanoic acid or |
| | | | neodecanoic acid |
| unsaturated acids, in | unsaturated acids, in | saturated acids, in | saturated acids, in |
| particular ricinoleic | particular ricinoleic | particular 12- | particular |
| acid or lesquerolic acid | acid or lesquerolic acid | hydroxystearic acid | octadecanoic acid or |
| | | | neodecanoic acid |
| unsaturated acids, in | saturated acids, in | saturated acids, in | saturated acids, in |
| particular ricinoleic | particular 12- | particular 12- | particular |
| acid or lesquerolic acid | hydroxystearic acid | hydroxystearic acid | octadecanoic acid or |
| | | | neodecanoic acid |
| saturated acids, in | saturated acids, in | saturated acids, in | saturated acids, in |
| particular 12- | particular 12- | particular 12- | particular |
| hydroxystearic acid | hydroxystearic acid | hydroxystearic acid | octadecanoic acid or |
| | | | neodecanoic acid |
| saturated acids, in | saturated acids, in | saturated acids, in | unsaturated acids, in |
| particular 12- | particular 12- | particular 12- | particular oleic acid |
| hydroxystearic acid | hydroxystearic acid | hydroxystearic acid | |
| saturated acids, in | saturated acids, in | unsaturated acids, in | unsaturated acids, in |
| particular 12- | particular 12- | particular ricinoleic | particular oleic acid |
| hydroxystearic acid | hydroxystearic acid | acid or lesquerolic acid | |
| saturated acids, in | unsaturated acids, in | unsaturated acids, in | unsaturated acids, in |
| particular 12- | particular ricinoleic | particular ricinoleic | particular oleic acid |
| hydroxystearic acid | acid or lesquerolic acid | acid or lesquerolic acid | |
| saturated acids, in | unsaturated acids, in | saturated acids, in | unsaturated acids, in |
| particular 12- | particular ricinoleic | particular 12- | particular oleic acid |
| hydroxystearic acid | acid or lesquerolic acid | hydroxystearic acid | |
| unsaturated acids, in | saturated acids, in | unsaturated acids, in | saturated acids, in |
| particular ricinoleic | particular 12- | particular ricinoleic | particular |
| acid or lesquerolic acid | hydroxystearic acid | acid or lesquerolic acid | octadecanoic acid or |
| | | | neodecanoic acid. |

According to this embodiment, it is particularly preferred when R⁶, R⁶¹, R⁶² and R⁷ are selected according to the specific compounds given in a line of the above table showing the preferred combinations of R⁶, R⁶¹, R⁶² and R⁷ in a general way, it is even more preferable when ricinoleic acid is selected whenever there is the alternative of ricinoleic acid and lesquerolic acid.

In another preferred embodiment according to the present invention, a hair care formulation is provided containing at least one compound of the formula (I), which may be represented by the general formula (III), wherein in the compound of formula (I) or (III) the groups F contain at least one moiety of the formula (VI):

—R⁶(—X—C(O)—R⁶¹)ₘ₁(—X—C(O)—R⁶²)ₘ₂—X—C(O)—R⁷ (VI)

wherein X, m1, m2, R⁶, R⁶¹, R⁶² and R⁷ are each as defined above, and

the total number of carbon atoms in R⁶, R⁶¹, R⁶² and R⁷ (Σcarbon atoms R⁶, R⁶¹, R⁶² and R⁷) per group F is about 19 to 300, preferred 25 to 300, more preferred 30 to 300, specifically 30 to 200, more specifically 30 to 150.

In a further preferred embodiment according to the invention, a hair care formulation is provided containing at least one compound of the formula (I), which may be represented by the formula (III), or of the general formula (IV), wherein in the compound of formula (I), (III) or (IV): R⁶ is independently derived from mono- or poly-(such as di-, tri-, tetra-)hydroxy carboxylic acids selected from the group consisting of lactic acid, ricinoleic acid, lesquerolic acid, 10-hydroxy stearic acid, and 12-hydroxy stearic acid, 14-hydroxy tetradecanoic acid, 2,2′-di-hydroxymethyl propanoic acid, and 9,10-dihydroxy stearic acid, and gluconic acid, preferably at least one or all R⁶ are derived from ricinoleic acid.

In a preferred embodiment according to the invention, a hair care formulation is provided containing at least one compound of the formula (I), which may be represented by the general formula (III), wherein in the compound of formula (I) or (III)

R⁷ is independently derived from octadecanoic acid, eicosanoic acid, docosanoic acid, 2-ethyl hexanoic acid, 2,2-dimethyl propionic acid, neodecanoic acid, oleic acid, preferably all R⁷ are derived from oleic acid.

According to this embodiment, it is preferred when the above-cited R⁷ are combined with R⁶ moieties derived from ricinoleic acid, lesquerolic acid, 10-hydroxy stearic acid and 12-hydroxy stearic acid in one or more moieties of the formula (II), even more preferably more than 50% by number of the moieties of the formula (II) comprise R⁶ and R⁷ groups as described above, and most preferably in all moieties of the formula (II) each R⁶ is derived from ricinoleic acid or 10-hydroxy stearic acid, and each R⁷ is derived from oleic acid.

In a further preferred embodiment of the invention, a hair care formulation is provided containing at least one compound of the formula (I), (III) or (IV) as defined above, wherein low melting fatty acids ≥C5 and high melting fatty acids ≥C5 are specifically positioned within the R⁶ and R⁷ containing ester elements of the general formula (II) and the R⁶ and R¹¹ containing ester elements of the general formula (V):

—R⁶(—X—C(O)—R⁶)ₘ—X—C(O)—R⁷ (II) and

—R⁶(—C(O)—X—R⁶)ₘ—C(O)—X—R¹¹ (V),

in particular in the moieties

—O—C(O)—R⁶—(O—C(O)—R⁶)ₘ—O—C(O)—R⁷ and

—NR¹⁰—C(O)—R⁶—(O—C(O)—R⁶)ₘ—O—C(O)—R⁷,

wherein X, R⁶, R⁷, R¹¹ and R¹⁰ are as defined above, or

wherein in the compound of the formula (I), (III) or (IV) low melting and high melting fatty acids ≥C5 are specifically positioned within the R⁶ and R⁷* containing ester elements of the general formula (II*)

—R⁶(—X—C(O)—R⁶)ₘ—X—C(O)—R⁷* (II*)

in particular in the moieties

—O—C(O)—R⁶—(O—C(O)—R⁶)ₘ—O—C(O)—R⁷* and

—NR¹⁰—C(O)—R⁶—(O—C(O)—R⁶)ₘ—O—C(O)—R⁷*,

wherein X, R⁶, and R⁷* and R¹⁰ are as defined above.

It is in general within the scope of the invention that low melting and high melting fatty acids ≥C5 are specifically positioned independently for individual ester groups of moieties selected from the moieties of the general formulas (II), (II*) and (V) present in the compounds of the general formulas (I), (III) and (IV). The term “≥C5” indicates that the respective high melting fatty acids and low melting fatty acids have 5 or more carbon atoms. As the carbonyl group of the fatty acids is formally excluded from the groups R⁶ and R⁷, the groups R⁶ and R⁷ derived from said high melting and low melting fatty acids ≥C5 have 4 or more carbon atoms.

For instance, it is preferred according to this embodiment of the invention if a number of moieties of the general formula (II) of the compounds of the formulas (I) and (III) present in the hair care formulation displays the specific positioning of low melting fatty acids and high melting fatty acid scaffolds as described in the following, while other moieties of the general formula (II) do not. This may be in particular the case for moieties present in different residues F as defined above.

Within the frame of the present invention low melting fatty acids ≥C5 are defined by a melting point ≤40° C. Preferred examples are in particular oleic acid, lesquerolic acid, ricinoleic acid, octanoic acid, decanoic acid, pivalinic acid, and neodecanoic acid.

Within the frame of the present invention high melting fatty acids ≥C5 are defined by a melting point >40° C. Preferred examples are in particular dodecanoic acid, tetradecanoic acid, hexadecanoic acid, octadecanoic acid, arachidic acid, behenic acid, 10-hydroxy octadecanoic acid, 12-hydroxy octadecanoic acid, and 14-hydroxy tetradecanoic acid.

The corresponding melting points can be taken from the literature (G. Knothe et al., J. Am. Oil Chem. Soc., 2009, 86, 844-856).

In a further preferred embodiment according to the invention, a hair care formulation is provided containing at least one compound of the formula (I) or (III) as defined above, wherein in one or more moieties of the formula (II) at least one, preferred more than one, more preferred one, two or three low melting fatty acids ≥C5 each forming a group R⁶ are contained in the radical or the radicals R⁶ adjacent to R⁷, while at least one, preferred more than one, more preferred one, two or three high melting fatty acids ≥C5 form the radical or radicals R⁶ at the opposite terminus of a R⁶- and R⁷-containing ester element of the formula (II), or in such a manner that in one or more moieties of the formula (II) at least one, preferred more than one, more preferred one, two or three high melting fatty acids ≥C5 each forming R⁶ form the radical or radicals R⁶ adjacent to R⁷, while at least one, preferred more than one, more preferred one, two or three low melting fatty acids ≥C5 form the radical or radicals R⁶ at the opposite terminus of a R⁶- and R⁷-containing ester element of the formula (II). It is preferable when at least 50 of all moieties of the formula (II) display such a positioning of R⁶-radicals derived from high-melting and low-melting fatty acids as described above, and it is more preferred when more than 80% of all moieties of the formula (II) display such a positioning of R⁶-radicals derived from high-melting and low-melting fatty acids as described above, and most preferably all moieties of the general formula (II) display such positioning of residues R⁶ and R⁷ as described above.

In a likewise preferred embodiment, a hair care formulation is provided containing at least one compound of the formula (IV) as defined above, wherein in one or more moieties of the formula (V) at least one, preferred more than one, more preferred one, two or three low melting fatty acids ≥C5 each forming a group R⁶ are contained in the radical or the radicals R⁶ adjacent to R¹¹, while at least one, preferred more than one, more preferred one, two or three high melting fatty acids ≥C5 form the radical or radicals R⁶ at the opposite terminus of a R⁶- and R¹¹-containing ester element of the formula (V), or in such a manner that in one or more moieties of the formula (V) least one, preferred more than one, more preferred one, two or three high melting fatty acids ≥C5 each forming R⁶ form the radical or radicals R⁶ adjacent to R¹¹, while at least one, preferred more than one, more preferred one, two or three low melting fatty acids C5 form the radical or radicals R⁶ at the opposite terminus of a R⁶- and R¹¹-containing ester element of the formula (V).

It is preferable when at least 50% of all moieties of the formula (V) display such a positioning of R⁶-radicals derived from high-melting and low-melting fatty acids as described above, and it is more preferred when more than 80% of all moieties of the formula (V) display such a positioning of R⁶-radicals derived from high-melting and low-melting fatty acids as described above, and most preferably all moieties of the general formula (V) display such positioning of residues R⁶ and R¹¹ as described above.

In another likewise preferred embodiment, a hair care formulation is provided containing at least one compound of the formula (I) or (III) as defined above, wherein in one or more moieties of the formula (II*) at least one, preferred more than one, more preferred one, two or three low melting fatty acids with 5 or more carbon atoms and a melting point of 40° C. or below each forming a group R⁶ are contained in the radical or the radicals R⁶ adjacent to R⁷*, while at least one, preferred more than one, more preferred one, two or three high melting fatty acids with 5 or more carbon atoms and a melting point above 40° C. form the radical or radicals R⁶ at the opposite terminus of a R⁶- and R⁷*-containing ester element of the formula (II*), or in one or more moieties of the formula (II*) at least one, preferred more than one, more preferred one, two or three high melting fatty acids ≥C5 each forming R⁶ form the radical or radicals R⁶ adjacent to R⁷*, while at least one, preferred more than one, more preferred one, two or three low melting fatty acids with 5 or more carbon atoms and a melting point below 40° C. form the radical or radicals R⁶ at the opposite terminus of a R⁶- and R⁷*-containing ester element of the formula (II*), and it is preferable when at least 50% of all moieties of the formula (II*) display such a positioning of R⁶-radicals derived from high-melting and low-melting fatty acids as described above, it is more preferred when more than 80% of all moieties of the formula (II*) display such a positioning of R⁶-radicals derived from high-melting and low-melting fatty acids as described above, and it is most preferably when all moieties of the general formula (II) display such positioning of residues R⁶ and R⁷* as described above.

As already stated above, the specific positioning of high and low melting fatty acids may be independently varied for each individual R⁶- and R⁷- containing ester moiety of the formula (II) of a compound of the general formula (I) or (III), or for each individual R⁶- and R⁷* containing ester moiety of the formula (II*) of a compound of the formula (I) or (III), and in a fully analogous way for each individual R⁶- and R¹¹-containing ester moiety of the formula (V) of a compound of the general formula (IV).

The above outlined preferred embodiments allow the incorporation of R⁶- and R⁷-containing ester elements or of R⁶- and R¹¹-containing ester elements each having a locally varying tendency towards crystallization, viscosity build up and phase formation over the whole length of these ester elements in

—R⁶(—X—C(O)—R⁶)ₘ—X—C(O)—R⁷ (II) and

—R⁶(—C(O)—X—R⁶)ₘ—C(O)—X—R¹¹ (V),

in particular in the moieties

—O—C(O)—R⁶—(O—C(O)—R⁶)ₘ—O—C(O)—R⁷ and

—NR¹⁰—C(O)—R⁶—(O—C(O)—R⁶)ₘ—O—C(O)—R⁷,

wherein X, R⁶, R⁷, R₁₀ and R¹¹ are as defined above,

or

the incorporation of R⁶- and R⁷*-containing ester elements each having a locally varying tendency towards crystallization, viscosity build up and phase formation over the whole length of these ester elements in

—R⁶(—X—C(O)—R⁶)ₘ—X—C(O)—R⁷* (II*),

in particular in the moieties

—O—C(O)—R⁶—(O—C(O)—R⁶)ₘ—O—C(O)—R⁷* and

—NR¹⁰—C(O)—R⁶—(O—C(O)—R⁶)ₘ—O—C(O)—R⁷*,

wherein X, R⁶, R⁷*, R¹⁰ and R¹¹ are as defined above.

The combination of the above mentioned carboxylic acids and synthetic concepts gives access to ester condensates having defined molecular weights, molecular weight distributions, carboxylic acid sequences and properties such as viscosity.

The radicals R¹, R² or R³ can be linked to the R⁶- and R⁷-containing ester elements in the moieties of the general formula (II) present in the groups F,

in particular by formation of groups of the general structure

—R⁶—(O—C(O)—R⁶)ₘ—O—C(O)—R⁷ and

—NR¹⁰—C(O)—R⁶—(O—C(O)—R⁶)ₘ—O—C(O)—R⁷,

preferably by esterification of
mono hydroxyl carboxylic acids, in particular lactic acid, 4-hydroxy butanoic acid,
dihydroxy carboxylic acids, in particular 2,2-bis(hydroxymethyl) propanoic acid,
poly hydroxy carboxylic acids, in particular gluconic acid or the dendrimeric oligomers of dihydroxy carboxylic acid oligomers, in particular dendrimeric oligomers of 2,2-bis(hydroxymethyl) propanoic acid,

with the corresponding acid chloride precursors Cl—C(O)—R⁶—(O—C(O)—R⁶)ₘ—O—C(O)—R⁷,

wherein X, R⁶, and R⁷ and R¹⁰ are as defined above,

and in the analogous manner the radicals R¹, R² or R³ can be linked to the R⁶- and R⁷*-containing ester elements in the moieties of the general formula (II*) present in the groups F, in particular by formation of groups of the general structure

—R⁶—(O—C(O)—R⁶)ₘ—O—C(O)—R⁷* and

—NR¹⁰—C(O)—R⁶—(O—C(O)—R⁶)ₘ—O—C(O)—R⁷*,

preferably by esterification of
mono hydroxyl carboxylic acids, in particular lactic acid, 4-hydroxy butanoic acid,
dihydroxy carboxylic acids, in particular 2,2-bis(hydroxymethyl) propanoic acid,
poly hydroxy carboxylic acids, in particular gluconic acid or the dendrimeric oligomers of dihydroxy carboxylic acid oligomers, in particular dendrimeric oligomers of 2,2-bis(hydroxymethyl) propanoic acid,

with the corresponding acid chloride precursors Cl—C(O)—R⁶—(O—C(O)—R⁶)ₘ—O—C(O)—R⁷*, wherein X, R⁶, and R⁷* and R¹⁰ are as defined above.

In a further preferred embodiment according to the invention, a hair care formulation is provided containing at least one compound of the general formula (I) represented by the formula (III), wherein R⁴ is selected from divalent to tetravalent, such as divalent, trivalent, tetravalent, preferably divalent, optionally substituted straight-chain, cyclic or branched, saturated, unsaturated or aromatic hydrocarbon radicals which have up to 200 carbon atoms, more preferred up to 150 carbon atoms, even more preferred up to 100 carbon atoms, specifically up to 80 carbon atoms, and preferably has at least 2, more preferred at least 10, more preferred as least 14 carbon atoms, which optionally contain one or more groups selected from —O—, —NH—, and —C(O)—, and wherein if a plurality of R⁴ is present in formula (III), they can be the same or different.

According to this embodiment, it is preferred when R⁴ is derived a dicarboxylic acid and one or two hydroxy carboxylic acids by cross-condensation, or by cross-condensation of a di-, tri or tetraol and an excess of dicarboxylic acids, more preferably by cross-condensation of one equivalent of a dicarboxylic acid and a monohydroxy carboxylic acid.

In another preferred embodiment according to the invention, a hair care formulation is provided containing at least one compound of the general formula (I) represented by the general formula (III), wherein R⁴ comprises at least one ester group (—O—C(O)—, or —C(O)—O—, respectively).

According to this embodiment, R⁴ is preferably derived from at least one dicarboxylic acid and at least one C8-C22 hydroxy carboxylic acid or C2-C20 di-tetraol, more preferably from at least one dicarboxylic acid and at least one C14-C22 monohydroxy carboxylic acid.

In still another preferred embodiment according to the invention, a hair care formulation is provided containing at least one compound of the formula (I) represented by the general formula (III), wherein R⁴ is derived from dicarboxylic acids, tricarboxylic acids or tetracarboxylic acids, in particular dicarboxylic acids, such as succinic acid, oxalic acid, malonic acid, malic acid, tartaric acid, maleic acid, itaconic acid, succinic acid, sebacic acid, dimer acids, amino-functional dicarboxylic acids, such as D-glutamic acid, and a dicarboxylic acid of the formula:

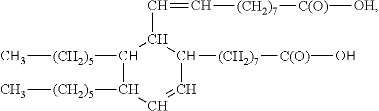

and condensation products of hydroxy carboxylic acids, in particular, from ricinoleic acid or lesquerolic acid, and dicarboxylic acids, such as succinic acid, oxalic acid, malonic acid, malic acid, tartaric acid, maleic acid, itaconic acid, succinic acid, sebacic acid, dimer acids, amino-functional dicarboxylic acids, such as D-glutamic acid, and a dicarboxylic acid of the formula:

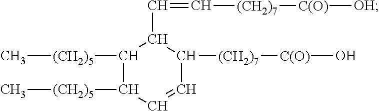

or

R⁴ is derived from amide condensation products of amino acids with maleic acid or succinic acid, such as N-maleoyl-β-alanine ((E)-4-(2-carboxyethylamino)-4-oxo-but-2-enoic acid), N-succinyl-β-alanine (4-[(2-hydroxy-1-methyl-2-oxo-ethyl)amino]-4-oxo-butanoic acid), N-maleoyl-asparagine (4-amino-2-[[(E)-4-hydroxy-4-oxo-but-2-enoyl]amino]-4-oxo-butanoic acid); or

R⁴ is derived from the ester condensation products of divalent alcohols, i.e. ethylene glycol, 1,2-propylene glycol, 1,3-propylene glycol, 1,3-butane diol, 1,4-butanediol, with dicarboxylic acid anhydrides, i.e. maleic anhydride, succinic anhydride, phthalic anhydride; or

R⁴ is derived from tri- or tetracarboxylic acids, such as citric acid, isocitric acid, trimelletic acid, pyromellitic acid, cyclobutane tetracarboxylic acid; or

R⁴ is derived from the bis-amide condensation products of amino acids with maleic acid or succinic acid, i.e. bis-(N-maleoyl)-lysine; or

R⁴ is derived from the ester condensation products of trivalent alcohols, such as glycerol, trimethylolpropane with dicarboxylic acid anhydrides, such as maleic anhydride, succinic anhydride, phthalic anhydride; or

R⁴ is derived from the ester condensation products of tetravalent alcohols, such as diglycerol, pentaerythritol with dicarboxylic acid anhydrides, such as maleic anhydride, succinic anhydride, phthalic anhydride, and

most preferred R⁴ is derived from ricinoleic acid or and succinic acid.

In a preferred embodiment of the hair care formulation according to the invention containing at least one compound of the formula (I), (III) or (IV), in at least one of the moieties of the formula (II), (111 or (V) two or more different R⁶ groups are present.

The presence of at least two different groups R⁶ in the moieties of the formula (II), (II*) or (V) results when at least two different types of hydroxy-substituted or amino-substituted carboxylic acid derivatives are used in the preparation of these chain structures. The different groups R⁶ may differ from each other in the number of C atoms, but also with regards to the number and position of double bonds, if any, and/or the position of substituents and the position of the linkage to the adjacent groups. They may differ with regards to if they are linear or branched. It is preferred when in at least one group F R⁶ independently represents hydrocarbon groups derived from ricinoleic acid and 12-hydroxy stearic acid.

In another preferred embodiment of the hair care formulation according to the invention containing at least one compound of the formula (I), (III) or (IV), in at least one of the moieties of the formula (II), (II*) or (V) the groups R⁶ and R⁷ in formula (II), the groups R⁶ and R⁷* in formula (II*), or R⁶ and R¹¹ in formula (V) are not based on the same carboxylic acid structure. It is preferred that R⁶ and R⁷, R⁶ and R⁷* or R⁶ and R¹¹ differ from each other regarding their number of carbon atoms, the number or position of double bonds, if any, in the carbon chain, or regarding the position of oxygen or nitrogen atoms bonded to the carbon chain of the groups. The carboxylic acid structures from which said groups are derived may also differ by two or more of the above-mentioned features.

In still another preferred embodiment of the hair care formulation according to the invention containing at least one compound of the formula (I) or (III), in the compound of the general formula (I)

p is 2-6,

R¹ is selected from di- to hexavalent linear, branched or cyclic alkylene groups, linear, branched or cyclic alkenylene groups, linear, branched or cyclic alkynylene groups, linear, branched or cyclic alkarylene groups, linear, branched or cyclic aralkylene groups and linear, branched or cyclic arylene groups, for instance phenylene, benzylene or tolylene groups, in particular from such groups having 1 to 1000 carbon atoms, more particular 1 to 150 carbon atoms,

and at least one group F contains one or more moieties of the general formula (II*)

—R⁶(—X—C(O)—R⁶)ₘ—X—C(O)—R⁷*

wherein R⁶, R⁷* and m are as defined above,

or wherein in a compound of the general formula (III)

r+s=2-6,

R² is selected from di- to hexavalent linear, branched or cyclic alkylene groups, linear, branched or cyclic alkenylene groups, linear, branched or cyclic alkynylene groups, linear, branched or cyclic alkarylene groups, linear, branched or cyclic aralkylene groups and linear, branched or cyclic arylene groups, for instance phenylene, benzylene or tolylene groups, in particular from such groups having 1 to 1000 carbon atoms, more particular 1 to 150 carbon atoms,

and at least one group F contains one or more moieties of the general formula (II*),

wherein R⁶, R⁷* and m are as defined above.

According to this embodiment, it is preferred that in formula (I) p is 2, 3 or 4, most preferably p is 2, or it is preferred that in formula (III) r+s=2, 3 or 4, most preferably r+s is 2.

It is also preferred that preferred that in the compound of the general formula (I) R¹ is selected from linear, branched or cyclic alkylene groups having 1 to 150 carbon atoms, more preferably linear alkylene groups having 1 to 12 carbon atoms, or it is preferred that in formula (III) R² is selected from linear, branched or cyclic alkylene groups having 1 to 150 carbon atoms, more preferably linear alkylene groups having 1 to 12 carbon atoms.

In a further preferred embodiment of the hair care formulation according to the invention containing at least one compound of the formula (I) or (III), in the least one compound of the general formula (I)

X═O, p is 2,

R¹ is selected from divalent linear, branched and cyclic alkylene groups, in particular from linear C1-C22 alkyl groups such as methylene, ethylene, n-propylene, n-butylene, n-pentylene, n-hexylene, n-heptylene or n-octylene groups, branched C1-C22 alkylene groups iso-propylene, iso-butylene, tert-butylene, iso-butylene, tert-pentylene, neo-pentylene, and 2-ethylhexylene groups, preferably from ethylene, n-propylene, n-butylene, n-pentylene and n-hexylene, and at least one group F contains one or more moieties of the general formula (II*)

—R⁶(—X—C(O)—R⁶)ₘ—X—C(O)—R⁷* (II*)

wherein R⁶, R⁷* and m are as defined above,

or wherein in the compound of the general formula (III)

X═O,

r+s=2,

R² is selected from divalent linear, branched and cyclic alkylene groups, in particular from linear C1-C22 alkyl groups such as methylene, ethylene, n-propylene, n-butylene, n-pentylene, n-hexylene, n-heptylene or n-octylene groups, branched C1-C22 alkylene groups iso-propylene, iso-butylene, tert-butylene, iso-butylene, tert-pentylene, neo-pentylene, and 2-ethylhexylene groups, preferably from ethylene, n-propylene, n-butylene, n-pentylene and n-hexylene, and at least one group F contains one or more moieties of the general formula (II*), wherein R⁶, R⁷* and m are as defined above.

In still another preferred embodiment of the hair care formulation according to the invention containing at least one compound of the formula (I), (III) or (IV), the compound of the formula (I) or (III) contains one or more groups R⁷* each terminated by three or more groups —O—C(O)-T, preferably by 4 or more groups —O—C(O)-T, most preferably by 4 to 12 groups —O—C(O)-T.

Therein, it is preferred that branched structures of R⁷* containing one branching structure as defined above are terminated by 3 to 10 groups —O—C(O)-T, while dendrimeric structures containing at least two branching structures as defined above are preferably terminated by 4 to 20 groups —O—C(O)-T.

In a further preferred embodiment of the hair care formulation according to the invention containing at least one compound of the formula (I), (III) or (IV), in the compound of the formula (I) or (III) one or more groups R⁷* each contain at least two branching structures of the general formula

—C(O)—B(—O—)_{b},

wherein B is a linear or branched hydrocarbon group having 2-20 carbon atoms, and b is 2 or more, and wherein the b groups (—O—) linked to the group B on the one side are linked to a C atom which may be the C atom of a CH₂ group or of a carbonyl group on the other side.

While the presence of a branching structure of the general formula

—B(—O—)_{b}

as defined above is mandatory in the group R⁷* in order to enable a branched structure which may be terminated by two or more groups —O—C(O)-T, the presence of two or more further branching structures of the general formula

—C(O)—B(—O—)_{b}

as defined above results in the formation of a dendrimeric structure, i.e. a structure having several branching points which may be arranged consecutively or parallel when moving from the bond linking R⁷* to the rest of the molecule to the terminal groups of R⁷*. However, deviating from the IUPAC definition of a dendrimer molecule [see A. Fradet et al., Pure and Applied Chemistry, 91(3), 523-561: Nomenclature and terminology for dendrimers with regular dendrons and for hyperbranched polymers (IUPAC Recommendations 2017)] the dendrons do not have to comprise exclusively dendritic and terminal constitutional repeating units, and it is not required that each path from the free valence of R⁷*, i.e. the valence bonding R⁷* to the rest of the molecule, to any end-group comprises the same number of constitutional repeating units.

It is further preferred that all (—O—) groups of the branching structure of the general formula —B(—O—)_{b} as defined above are substituted by the branching structures of the general formula

—C(O)—B(—O—)_{b} as defined above.

It is also preferred that one or more groups R⁷* contain 3 or more branching structures —C(O)—B(—O—)_{b}, more preferred 3-5 of said branching structures.

In this embodiment, it is preferred that b for both branching structures is independently selected from the range of 2-6, more preferably from the range of 2-4.

In a preferred embodiment of the hair care formulation according to the invention containing at least one compound of the formula (I), (III) or (IV), in the compound of the formula (I) or (III) the one or more branching structures of the general formal —B(—O—)_{b} or —C(O)—B(—O—)_{b} as defined above of at least one group R⁷* are independently derived from glyceric acid, 2,2-di-hydroxymethyl propionic acid, gluconic acid, maltobionic acid, lactobionic acid

It is preferred that all branching structures present in a group R⁷* are independently derived from 2,2-di-hydroxymethyl propionic acid, more preferably all branching structures in at least one group R⁷* are derived from 2,2-di-hydroxymethyl propionic acid.

It is even further preferred that all branching structure present in all groups R⁷* of a compound of the formula (I) or (III) are derived from the same polyhydroxy carboxylic acid.

In another preferred embodiment of the hair care formulation according to the invention, in the compound of the formula (I) or (III) one or more groups R⁷* are each terminated by two or more groups of the general formula

—R⁶(—X—C(O)—R⁶)ₜ—X—C(O)-T,

wherein R⁶, and T are as defined above, and

X═O,

t is independently 0-12, preferably t is independently 0-6, most preferably t is independently 0, 1, 2 or 3.

In this embodiment, two or more of the terminal groups as defined above are positioned at the terminus of an estolide chain. It is preferred that the one or more groups R⁷* are each terminated by 2-48 groups of the general formula —R⁶(—X—C(O)—R⁶)ₜ—X—C(O)-T, more preferable by 2-27 groups of the formula —R⁶(—X—C(O)—R⁶)ₜ—X—C(O)-T, and most preferable by 4-16 groups of the formula —R⁶(—X—C(O)—R⁶)ₜ—X—C(O)-T.

In a further preferred embodiment of the hair care formulation according to the invention, one or more groups R⁷* of the compound of the formula (I) or (III) are terminated by two or more groups, preferably 4 to 12 groups of the structure

—R⁶(—X—C(O)—R⁶)ₜ—X—C(O)-T,

wherein R⁶ is independently derived from C8-C24 monocarboxy-monohydroxy carboxylic acids, in particular ricinoleic acid, 12-hydroxy stearic acid, lesquerolic acid, 11-hydroxy-undecanoic acid,

X is O, and

T is independently derived from C2 to C24, preferred C8 to C24 fatty acids, in particular lauric acid, myristic acid, palmitic acid, oleic acid, stearic acid, behenic acid, arachidic acid, and t is 0-6, preferably 0, 1, 2 or 3.

According to this embodiment it is preferred when R⁶ is derived from ricinoleic acid, and T is derived from stearic acid or oleic acid.

More preferably, the R⁶ of all groups R⁷* are derived from ricinoleic acid, and even more preferably in all groups R⁷* R⁶ is derived from ricinoleic acid, T is derived from stearic acid or oleic acid, and t is 0, 1, 2 or 3.

In another preferred embodiment of the hair care formulation according to the invention, one or more groups R⁷* of the compound of the formula (I) or (III) are independently selected from one of the following branched or dendrimeric fatty acid structures:

—R¹²—O—C(O)—R⁶—(O—C(O)—R⁶¹)ₘ₁—(O—C(O)—R⁶²)ₘ₂—O—C(O)-T or

—R¹²—NR¹⁰—C(O)—R⁶—(O—C(O)—R⁶¹)ₘ₁—(O—C(O)—R⁶²)ₘ₂—O—C(O)-T, wherein

R¹² is selected from divalent optionally substituted hydrocarbon radicals which 2 to and 50 carbon atoms, specifically 2 to 20 carbon atoms, more specifically 2 to 10 carbon atoms and may contain optionally one or more groups selected from —O—, —NH—, —C(O)—, —C(S)—, tertiary amino groups

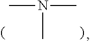

and can be substituted by —OH or halide groups, wherein the radical R¹⁰ cannot contain a combination of a —C(O)— group and a —O— group or a combination of a —C(O)— group and a —NH— or tertiary amino group forming an internal carboxylate group or an internal amide group, and preferably represents C1-C24 n-alkylene groups and CC2-C24 n-alkenylene groups, in particular —CH₂—, —CH₂CH₂—, —CH₂CH₂CH₂—,

R⁶ is as defined above,

m1 is 0 to 12, preferred 0 to 10, more preferred 0 to 6, even more preferred 1 to 6, specifically 0, 1, 2, 3, 4, 5, 6,

m2 is 0 to 12, preferred 0 to 10, more preferred 0 to 6, even more preferred 1 to 6, specifically 0, 1, 2, 3, 4, 5, 6,

and m1+m2 is t, wherein t is 0 to 12, preferred 0 to 10, more preferred 0 to 6, even more

preferred 1 to 6, specifically 0, 1, 2, 3, 4, 5, 6 and

T is as defined above,

R⁶¹ and R⁶² are selected from the groups R⁶ as defined above.

Preferably, R⁶, R⁶¹, R⁶² and T are selected as follows:

| R⁶ adjacent to R¹² is | R⁶¹ adjacent to R⁶ is | R⁶² adjacent to R⁷ is | |
|---|---|---|---|
| derived from | derived from | derived from | T is derived from |
| di- or polyhydroxylated | unsaturated acid, in | unsaturated acid, in | unsaturated acid, in |
| acid, in particular | particular ricinoleic | particular ricinoleic | particular oleic acid |
| 2,2′-di-hydroxymethyl | acid | acid | |
| propanoic acid | | | |
| di- or polyhydroxylated | unsaturated acid, in | unsaturated acid, in | saturated acid, in |
| acid, in particular | particular ricinoleic | particular ricinoleic | particular octadecanoic |
| 2,2′-di-hydroxymethyl | acid | acid | acid, neodecanoic acid |
| propanoic acid | | | |
| di- or polyhydroxylated | unsaturated acid, in | saturated acid, in | saturated acid, in |
| acid, in particular | particular ricinoleic | particular 12- | particular octadecanoic |
| 2,2′-di-hydroxymethyl | acid | hydroxystearic acid | acid, neodecanoic acid |
| propanoic acid | | | |
| di- or polyhydroxylated | saturated acid, in | saturated acid, in | saturated acid, in |
| acid, in particular | particular 12- | particular 12- | particular octadecanoic |
| 2,2′-di-hydroxymethyl | hydroxystearic acid | hydroxystearic acid | acid, neodecanoic acid |
| propanoic acid | | | |
| di- or polyhydroxylated | saturated acid, in | saturated acid, in | unsaturated acid, in |
| acid, in particular | particular 12- | particular 12- | particular oleic acid |
| 2,2′-di-hydroxymethyl | hydroxystearic acid | hydroxystearic acid | |
| propanoic acid | | | |
| di- or polyhydroxylated | saturated acid, in | unsaturated acid, in | unsaturated acid, in |
| acid, in particular | particular 12- | particular ricinoleic | particular oleic acid |
| 2,2′-di-hydroxymethyl | hydroxystearic acid | acid | |
| propanoic acid | | | |
| di- or polyhydroxylated | di- or polyhydroxylated | unsaturated acid, in | unsaturated acid, in |
| acid, in particular | acid, in particular | particular ricinoleic | particular oleic acid |
| 2,2′-di-hydroxymethyl | 2,2′-di-hydroxymethyl | acid | |
| propanoic acid | propanoic acid | | |
| di- or polyhydroxylated | di- or polyhydroxylated | unsaturated acid, in | saturated acid, in |
| acid, in particular | acid, in particular | particular ricinoleic | particular 12- |
| 2,2′-di-hydroxymethyl | 2,2′-di-hydroxymethyl | acid | hydroxystearic acid |
| propanoic acid | propanoic acid | | |
| di- or polyhydroxylated | di- or polyhydroxylated | saturated acid, in | unsaturated acid, in |
| acid, in particular | acid, in particular | particular 12- | particular oleic acid |
| 2,2′-di-hydroxymethyl | 2,2′-di-hydroxymethyl | hydroxystearic acid | |
| propanoic acid | propanoic acid | | |
| di- or polyhydroxylated | di- or polyhydroxylated | saturated acid, in | saturated acid, in |
| acid, in particular | acid, in particular | particular 12- | particular 12- |
| 2,2′-di-hydroxymethyl | 2,2′-di-hydroxymethyl | hydroxystearic acid | hydroxystearic acid |
| propanoic acid | propanoic acid | | |
| di- or polyhydroxylated | unsaturated acid, in | di- or polyhydroxylated | unsaturated acid, in |
| acid, in particular | particular ricinoleic | acid, in particular | particular oleic acid or |
| 2,2′-di-hydroxymethyl | acid | 2,2′-di-hydroxymethyl | unsaturated acid |
| propanoic acid | | propanoic acid | terminated oligoester |
| di- or polyhydroxylated | saturated acid, in | di- or polyhydroxylated | saturated acid, in |
| acid, in particular | particular 12- | acid, in particular | particular octadecanoic |
| 2,2′-di-hydroxymethyl | hydroxystearic acid | 2,2′-di-hydroxymethyl | acid, neodecanoic acid |
| propanoic acid | | propanoic acid | or saturated acid |
| | | | terminated oligoester |

wherein the total number of carbon atoms in R⁶+R⁷ (Σcarbon atoms R⁶, R⁷) is 19 to 300, preferred 25 to 300, more preferred 35 to 300, even more preferred 50 to 300, specifically 35 to 200, more specifically 35 to 150, even more specifically 50 to 150,

with the proviso that for R⁶, R⁶¹ and R⁶² being derived from di- or polyhydroxylated carboxylic acids at least one, preferred one to two, more preferred two, even more preferred all OH groups are esterified.

According to this embodiment, it is also preferred that one or more groups R⁷* of the compound of the formula (I) or (III) are independently derived from branched or dendrimeric fatty acid structures obtained by the esterification of 2,2′-di-hydroxymethyl propanoic acid with di-hydroxymethyl propanoic acid itself, C2 to C24, preferred C8 to C24 fatty acids, further preferred lauric acid, myristic acid, palmitic acid, oleic acid, stearic acid, behenic acid, arachidic acid and optionally mono hydroxy fatty acids, in particular ricinoleic acid, as exemplified by the structural formula

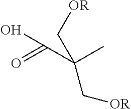

with R:

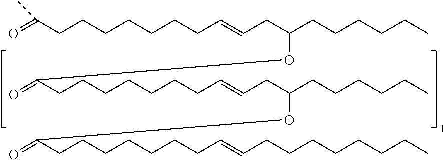

or the structure

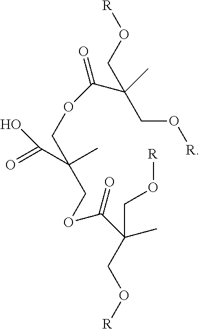

with R as displayed above.

Preferably, all groups R⁷* of the compound of the formula (I) or (III) are independently selected from the above-cited group of structures.

Even more preferably, all groups R⁷* of the compound of the formula (I) or (III) are represented by a single formula selected from the above-cited group of structures.

In a preferred embodiment of the hair care formulation according to the invention, at least one compound of the general formula (I) or (III) is represented by one of the following specific structures:

i) (fatty acid)-C(O)—O-(mono or oligo C8-C24 hydroxy fatty acid)-C(O)—O—(C2-C10 hydrocarbon)-O—C(O)-(mono or oligo C8-C24 hydroxy fatty acid)-O—C(O)-(fatty acid)

or

ii) (branched fatty acid)-C(O)—O-(mono or oligo C8-C24 hydroxy fatty acid)-C(O)—O—(C2-C10 hydrocarbon)-O—C(O)-(mono or oligo C8-C24 hydroxy fatty acid)-O—C(O)-(branched fatty acid)

or

iii) (dendrimeric fatty acid)-C(O)—O-(mono or oligo C8-C24 hydroxy fatty acid)-C(O)—O—(C2-C10 hydrocarbon)-O—C(O)-(mono or oligo C8-C24 hydroxy fatty acid)-O—C(O)-(dendrimeric fatty acid),

wherein
C2-C10 hydrocarbon is a C2-C10 hydrocarbylene group, in particular derived from ethylene glycol, 1,3 propylene glycol, 1,4 butanediol, 1,6 hexanediol, 1,2 propylene glycol, 1,3 butanediol,
mono or oligo C8-C24 hydroxy fatty acid is a group derived from a C8-C24 hydroxy-substituted carboxylic acid monomer or an oligomer of up to 20 C8-C24 hydroxy-substituted carboxylic acid monomers formed via esterification, in particular derived from mono or oligo ricinoleic acid with a degree of oligomerization of 2 to 20, preferred, 2 to 10, more preferred 2 to 6, even more preferred 2 to 4,
fatty acid is a saturated or unsaturated hydrocarbyl residue derived from a C2-C24 carboxylic acid with such residue, preferably derived from such C8 to C24 fatty acids, in particular from lauric acid, myristic acid, palmitic acid, oleic acid, stearic acid, behenic acid, arachidic acid,
branched fatty acid is a residue obtained by the esterification of a polyhydroxymonocarboxylic acid, in particular of 2,2′-di-hydroxymethyl propanoic acid, with C2 to C24, preferred C8 to C24 fatty acids, in particular lauric acid, myristic acid, palmitic acid, oleic acid, stearic acid, behenic acid, arachidic acid, and optionally mono hydroxy fatty acids, in particular. ricinoleic acid,
dendrimeric fatty acid is a residue obtained by the esterification of derived from i.e. the esterification a branched fatty acid residue as described above with at least one further polyhydroxymonocarboxylic acid, in particular 2,2′-di-hydroxymethyl propanoic acid, with C2 to C24, preferred C8 to C24 fatty acids, in particular lauric acid, myristic acid, palmitic acid, oleic acid, stearic acid, behenic acid, arachidic acid and optionally mono hydroxy fatty acids, in particular ricinoleic acid.

The branched fatty acid group is exemplified by the structure

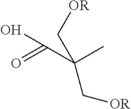

with R:

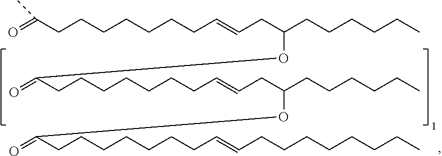

the dendrimeric fatty acid group is exemplified by the structure

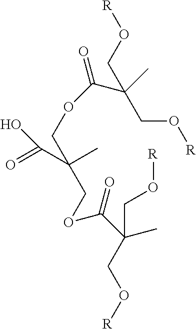

with R as displayed above for the branched fatty acid group.

In a further preferred embodiment of the hair care formulation according to the invention, the compound of the formula (I), (III) or (IV) comprises at least one moiety of the general formula

—([—O—C(O)—R⁶(—O—C(O)—R⁶)ₗ—O—C(O)-L-C(O)—O—(R⁶—C(O)—O)ₗ—R⁶—C(O)O])—

wherein R⁶ is as defined above,

l is an integer independently selected from 0-20, more preferably from 1-12, even more preferably from 2 to 10, and

L is a divalent hydrocarbon radical which may have 1 to 30 carbon atoms and may contain optionally one or more groups selected from —O—, —S—, —NH—, —C(O)—, —C(S)—, tertiary amino groups

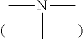

and quaternary ammonium groups, preferably L is a divalent alkylene or alkenylene radical having 1 to 30 carbon atoms,

more preferably L is selected from methylene, ethylene, propylene, butylene, pentylene, hexylene, heptylene, octylene, nonylene, ethenylene, propenylene, butenylene, pentenylene, hexenylene, heptenylene, octenylene, nonenylene,

most preferably L is selected from methylene, ethylene, ethenylene or butenylene.

According to the embodiment, preferably R⁶ is independently derived from C8-C24 monocarboxy-monohydroxy carboxylic acids, more preferably from ricinoleic acid, 12-hydroxy stearic acid, lesquerolic acid, 11-hydroxy-undecanoic acid, and even more preferably every R⁶ is independently derived from ricinoleic acid, 12-hydroxy stearic acid, lesquerolic acid, 11-hydroxy-undecanoic acid, and most preferably every R⁶ is the same group derived from one carboxylic acid selected from the group consisting of ricinoleic acid, 12-hydroxy stearic acid, lesquerolic acid, and 11-hydroxy-undecanoic acid.

It is explicitly noted that there may be an overlap of the structures of the moieties of the formula (V)

—R⁶(—C(O)—X—R⁶)ₘ—C(O)—X—R¹¹ (V)

and of the general formula

—([—O—C(O)—R⁶(—O—C(O)—R⁶)ₗ—O—C(O)-L-C(O)—O—(R⁶—C(O)—O)ₗ—R⁶—C(O)O])—

i.e. according to the invention a structure of the general formula

([—O—C(O)—R⁶(—O—C(O)—R⁶)ₗ—O—C(O)-L-C(O)—O—(R⁶—C(O)—O)ₗ—R⁶—C(O)O])—R¹¹

fulfills the requirements of the radical G of Formula (IV).

In another preferred embodiment of the hair care formulation according to the invention, the compound of the formula (I), (III) or (IV) comprises at least one moiety of the general formula

([—O—C(O)—R⁶(—O—C(O)—R⁶)ₗ—O—C(O)-L-C(O)—O—(R⁶—C(O)—O)ₗ—R⁶—C(O)O])—,

wherein L and l are as defined above,

and R⁶ is independently derived from C8-C24 monocarboxy-monohydroxy carboxylic acids, in particular from ricinoleic acid, 12-hydroxy stearic acid, lesquerolic acid, 11-hydroxy-undecanoic acid, most preferably R⁶ is derived from ricinoleic acid.

Preferably, L is selected from C1 to C10, preferably methylene, ethylene, butylene, octylene, decylene, l is independently in the range of 0 to 4, and R⁶ is derived from ricinoleic acid.

In still another preferred embodiment of the hair care formulation according to the invention, the compound of the formula (I), (III) or (IV) comprises at least one moiety of the general formula

([—O—C(O)—R⁶(—O—C(O)—R⁶)ₗ—O—C(O)-L-C(O)—O—(R⁶—C(O)—O)ₗ—R⁶—C(O)O])—

wherein L is selected from methylene, ethylene, and ethenylene,

R⁶ is derived from ricinoleic acid, and

l is independently selected from 0, 1, 2 and 3, and the sum of l is in the range of 0-4.

Preferably, L is an ethylene group, R⁶ is derived from ricinoleic acid, and l is independently selected from 0 or 1.

In a preferred embodiment of the hair care formulation according to the invention, the compound of the formula (I), (III) or (IV) comprises at least one moiety of the following structure:

(fatty alcohol)-O—C(O)-(mono or oligo C8-C24 hydroxy fatty acid)-O—C(O)—(C1-C12 hydrocarbon)-C(O)—O-(mono or oligo C8-C24 hydroxy fatty acid)-C(O)—O-(fatty alcohol), wherein
C2-C12 hydrocarbon is a C2-C12 hydrocarbylene group, in particular derived from succinic acid, maleic acid, itaconic acid, adipic acid, sebacic acid, dodecanedioic acid,
mono or oligo C8-C24 hydroxy fatty acid is a group derived from a C8-C24 hydroxy-substituted carboxylic acid monomer or an oligomer of up to 20 C8-C24 hydroxy-substituted carboxylic acid monomers formed via esterification, in particular derived from mono or oligo ricinoleic acid with a degree of oligomerization of 2 to 20, preferred, 2 to 10, more preferred 2 to 6, even more preferred 2 to 4,
fatty alcohol is a group derived from i.e. C2 to C24, preferred C8 to C24 fatty alcohols, in particular from n-octanol, n-decanol, n-dodecanol, n-tetradecanol, n-hexacedanol, oleyl alcohol, stearyl alcohol, behenyl alcohol, arachidyl alcohol.

Such kind of compound is exemplified by the following structure:

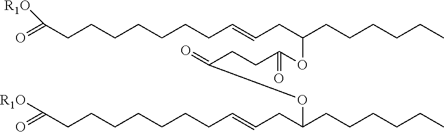

with R¹:

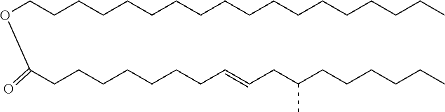 [SMILES: CCCCCCCCCCCCCCCCCCOC(=O)CCCCCCC/C=C/C[C@@H](C)CCCCCC]

(The dashed bond indicates R¹'s bond to the O atom of the structure above R¹.

In another preferred embodiment of the hair care formulation according to the invention, the compound of the formula (I), (III) or (IV) comprises at least one moiety of the structure

—([—O—C(O)—R⁶(—O—C(O)—R⁶)ₗ—O—C(O)-L-C(O)—O—(R⁶—C(O)—O)ₗ—R⁶—C(O))])—R¹¹,

wherein L, l, R⁶ and R¹¹ are as defined above.

Preferably, L, l and R⁶ are as defined above, and R¹¹ is selected from C1-C23 hydrocarbyl groups, preferably C1-C18 hydrocarbyl groups, more preferably C7-C19 hydrocarbyl groups, even more preferably C11-C17 hydrocarbyl groups, most preferably derived from lauric acid, myristic acid, palmitic acid, oleic acid, and stearic acid.

Further preferably, the compound of the formula (IV) has the following structure

R¹¹—([—O—C(O)—R⁶(—O—C(O)—R⁶)ₗ—O—C(O)-L-C(O)—O—(R⁶—C(O)—O)ₗ—R⁶—C(O))])-R¹¹,

wherein L, l and R⁶ are as defined above, and R¹¹ is independently selected C1-C23 hydrocarbyl groups, preferably C1-C18 hydrocarbyl groups, more preferably C7-C19 hydrocarbyl groups, even more preferably C11-C17 hydrocarbyl groups, and most preferably derived from lauric acid, myristic acid, palmitic acid, oleic acid, and stearic acid.

Even further preferably, L is selected from methylene, ethylene or ethenylene, butylene, hexylene, octylene, decylene or derived from itaconic acid,

l is independently selected from the range of 0-4,

R⁶ is selected derived from ricinoleic acid, and

R¹¹ is selected derived from oleic acid or stearic acid.

The present invention also relates to a compound of the general formula (Ia)

R¹(—X—C(O)—F)ₚ (Ia)

wherein

R¹ in formula (Ia) is selected from a p-valent, optionally substituted hydrocarbon radical and may contain optionally one or more groups selected from —O—, —NH—, —C(O)—, —C(S)—, tertiary amino groups

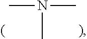

quaternary ammonium groups, and can be optionally substituted by one or more selected from carboxyl groups or hydroxyl groups,

p≥2, more preferably 2-811,

X can be the same or different and is selected from —O—, or —NR¹⁰—, wherein R¹⁰ is selected from the group consisting of hydrogen, or optionally substituted straight-chain, cyclic or branched, saturated, unsaturated or aromatic hydrocarbon radicals which have up to 100 carbon atoms which optionally contain one or more groups selected from —O—, —NH—, —C(O)—, —C(S)—, tertiary amino groups

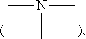

or in formula (Ia) R¹⁰ may form a bond to R¹ to form a cyclic structure,

F can be the same or different and is selected from optionally substituted straight-chain, cyclic or branched, saturated, unsaturated or aromatic hydrocarbon radicals which have up to 1005 carbon atoms, which optionally contain one or more groups selected from —O—, —NH—, —C(O)—, —C(S)—, and tertiary amino groups

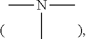

and can be optionally substituted by one or more selected from carboxyl, hydroxyl or halide groups, with the proviso that at least one of the radicals F contains at least one moiety of the formula (IIa) or formula (IIa*):

—R⁶(—X—C(O)—R⁶)ₘ—X—C(O)—R⁷ (IIa)

—R⁶(—X—C(O)—R⁶)ₘ—X—C(O)—R⁷* (IIa*)

wherein

X is as defined above,

m=1 to 20, preferably 2 to 20,

R⁶ is independently selected from a divalent optionally substituted straight-chain, cyclic or branched, saturated or unsaturated hydrocarbon radical which have up to 36 carbon atoms,

R⁷ is independently selected from optionally substituted straight-chain, cyclic or branched, saturated or unsaturated hydrocarbon radicals which have 1 to 1000 carbon atoms, optionally containing one or more groups selected from —O—, —NH—, —C(O)—, —C(S)—, tertiary amino groups

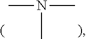

quaternary ammonium groups

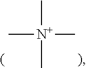

and which can be substituted with carboxyl, hydroxyl, or halide groups, wherein the radical R⁷ cannot contain an internal carboxy group or amide, i.e. R⁷ cannot contain a combination of a —C(O)— group and a —O— group or a combination of a —C(O)— group and a —NH— or tertiary amino group, with the proviso that in at least one moiety of the formula (IIa) R⁷ has at least 2, preferably at least 6 carbon atoms, and

in the same moiety of the formula (IIa) at least one R⁶ has at least 6, preferably at least 8 carbon atoms,

R⁷* is independently selected from optionally substituted branched or dendrimeric hydrocarbon radicals which have 1 to 1000 carbon atoms, optionally containing one or more groups selected from —O—, —NH—, —C(O)—, —C(S)—, tertiary amino groups

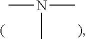

quaternary ammonium groups

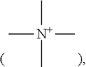

and which can be substituted with carboxyl, hydroxyl, or halide groups, wherein the radical R⁷* is terminated by two or more groups of the general structure

—X—C(O)-T

wherein X is as defined above, and

T is a monovalent straight-chain, cyclic or branched, saturated or unsaturated hydrocarbon radical optionally substituted with carboxyl, hydroxyl, or halide groups with up to 36 carbon atoms,

with the proviso that in at least one moiety of the formula (II*) R⁷* is terminated by one or more groups T having at least 2, preferably at least 6 carbon atoms, and in the same moiety of the formula (IIa*) at least one R⁶ has at least 6, preferably at least 8 carbon atoms,

or

containing at least one compound of the general formula (IVa)

R¹(—C(O)—X-G)_{q} (IVa),

wherein X is as defined above,

R¹ in formula (IVa) is selected from q-valent, optionally substituted hydrocarbon radicals which preferably have up to 1000 carbon atoms, and may contain optionally one or more groups selected from —O—, —NH—, —C(O)—, —C(S)—, tertiary amino groups

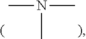

quaternary ammonium groups, and can be optionally substituted by one or more selected from carboxyl groups or hydroxyl groups,

q=2 to 55, preferably 2 to 40, more preferably 2 to 4, and

G can be the same or different and is selected from optionally substituted straight-chain, cyclic or branched, saturated, unsaturated or aromatic hydrocarbon radicals which have up to 1005 carbon atoms, which optionally contain one or more groups selected from —O—, —NH—, —C(O)—, —C(S)—, and tertiary amino groups

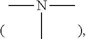

and can be substituted by one or more selected from carboxyl, hydroxyl or halide groups, with the proviso that at least one of the radicals G contains at least one moiety of the formula (Va):

—R⁶(—C(O)—X—R⁶)ₘ—C(O)—X—R¹¹ (Va)

wherein X is as defined above,

m=1 to 20, preferably 2 to 20,

R⁶ in formula (Va) is as defined above for formula (Ia),

R¹¹ is independently selected from optionally substituted straight-chain, cyclic or branched, saturated or unsaturated hydrocarbon radicals which have 1 to 1000 carbon atoms, optionally containing one or more groups selected from O—, —NH—, —C(O)—, —C(S)—, tertiary amino groups

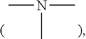

quaternary ammonium groups

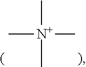

and which can be substituted with carboxyl, hydroxyl, or halide groups, wherein the radical R¹¹ cannot contain an internal carboxy group or amide, i.e. R¹¹ cannot contain a combination of a —C(O)— group and a —O— group or a combination of a —C(O)— group and a —NH— or tertiary amino group,

with the proviso that in at least one moiety of the formula (Va) R¹¹ has at least 2, preferably at least 6 carbon atoms, and in the same moiety of the formula (Va) at least one R⁶ has at least 6, preferably at least 8 carbon atoms,

with the general proviso that the compound of the formula (Ia) is not exclusively composed of glycerol and ricinoleic acid moieties, i.e. polyglycerol polyricinoleate (PGPR).

According to the invention, the residue R¹ in formula (Ia) is selected from a p-valent, optionally substituted hydrocarbon radical and may contain optionally one or more groups selected from —O—, —NH—, —C(O)—, —C(S)—, tertiary amino groups

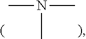

quaternary ammonium groups, and can be optionally substituted by one or more selected from carboxyl groups or hydroxyl groups.

According to the invention, the residue R¹ is p-valent, wherein p is 2 to 811, preferably 2 to 100, more preferably 2-50, even more preferably p is 2 to 30, which indicates that the residue R¹ bears p residues of the structure (—X—C(O)—F), with F as defined below. Accordingly, the term “p-valent” does not refer to or restrict the number of optional further substituents other than (—X—C(O)—F) of the residue R¹, which can be carboxylic groups or hydroxyl groups

The hydrocarbyl structure of R¹, which is p-valent regarding the residues (—X—C(O)—F), is preferably selected from the group consisting of linear, branched or cyclic alkylene groups, linear, branched or cyclic alkenylene groups, linear, branched or cyclic alkynylene groups, linear, branched or cyclic alkarylene groups, linear, branched or cyclic aralkylene groups and linear, branched or cyclic arylene groups, for instance phenylene, benzylene or tolylene groups, in particular from such groups having 1 to 1000 carbon atoms, more particular 1 to 150 carbon atoms.

Preferably, the hydrocarbon structures are linear or branched alkylene groups, or linear or branched alkylene groups interrupted by ether groups, ester groups or both ether and ester groups in particular branched structures derived from products as obtained by esterification of polyols with mono- or polyhydroxycarboxylic acids with up to 150 carbon atoms, or linear alkylene groups with up to 22 carbon atoms.

More preferably, the p-valent R¹ radical is selected from alkylene groups, which may be selected from the group consisting of linear, branched and cyclic alkylene groups, in particular from linear C1-C22 alkyl groups such as methylene, ethylene, n-propylene, n-butylene, n-pentylene, n-hexylene, n-heptylene or n-octylene groups, branched C1-C22 alkylene groups iso-propylene, iso-butylene, tert-butylene, iso-butylene, tert-pentylene, neo-pentylene, and 2-ethylhexylene groups.

There is no limitation regarding which C-atoms of the hydrocarbyl radicals bear the (—X—C(O)—F) groups attached to R¹.

Regarding the presence of functional groups optionally contained in R¹ and optional substituents, it is preferred that R¹ is derived from glycidyl compounds, glycerol and glycerol derivatives, in particular glycidol, glycerol diglycidyl ether, diglycidyl ether and polyglycerol compounds, or when R¹ is a linear alkylene group, in particular an alkylene group not bearing further substituents in addition to the (—X—C(O)—F) groups and even more preferred when R¹ is derived from the condensation product of glycidol, glycerol, glycerol diglycidyl ether, diglycidyl ether and polyglycerol compounds and C8-C24 monohydroxy fatty acids, in particular ricinoleic acid, lesquerolic acid or 12-hydroxyl stearic acid.

It is clear to the skilled person that usually the groups (—X—C(O)—F) are attached to the radical R¹ via —X—C(O)— units, in particular —O—C(O)— units, at positions which are substituted by —OH or —NHR¹⁰ groups in a parent compound from which R¹ is derived.

For example, the R¹ group derived from glycerol is a 1,2,3-propylene radical, wherein “1,2,3” indicates the positions at which the radical is substituted by the (—X—C(O)—F)-groups.

According to the invention, the group X can be the same or different and is selected from —O—, or —NR¹⁰—, wherein R¹⁰ is selected from the group consisting of hydrogen, or optionally substituted straight-chain, cyclic or branched, saturated, unsaturated or aromatic hydrocarbon radicals which have up to 100 carbon atoms which optionally contain one or more groups selected from —O—, —NH—, —C(O)—, —C(S)—, tertiary amino groups

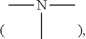

or in formula (Ia) R¹⁰ may form a bond to R¹ to form a cyclic structure.

Preferred examples for R¹⁰ are C1-C10 alkyl groups, in particular methyl, ethyl, n-propyl, iso-propyl, n-butyl, iso-butyl, tert-butyl, n-pentane and n-hexane groups, cyclopentyl groups and cyclohexane groups, C2-C10 alkenyl groups, in particular vinyl groups and allyl groups, and C6-C12 aromatic groups, in particular phenyl groups, tolyl groups, and benzyl groups, wherein each of the named groups may be substituted by hydroxyl groups or halide groups.

According to the invention, the residue F can be the same or different and is selected from optionally substituted straight-chain, cyclic or branched, saturated, unsaturated or aromatic hydrocarbon radicals which have up to 1005 carbon atoms, which optionally contain one or more groups selected from —O—, —NH—, —C(O)—, —C(S)—, tertiary amino groups

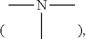

and can be optionally substituted by one or more selected from carboxyl, hydroxyl or halide groups, with the proviso that the compound contains at least one moiety of the formula (IIa) or of the formula (IIa*)

—R⁶(—X—C(O)—R⁶)ₘ—X—C(O)—R⁷ (IIa)

—R⁶(X—C(O)—R⁶)ₘ—X—C(O)—R^{7*} (IIa*).

Preferably, the group F only consists of a group of the formula (IIa),

or the group F only consists of a group of the formula (IIa*).

According to the invention, R⁶ is independently selected from an optionally substituted straight-chain, cyclic or branched, saturated or unsaturated hydrocarbon radical which have up to 36 carbon atoms, and thus R⁶ can be a divalent, optionally substituted straight-chain, cyclic or branched, saturated, unsaturated or aromatic hydrocarbon radical, i.e. R⁶ can represent a hydrocarbyl group selected from the group consisting of linear, branched or cyclic alkylene groups, linear, branched or cyclic alkenylene groups, linear, branched or cyclic alkynylene groups, linear, branched or cyclic alkarylene groups, linear, branched or cyclic aralkylene groups and linear, branched or cyclic arylene groups, for instance phenylene, benzylene or tolylene groups, in particular from such groups having 1 to 100 carbon atoms, each optionally containing one or more functional groups as indicated above.

More preferably, the R⁶ radical is selected from linear alkylene groups and linear alkenylene groups, in particular from linear C6-C24 alkylene such as hexylene, heptylene, octylene, nonylene, decylene, undecylene, dodecylene, tridecylene, tetradecylene, pentadecylene, hexadecylene, heptadecylene, octadecylene, nonadecylene, eicosylene, henicosylene, doicosylene, tricosylene, and tetraicosylene, or linear C6-C24 alkenylene groups such as hexenylene, heptenylene, octenylene, nonenylene, decenylene, undecenylene, dodecenylene, tridecenylene, tetradecenylene, pentadecenylene, hexadecenylene, heptadecenylene, octadecenylene, nonadecenylene, eicosenylene, henicosenylene, doicosenylene, tricosenylene, and tetraicosenylene, wherein the groups are most preferably bonded to the adjacent C(O) group by a terminal C-atom.

There is no limitation regarding at which C-atoms of the hydrocarbyl radicals the adjacent group C(O) group and X group are attached to R⁶.

However, R⁶ is preferably derived from a hydroxycarboxylic acid bearing one or more hydroxylic groups, more preferably from a monohydroxy carboxylic acid, most preferably from C7-C25 fatty acids bearing one hydroxyl group as substituent. Accordingly, R⁶ preferably represents the alkylene or alkenylene chain of such carboxylic acids.

Preferred examples for R⁶ are the structures derived from a corresponding hydroxyl carboxylic acid by abstraction of the carboxylate group and one OH group, wherein the hydroxyl carboxylic acid is preferably selected from ricinoleic acid, lesquerolic acid, 10-hydroxy octadecanoic acid, 12-hydroxy octadecanoic acid, 14-hydroxy tetradecanoic acid, 10-hydroxy stearic acid, 12-hydroxy stearic acid, or dihydroxy carboxylic acids, in particular 2,2′-di-hydroxymethyl propanoic acid, 9,10-dihydroxy stearic acid, or polyhydroxy carboxylic acids, in particular gluconic acid. Most preferably, R⁶ is derived in the above-stated manner from lesquerolic acid or ricinoleic acid. In both cases the naturally occurring enantiomers of the compounds, i.e. (9Z,12R)-12-hydroxyoctadec-9-enoic acid obtained by saponification or fractional distillation of hydrolysed castor oil, which is the seed oil of the castor plant, and (11Z, 14R)-14-hydroxyicos-11-enoic acid as isolated from Paysonia and Physaria species, are particularly preferred. However, the racemates, the S enantiomers as well as the E-configured isomers of the compounds, the racemates, the enantiomers and any possible mixture thereof are also preferred according to the invention.

The number m of the R⁶-containing repeating units (—X—C(O)—R⁶) of the at least one moiety present in a group F of the compound of the general formula (Ia) is from 1 to 20, preferably from 1 to 15, 1 to 12, 1 to 10, 1 to 8, or from 2 to 20, from 3 to 20, from 4 to 20, from 5 to 20, specifically 1, 2, 3, 4, 5, 6, 7, 8, 9, or 10.

R⁷ is independently selected from optionally substituted straight-chain, cyclic or branched, saturated or unsaturated hydrocarbon radicals which have 1 to 1000 carbon atoms, optionally containing one or more groups selected from —O—, —NH—, —C(O)—, —C(S)—, tertiary amino groups

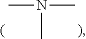

quaternary ammonium groups

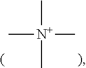

and which can be substituted with carboxyl, hydroxyl, or halide groups, wherein the radical R⁷ cannot contain an internal carboxy group or amide, i.e. R⁷ cannot contain a combination of a —C(O)— group and a —O— group or a combination of a —C(O)— group and a —NH— or tertiary amino group,

According to the invention, the radicals R⁷ can be the same or different selected from optionally substituted straight-chain, cyclic or branched, saturated or unsaturated hydrocarbon radicals which have 1 to 36 carbon atoms, and can thus represent a hydrocarbyl group selected from the group consisting of linear, branched or cyclic alkyl groups, linear, branched or cyclic alkenyl groups, linear, branched or cyclic alkynyl groups, linear, branched or cyclic alkaryl groups, linear, branched or cyclic aralkyl groups and linear, branched or cyclic aryl groups, for instance phenyl, benzyl or tolyl, in particular from such groups having 6 to 24 carbon atoms, each optionally containing one or more functional groups as indicated above.

More preferably, the R⁷ radical is selected from linear alkyl groups and linear alkenyl groups, in particular from linear C6-C24 alkyl groups such as hexyl, heptyl, octyl, nonyl, decyl, undecyl, dodecyl, tridecyl, tetradecyl, pentadecyl, hexadecyl, heptadecyl, octadecylene, nonadecyl, eicosyl, henicosyl, doicosyl, tricosyl, and tetraicosyl, or linear C6-C24 alkenyl groups such as hexenyl, heptenyl, octenyl, nonenyl, decenyl, undecenyl, dodecenyl, tridecenyl, tetradecenyl, pentadecenyl, hexadecenyl, heptadecenyl, octadecenyl, nonadecenyl, eicosenyl, henicosenyl, doicosenyl, tricosenyl, and tetraicosenyl, wherein the groups are most preferably bonded to the adjacent C(O) group or X group by a terminal C-atom.

There is no limitation regarding at which C-atom of the hydrocarbyl radicals the adjacent group C(O) group is attached to R⁷.

However, R⁷ is preferably derived from a carboxylic acid or a hydroxycarboxylic acid bearing one or more hydroxylic groups, more preferably from a carboxylic acid or monohydroxy carboxylic acid, most preferably from C7-C25 fatty acid bearing no hydroxyl group as substituent. Accordingly, R⁷ preferably represents the alkyl or alkenyl chain of such carboxylic acids.

Preferred examples for R⁷ are the structures derived from a corresponding carboxylic acid or hydroxyl carboxylic acid by abstraction of the carboxylate group, wherein the carboxylic acid may be selected from acetic acid, propionic acid, butyric acid, valeric acid, caproic acid, enanthic acid, caprylic acid, pelargonic acid, capric acid, undecanoic acid, lauric acid, tridecanoic acid, myristic acid, pentadecanoic acid, palmitic acid, margaric acid, stearic acid, linoleic acid, α-linolenic acid, γ-linolenic acid, nonadecylic acid, arachidic acid, mead's acid, arachidonic acid, heneicosanoic acid, docosanoic acid, tricosylic acid and lignoceric acid, from hydroxyl carboxylic acid such as lesquerolic acid, ricinoleic acid, 10-hydroxy octadecanoic acid, 12-hydroxy octadecanoic acid, 14-hydroxy tetradecanoic acid, 10-hydroxy stearic acid, 12-hydroxy stearic acid, or from dihydroxy carboxylic acids, in particular 2,2′-di-hydroxymethyl propanoic acid, 9,10-dihydroxy stearic acid, or polyhydroxy carboxylic acids, in particular gluconic acid.

Although the radical R⁷ can optionally contain one or more groups selected from —O—, —NH—, —C(O)—, —C(S)—, tertiary amino groups

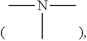

quaternary ammonium groups

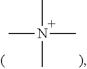

and may be substituted with OH groups or halide groups, the radical R⁷ cannot contain a combination of a —C(O)— group and a —O— group or a combination of a —C(O)— group and a —NH— or tertiary amino group forming an internal carboxylate group, i.e. an internal ester group, or an internal amide group.

According to the invention, it is mandatory that the compound at least one moiety of the formula (IIa) R⁷ has at least 2, preferably at least 6 carbon atoms, and in the same moiety of the formula (IIa) at least one R⁶ has at least 6, preferably at least 8 carbon atoms.

According to the invention, the group R⁷* of the formula (IIa*) is independently selected from optionally substituted branched or dendrimeric hydrocarbon radicals which have 1 to 1000 carbon atoms, optionally containing one or more groups selected from —O—, —NH—, —C(O)—, — C(S)—, tertiary amino groups

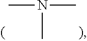

quaternary ammonium groups

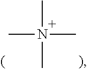

and which can be substituted with carboxyl, hydroxyl, or halide groups, wherein the radical R⁷* is terminated by two or more groups of the general structure

—X—C(O)-T

wherein X is as defined above, and

T is a monovalent straight-chain, cyclic or branched, saturated or unsaturated hydrocarbon radical optionally substituted with carboxyl, hydroxyl, or halide groups with up to 36 carbon atoms,

with the proviso that in at least one moiety of the formula (IIa*) R⁷* is terminated by one or more groups T having at least 2, preferably at least 6 carbon atoms, and in the same moiety of the formula (IIa*) at least one R⁶ has at least 6, preferably at least 8 carbon atoms.

As R⁷* is defined as a monovalent group, in order to provide the structural feature of being terminated by at least two groups of the general structure

—X—C(O)-T,

the presence of at least one branching structure is required in the residue R⁷*.

In the case R⁷* is a branched hydrocarbon radical, as the branching structure, the group comprises at least one moiety of the general formula

—B(—O—)_{b},

wherein B is a linear or branched hydrocarbon group having 3-20 carbon atoms, 3-10 and b is 2 or more, preferably 2-6, more preferably 2-4, and wherein the groups (—O—) linked to the group B on the one side are linked to a C atom on the other side.

Therein, the C atom may be of a CH₂ group or of a carbonyl group.

In the case R⁷* is a dendrimeric hydrocarbon radical, the group comprises at least one moiety of the general formula

—B(—O—)_{b},

wherein B and b are as defined above, and the groups (—O—) linked to the group B on the one side are linked to a C atom on the other side, and

at least one further moiety acting as branching structure of the general formula

—C(O)—B(—O—)_{b},

wherein B and b are as defined above, and the groups (—O—) linked to the group B on the one side are linked to a C atom on the other side, wherein the C atom may be of a CH₂ group or of a carbonyl group. The term dendrimeric hydrocarbon structure thus refers to a branched structure containing at least two consecutive branching structures.

Each group T constitutes one of at least two terminal groups of a R⁷* group and is typically derived from a fatty acid. Accordingly, the group T is preferably a linear saturated or monounsaturated hydrocarbon radical having 2 to 24 carbon atoms.

The group T is preferably linked to an (—O—) group of a branching structure of the general formula —B(—O—)_{b}, or —C(O)—B(—O—)_{b} via a carbonyl group or via an estolide chain.

By the presence of the above-described branching structures, R⁷* adopts a branched or even dendrimeric structure.

The following structure is an example of R⁷* being a branched hydrocarbon radical as defined above:

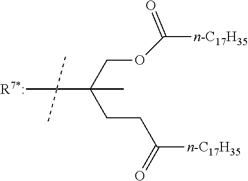

Therein, the branching structure of the general formula B(—O—)_{b} is derived from 2,2′-dihydroxymethylpropionic acid, and the group T is a n-heptadecanyl group linked to the branching structure. It is derived from stearic acid and linked to the group B by a —C(O)—O-unit.

Accordingly, the structure is terminated by two groups of the general structure —X—(CO)-T and contains a branching structure of the formula —B(—O—)₂.

An example of a group from which a branched group R⁷* may be derived is displayed below:

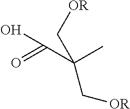

with R:

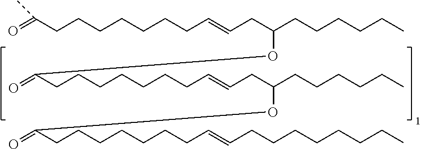

In the corresponding group R⁷*, the branching structure is as in the previous structure, however, the two terminal groups T, which are n-heptadec-9-enyl groups derived from oleic acid, are attached to the branching structure via a ricinoleic-acid derived estolide chain structure.

Another example of a group R⁷* according to the invention being a dendrimeric hydrocarbon is displayed below:

Therein, the branching structure —B(—O—)_{b} is directly followed by two further branching structures —(C(O)—B(—O—)_{b}, resulting in a further increase of terminating groups of the general structure —X—C(O)-T:

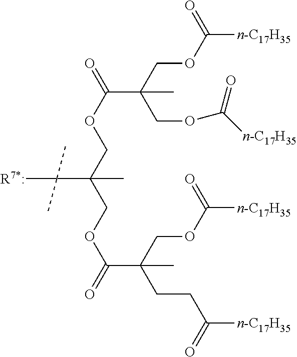

Therein, the branching structures are derived from 2,2′-dihydroxymethylpropionic acid, and the terminal groups are based on stearic acid.

It is also within the scope of the invention as defined above that in the branched or dendrimeric group R⁷*, the terminal groups —X—C(O)-T are not linked directly to the groups B of a branching structure, but are linked to the (—O—) groups of the branching structures by hydrocarbon groups such as optionally substituted or heteroatom-group-containing alkylenes or alkenylenes, preferably n-alkylenes having 2 to 10 carbon atoms, poly(alkylene oxide) groups such as poly (ethylene oxide) or poly (propylene oxide) groups, or in particular by oligo- or polyester groups, i.e. by estolide chains.

In the following example, the stearic acid-based groups —X—C(O)-T are linked to the branching structures by an estolide chain:

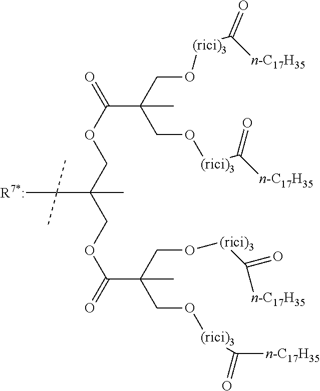

with

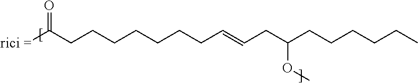

In the same manner, it is also within the scope of the invention as defined above that in the dendrimeric group R⁷*, the one or more branching elements of the structure —(C(O)—B(—O—)_{b} are not directly attached to a branching element of the structure —B(—O—)_{b} or —(C(O)—B(—O—)_{b}, but via hydrocarbon groups such as optionally substituted or heteroatom-group-containing alkylenes or alkenylenes, preferably n-alkylenes having 2 to 10 carbon atoms, poly(alkylene oxide) groups such as poly (ethylene oxide) or poly (propylene oxide) groups, or in particular by oligo- or polyester groups, i.e. by estolide chains.

In the following example, the branching structures are linked by estolide chains:

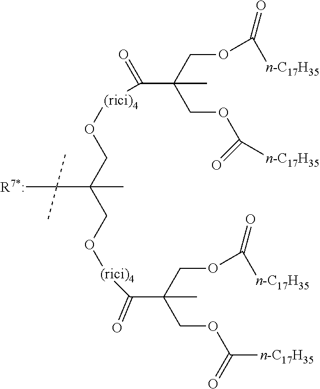

with

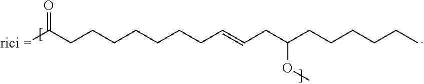

According to the invention, the residue R¹ in formula (IVa) is defined in the same manner as for formula (Ia), except for it is a q-valent residue, and q is 2 to 55, preferably 2 to 40, more preferably 2 to 4, which indicates that the residue R¹ in formula (IVa) bears q residues of the structure (—CO—X-G), with G being as defined below. Accordingly, the term “q-valent” does not refer to or restrict the number of optional further substituents other than (—CO—X-G) of the residue R¹ in formula (IVa), which can be carboxylic groups or hydroxyl groups.

The hydrocarbyl structure of R¹ of formula (IVa), which is q-valent regarding the residues (—CO—X-G), is preferably selected from the group consisting of linear, branched or cyclic alkylene groups, linear, branched or cyclic alkenylene groups, linear, branched or cyclic alkynylene groups, linear, branched or cyclic alkarylene groups, linear, branched or cyclic aralkylene groups and linear, branched or cyclic arylene groups, for instance phenylene, benzylene or tolylene groups, in particular from such groups having 1 to 1000 carbon atoms, more particular 1 to 150 carbon atoms.

Preferably, the hydrocarbon structures are linear or branched alkylene groups, or linear or branched alkylene groups interrupted by ether groups, ester groups or both ether and ester groups in particular branched structures derived from products as obtained by esterification of polyols with mono- or polyhydroxycarboxylic acids with up to 150 carbon atoms, or linear alkylene groups with up to 22 carbon atoms. Also preferred are C2-C₆ linear alkenylene groups, in particular 1,2-ethenylene radicals derived from maleic acid or fumaric acid.

More preferably, the q-valent R¹ radical of formula (IVa) is selected from alkylene groups, which may be selected from the group consisting of linear, branched and cyclic alkylene groups, in particular from linear C1-C22 alkyl groups such as methylene, ethylene, n-propylene, n-butylene, n-pentylene, n-hexylene, n-heptylene or n-octylene groups, branched C1-C22 alkylene groups iso-propylene, iso-butylene, tert-butylene, iso-butylene, tert-pentylene, neo-pentylene, and 2-ethylhexylene groups. Particularly preferred are 1,1-methylene, 1,2-ethylene, 1,3-propylene, 1,2,3-propylene and 1,4-butylene radicals.

There is no limitation regarding which C-atoms of the hydrocarbyl radicals bear the (—CO—X-G) groups attached to R¹ in formula (IVa).

Regarding the presence of functional groups optionally contained in R¹ of formula (IVa) and optional substituents, it is preferred that R¹ of the formula (IVa) is derived from glycidyl compounds, glycerol and glycerol derivatives, in particular glycidol, glycerol diglycidyl ether, diglycidyl ether and polyglycerol compounds, more particular from compounds derived from the condensation product of glycidol, glycerol, glycerol diglycidyl ether, diglycidyl ether and polyglycerol compounds and C8-C24 monohydroxy fatty acids, in particular ricinoleic acid, lesquerolic acid or 12-hydroxyl stearic acid, or when R¹ is a linear alkylene group, in particular an alkylene group not bearing further substituents in addition to the (—C(O)—X-G) groups.

It is clear to the skilled person that the groups (—C(O)—X-G) are usually attached to the radical R1 via —C(O)—X— units, in particular —C(O)—X— units, at positions which are substituted by —C(O)OH groups in a parent compound from which R¹ is derived.

For example, the R¹ group derived from succinic acid is a 1,2-ethylene radical, wherein “1,2” indicates the positions at which the radical is substituted by the (—C(O)—X-G)-groups.

According to the invention, X, R¹⁰, R⁶ and m are defined in the same manner for formula (IVa) and (Va) as for formula (Ia) and (IIa).

As defined above, R¹¹ is independently selected from optionally substituted straight-chain, cyclic or branched, saturated or unsaturated hydrocarbon radicals which have 1 to 1000 carbon atoms, optionally containing one or more groups selected from —O—, —NH—, —C(O)—, —C(S)—, tertiary amino groups

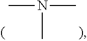

quaternary ammonium groups

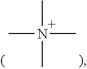

and which can be substituted with carboxyl, hydroxyl, or halide groups.

Thus R¹¹ may represent a hydrocarbyl group selected from the group consisting of linear, branched or cyclic alkyl groups, linear, branched or cyclic alkenyl groups, linear, branched or cyclic alkynyl groups, linear, branched or cyclic alkaryl groups, linear, branched or cyclic aralkyl groups and linear, branched or cyclic aryl groups, for instance phenyl, benzyl or tolyl, in particular from such groups having 6 to 24 carbon atoms, each optionally containing one or more functional groups as indicated above.

More preferably, the R¹¹ radical is selected from linear alkyl groups and linear alkenyl groups, in particular from linear C6-C24 alkyl groups such as hexyl, heptyl, octyl, nonyl, decyl, undecyl, dodecyl, tridecyl, tetradecyl, pentadecyl, hexadecyl, heptadecyl, octadecylene, nonadecyl, eicosyl, henicosyl, doicosyl, tricosyl, and tetraicosyl, or linear C6-C24 alkenyl groups such as hexenyl, heptenyl, octenyl, nonenyl, decenyl, undecenyl, dodecenyl, tridecenyl, tetradecenyl, pentadecenyl, hexadecenyl, heptadecenyl, octadecenyl, nonadecenyl, eicosenyl, henicosenyl, doicosenyl, tricosenyl, and tetraicosenyl, wherein the residues may bear one or more hydroxyl or carboxyl substituents, and wherein the groups are most preferably bonded to the adjacent C(O) group or X group by a terminal C-atom.

There is no limitation regarding at which C-atoms of the hydrocarbyl radicals the adjacent groups X group are attached to R¹¹.

However, R¹¹ is preferably derived from monoalcohols, diols or polyols bearing more than two OH-groups, or from monohydroxy carboxylic acid or carboxylic acids bearing more than one hydroxylic groups, more preferably from a monoalcohol or a monohydroxy carboxylic acid, most preferably from C6-C24 fatty acids with one hydroxyl group as substituent. Accordingly, R¹¹ preferably represents the alkyl or alkenyl chain of such carboxylic acids.

Preferred examples of R¹¹ are the structures formally derived from a corresponding hydroxyl carboxylic acid by abstraction of an hydroxyl group, wherein the hydroxyl carboxylic acid may be selected from monohydroxy carboxylic acids, such as lesquerolic acid, ricinoleic acid, 10-hydroxy octadecanoic acid, 12-hydroxy octadecanoic acid, 14-hydroxy tetradecanoic acid, 10-hydroxy stearic acid, 12-hydroxy stearic acid, or from dihydroxy carboxylic acids, in particular 2,2′-di-hydroxymethyl propanoic acid, 9,10-dihydroxy stearic acid, or from polyhydroxy carboxylic acids, in particular gluconic acid.

Likewise, preferred examples of R¹¹ are the structures formally derived from the corresponding mono- and diols by formal abstraction of one hydroxyl group, wherein the alcohols may be selected from the group consisting of methanol, ethanol, 2-propanol, 1-butanol, t-butanol, undec-10-en-ol, oleyl alcohol, stearyl alcohol, 1,2,-propanediol, 1,3-propanediol, 1,3-butanediol, 1,4-butanediol, 1,2 hexanediol, 1,6-hexanediol.

Further preferred examples of R¹¹ are compounds formally derived as described above from glycerol, diglycerol, triglycerol, and linear or branched oligoglycerols formally comprising from 4 to 6 glycerol units, trimethylol propane, castor oil (ricinoleic acid triglyceride), lesquerella oil (lesquerolic acid triglyceride), pentaerythritol, sorbitol, polyalkylene oxides, such as ethylene oxide-, propylene oxide- and/or butylene oxide-based polyethers.

Although the radical R¹¹ can optionally contain one or more groups selected from —O—, —NH—, —C(O)—, —C(S)—, tertiary amino groups

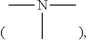

quaternary ammonium groups

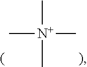

and may be substituted with OH groups or carboxyl groups, the radical R⁷ cannot contain a combination of a —C(O)— group and a —O— group or a combination of a —C(O)— group and a —NH— or tertiary amino group forming an internal carboxylate group, i.e. an internal ester group, or an internal amide group.

According to the invention, it is mandatory that in the compound of the formula (IVa) in at least one moiety of the formula (Va) R¹¹ has at least 2, preferably at least 6 carbon atoms, and in the same moiety of the formula (Va) at least one R⁶ has at least 6, preferably at least 8 carbon atoms.

According to the invention, it is also mandatory that the compound of the formula (Ia) is not exclusively composed of glycerol and ricinoleic acid moieties, i.e. it is not a polyglycerol polyricinolate (PGPR). Therein, polyglycerol polyricinolate (PGPR) is as defined above according to the invention.

In general, the compound according to the invention and the embodiments in which it is provided are the same as the compound of the above described hair care composition and its embodiments, except that for the compound according to the invention, m cannot be 0 but is at least 1.

In a preferred embodiment according to the invention, a compound according to the general formula (Ia) or (IVa) is provided wherein R¹ is as defined above, having up to 10000 carbon atoms, preferably up to 1000, more preferably up to 300, even more preferably up to 100, most preferably up to 50 carbon atoms.

Preferably, R¹ of the formula (Ia) according to this embodiment is selected from linear or branched alkylene groups, or linear or branched alkylene groups interrupted by ether groups, ester groups or both ether and ester groups, in particular branched structures derived from products as obtained by esterification of polyols with mono- or polyhydroxycarboxylic acids, or linear alkylene groups.

More preferably, the R¹ radical of the formula (Ia) according to this embodiment is selected from alkylene groups selected from the group consisting of linear and branched alkylene groups, in particular from linear C1-C22 alkyl groups such as methylene, ethylene, n-propylene, n-butylene, n-pentylene, n-hexylene, n-heptylene or n-octylene groups, branched C1-C22 alkylene groups iso-propylene, iso-butylene, tert-butylene, iso-butylene, tert-pentylene, neo-pentylene, and 2-ethylhexylene groups.

Regarding the presence of functional groups optionally contained in the R¹ radical of the formula (Ia) according to this embodiment and its optional substituents, it is preferred that R¹ is derived from glycidyl compounds, glycerol and glycerol derivatives, in particular glycidol, glycerol, glycerol diglycidyl ether, diglycidyl ether and polyglycerol compounds, or when R¹ is a linear alkylene group, in particular an alkylene group not bearing further substituents in addition to the (—X—C(O)—F) groups is preferred, and it is even more preferred when R¹ is derived from the condensation product of glycidol, glycerol, glycerol diglycidyl ether, diglycidyl ether and polyglycerol compounds and C8-C24 monohydroxy fatty acids, in particular ricinoleic acid, lesquerolic acid or 12-hydroxyl stearic acid.

Preferably, R¹ of the formula (IVa) according to this embodiment is selected from linear or branched alkylene groups, or linear or branched alkylene groups interrupted by ether groups, ester groups or both ether and ester groups in particular branched structures derived from products as obtained by esterification of polyols with mono- or polyhydroxycarboxylic acids, or linear alkylene groups. Also preferred are C2-C₆ linear alkenylene groups, in particular 1,2-ethenylene radicals derived from maleic acid or fumaric acid.

More preferably, the R¹ radical of formula (IVa) is selected from alkylene groups, which may be selected from the group consisting of linear or branched alkylene groups, in particular from linear C1-C22 alkyl groups such as methylene, ethylene, n-propylene, n-butylene, n-pentylene, n-hexylene, n-heptylene or n-octylene groups, branched C1-C22 alkylene groups iso-propylene, iso-butylene, tert-butylene, iso-butylene, tert-pentylene, neo-pentylene, and 2-ethylhexylene groups. Particularly preferred are 1,1-methylene, 1,2-ethylene, 1,3-propylene, 1,2,3-propylene and 1,4-butylene radicals.

Regarding the presence of functional groups optionally contained in R¹ of formula (IVa) and optional substituents, it is preferred that R¹ of the formula (IVa) is derived from glycidyl compounds, glycerol and glycerol derivatives, in particular glycidol, glycerol diglycidyl ether, diglycidyl ether and polyglycerol compounds, more particular from compounds derived from the condensation product of glycidol, glycerol, glycerol diglycidyl ether, diglycidyl ether and polyglycerol compounds and C8-C24 monohydroxy fatty acids, in particular ricinoleic acid, lesquerolic acid or 12-hydroxyl stearic acid, or when R¹ is a linear alkylene group, in particular an alkylene group not bearing further substituents in addition to the (—C(O)—X-G) groups.

In another preferred embodiment according to the invention, a compound according to the general formula (Ia) or (IVa) is provided wherein the number of carbon atoms in any R⁷ or R¹¹ of the compound is from 3 to 300, preferably 3 to 100, more preferably 3 to 50, even more preferably 3 to 36, further preferably 3 to 24, and most preferably 11 to 24.

According to this embodiment, R⁷ is preferably independently selected from optionally substituted linear, branched or cyclic alkyl groups, linear, branched or cyclic alkenyl groups, linear, branched or cyclic alkynyl groups, linear, branched or cyclic alkaryl groups, linear, branched or cyclic aralkyl groups and linear, branched or cyclic aryl groups, for instance phenyl, benzyl or tolyl, in particular from such groups having 6 to 24 carbon atoms, each optionally containing one or more functional groups as indicated above.

More preferably, the R⁷ radical is selected from linear alkyl groups and linear alkenyl groups, in particular from linear C6-C24 alkyl groups such as hexyl, heptyl, octyl, nonyl, decyl, undecyl, dodecyl, tridecyl, tetradecyl, pentadecyl, hexadecyl, heptadecyl, octadecylene, nonadecyl, eicosyl, henicosyl, doicosyl, tricosyl, and tetraicosyl, or linear C6-C24 alkenyl groups such as hexenyl, heptenyl, octenyl, nonenyl, decenyl, undecenyl, dodecenyl, tridecenyl, tetradecenyl, pentadecenyl, hexadecenyl, heptadecenyl, octadecenyl, nonadecenyl, eicosenyl, henicosenyl, doicosenyl, tricosenyl, and tetraicosenyl, wherein the groups are most preferably bonded to the adjacent C(O) group or X group by a terminal C-atom.

According to this embodiment, R¹¹ is preferably independently selected from optionally substituted linear, branched or cyclic alkyl groups, linear, branched or cyclic alkenyl groups, linear, branched or cyclic alkynyl groups, linear, branched or cyclic alkaryl groups, linear, branched or cyclic aralkyl groups and linear, branched or cyclic aryl groups, for instance phenyl, benzyl or tolyl, in particular from such groups having 6 to 24 carbon atoms, each optionally containing one or more functional groups as indicated above.

More preferably, the R¹¹ radical is selected from linear alkyl groups and linear alkenyl groups, in particular from linear C6-C24 alkyl groups such as hexyl, heptyl, octyl, nonyl, decyl, undecyl, dodecyl, tridecyl, tetradecyl, pentadecyl, hexadecyl, heptadecyl, octadecylene, nonadecyl, eicosyl, henicosyl, doicosyl, tricosyl, and tetraicosyl, or linear C6-C24 alkenyl groups such as hexenyl, heptenyl, octenyl, nonenyl, decenyl, undecenyl, dodecenyl, tridecenyl, tetradecenyl, pentadecenyl, hexadecenyl, heptadecenyl, octadecenyl, nonadecenyl, eicosenyl, henicosenyl, doicosenyl, tricosenyl, and tetraicosenyl, wherein the residues may bear one or more hydroxyl or carboxyl substituents, and wherein the groups are most preferably bonded to the adjacent C(O) group or X group by a terminal C-atom.

In still another preferred embodiment according to the invention, a compound according to the general formula (Ia) or (IVa) is provided, wherein the compound of the formula (Ia) or (IVa) has a molecular weight in the range of from 1500 to 200000 g/mol, preferably 1500 to 100000 g/mol, more preferably 1500 to 30000 g/mol, even more preferably 1500 to 10000 g/mol, further preferably 1500 to 5000 g/mol, and most preferably 1500 to 3000 g/mol.

In a more preferred embodiment according to the invention, a compound according to the general formula (Ia) or (IVa) is provided, wherein the compound of the formula (Ia) contains 2 to 100 moieties of the formula (IIa), more preferably 2 to 50 moieties of the formula (IIa), even more preferably 2 to 20, further preferably 2 to 10, and even further preferably 2 to 6, and most preferably 2 to 4 moieties of the formula (IIa), or wherein the compound of the formula (Ia) contains 2 to 100 moieties of the formula (IIa*), more preferably 2 to 50 moieties of the formula (IIa*), even more preferably 2 to 20, further preferably 2 to 10, and even further preferably 2 to 6, and most preferably 2 to 4 moieties of the formula (IIa*), or wherein the compound of the formula (IVa) contains 2 to 100 moieties of the formula (Va), more preferably 2 to 50 moieties of the formula (Va), even more preferably 2 to 20, further preferably 2 to 10, and even further preferably 2 to 6, and most preferably 2 to 4 moieties of the formula (Va).

In the present invention, the presence of the moieties of the formula (IIa) or (Va), i.e. the estolide moieties of the compounds, is decisive for obtaining and modulating the desired properties of the preferably fatty-acid based compounds and thus of the hair care formulations containing these compounds.

In a preferred embodiment according to the invention, a compound according to the general formula (Ia) is provided, which is represented by the general formula (IIIa):

{[(F—C(O)—X—)₁₋₅R³(—X—C(O)—)]₁₋₃R⁴—C(O)—X—}ᵣ—R²(—X—C(O)—F)ₛ (IIIa)

wherein

X can be the same or different and is selected from —O—, or —NR¹⁰—, wherein R¹⁰ is selected from the group consisting of hydrogen, or optionally substituted straight-chain, cyclic or branched, saturated, unsaturated or aromatic hydrocarbon radicals which have up to 100 carbon atoms which optionally contain one or more groups selected from —O—, —NH—, —C(O)—, —C(S)—, tertiary amino groups

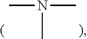

or in formula (IIIa) R¹⁰ may form a bond to R² to form a cyclic structure,

R² in formula (IIIa) is selected from (r+s)-valent, optionally substituted hydrocarbon radicals which have up to 1000 carbon atoms, and may contain optionally one or more groups selected from —O—, —NH—, —C(O)—, —C(S)—, tertiary amino groups and quaternary ammonium groups, and can be optionally substituted by one or more carboxylic groups or hydroxyl groups, and optionally forms a bond to the nitrogen atom in the group —NR¹⁰—, in case R¹⁰ is a bond to R²,

R³ is selected from di- to hexavalent, optionally substituted hydrocarbon radicals which have up to 1000 carbon atoms, and may contain optionally one or more groups selected from —O—, —NH—, —C(O)—, —C(S)—, tertiary amino groups

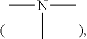

wherein if a plurality of R³ is present in formula (IIIa), they can be the same or different,

R⁴ is selected from divalent to tetravalent optionally substituted straight-chain, cyclic or branched, saturated, unsaturated or aromatic hydrocarbon radicals which have up to 300 carbon atoms, which optionally contain one or more groups selected from —O—, —NH—, —C(O)—, —C(S)—, tertiary amino groups

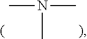

wherein if a plurality of R⁴ is present in formula (IIIa), they can be the same or different,

and wherein

r+s=2 to 55,

r=0 to 54,

s≥1, and in formula (IIIa) F can be the same or different and is selected from optionally substituted straight-chain, cyclic or branched, saturated, unsaturated or aromatic hydrocarbon radicals which have up to 1005 carbon atoms, which optionally contain one or more groups selected from —NH—, —C(O)—, —C(S)— and tertiary amino groups

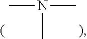

and can be optionally substituted by one or more selected from carboxyl, hydroxyl or halide groups,

with the proviso that at least one of the radicals F contains at least one moiety of the formula (IIa) or (IIa*):

—R⁶(—X—C(O)—R⁶)ₘ—X—C(O)—R⁷ (IIa)

—R⁶(—X—C(O)—R⁶)ₘ—X—C(O)—R⁷* (IIa*)

wherein

X is as defined above,

with m=1 to 20, preferably m=2 to 20,

R⁶ is independently selected from a divalent optionally substituted straight-chain, cyclic or branched, saturated or unsaturated hydrocarbon radical which have up to 36 carbon atoms,

R⁷ is independently selected from optionally substituted straight-chain, cyclic or branched, saturated or unsaturated hydrocarbon radicals which have 1 to 1000 carbon atoms, which optionally contain one or more groups selected from —O—, —NH—, —C(O)—, —C(S)—, tertiary amino groups

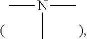

quaternary ammonium groups

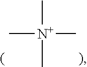

and which can be substituted with carboxyl, hydroxyl, or halide groups, wherein the radical R⁷ cannot contain an internal carboxy group or amide, i.e. R⁷ cannot contain a combination of a —C(O)— group and a —O— group or a combination of a —C(O)— group and a —NH— or tertiary amino group, and with the proviso that in at least one moiety of the formula (IIa) R⁷ has at least 2, preferably at least 6 carbon atoms,

and in the same moiety of the formula (IIa) at least one R⁶ has at least 6, preferably at least 8 carbon atoms, R⁷* is independently selected from optionally substituted branched or dendrimeric hydrocarbon radicals which have 1 to 1000 carbon atoms, optionally containing one or more groups selected from —O—, —NH—, —C(O)—, —C(S)—, tertiary amino groups

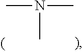

quaternary ammonium groups

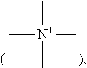

and which can be substituted with carboxyl, hydroxyl, or halide groups, wherein the radical R⁷* is terminated by two or more groups of the general structure

—X—C(O)-T

wherein X is as defined above, and

T is a monovalent straight-chain, cyclic or branched, saturated or unsaturated hydrocarbon radical optionally substituted with carboxyl, hydroxyl, or halide groups with up to 36 carbon atoms,

with the proviso that in at least one moiety of the formula (IIa*) R⁷* is terminated by one or more groups T having at least 2, preferably at least 6 carbon atoms, and in the same moiety of the formula (IIa*) at least one R⁶ has at least 6, preferably at least 8 carbon atoms.

According to the invention, R², R³, R⁴, X, R⁶, R⁷, R⁷* r and s for the compound of the formula (IIIa) are as defined above for the compound of the formula (III) of the hair care formulation, and m is also defined in the same manner as for formula (III) except that it cannot be 0, but is at least 1.

In a further preferred embodiment according to the invention, a compound of the formula (Ia), (IIIa) or (IVa) is provided, wherein R¹⁶ is selected from the group consisting of hydrogen, n-, iso-, or tert.-C₁-C₂₂-alkyl, C₂-C₂₂-alkoxyalkyl, C₅-C₃₀-cycloalkyl, C₆-C₃₀-aryl, C₆-C₃₀-aryl(C1-C₆)alkyl, C₆-C₃₀-alkylaryl, C₂-C₂₂-alkenyl, C₂-C₂₂-alkenyloxyalkyl, which optionally can be each substituted by hydroxyl and halogen, and which optionally can contain one or more ether groups (—O—), preferably R¹⁶ is selected from hydrogen or n-, iso-, and tert.-C₁-C₂₂-alkyl.

According to this embodiment, the most preferred C1-C22-alkyl groups are methyl, ethyl, n-propyl, iso-propyl, n-butyl, iso-butyl, tert-butyl, n-pentane and n-hexane groups, cyclopentyl groups and cyclohexane groups.

In another preferred embodiment according to the invention, a compound of the formula (IIIa) or (IVa) is provided, wherein R¹ in the compound of formula (IVa) or R² in the compound of formula (IIIa) is selected from optionally substituted hydrocarbon radicals which have up to 2 to 300 carbon atoms, more preferred 3 to 200 carbon atoms, even more preferred 3 to and 150 carbon atoms, specifically 3 to 50 carbon atoms, more specifically 3 to 20 carbon atoms may contain optionally one or more groups selected from —O—, —NH—, —C(O)—, —C(S)—, and tertiary amino groups

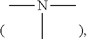

and can be optionally substituted with carboxy groups, hydroxyl groups, and quaternary ammonium groups.

According to this embodiment, R¹ in formula (IVa) or R² in the compound of the formula (IIIa) is preferably selected from the group consisting of optionally substituted linear or branched alkylene groups, or linear or branched alkylene groups interrupted by ether groups, ester groups or both ether and ester groups in particular branched structures derived from products as obtained by esterification of polyols with mono- or polyhydroxycarboxylic acids with up to 150 carbon atoms, or linear alkylene groups with up to 22 carbon atoms.

More preferably, the R¹ radical of formula (IVa) or the R² radical of formula (IIIa) is selected from alkylene groups, which may be selected from the group consisting of linear, branched alkylene groups, in particular from linear alkylene groups such as methylene, ethylene, n-propylene, n-butylene, n-pentylene, n-hexylene, n-heptylene or n-octylene groups, branched alkylene groups iso-propylene, iso-butylene, tert-butylene, iso-butylene, tert-pentylene, neo-pentylene, and 2-ethylhexylene groups.

Regarding the presence of functional groups optionally contained in R¹ of formula (IVa) or R² of the formula (IIIa) and optional substituents, it is preferred that the R¹ and R² radicals are derived from glycidyl compounds, glycerol and glycerol derivatives, in particular glycidol, glycerol, glycerol diglycidyl ether, diglycidyl ether and polyglycerol compounds, or when R¹ of formula (IVa) or R² of the formula (IIIa) is a linear alkylene group, in particular an alkylene group not bearing further substituents in addition to the (—X—C(O)—F) groups and even more preferred when R¹ is derived from the condensation product of glycidol, glycerol, glycerol diglycidyl ether, diglycidyl ether and polyglycerol compounds and C8-C24 monohydroxy fatty acids, in particular ricinoleic acid, lesquerolic acid or 12-hydroxyl stearic acid.

It is clear to the skilled person that usually the groups (—X—C(O)—F) are attached to the radical R¹ via —X—C(O)— units, in particular —O—C(O)— units, at positions which are substituted by —OH or —NHR¹⁰ groups in a parent compound from which R¹ is derived.

For example, the R¹ group derived from glycerol is a 1,2,3-propylene radical, wherein “1,2,3” indicates the positions at which the radical is substituted by the (—X—C(O)—F)-groups.

In a further preferred embodiment according to the invention, a compound of the formula (IIIa) or (IVa) is provided, wherein R¹ in the compound of formula (IVa) or R² in the compound of the formula (IIIa) is selected from divalent to hexavalent, preferred divalent to tetravalent, more preferred divalent to trivalent, in particular divalent, trivalent, tetravalent, pentavalent, or hexavalent optionally substituted hydrocarbon radicals.

In a preferred embodiment according to the invention, a compound of the formula (IIIa) is provided, wherein R³ is selected from di- to hexavalent residues.

In a further preferred embodiment according to the invention, a compound of the formula (IIIa) is provided, wherein R³ is selected from optionally substituted straight-chain, cyclic or branched, saturated, unsaturated or aromatic hydrocarbon radicals which have up to 300 carbon atoms, preferred 1 to 200 carbon atoms, more preferred 1 to 150 carbon atoms, even more preferred 1 to 50 carbon atoms, specifically 1 to 20 carbon atoms, more specifically 1 to 10 carbon atoms which optionally contain one or more groups selected from —O—, —NH—, —C(O)—, —C(S)—, tertiary amino groups

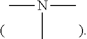

Preferred groups R³ according to this embodiment are linear C1-C22 alkylene groups such as methylene, ethylene, n-propylene, n-butylene, n-pentylene, n-hexylene, n-heptylene or n-octylene groups, branched C1-C22 alkylene groups such as iso-propylene, iso-butylene, tert-butylene, iso-butylene, tert-pentylene, neo-pentylene, and 2-ethylhexylene groups, it is more preferred when R³ when R³ is a 1,2-ethylene radical, a 1,3-propylene radical, a 1,4-butylene radical, a 1,5-pentylene radical, a 1,12-octadecylene radical, a 1,14-octadecylene radical, 1,2,3-propylene radical, a 1,2,4-butylene radical, a 1,2,5-pentylene radical, a 1,3,5-pentylene radical, a 1,2,3,4-butylene radical, a 1,2,3,4-pentylene radical, a 1,2,4,5-pentylene radical, a 1,2,3,4,5-pentylene group, or a 1,2,3,4,5,6-hexylene radical.

In another preferred embodiment according to the invention, a compound of the formula (IIIa) is provided, wherein R⁴ is selected from divalent to tetravalent, preferred divalent, trivalent, tetravalent optionally substituted straight-chain, cyclic or branched, saturated, unsaturated or aromatic hydrocarbon radicals which have 2 to 300 carbon atoms, preferred 5 to 200 carbon atoms, more preferred 8 to 150 carbon atoms, even more preferred 10 to 120 carbon atoms, which optionally contain one or more groups selected from —O—, —NH—, —C(O)—, —C(S)—, tertiary amino groups

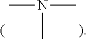

In yet another preferred embodiment according to the invention, a compound of the formula (IIIa) is provided, wherein r=0 to 50, preferred 0 to 20, more preferred 0 to 10, even more preferred 1 to 10, specifically 1 to 5, more specifically 0, 1, 2, 3, 4, 5, even more specifically r=2.

In a further preferred embodiment according to the invention, a compound of the formula (Ia) or specifically of the formula (IIIa) is provided, wherein at least one of the radicals F contains at least one moiety

—R⁶(—O—C(O)—R⁶)ₘ—O—C(O)—R⁷,

—R⁶(—NR¹⁰—C(O)—R⁶)ₘ—O—C(O)—R⁷,

—R⁶(—NR¹⁰—C(O)—R⁶)ₘ—NR¹⁰—C(O)—R⁷,

preferably —R⁶(—O—C(O)—R⁶)ₘ—O—C(O)—R⁷, wherein R¹⁰, R⁶, R⁷, and m are as defined above,

or wherein in the compound of formula (Ia) or (IIIa):

at least one of the radicals F contains at least one moiety selected from the moieties

—R⁶(—O—C(O)—R⁶)ₘ—O—C(O)—R⁷*,

—R⁶(—NR¹⁰—C(O)—R⁶)ₘ—O—C(O)—R⁷*,

—R⁶(—NR¹⁰—C(O)—R⁶)ₘ—NR¹⁰—C(O)—R^{7*},

preferably —R⁶(—O—C(O)—R⁶)ₘ—O—C(O)—R⁷*, wherein R¹⁰, R⁶, R⁷*, and m are as defined above.

Preferably, in the above structures R⁶ is independently selected from optionally hydroxyl-substituted hexylene, heptylene, octylene, nonylene, decylene, undecylene, dodecylene, tridecylene, tetradecylene, pentadecylene, hexadecylene, heptadecylene, octadecylene, nonadecylene, eicosylene, henicosylene, doicosylene, tricosylene, and tetraicosylene, or hexenylene, heptenylene, octenylene, nonenylene, decenylene, undecenylene, dodecenylene, tridecenylene, tetradecenylene, pentadecenylene, hexadecenylene, heptadecenylene, octadecenylene, nonadecenylene, eicosenylene, henicosenylene, doicosenylene, tricosenylene, and tetraicosenylene, wherein the groups are most preferably bonded to the adjacent C(O) group or X group by a terminal C-atom,

R⁷ is independently selected from optionally hydroxyl-substituted hexyl, heptyl, octyl, nonyl, decyl, undecyl, dodecyl, tridecyl, tetradecyl, pentadecyl, hexadecyl, heptadecyl, octadecylene, nonadecyl, eicosyl, henicosyl, doicosyl, tricosyl, and tetraicosyl, or hexenyl, heptenyl, octenyl, nonenyl, decenyl, undecenyl, dodecenyl, tridecenyl, tetradecenyl, pentadecenyl, hexadecenyl, heptadecenyl, octadecenyl, nonadecenyl, eicosenyl, henicosenyl, doicosenyl, tricosenyl, and tetraicosenyl, wherein the groups are most preferably bonded to the adjacent C(O) group by a terminal C-atom, and

R⁷* is as defined in any of the embodiments according to the invention,

m is 1-10, preferably 1, 2, 3, 4 or 5.

In an also preferred embodiment according to the invention, a compound of the formula (IVa) is provided, wherein at least one of the radicals G contains at least one moiety

—R⁶(—C(O)—O—R⁶)ₘ—C(O)—O—R¹¹,

—R⁶(—C(O)—NR¹⁰—R⁶)ₘ—C(O)—O—R¹¹,

—R⁶(—C(O)—NR¹⁰—R⁶)ₘ—C(O)—NR¹⁰—R¹¹,

preferably —R⁶(—C(O)—O—R⁶)ₘ—C(O)—O—R¹¹, wherein R¹⁰, R⁶, R¹¹, and m are as defined above for formula (IVa).

Preferably, in the above structures according to this embodiment

R⁶ is selected from independently selected from optionally hydroxyl-substituted hexylene, heptylene, octylene, nonylene, decylene, undecylene, dodecylene, tridecylene, tetradecylene, pentadecylene, hexadecylene, heptadecylene, octadecylene, nonadecylene, eicosylene, henicosylene, doicosylene, tricosylene, and tetraicosylene, or hexenylene, heptenylene, octenylene, nonenylene, decenylene, undecenylene, dodecenylene, tridecenylene, tetradecenylene, pentadecenylene, hexadecenylene, heptadecenylene, octadecenylene, nonadecenylene, eicosenylene, henicosenylene, doicosenylene, tricosenylene, and tetraicosenylene, wherein the groups are most preferably bonded to the adjacent C(O) group or X group by a terminal C-atom,

R¹¹ is independently selected from optionally hydroxyl-substituted hexyl, heptyl, octyl, nonyl, decyl, undecyl, dodecyl, tridecyl, tetradecyl, pentadecyl, hexadecyl, heptadecyl, octadecylene, nonadecyl, eicosyl, henicosyl, doicosyl, tricosyl, and tetraicosyl, or hexenyl, heptenyl, octenyl, nonenyl, decenyl, undecenyl, dodecenyl, tridecenyl, tetradecenyl, pentadecenyl, hexadecenyl, heptadecenyl, octadecenyl, nonadecenyl, eicosenyl, henicosenyl, doicosenyl, tricosenyl, and tetraicosenyl, wherein the residues may bear one or more hydroxyl or carboxyl substituents, and wherein the groups are most preferably bonded to the adjacent C(O) group by a terminal C-atom, and

m is 1-10, preferably 1, 2, 3, 4 or 5.

The R⁶ and R⁷ containing ester elements in the moieties (II) and (IIa) or the R⁶ and R¹¹ containing ester elements in the moieties (V) and (Va):

—R⁶(—X—C(O)—R⁶)ₘ—X—C(O)—R⁷ (II) or (IIa) and

—R⁶(—C(O)—X—R⁶)ₘ—C(O)—X—R¹¹ (V) or (Va),

in particular in the moieties

—O—C(O)—R⁶—(O—C(O)—R⁶)ₘ—O—C(O)—R⁷ and

—NR¹⁰—C(O)—R⁶—(O—C(O)—R⁶)ₘ—O—C(O)—R⁷,

and the R⁶ and R⁷* containing ester elements in the moieties (II*) and (IIa*)

—R⁶(—X—C(O)—R⁶)ₘ—X—C(O)—R⁷* (II) or (IIa),

in particular in the moieties

O—C(O)—R⁶—(O—C(O)—R⁶)ₘ—O—C(O)—R⁷* and

—NR¹⁰—C(O)—R⁶—(O—C(O)—R⁶)ₘ—O—C(O)—R⁷*,

can be synthesized from the corresponding carboxylic acids by esterification using methods known in the prior art. In a preferred embodiment these esterifications can be carried out thermally at 150-350° C. preferred at 180 to 250° C. under reduced pressure (US2011/0282084, GB 841554, DE 694943). Additionally, catalysts can be used to run the esterifications (EP 3009494, WO 2012069386, DD 150064, CH 151317, T. A. Isbell, Grasas y Aceites, 2011, 62(1), 8-20).

In another preferred embodiment, enzymes are used to condensate the carboxylic acids (JP 05304966, JP 05211878, JP 01016591, A. Bodalo et al., Biochem. Eng. J., 2008, 39(3), 450-456, A. Bodalo et al., Biochem. Eng. J. 2005, 26(2-3), 155-158, Y. Yasuko et al., J. Am. Oil Chem. Soc., 1997, 74(3), 261-267). In general, the above described methods provide polymodal condensates.

In general monomodal condensates can be synthesized by a condensation sequence based on the stepwise esterification of carboxylic acid anhydrides (K. Meier, Farbe and Lack, 1951, 57, 437-439, F. H. H. Valentin, J. South African Chem. Inst. 1949, 2, 59-61) or, preferred, carboxylic acid chlorides (K. D. Pathak et al., J. Scientific & Industrial Research, 1955, 14B, 637-639) with OH groups of hydroxylated carboxylic acids and their derivatives.

Repetitions of a cycle based on an esterification and an acid chloride synthesis provide in general monomodal ester condensates. Further details will be outlined in the example section.

Below, a schematic representation of a sequence for the synthesis of ester condensates based on the stepwise esterification of carboxylic acid chlorides and OH groups of hydroxylated carboxylic acids and their derivatives is given:

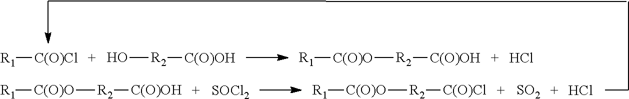

Herein, the arrow indicates that the product obtained by esterification of an acyl chloride of a fatty acid R₁—C(O)Cl by reaction with the hydroxyl-carboxylic acid HO—R₂—C(O)OH and subsequent formation of an acyl chloride by reaction with SOCl₂ can be resubmitted to such reaction sequence. Accordingly, in the next reaction sequence R₁ of the starting material R₁—C(O)Cl is “R₁—C(O)O—R₂” of the previous reaction sequence. Thus, estolide structures can be obtained in an iterative manner, and the number of fatty acid residues comprised by the final estolide moiety is determined by the number of iteration steps.

Carboxylic acids free of OH groups terminate the chains of the ester condensates, and according to the above scheme, the reaction sequence needs to start by formation of an acyl chloride of such “terminal” carboxylic acid. Monohydroxy carboxylic acids extend the chains in the ester condensates. Di- and polyhydroxy carboxylic acids can provide branched and dendrimeric (self-repeating) elements within the ester condensates. The synthesis of dendrimeric structures of 2,2′-di-hydroxymethyl propanoic acid is described in US 2016/0102179.

In a further preferred embodiment of the invention, a compound of the formula (Ia), (IIIa) or (IVa) as defined above is provided, wherein low melting and high melting fatty acids ≥C5 are specifically positioned within the R⁶ and R⁷ containing ester elements of the general formula (IIa) and within the R⁶ and R¹¹ containing ester elements of the general formula (Va):

—R⁶(—X—C(O)—R⁶)ₘ—X—C(O)—R⁷ (IIa) and

—R⁶(—C(O)—X—R⁶)ₘ—C(O)—X—R¹¹ (Va),

in particular in the moieties

—O—C(O)—R⁶—(O—C(O)—R⁶)ₘ—O—C(O)—R⁷ and

—NR¹⁰—C(O)—R⁶—(O—C(O)—R⁶)ₘ—O—C(O)—R⁷,

wherein X, R⁶, R⁷, R¹⁰ and R¹¹ are as defined above,

or within the R⁶ and R⁷* containing ester elements of the general formula (IIa*)

—R⁶(X—C(O)—R⁶)ₘ—X—C(O)—R^{7*} (IIa*),

in particular in the moieties

—O—C(O)—R⁶—(O—C(O)—R⁶)ₘ—O—C(O)—R⁷*,

wherein X, R⁶, R⁷* are as defined above.

It is in general within the scope of the invention that low melting and high melting fatty acids ≥C5 are specifically positioned independently for individual ester groups of moieties selected from the moieties of the general formulas (IIa) and (Va) present in the compounds of the general formulas (Ia), (IIIa) and (IVa), or for individual ester groups of moieties selected from the moieties of the general formulas (IIa*) present in the compounds of the general formulas (Ia), (IIIa) and (IVa).

For instance, it is according to this embodiment of the invention if a number of moieties of the general formula (IIa) displays the specific positioning of low melting fatty acids and high melting fatty acid scaffolds as described in the following while other moieties of the general formula (IIa) do not. This may be in particular the case for moieties present in different residues F as defined above.

Within the frame of the present invention, low melting fatty acids ≥C5 are defined by a melting point ≤40° C. Preferred examples are in particular oleic acid, ricinoleic acid, octanoic acid, decanoic acid, pivalinic acid, and neodecanoic acid.

Within the frame of the present invention, high melting fatty acids ≥C5 are defined by a melting point >40° C. Preferred examples are in particular dodecanoic acid, tetradecanoic acid, hexadecanoic acid, octadecanoic acid, arachidic acid, behenic acid, 10-hydroxy octadecanoic acid, 12-hydroxy octadecanoic acid, and 14-hydroxy tetradecanoic acid.

The corresponding melting points can be taken from the literature (G. Knothe et al., J. Am. Oil Chem. Soc., 2009, 86, 844-856).

In a further preferred embodiment according to the invention, a compound of the formula (Ia) or (IIIa) as defined above is provided, wherein at least one, wherein in one or more moieties of the formula (IIa) at least one, preferred more than one, more preferred one, two or three low melting fatty acids ≥C5 each forming a group R⁶ are contained in the radical or the radicals R⁶ adjacent to R⁷, while at least one, preferred more than one, more preferred one, two or three high melting fatty acids ≥C5 form the radical or radicals R⁶ at the opposite terminus of a R⁶- and R⁷-containing ester element of the formula (IIa), or in such a manner that in one or more moieties of the formula (IIa) at least one, preferred more than one, more preferred one, two or three high melting fatty acids ≥C5 each forming R⁶ form the radical or radicals R⁶ adjacent to R⁷, while at least one, preferred more than one, more preferred one, two or three low melting fatty acids ≥C5 form the radical or radicals R⁶ at the opposite terminus of a R⁶— and R⁷-containing ester element of the formula (IIa).

According to this embodiment, it is preferable when at least 50% of all moieties of the formula (IIa) display such a positioning of R⁶-radicals derived from high-melting and low-melting fatty acids as described above, and it is more preferred when more than 80% of all moieties of the formula (IIa) display such a positioning of R⁶-radicals derived from high-melting and low-melting fatty acids as described above, and most preferably all moieties of the general formula (IIa) display such positioning of residues R⁶ and R⁷ as described above.

In a likewise preferred embodiment, a compound of the formula (IVa) as defined above is provided, wherein in one or more moieties of the formula (Va) at least one, preferred more than one, more preferred one, two or three low melting fatty acids ≥C5 each forming a group R⁶ are contained in the radical or the radicals R⁶ adjacent to R¹¹, while at least one, preferred more than one, more preferred one, two or three high melting fatty acids ≥C5 form the radical or radicals R⁶ at the opposite terminus of a R⁶- and R¹¹-containing ester element of the formula (Va), or in such a manner that in one or more moieties of the formula (Va) least one, preferred more than one, more preferred one, two or three high melting fatty acids ≥C5 each forming R⁶ form the radical or radicals R⁶ adjacent to R¹¹, while at least one, preferred more than one, more preferred one, two or three low melting fatty acids ≥C5 form the radical or radicals R⁶ at the opposite terminus of a R⁶- and R¹¹-containing ester element of the formula (Va).

It is preferable when at least 50% of all moieties of the formula (Va) display such a positioning of R⁶-radicals derived from high-melting and low-melting fatty acids as described above, and it is more preferred when more than 80% of all moieties of the formula (Va) display such a positioning of R⁶-radicals derived from high-melting and low-melting fatty acids as described above, and most preferably all moieties of the general formula (Va) display such positioning of residues R⁶ and R¹¹ as described above.

In another likewise preferred embodiment, a compound of the formula (Ia) or (IIIa) as defined above is provided, wherein in one or more moieties of the formula (IIa*) at least one, preferred more than one, more preferred one, two or three low melting fatty acids with 5 or more carbon atoms and a melting point of 40° C. or below each forming a group R⁶ are contained in the radical or the radicals R⁶ adjacent to R⁷*, while at least one, preferred more than one, more preferred one, two or three high melting fatty acids with 5 or more carbon atoms and a melting point above 40° C. form the radical or radicals R⁶ at the opposite terminus of a R⁶- and R⁷*-containing ester element of the formula (IIa*), or in one or more moieties of the formula (IIa*) at least one, preferred more than one, more preferred one, two or three high melting fatty acids ≥C5 each forming R⁶ form the radical or radicals R⁶ adjacent to R⁷*, while at least one, preferred more than one, more preferred one, two or three low melting fatty acids with 5 or more carbon atoms and a melting point below 40° C. form the radical or radicals R⁶ at the opposite terminus of a R⁶- and R⁷*-containing ester element of the formula (IIa*), and it is preferable when at least 50% of all moieties of the formula (IIa*) display such a positioning of R⁶-radicals derived from high-melting and low-melting fatty acids as described above, it is more preferred when more than 80% of all moieties of the formula (IIa*) display such a positioning of R⁶-radicals derived from high-melting and low-melting fatty acids as described above, and it is most preferably when all moieties of the general formula (IIa) display such positioning of residues R⁶ and R⁷* as described above.

As already stated above, the specific positioning of high and low melting fatty acids may be independently varied for each individual R⁶- and R⁷-containing ester moiety of the formula (IIa) of a compound of the general formula (Ia) or (IIIa), and in a fully analogous way each individual R⁶- and R¹¹-containing ester moiety of the formula (Va) of a compound of the general formula (IVa).

The above outlined preferred embodiments allow the incorporation of R⁶ and R⁷ containing ester elements and R⁶- and R¹¹-containing ester elements having a locally varying tendency towards crystallization, viscosity build up and phase formation over the whole length of these ester elements in

—R⁶(—X—C(O)—R⁶)ₘ—X—C(O)—R⁷ (IIa) and

—R⁶(—C(O)—X—R⁶)ₘ—C(O)—X—R¹¹ (Va),

in particular in the moieties

—O—C(O)—R⁶—(O—C(O)—R⁶)ₘ—O—C(O)—R⁷ and

—NR¹⁰—C(O)—R⁶—(O—C(O)—R⁶)ₘ—O—C(O)—R⁷,

wherein X, R⁶, R⁷, R¹⁰ and R¹¹ are as defined above,

or

the incorporation of R⁶- and R⁷*-containing ester elements each having a locally varying tendency towards crystallization, viscosity build up and phase formation over the whole length of these ester elements in

—R⁶(—X—C(O)—R⁶)ₘ—X—C(O)—R⁷* (IIa*),

in particular in the moieties

—O—C(O)—R⁶—(O—C(O)—R⁶)ₘ—O—C(O)—R⁷* and

—NR¹⁰—C(O)—R⁶—(O—C(O)—R⁶)ₘ—O—C(O)—R⁷,

wherein X, R⁶, R⁷*, R¹⁰ and R¹¹ are as defined above.

The combination of the above mentioned carboxylic acids and synthetic concepts gives access to ester condensates having defined molecular weights, molecular weight distributions, carboxylic acid sequences and properties such as viscosity.

The radicals R¹, R² or R³ can be linked to the R⁶ and R⁷-containing ester elements in the moieties of the general formula (IIa) present in the groups F,

in particular by formation of groups of the general structure

—R⁶—(O—C(O)—R⁶)ₘ—O—C(O)—R⁷ and

—NR¹⁰—C(O)—R⁶—(O—C(O)—R⁶)ₘ—O—C(O)—R⁷,

preferably by esterification of
monohydroxyl carboxylic acids, in particular lactic acid, 4-hydroxy butanoic acid,
dihydroxy carboxylic acids, in particular 2,2-bis(hydroxymethyl) propanoic acid,
polyhydroxy carboxylic acids, in particular gluconic acid or the dendrimeric oligomers of dihydroxy carboxylic acid oligomers, in particular dendrimeric oligomers of 2,2-bis(hydroxymethyl) propanoic acid,

with the corresponding acid chloride precursors Cl—C(O)—R⁶—(O—C(O)—R⁶)ₘ—O—C(O)—R⁷ wherein X, R⁶, and R⁷ and R¹⁰ are as defined above.

In general,

the R⁶ and R^{x} containing ester elements of the structures

{[—R⁶—O—C(O)-]ₘ}ₙR^{x} or

—[R⁶—O—C(O)]ₘ—R^{x}—,

which may for instance be present in or constitute the residue R⁴ in the general formulas (III) or (IIIa),

wherein R⁶ is as defined above,

m is 1-10,

n is 2 to 4, and

R^{x} is a single bond or a C1 to C34 hydrocarbon radical, preferably derived from substituted straight-chain, cyclic or branched, saturated or unsaturated poly carboxylic acids which have 2 to 36 carbon atoms, preferred 2 to 24 carbon atoms, more preferred 2 to 18 carbon atoms, even more preferred 4 to 18 carbon atoms,
preferably derived from dicarboxylic acids, in particular from oxalic acid, malonic acid, malic acid, tartaric acid, maleic acid, itaconic acid, succinic acid, sebacic acid, dimer acid,

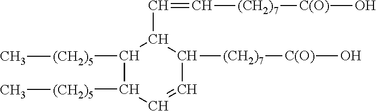

the amide condensation products of amino acids with maleic acid or succinic acid, in particular of N-maleoyl-μ-alanine, N-succinyl-μ-alanine, N-maleoyl-asparagine, the ester condensation products of divalent alcohols, in particular of ethylene glycol, 1,2-propylene glycol, 1,3-propylene glycol, 1,3-butane diol, 1,4-butanediol with dicarboxylic acid anhydrides, in particular maleic anhydride, succinic anhydride, phthalic anhydride,
preferably derived from tricarboxylic acids, in particular citric acid, isocitric acid, mellitic acid, the bis-amide condensation products of amino acids with maleic acid or succinic acid, in particular bis-(N-maleoyl)-lysine, the ester condensation products of trivalent alcohols, in particular glycerol, trimethylolpropane with dicarboxylic acid anhydrides, in particular maleic anhydride, succinic anhydride, phthalic anhydride,
preferably derived from tetracarboxylic acids, in particular pyromellitic acid, cyclobutane tetracarboxylic acid, the ester condensation products of tetravalent alcohols, in particular diglycerol, pentaerythritol with dicarboxylic acid anhydrides, in particular with maleic anhydride, succinic anhydride, phthalic anhydride,

can be synthesized using the principals outlined for the R⁶ and R⁷ containing ester elements. The carboxylic acids providing R⁷ are replaced by the ones providing R^{x}.

Preferably, intermediates finally yielding the radical {[—R⁶—O—C(O)-]ₘ}ₙR^{x} can be synthesized by reaction of the acid chloride precursors bearing R^{X} with hydroxylated precursors bearing R^{x}.

Also preferably, intermediates finally yielding the radical —[R⁶—O—C(O)]ₘ—R^{x}—, can be synthesized by reaction of the acid anhydride precursors bearing R^{x} with the hydroxylated precursors bearing R⁶.

In a further preferred embodiment according to the invention, a compound of the general formula (Ia), (IIIa) or (IVa) is provided, wherein m=1 to 10, more preferred 1 to 6, even more preferred 2 to 6, specifically 1, 2, 3, 4, 5, 6, more specifically 1 or 2.

In a further preferred embodiment according to the invention, a compound of the general formula (Ia) or of the formula (IIIa) or (IVa) is provided, wherein:

R⁶ is independently selected from optionally substituted straight-chain, cyclic or branched, saturated or unsaturated hydrocarbon radicals which have up to 24 carbon atoms, preferred 1 to 24, more preferred 2 to 20 carbon atoms, even more preferred 8 to 18 carbon atoms.

According to the embodiment, preferably, R⁶ is selected from radicals derived from lesquerolic acid, ricinoleic acid, 12-hydroxy stearic acid or 14-hydroxy stearic acid, specifically from ricinoleic acid.

In another further preferred embodiment according to the invention, a compound of the general formula (Ia) or specifically of the formula (IIIa) is provided, wherein R⁷ is independently selected from optionally substituted straight-chain, cyclic or branched, saturated or unsaturated hydrocarbon radicals which have 1 to 36 carbon atoms, preferred 1 to 24 carbon atoms, more preferred 1 to 18 carbon atoms, even more preferred 8 to 18 carbon atoms.

The halide groups are independently selected from fluoro, chloro, bromo or iodo groups, wherein chloro groups are preferred.

According to this embodiment, preferably the R⁷ radical is derived from oleic acid or stearic acid, specifically from oleic acid.

In a further preferred embodiment according to the invention, a compound of the general formula (Ia) or specifically of the formula (IIIa) is provided, wherein the total number of carbon atoms in R⁶+R⁷ (Σcarbon atoms R⁶, R⁷) in each single moiety of the general formula (IIa) composed of R⁶ and R⁷ is 10 to 300, preferred 15 to 200, more preferred 20 to 150, even more preferred 30 to 100, or

wherein in the compound of formula (Ia) or (IIIa) the total number of carbon atoms in R⁶+R⁷* (Σcarbon atoms R⁶, R⁷*) in each single moiety of the general formula (IIa*) composed of R⁶ and R⁷* is 10 to 300, preferred 15 to 200, more preferred 20 to 150, even more preferred 30 to 100.

In an also preferred embodiment according to the invention, a compound of the formula (IVa) is provided, wherein the total number of carbon atoms in R⁶+R¹¹ (Σcarbon atoms R⁶, R¹¹) in each single moiety of the general formula (a) composed of R⁶ and R⁷ is 10 to 300, preferred 15 to 200, more preferred 20 to 150, even more preferred 30 to 100.

In a still further preferred embodiment according to the invention, a compound of the general formula (Ia) or specifically of the formula (IIIa) or (IVa) is provided, wherein R⁶ is derived from monohydroxy carboxylic acids with up to 25 carbon atoms, preferably independently selected from the group consisting of glycolic acid, lactic acid, 2-hydroxy butyric acid, 3-hydroxy-butyric acid, 4-hydroxy butyric acid, 14-hydroxy tetradecanoic acid, 10-hydroxy stearic acid, 12-hydroxy stearic acid, ricinoleic acid, and lesquerolic acid.

In another preferred embodiment according to the invention, a compound of the general formula (Ia) or specifically of the formula (IIIa) is provided, wherein R⁷ is derived from carboxylic acids with up to 25 carbon atoms which do not have hydroxyl substituent, preferably independently selected from the group consisting of acetic acid, propanoic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, docosanoic acid, 2-ethyl hexanoic acid, 2,2-dimethyl propionic acid, 2,2-dimethyl heptanoic acid, 2,2-dimethyl octanoic acid, neodecanoic acid, undecyl-10-en-ic acid, oleic acid, linoleic acid, linolenic acid, and erucic acid.

Most preferably, according to this embodiment R⁷ is derived from oleic acid, linoleic acid or linolenic acid, in particular when R⁷ is combined in a moiety of the formula (IIa) with one or more R⁶ groups derived from ricinoleic acid or lesquerolic acid.

In yet another preferred embodiment according to the invention, a compound of the general formula (Ia), which may be specifically represented by the formula (IIIa), is provided, wherein at least one, preferably both of R⁶ and R⁷ of the moieties of the general formula (IIa) are derived from unsaturated carboxylic acids.

In a further preferred embodiment according to the invention, a compound of the formula (IIIa) is provided wherein R⁴ is selected from divalent to tetravalent, such as divalent, trivalent, tetravalent, preferably divalent, optionally substituted straight-chain, cyclic or branched, saturated, unsaturated or aromatic hydrocarbon radicals which have up to 200 carbon atoms, more preferred up to 150 carbon atoms, even more preferred up to 100 carbon atoms, specifically up to 80 carbon atoms, and preferably has at least 2, more preferred at least 10, more preferred as least 14 carbon atoms, which optionally contain one or more groups selected from —O—, —NH—, and —C(O)—, and wherein if a plurality of R⁴ is present in formula (IIIa), they can be the same or different.

In another preferred embodiment according to the invention, a compound of the formula (IIIa) is provided wherein in the compound of the general formula (IIIa) R⁴ comprises at least one ester group (—O—C(O)—, or —C(O)—O—, respectively).

In still another preferred embodiment according to the invention, a compound of formula (IIIa) is provided, wherein in the compound of formula (IIIa) R⁴ is derived from dicarboxylic acids, tricarboxylic acids or tetracarboxylic acids, in particular dicarboxylic acids, such as succinic acid, oxalic acid, malonic acid, malic acid, tartaric acid, maleic acid, itaconic acid, succinic acid, sebacic acid, dimer acids, amino-functional dicarboxylic acids, such as D-glutamic acid, and a dicarboxylic acid of the formula:

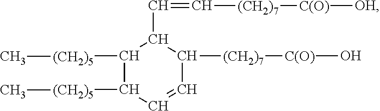

and condensation products of hydroxy carboxylic acids, in particular, from ricinoleic acid or lesquerolic acid, and dicarboxylic acids, such as succinic acid, oxalic acid, malonic acid, malic acid, tartaric acid, maleic acid, itaconic acid, succinic acid, sebacic acid, dimer acids, amino-functional dicarboxylic acids, such as D-glutamic acid, and a dicarboxylic acid of the formula:

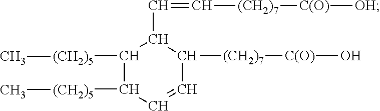

or

R⁴ is derived from amide condensation products of amino acids with maleic acid or succinic acid, such as N-maleoyl-β-alanine ((E)-4-(2-carboxyethylamino)-4-oxo-but-2-enoic acid), N-succinyl-β-alanine (4-[(2-hydroxy-1-methyl-2-oxo-ethyl)amino]-4-oxo-butanoic acid), N-maleoyl-asparagine (4-amino-2-[[(E)-4-hydroxy-4-oxo-but-2-enoyl]amino]-4-oxo-butanoic acid); or R⁴ is derived from the ester condensation products of divalent alcohols, i.e. ethylene glycol, 1,2-propylene glycol, 1,3-propylene glycol, 1,3-butane diol, 1,4-butanediol, with dicarboxylic acid anhydrides, i.e. maleic anhydride, succinic anhydride, phthalic anhydride; or

R⁴ is derived from tri- or tetracarboxylic acids, such as citric acid, isocitric acid, trimelletic acid, pyromellitic acid, cyclobutane tetracarboxylic acid; or

R⁴ is derived from the bisamide condensation products of amino acids with maleic acid or succinic acid, i.e. bis-(N-maleoyl)-lysine; or

R⁴ is derived from the ester condensation products of trivalent alcohols, such as glycerol, trimethylolpropane with dicarboxylic acid anhydrides, such as maleic anhydride, succinic anhydride, phthalic anhydride; or

R⁴ is derived from the ester condensation products of tetravalent alcohols, such as diglycerol, pentaerythritol with dicarboxylic acid anhydrides, such as maleic anhydride, succinic anhydride, phthalic anhydride, and

most preferred R⁴ is derived from the condensation products of ricinoleic acid or lesquerolic acid and succinic acid.

In a preferred embodiment of the hair care formulation according to the invention containing at least one compound of the formula (Ia), (IIIa) or (IVa), in at least one of the moieties of the formula (IIa), (IIa*) or (Va) two or more different R⁶ groups are present.

The presence of at least two different groups R⁶ in the moieties of the formula (IIa), (IIa*) or (Va) results when at least two different types of hydroxy-substituted or amino-substituted carboxylic acid derivatives are used in the preparation of these chain structures. The different groups R⁶ may differ from each other in the number of C atoms, but also with regards to the number and position of double bonds, if any, and/or the position of substituents and the position of the linkage to the adjacent groups. They may differ with regards to if they are linear or branched. It is preferred when in at least one group F R⁶ independently represents hydrocarbon groups derived from ricinoleic acid and 12-hydroxy stearic acid.

In another preferred embodiment of the hair care formulation according to the invention containing at least one compound of the formula (Ia), (IIIa) or (IVa), in at least one of the moieties of the formula (IIa), (IIa*) or (Va) the groups R⁶ and R⁷ in formula (IIa), the groups R⁶ and R⁷* in formula (IIa*), or R⁶ and R¹¹ in formula (Va) are not based on the same carboxylic acid structure.

It is preferred that R⁶ and R⁷, R⁶ and R⁷* or R⁶ and R¹¹ differ from each other regarding their number of carbon atoms, the number or position of double bonds, if any, in the carbon chain, or regarding the position of oxygen or nitrogen atoms bonded to the carbon chain of the groups. The carboxylic acid structures from which said groups are derived may also differ by two or more of the above-mentioned features.

In still another preferred embodiment of the hair care formulation according to the invention containing at least one compound of the formula (Ia) or (IIIa), in the compound of the general formula (Ia)

p is 2-6,

R¹ is selected from di- to hexavalent linear, branched or cyclic alkylene groups, linear, branched or cyclic alkenylene groups, linear, branched or cyclic alkynylene groups, linear, branched or cyclic alkarylene groups, linear, branched or cyclic aralkylene groups and linear, branched or cyclic arylene groups, for instance phenylene, benzylene or tolylene groups, in particular from such groups having 1 to 1000 carbon atoms, more particular 1 to 150 carbon atoms,

and at least one group F contains one or more moieties of the general formula (IIa*)

—R⁶(—X—C(O)—R⁶)ₘ—X—C(O)—R⁷* (IIa*),

wherein R⁶, R⁷* and m are as defined above,

or wherein in a compound of the general formula (IIIa)

r+s=2-6,

R² is selected from di- to hexavalent linear, branched or cyclic alkylene groups, linear, branched or cyclic alkenylene groups, linear, branched or cyclic alkynylene groups, linear, branched or cyclic alkarylene groups, linear, branched or cyclic aralkylene groups and linear, branched or cyclic arylene groups, for instance phenylene, benzylene or tolylene groups, in particular from such groups having 1 to 1000 carbon atoms, more particular 1 to 150 carbon atoms,

and at least one group F contains one or more moieties of the general formula (IIa*),
wherein R⁶, R⁷* and m are as defined above.

According to this embodiment, it is preferred that in formula (Ia) p is 2, 3 or 4, most preferably p is 2, or it is preferred that in formula (IIIa) r+s=2, 3 or 4, most preferably r+s is 2.

It is also preferred that preferred that in the compound of the general formula (Ia) R¹ is selected from linear, branched or cyclic alkylene groups having 1 to 150 carbon atoms, more preferably linear alkylene groups having 1 to 12 carbon atoms, or it is preferred that in formula (IIIa) R² is selected from linear, branched or cyclic alkylene groups having 1 to 150 carbon atoms, more preferably linear alkylene groups having 1 to 12 carbon atoms.

In a further preferred embodiment of the hair care formulation according to the invention containing at least one compound of the formula (Ia) or (IIIa), in the least one compound of the general formula (Ia)

X═O,

p is 2,

R¹ is selected from divalent linear, branched and cyclic alkylene groups, in particular from linear C1-C22 alkyl groups such as methylene, ethylene, n-propylene, n-butylene, n-pentylene, n-hexylene, n-heptylene or n-octylene groups, branched C1-C22 alkylene groups iso-propylene, iso-butylene, tert-butylene, iso-butylene, tert-pentylene, neo-pentylene, and 2-ethylhexylene groups, preferably from ethylene, n-propylene, n-butylene, n-pentylene and n-hexylene,

and at least one group F contains one or more moieties of the general formula (IIa*)

—R⁶(—X—C(O)—R⁶)ₘ—X—C(O)—R⁷* (IIa*),

wherein R⁶, R⁷* and m are as defined above,

or wherein in the compound of the general formula (IIIa)

X═O,

r+s=2,

R² is selected from divalent linear, branched and cyclic alkylene groups, in particular from linear C1-C22 alkyl groups such as methylene, ethylene, n-propylene, n-butylene, n-pentylene, n-hexylene, n-heptylene or n-octylene groups, branched C1-C22 alkylene groups iso-propylene, iso-butylene, tert-butylene, iso-butylene, tert-pentylene, neo-pentylene, and 2-ethylhexylene groups, preferably from ethylene, n-propylene, n-butylene, n-pentylene and n-hexylene, and at least one group F contains one or more moieties of the general formula (IIa*), wherein R⁶, R⁷* and m are as defined above.

In still another preferred embodiment of the hair care formulation according to the invention containing at least one compound of the formula (Ia), (IIIa) or (IVa), the compound of the formula (Ia) or (IIIa) contains one or more groups R⁷* each terminated by three or more groups —O—C(O)-T, preferably by 4 or more groups —O—C(O)-T, most preferably by 4 to 12 groups —O—C(O)-T.

Therein, it is preferred that branched structures of R⁷* containing one branching structure as defined above are terminated by 3 to 10 groups —O—C(O)-T, while dendrimeric structures containing at least two branching structures as defined above are preferably terminated by 4 to 20 groups —O—C(O)-T.

In a further preferred embodiment of the hair care formulation according to the invention containing at least one compound of the formula (Ia), (IIIa) or (IVa), in the compound of the formula (Ia) or (IIIa) one or more groups R⁷* each contain at least two branching structures of the general formula

—C(O)—B(—O—)_{b},

wherein B is a linear or branched hydrocarbon group having 2-20 carbon atoms, and b is 2 or more, and wherein the b groups (—O—) linked to the group B on the one side are linked to a C atom which may be the C atom of a CH₂ group or of a carbonyl group on the other side.

While the presence of a branching structure of the general formula

—B(—O—)_{b}

as defined above is mandatory in the group R⁷* in order to enable a branched structure which may be terminated by two or more groups —O—C(O)-T, the presence of two or more further branching structures of the general formula

—C(O)—B(—O—)_{b}

as defined above results in the formation of a dendrimeric structure, i.e. a structure having several branching points which may be arranged consecutively or parallel when moving from the bond linking R⁷* to the rest of the molecule to the terminal groups of R⁷*. However, deviating from the IUPAC definition of a dendrimer molecule [see A. Fradet et al., Pure and Applied Chemistry, 91(3), 523-561: Nomenclature and terminology for dendrimers with regular dendrons and for hyperbranched polymers (IUPAC Recommendations 2017)] the dendrons do not have to comprise exclusively dendritic and terminal constitutional repeating units, and it is not required that each path from the free valence of R⁷*, i.e. the valence bonding R⁷* to the rest of the molecule, to any end-group comprises the same number of constitutional repeating units.

It is further preferred that all (—O—) groups of the branching structure of the general formula —B(—O—)_{b} as defined above are substituted by the branching structures of the general formula

—C(O)—B(—O—)_{b} as defined above.

It is also preferred that one or more groups R⁷* contain 3 or more branching structures —C(O)—B(—O—)_{b}, more preferred 3-5 of said branching structures.

In this embodiment, it is preferred that b for both branching structures is independently selected from the range of 2-6, more preferably from the range of 2-4.

In a preferred embodiment of the hair care formulation according to the invention containing at least one compound of the formula (Ia), (IIIa) or (IVa), in the compound of the formula (Ia) or (IIIa) the one or more branching structures of the general formal —B(—O—)_{b} or —C(O)—B(—O—)_{b} as defined above of at least one group R⁷* are independently derived from glyceric acid, 2,2-di-hydroxymethyl propionic acid, gluconic acid, maltobionic acid, lactobionic acid

It is preferred that all branching structures present in a group R⁷* are independently derived from 2,2-di-hydroxymethyl propionic acid, more preferably all branching structures in at least one group R⁷* are derived from 2,2-di-hydroxymethyl propionic acid.

It is even further preferred that all branching structure present in all groups R⁷* of a compound of the formula (Ia) or (IIIa) are derived from the same polyhydroxy carboxylic acid.

In another preferred embodiment of the hair care formulation according to the invention, in the compound of the formula (Ia) or (IIIa) one or more groups R⁷* are each terminated by two or more groups of the general formula

—R⁶(—X—C(O)—R⁶)ₜ—X—C(O)-T,

wherein R⁶, and T are as defined above, and

X═O,

t is independently 0-12, preferably t is independently 0-6, most preferably t is independently 0, 1, 2 or 3.

In this embodiment, two or more of the terminal groups as defined above are positioned at the terminus of an estolide chain. It is preferred that the one or more groups R⁷* are each terminated by 2-48 groups of the general formula —R⁶(—X—C(O)—R⁶)ₜ—X—C(O)-T, more preferable by 2-27 groups of the formula —R⁶(—X—C(O)—R⁶)ₜ—X—C(O)-T, and most preferable by 4-16 groups of the formula —R⁶(—X—C(O)—R⁶)ₜ—X—C(O)-T.

In a further preferred embodiment of the hair care formulation according to the invention, one or more groups R⁷* of the compound of the formula (Ia) or (IIIa) are terminated by two or more groups, preferably 4 to 12 groups of the structure

—R⁶(—X—C(O)—R⁶)ₜ—X—C(O)-T,

wherein R⁶ is independently derived from C8-C24 monocarboxy-monohydroxy carboxylic acids, in particular ricinoleic acid, 12-hydroxy stearic acid, lesquerolic acid, 11-hydroxy-undecanoic acid,

X is O, and

T is independently derived from C2 to C24, preferred C8 to C24 fatty acids, in particular lauric acid, myristic acid, palmitic acid, oleic acid, stearic acid, behenic acid, arachidic acid, and t is 0-6, preferably 0, 1, 2 or 3.

According to this embodiment it is preferred when R⁶ is derived from ricinoleic acid, and T is derived from stearic acid or oleic acid.

More preferably, the R⁶ of all groups R⁷* are derived from ricinoleic acid, and even more preferably in all groups R⁷* R⁶ is derived from ricinoleic acid, T is derived from stearic acid or oleic acid, and t is 0, 1, 2 or 3.

In another preferred embodiment of the hair care formulation according to the invention, one or more groups R⁷* of the compound of the formula (Ia) or (IIIa) are independently selected from one of the following branched or dendrimeric fatty acid structures:

—R¹²—O—C(O)—R⁶—(O—C(O)—R⁶¹)ₘ₁—O—C(O)—R⁶²)ₘ₂—O—C(O)-T or

—R¹²—NR¹⁰—C(O)—R⁶—(O—C(O)—R⁶¹)ₘ₁—(O—C(O)—R⁶²)ₘ₂—O—C(O)-T, wherein

R¹² is selected from divalent optionally substituted hydrocarbon radicals which 2 to and 50 carbon atoms, specifically 2 to 20 carbon atoms, more specifically 2 to 10 carbon atoms and may contain optionally one or more groups selected from —O—, —NH—, —O(O)—, —C(S)—, tertiary amino groups

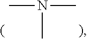

and can be substituted by —OH or halide groups, wherein the radical R¹⁰ cannot contain a combination of a —C(O)— group and a —O— group or a combination of a —C(O)— group and a —NH— or tertiary amino group forming an internal carboxylate group or an internal amide group, and preferably represents C1-024 n-alkylene groups and CC2-C24 n-alkenylene groups, in particular-CH₂—, —CH₂CH₂—, —CH₂CH₂CH₂—,

R⁶ is as defined above,

m1 is 0 to 12, preferred 0 to 10, more preferred 0 to 6, even more preferred 1 to 6, specifically 0, 1, 2, 3, 4, 5, 6,

m2 is 0 to 12, preferred 0 to 10, more preferred 0 to 6, even more preferred 1 to 6, specifically 0, 1, 2, 3, 4, 5, 6,

and m1+m2 is t, wherein t is 0 to 12, preferred 0 to 10, more preferred 0 to 6, even more

preferred 1 to 6, specifically 0, 1, 2, 3, 4, 5, 6 and

T is as defined above,

R⁶¹ and R⁶² are selected from the groups R⁶ as defined above.

Preferably, R⁶, R⁶¹, R⁶² and T are selected as follows:

| R⁶ adjacent to R¹² is | R⁶¹ adjacent to R⁶ is | R⁶² adjacent to R⁷ is | |
|---|---|---|---|
| derived from | derived from | derived from | T is derived from |
| di- or polyhydroxylated | unsaturated acid, in | unsaturated acid, in | unsaturated acid, in |
| acid, in particular | particular ricinoleic | particular ricinoleic | particular oleic acid |
| 2,2′-di-hydroxymethyl | acid | acid | |
| propanoic acid | | | |
| di- or polyhydroxylated | unsaturated acid, in | unsaturated acid, in | saturated acid, in |
| acid, in particular | particular ricinoleic | particular ricinoleic | particular octadecanoic |
| 2,2′-di-hydroxymethyl | acid | acid | acid, neodecanoic acid |
| propanoic acid | | | |
| di- or polyhydroxylated | unsaturated acid, in | saturated acid, in | saturated acid, in |
| acid, in particular | particular ricinoleic | particular 12- | particular octadecanoic |
| 2,2′-di-hydroxymethyl | acid | hydroxystearic acid | acid, neodecanoic acid |
| propanoic acid | | | |
| di- or polyhydroxylated | saturated acid, in | saturated acid, in | saturated acid, in |
| acid, in particular | particular 12- | particular 12- | particular octadecanoic |
| 2,2′-di-hydroxymethyl | hydroxystearic acid | hydroxystearic acid | acid, neodecanoic acid |
| propanoic acid | | | |
| di- or polyhydroxylated | saturated acid, in | saturated acid, in | unsaturated acid, in |
| acid, in particular | particular 12- | particular 12- | particular oleic acid |
| 2,2′-di-hydroxymethyl | hydroxystearic acid | hydroxystearic acid | |
| propanoic acid | | | |
| di- or polyhydroxylated | saturated acid, in | unsaturated acid, in | unsaturated acid, in |
| acid, in particular | particular 12- | particular ricinoleic | particular oleic acid |
| 2,2′-di-hydroxymethyl | hydroxystearic acid | acid | |
| propanoic acid | | | |
| di- or polyhydroxylated | di- or polyhydroxylated | unsaturated acid, in | unsaturated acid, in |
| acid, in particular | acid, in particular | particular ricinoleic | particular oleic acid |
| 2,2′-di-hydroxymethyl | 2,2′-di-hydroxymethyl | acid | |
| propanoic acid | propanoic acid | | |
| di- or polyhydroxylated | di- or polyhydroxylated | unsaturated acid, in | saturated acid, in |
| acid, in particular | acid, in particular | particular ricinoleic | particular 12- |
| 2,2′-di-hydroxymethyl | 2,2′-di-hydroxymethyl | acid | hydroxystearic acid |
| propanoic acid | propanoic acid | | |
| di- or polyhydroxylated | di- or polyhydroxylated | saturated acid, in | unsaturated acid, in |
| acid, in particular | acid, in particular | particular 12- | particular oleic acid |
| 2,2′-di-hydroxymethyl | 2,2′-di-hydroxymethyl | hydroxystearic acid | |
| propanoic acid | propanoic acid | | |
| di- or polyhydroxylated | di- or polyhydroxylated | saturated acid, in | saturated acid, in |
| acid, in particular | acid, in particular | particular 12- | particular 12- |
| 2,2′-di-hydroxymethyl | 2,2′-di-hydroxymethyl | hydroxystearic acid | hydroxystearic acid |
| propanoic acid | propanoic acid | | |
| di- or polyhydroxylated | unsaturated acid, in | di- or polyhydroxylated | unsaturated acid, in |
| acid, in particular | particular ricinoleic | acid, in particular | particular oleic acid or |
| 2,2′-di-hydroxymethyl | acid | 2,2′-di-hydroxymethyl | unsaturated acid |
| propanoic acid | | propanoic acid | terminated oligoester |
| di- or polyhydroxylated | saturated acid, in | di- or polyhydroxylated | saturated acid, in |
| acid, in particular | particular 12- | acid, in particular | particular octadecanoic |
| 2,2′-di-hydroxymethyl | hydroxystearic acid | 2,2′-di-hydroxymethyl | acid, neodecanoic acid |
| propanoic acid | | propanoic acid | or saturated acid |
| | | | terminated oligoester |

wherein the total number of carbon atoms in R⁶+R⁷ (Σcarbon atoms R⁶, R⁷) is 19 to 300, preferred 25 to 300, more preferred 35 to 300, even more preferred 50 to 300, specifically 35 to 200, more specifically 35 to 150, even more specifically 50 to 150,

with the proviso that for R⁶, R⁶¹ and R⁶² being derived from di- or polyhydroxylated carboxylic acids at least one, preferred one to two, more preferred two, even more preferred all OH groups are esterified.

According to this embodiment, it is also preferred that one or more groups R⁷* of the compound of the formula (Ia) or (IIIa) are independently derived from branched or dendrimeric fatty acid structures obtained by the esterification of 2,2′-di-hydroxymethyl propanoic acid with di-hydroxymethyl propanoic acid itself, C2 to C24, preferred C8 to C24 fatty acids, further preferred lauric acid, myristic acid, palmitic acid, oleic acid, stearic acid, behenic acid, arachidic acid and optionally mono hydroxy fatty acids, in particular ricinoleic acid, as exemplified by the structural formula

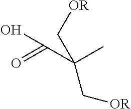

with R:

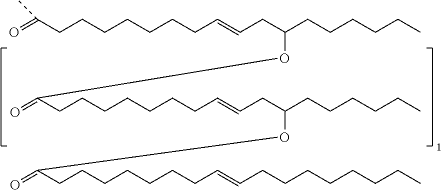

or the structure

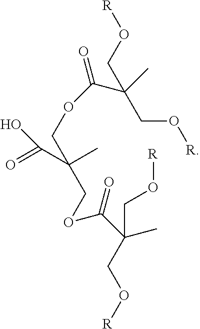

with R as displayed above.

Preferably, all groups R⁷* of the compound of the formula (Ia) or (IIIa) are independently selected from the above-cited group of structures.

Even more preferably, all groups R⁷* of the compound of the formula (Ia) or (IIIa) are represented by a single formula selected from the above-cited group of structures.

In a preferred embodiment of the hair care formulation according to the invention, at least one compound of the general formula (Ia) or (IIIa) is represented by one of the following specific structures:

i) (fatty acid)-C(O)—O-(mono or oligo C8-C24 hydroxy fatty acid)-C(O)—O-(C2-C10 hydrocarbon)-O—C(O)-(mono or oligo C8-C24 hydroxy fatty acid)-O—C(O)-(fatty acid)

or

ii) (branched fatty acid)-C(O)—O-(mono or oligo C8-C24 hydroxy fatty acid)-C(O)—O—(C2-C10 hydrocarbon)-O—C(O)-(mono or oligo C8-C24 hydroxy fatty acid)-O—C(O)-(branched fatty acid)

or

iii) (dendrimeric fatty acid)-C(O)—O-(mono or oligo C8-C24 hydroxy fatty acid)-C(O)—O—(C2-C10 hydrocarbon)-O—C(O)-(mono or oligo C8-C24 hydroxy fatty acid)-O—C(O)-(dendrimeric fatty acid),

wherein
C2-C10 hydrocarbon is a C2-C10 hydrocarbylene group, in particular derived from ethylene glycol, 1,3 propylene glycol, 1,4 butanediol, 1,6 hexanediol, 1,2 propylene glycol, 1,3 butanediol,
mono or oligo C8-C24 hydroxy fatty acid is a group derived from a C8-C24 hydroxy-substituted carboxylic acid monomer or an oligomer of up to 20 C8-C24 hydroxy-substituted carboxylic acid monomers formed via esterification, in particular derived from mono or oligo ricinoleic acid with a degree of oligomerization of 2 to 20, preferred, 2 to 10, more preferred 2 to 6, even more preferred 2 to 4,
fatty acid is a saturated or unsaturated hydrocarbyl residue derived from a C2-C24 carboxylic acid with such residue, preferably derived from such C8 to C24 fatty acids, in particular from lauric acid, myristic acid, palmitic acid, oleic acid, stearic acid, behenic acid, arachidic acid,
branched fatty acid is a residue obtained by the esterification of a polyhydroxymonocarboxylic acid, in particular of 2,2′-di-hydroxymethyl propanoic acid, with C2 to C24, preferred C8 to C24 fatty acids, in particular lauric acid, myristic acid, palmitic acid, oleic acid, stearic acid, behenic acid, arachidic acid, and optionally mono hydroxy fatty acids, in particular. ricinoleic acid,
dendrimeric fatty acid is a residue obtained by the esterification of derived from i.e. the esterification a branched fatty acid residue as described above with at least one further polyhydroxymonocarboxylic acid, in particular 2,2′-di-hydroxymethyl propanoic acid, with C2 to C24, preferred C8 to C24 fatty acids, in particular lauric acid, myristic acid, palmitic acid, oleic acid, stearic acid, behenic acid, arachidic acid and optionally mono hydroxy fatty acids, in particular ricinoleic acid.

The branched fatty acid group is exemplified by the structure

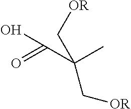

with R:

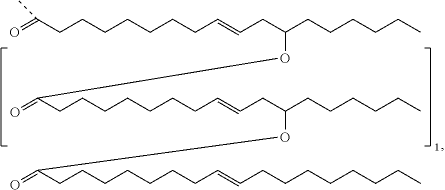

the dendrimeric fatty acid group is exemplified by the structure

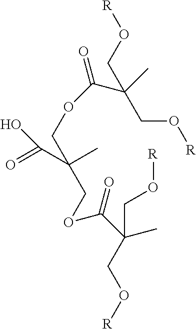

with R as displayed above for the branched fatty acid group.

In a further preferred embodiment of the hair care formulation according to the invention, the compound of the formula (Ia), (IIIa) or (IVa) comprises at least one moiety of the general formula

([—O—C(O)—R⁶(—O—C(O)—R⁶)ₗ—O—C(O)-L-C(O)—O—(R⁶—C(O)—O)ₗ—R⁶—C(O)O])—

wherein R⁶ is as defined above,

l is an integer independently selected from 0-20, more preferably from 1-12, even more preferably from 2 to 10, and

L is a divalent hydrocarbon radical which may have 1 to 30 carbon atoms and may contain optionally one or more groups selected from —O—, —S—, —NH—, —C(O)—, —C(S)—, tertiary amino groups

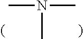

and quaternary ammonium groups, preferably L is a divalent alkylene or alkenylene radical having 1 to 30 carbon atoms, more preferably L is selected from methylene, ethylene, propylene, butylene, pentylene, hexylene, heptylene, octylene, nonylene, ethenylene, propenylene, butenylene, pentenylene, hexenylene, heptenylene, octenylene, nonenylene, most preferably L is selected from methylene, ethylene, ethenylene or butenylene.

According to the embodiment, preferably R⁶ is independently derived from C8-C24 monocarboxy-monohydroxy carboxylic acids, more preferably from ricinoleic acid, 12-hydroxy stearic acid, lesquerolic acid, 11-hydroxy-undecanoic acid, and even more preferably every R⁶ is independently derived from ricinoleic acid, 12-hydroxy stearic acid, lesquerolic acid, 11-hydroxy-undecanoic acid, and most preferably every R⁶ is the same group derived from one carboxylic acid selected from the group consisting of ricinoleic acid, 12-hydroxy stearic acid, lesquerolic acid, and 11-hydroxy-undecanoic acid.

It is explicitly noted that there may be an overlap of the structures of the moieties of the formula (Va)

—R⁶(—C(O)—X—R⁶)ₘ—C(O)—X—R¹¹ (Va)

and of the general formula

—([—O—C(O)—R⁶(—O—C(O)—R⁶)ₗ—O—C(O)-L-C(O)—O—(R⁶—C(O)—O)ₗ—R⁶—C(O)O])—

i.e. according to the invention a structure of the general formula

([—O—C(O)—R⁶(—O—C(O)—R⁶)ₗ—O—C(O)-L-C(O)—O—(R⁶—C(O)—O)ₗ—R⁶—C(O)O])—R¹¹

fulfills the requirements of the radical G of Formula (IVa).

In another preferred embodiment of the hair care formulation according to the invention, the compound of the formula (Ia), (IIIa) or (IVa) comprises at least one moiety of the general formula

([—O—C(O)—R⁶(—O—C(O)—R⁶)ₗ—O—C(O)-L-C(O)—O—(R⁶—C(O)—O)ₗ—R⁶—C(O)O])—,

wherein L and l are as defined above,

and R⁶ is independently derived from C8-C24 monocarboxy-monohydroxy carboxylic acids, in particular from ricinoleic acid, 12-hydroxy stearic acid, lesquerolic acid, 11-hydroxy-undecanoic acid, most preferably R⁶ is derived from ricinoleic acid.

Preferably, L is selected from C1 to C10, preferably methylene, ethylene, butylene, octylene, decylene, l is independently in the range of 0 to 4, and R⁶ is derived from ricinoleic acid.

In still another preferred embodiment of the hair care formulation according to the invention, the compound of the formula (Ia), (IIIa) or (IVa) comprises at least one moiety of the general formula

([—O—C(O)—R⁶(—O—C(O)—R⁶)ₗ—O—C(O)-L-C(O)—O—(R⁶—C(O)—O)ₗ—R⁶—C(O)O])—

wherein L is selected from methylene, ethylene, and ethenylene,

R⁶ is derived from ricinoleic acid, and

l is independently selected from 0, 1, 2 and 3, and the sum of l is in the range of 0-4.

Preferably, L is an ethylene group, R⁶ is derived from ricinoleic acid, and l is independently selected from 0 or 1.

In a preferred embodiment of the hair care formulation according to the invention, the compound of the formula (Ia), (IIIa) or (IVa) comprises at least one moiety of the following structure:

(fatty alcohol)-O—C(O)-(mono or oligo C8-C24 hydroxy fatty acid)-O—C(O)—(C1-C12 hydrocarbon)-C(O)—O-(mono or oligo C8-C24 hydroxy fatty acid)-C(O)—O-(fatty alcohol), wherein
C2-C12 hydrocarbon is a C2-C12 hydrocarbylene group, in particular derived from succinic acid, maleic acid, itaconic acid, adipic acid, sebacic acid, dodecanedioic acid,
mono or oligo C8-C24 hydroxy fatty acid is a group derived from a C8-C24 hydroxy-substituted carboxylic acid monomer or an oligomer of up to 20 C8-C24 hydroxy-substituted carboxylic acid monomers formed via esterification, in particular derived from mono or oligo ricinoleic acid with a degree of oligomerization of 2 to 20, preferred, 2 to 10, more preferred 2 to 6, even more preferred 2 to 4,
fatty alcohol is a group derived from i.e. C2 to C24, preferred C8 to C24 fatty alcohols, in particular from n-octanol, n-decanol, n-dodecanol, n-tetradecanol, n-hexacedanol, oleyl alcohol, stearyl alcohol, behenyl alcohol, arachidyl alcohol.

Such kind of compound is exemplified by the following structure:

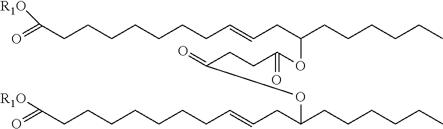

with R¹:

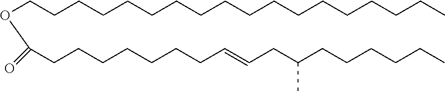 [SMILES: CCCCCCCCCCCCCCCCCCOC(=O)CCCCCCC/C=C/C[C@@H](C)CCCCCC]

(The dashed bond indicates R¹'s bond to the O atom of the structure above R¹.

In another preferred embodiment of the hair care formulation according to the invention, the compound of the formula (Ia), (IIIa) or (IVa) comprises at least one moiety of the structure

—([—O—C(O)—R⁶(—O—C(O)—R⁶)ₗ—O—C(O)-L-C(O)—O—(R⁶—C(O)—O)ₗ—R⁶—C(O))])—R¹¹,

wherein L, l, R⁶ and R¹¹ are as defined above.

Preferably, L, l and R⁶ are as defined above, and R¹¹ is selected from C1-C23 hydrocarbyl groups, preferably C1-C18 hydrocarbyl groups, more preferably C7-C19 hydrocarbyl groups, even more preferably C11-C17 hydrocarbyl groups, most preferably derived from lauric acid, myristic acid, palmitic acid, oleic acid, and stearic acid.

Further preferably, the compound of the formula (IVa) has the following structure

R¹¹—([—O—C(O)—R⁶(—O—C(O)—R⁶)ₗ—O—C(O)-L-C(O)—O—(R⁶—C(O)—O)ₗ—R⁶—C(O)O])—R¹¹,

wherein L, l and R⁶ are as defined above, and R¹¹ is independently selected C1-C23 hydrocarbyl groups, preferably C1-C18 hydrocarbyl groups, more preferably C7-C19 hydrocarbyl groups, even more preferably C11-C17 hydrocarbyl groups, and most preferably derived from lauric acid, myristic acid, palmitic acid, oleic acid, and stearic acid.

Even further preferably, L is selected from methylene, ethylene or ethenylene, butylene, hexylene, octylene, decylene or derived from itaconic acid,

l is independently selected from the range of 0-4,

R⁶ is selected derived from ricinoleic acid, and

R¹¹ is selected derived from oleic acid or stearic acid.

Use

The invention further relates to the use of the above-described polymeric fatty acid compounds of the formula (Ia), in particular of the formula (IIIa), and (IVa), as described in the above embodiments according to the invention in cosmetic formulations for skin and hair care, such as conditioners and shampoos.

The invention further relates to the use of the above-described polymeric fatty acid compounds of the formulas (I), (III), (IV), (Ia), (IIIa) and (IVa), as described in the above embodiments according to the invention for the treatment of fibers, preferred amino acid based fibers, more preferred human hair, in particular being useful for hair color retention, for hair shine enhancement, for hair color enhancement, for hair color protection, for hair conditioning, for hair smoothening or softening, for improving manageability of the hair, in particular for improving the combability of the hair, the anti-frizz and anti-static properties.

Formulations

The invention further relates to compositions that contain at least one of the polymeric fatty acid compounds of the formulas (I), (III), (IV), (Ia), (IIIa) and (IVa), as defined in the embodiments described above, together with at least one additional component that is commonly used in such a composition.

Preferred compositions containing at least one compound of the formula (I), (III) and (IV) or (Ia), (IIIa) and (Iva) as defined in the embodiments above for the treatment of hair according to the invention are selected from the group consisting of a hair shampoo composition, hair care composition, hair conditioning composition, hair coloration or dyeing composition, hair combability improving composition, anti-frizz composition, hair rinse-off and leave-on compositions.

It is generally preferred that the formulations and compositions according to the invention are aqueous formulations and compositions, i.e. they contain water, more preferably they contain more than 20 weight-% of water.

In particular, the invention relates to cosmetic compositions that contain at least one of the polymeric fatty acid compounds as defined in the embodiments described above, together with at least one additional component that is commonly used in such a composition.

Specifically, the invention relates to hair care formulations compositions that contain at least one of the polymeric fatty acid compounds as defined in the embodiments described above, together with at least one additional component that is commonly used in such a composition.

### Summary of Specific Preferred Embodiments According to the Invention

In the following, specific preferred embodiments according to the invention are summarized:

1. A hair care formulation containing at least one compound of the formula (I):

R¹(—X—C(O)—F)ₚ (I)

wherein

R¹ in formula (I) is selected from a p-valent, optionally substituted hydrocarbon radical and may contain optionally one or more groups selected from —O—, —NH—, —C(O)—, —C(S)—, tertiary amino groups

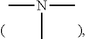

and quaternary ammonium groups, and can be optionally substituted by one or more selected from carboxyl groups or hydroxyl groups,

p≥2, more preferably 2-811,

X can be the same or different and is selected from —O—, or —NR¹⁰—, wherein R¹⁰ is selected from the group consisting of hydrogen, or optionally substituted straight-chain, cyclic or branched, saturated, unsaturated or aromatic hydrocarbon radicals which have up to 100 carbon atoms which optionally contain one or more groups selected from —O—, —NH—, —C(O)—, —C(S)—, tertiary amino groups

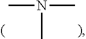

or in formula (I) R¹⁰ may form a bond to R¹ to form a cyclic structure,

F can be the same or different and is selected from optionally substituted straight-chain, cyclic or branched, saturated, unsaturated or aromatic hydrocarbon radicals which have up to 1005 carbon atoms, which optionally contain one or more groups selected from —O—, —NH—, —C(O)—, —C(S)—, and tertiary amino groups

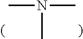

and can be optionally substituted by one or more selected from carboxyl, hydroxyl or halide groups,

with the proviso that at least one of the radicals F contains at least one moiety of the formula (II):

—R⁶(—X—C(O)—R⁶)ₘ—X—C(O)—R⁷ (II)

wherein

X is as defined above,

m=0 to 20, preferably 1 to 20,

R⁶ is independently selected from a divalent optionally substituted straight-chain, cyclic or branched, saturated or unsaturated hydrocarbon radical which have up to 36 carbon atoms,

R⁷ is independently selected from optionally substituted straight-chain, cyclic or branched, saturated or unsaturated hydrocarbon radicals which have 1 to 1000 carbon atoms, optionally containing one or more groups selected from —O—, —NH—, —C(O)—, —C(S)—, tertiary amino groups

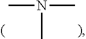

quaternary ammonium groups

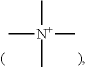

and which can be substituted with carboxyl, hydroxyl, or halide groups, wherein the radical R⁷ cannot contain an internal carboxy group or amide, i.e. R⁷ cannot contain a combination of a —C(O)— group and a —O— group or a combination of a —C(O)— group and a —NH— or tertiary amino group, with the proviso that in at least one moiety of the formula (II) R⁷ has at least 2, preferably at least 6 carbon atoms, and in the same moiety of the formula (II) at least one R⁶ has at least 6, preferably at least 8 carbon atoms,

or

containing at least one compound of the general formula (IV)

R¹(—C(O)—X-G)_{q} (IV),

wherein

X is as defined above,

R¹ in formula (IV) is selected from q-valent, optionally substituted hydrocarbon radicals which preferably have up to 1000 carbon atoms, and may contain optionally one or more groups selected from —O—, —NH—, —C(O)—, —C(S)—, tertiary amino groups

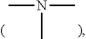

quaternary ammonium groups, and can be optionally substituted by one or more selected from carboxyl groups or hydroxyl groups,

q=2 to 55, preferably 2 to 40, more preferably 2 to 4, and

G can be the same or different and is selected from optionally substituted straight-chain, cyclic or branched, saturated, unsaturated or aromatic hydrocarbon radicals which have up to 1005 carbon atoms, which optionally contain one or more groups selected from —O—, —NH—, —C(O)—, —C(S)—, and tertiary amino groups

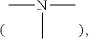

and can be substituted by one or more selected from carboxyl, hydroxyl or halide groups,

with the proviso that at least one of the radicals G contains at least one moiety of the formula (V):

—R⁶(—C(O)—X—R⁶)ₘ—C(O)—X—R¹¹ (V)

wherein

X is as defined above

m=0 to 20, preferably 1 to 20,

R⁶ in formula (V) is as defined above for formula (I),

R¹¹ is independently selected from optionally substituted straight-chain, cyclic or branched, saturated or unsaturated hydrocarbon radicals which have 1 to 1000 carbon atoms, optionally containing one or more groups selected from —O—, —NH—, —C(O)—, —C(S)—, tertiary amino groups

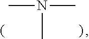

quaternary ammonium groups

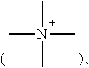

and which can be substituted with carboxyl, hydroxyl, or halide groups, wherein the radical R¹¹ cannot contain an internal carboxy group or amide, i.e. R¹¹ cannot contain a combination of a —C(O)— group and a —O-group or a combination of a —C(O)— group and a —NH— or tertiary amino group, with the proviso that in at least one moiety of the formula (V) R¹¹ has at least 2, preferably at least 6 carbon atoms, and in the same moiety of the formula (V) at least one R⁶ has at least 6, preferably at least 8 carbon atoms.

2. A hair care formulation according to embodiment 1, wherein in the compounds of formula (I) or (IV) R¹ is as defined above with up to 10000 carbon atoms, preferably up to 1000, more preferably up to 300, even more preferably up to 100, most preferably up to 50 carbon atoms.

3. A hair care formulation according to embodiment 1 or 2, wherein in the compounds of formula (I) or (IV) the number of carbon atoms in any R⁷ or R¹¹ of the compounds is from 3 to 300, preferably 3 to 100, more preferably 3 to 50, even more preferably 3 to 36, further preferably 3 to 24, and most preferably 11 to 24.

4. A hair care formulation according to any of the embodiments 1 to 3, wherein the compounds of the formula (I) or (IV) has a molecular weight in the range of from 1500 to 200000 g/mol, preferably 1500 to 100000 g/mol, more preferably 1500 to 30000 g/mol, even more preferably 1500 to 10000 g/mol, further preferably 1500 to 5000 g/mol, and most preferably 1500 to 3000 g/mol.

5. A hair care formulation according to any of the embodiments 1 to 4, wherein the compound of the formula (I) contains 2 to 100 moieties of the formula (II), more preferably 2 to 50 moieties of the formula (II), even more preferably 2 to 20, further preferably 2 to 10, and even further preferably 2 to 6, and most preferably 2 to 4 moieties of the formula (II), or wherein the compound of the formula (IV) contains 2 to 100 moieties of the formula (V), more preferably 2 to 50 moieties of the formula (V), even more preferably 2 to 20, further preferably 2 to 10, and even further preferably 2 to 6, and most preferably 2 to 4 moieties of the formula (V).

6. A hair care formulation according to any of the embodiments 1 to 5, wherein at least one compound of the general formula (I) is represented by the general formula (III)

{([(F—C(O)—X—)₁₋₅R³(—X—C(O)—)]₁₋₃R⁴—C(O)—X—}ᵣ—R²(—X—C(O)—F)ₛ (III)

wherein X is as defined above,

R² in formula (III) is selected from (r+s)-valent, optionally substituted hydrocarbon radicals which have up to 1000 carbon atoms, and may contain optionally one or more groups selected from —O—, —NH—, —C(O)—, —C(S)—, tertiary amino groups

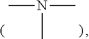

and quaternary ammonium groups, and can be optionally substituted by one or more carboxylic groups or hydroxyl groups, and optionally forms a bond to the nitrogen atom in the group —NR¹⁰—, in case R¹⁰ is a bond to R²,

R³ is selected from di- to hexavalent, optionally substituted hydrocarbon radicals which have up to 1000 carbon atoms, and may contain optionally one or more groups selected from —O—, —NH—, —C(O)—, —C(S)—, tertiary amino groups

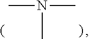

wherein if a plurality of R³ is present in formula (III), they can be the same or different,

R⁴ is selected from divalent to tetravalent optionally substituted straight-chain, cyclic or branched, saturated, unsaturated or aromatic hydrocarbon radicals which have up to 300 carbon atoms, which optionally contain one or more groups selected from —O—, —NH—, —C(O)—, —C(S)—, tertiary amino groups

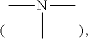

wherein if a plurality of R⁴ is present in formula (III), they can be the same or different,

and wherein

r+s=2 to 55,

r=0 to 54,

s≥1 and

in formula (III) F is as defined above with the proviso that at least one of the radicals F contains at least one moiety of the formula (II):

—R⁶(—X—C(O)—R⁶)ₘ—X—C(O)—R⁷ (II)

wherein

X, R⁶ and R⁷ are as defined above,

m=0 to 20, preferably 1 to 20,

and with the proviso that in at least one moiety of the formula (II) R⁷ has at least 2, preferably at least 6 carbon atoms, and in the same moiety of the formula (II) at least one R⁶ has at least 6, preferably at least 8 carbon atoms.

7. A hair care formulation according to any of the embodiments 1-6, wherein R¹ in the compound of formula (IV) or R² in the compound of formula (III) are selected from optionally substituted hydrocarbon radicals which have 2 to 300 carbon atoms, more preferred 3 to 200 carbon atoms, even more preferred 3 to and 150 carbon atoms, specifically 3 to 50 carbon atoms, more specifically 3 to 20 carbon atoms may contain optionally one or more groups selected from —O—, —NH—, —C(O)—, —C(S)—, and tertiary amino groups

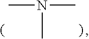

and can be optionally substituted with carboxy groups or hydroxyl groups.

8. A hair care formulation according to the embodiments 6 and 7, wherein in the compound of formula (III):

R³ is selected from di- to tetravalent residues, specifically divalent residues, trivalent residues, or tetravalent residues.

9. A hair care formulation according to the embodiments 6 to 8, wherein in the compound of formula (III):

R³ is selected from optionally substituted hydrocarbon radicals which have up to 300 carbon atoms, more preferred 3 to 200 carbon atoms, even more preferred 3 to and 150 carbon atoms, specifically 3 to 50 carbon atoms, more specifically 3 to 20 carbon atoms may contain optionally one or more groups selected from —O—, —NH—, —C(O)—, —C(S)—, tertiary amino groups

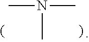

10. A hair care formulation according to any of the previous embodiments, wherein in the compound of formula (I), (III) or (IV):

F and G are selected from optionally substituted straight-chain, cyclic or branched, saturated, unsaturated or aromatic hydrocarbon radicals which have up to 200 carbon atoms, preferred 10 to 200 carbon atoms, more preferred 10 to 150, even more preferred 10 to 100 carbon atoms, which optionally contain one or more groups selected from —O—, —NH—, —C(O)—, —C(S)—, and tertiary amino groups

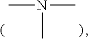

and can be optionally substituted by one or more carboxyl groups, hydroxyl groups or halide groups.

11. A hair care formulation according to the embodiments 6 to 10, wherein in the compound of formula (III):

R⁴ is selected from a divalent to tetravalent, preferred divalent, trivalent, tetravalent optionally substituted straight-chain, cyclic or branched, saturated, unsaturated or aromatic hydrocarbon radicals which have 2 to 300 carbon atoms, preferred 5 to 200 carbon atoms, more preferred 8 to 150 carbon atoms, even more preferred 10 to 120 carbon atoms, which optionally contain one or more groups selected from —O—, —NH—, —C(O)—, —C(S)—, tertiary amino groups

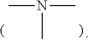

12. A hair care formulation according to the embodiments 6 to 11, wherein in the compound of formula (III):
r=0 to 50 or 1 to 50, preferred 0 to 20, more preferred 0 to 10, even more preferred 1 to 10, specifically 1 to 5, more specifically 0, 1, 2, 3, 4, 5, even more specifically 2.

13. A hair care formulation according to any of the previous embodiments, wherein in the compound of formula (I) or (III):

at least one of the radicals F contains at least one moiety selected from the moieties

—R⁶(—O—C(O)—R⁶)ₘ—O—C(O)—R⁷,

—R⁶(—NR¹⁰—C(O)—R⁶)ₘ—O—C(O)—R⁷,

—R⁶(—NR¹⁰—C(O)—R⁶)ₘ—NR¹⁰—C(O)—R⁷,

—R⁶(—NR¹⁰—C(O)—R⁶)ₘ—NR¹⁰—C(O)—R⁷,

preferably —R⁶(—O—C(O)—R⁶)ₘ—O—C(O)—R⁷, wherein R¹⁰, R⁶, R⁷, and m are as defined above.

14. A hair care formulation according to any of embodiments 1-5, 7 and 10, wherein in the compound of formula (IV):

at least one of the radicals G contains at least one moiety

—R⁶(—C(O)—O—R⁶)ₘ—C(O)—O—R¹¹,

—R⁶(—C(O)—NR¹⁶—R⁶)ₘ—C(O)—O—R¹¹,

—R⁶(—C(O)—NR¹⁶—R⁶)ₘ—C(O)—NR¹⁶—R¹¹,

preferably —R⁶(—C(O)—O—R⁶)ₘ—C(O)—O—R¹¹, wherein R¹⁰, R⁶, R¹¹, and m are as defined above.

15. A hair care formulation according to any of the previous embodiments, wherein in the formula (II) or (V) of the compound according to formula (I), (III) or (IV):

m=0 to 10, more preferred 0 to 6, even more preferred 1 to 6, specifically 0, 1, 2, 3, 4, 5, 6, more specifically 0 or 1.

16. A hair care formulation according to any of the previous embodiments, wherein in the compound of formula (I), (III) or (IV):

R¹⁰ is selected from the group consisting of hydrogen, n-, iso-, or tert.-C₁-C₂₂-alkyl, C₂-C₂₂-alkoxyalkyl, C₅-C₃₀-cycloalkyl, C₆-C₃₀-aryl, C₆-C₃₀-aryl(C₁-C₆)alkyl, O₆—O₃₀-alkylaryl, C2-C₂₂-alkenyl, C₂-C₂₂-alkenyloxyalkyl, which optionally can be each substituted by hydroxyl and halogen, and which optionally can contain one or more ether groups (—O—), preferably R¹⁰ is selected from hydrogen or n-, iso-, and tert.-C₁-C₂₂-alkyl.

17. A hair care formulation according to any of the previous embodiments, wherein in the compound of formula (I), (III) or (IV):

R⁶ is independently selected from optionally substituted straight-chain, cyclic or branched, saturated or unsaturated radicals which have up to 24 carbon atoms, preferred 1 to 24, more preferred 2 to 20 carbon atoms, even more preferred 8 to 18 carbon atoms.

18. A hair care formulation according to any of the previous embodiments 1 to 13 and 15 to 17, wherein in the compound of formula (I) or (III):

R⁷ is independently selected from optionally substituted straight-chain, cyclic or branched, saturated or unsaturated hydrocarbon radicals which have up to 24 carbon atoms, preferred 1 to 24, more preferred 2 to 20 carbon atoms, even more preferred 8 to 18 carbon atoms, which can be optionally substituted by one or more groups selected from carboxyl, hydroxyl, or halide groups.

19. A hair care formulation according to any of the embodiments 1 to 5, 7, 10 and 14 to 17, wherein in the compound of formula (IV):

R¹¹ is independently selected from optionally substituted straight-chain, cyclic or branched, saturated or unsaturated hydrocarbon radicals which have up to 24 carbon atoms, preferred 1 to 24, more preferred 2 to 20 carbon atoms, even more preferred 8 to 18 carbon atoms.

20. A hair care formulation according to any of the previous embodiments 1 to 13 and 15 to 18, wherein in the compound of formula (I) or (III)

the total number of carbon atoms in R⁶+R⁷ (Σcarbon atoms R⁶, R⁷) in each single moiety of the general formula (II) composed of R⁶ and R⁷ is 10 to 300, preferred 15 to 200, more preferred 20 to 150, even more preferred 30 to 100.

21. A hair care formulation according to any of the embodiments 1 to 5, 7, 10, 14 to 17 and 19, wherein in the compound of formula (IV):

the total number of carbon atoms in R⁶+R¹¹ (Σcarbon atoms R⁶, R¹¹) in each single moiety of the general formula (V) composed of R⁶ and R¹¹ is 10 to 300, preferred 15 to 200, more preferred 20 to 150, even more preferred 30 to 100.

22. A hair care formulation according to any of the previous embodiments, wherein R¹ in the compound of formula (IV) or R² the compound of formula (III):

are selected from divalent to hexavalent, preferred divalent to tetravalent, more preferred divalent to trivalent, in particular divalent, trivalent, tetravalent, pentavalent, even more preferably divalent, trivalent and tetravalent or hexavalent optionally substituted hydrocarbon radicals, preferably optionally hydroxyl, amino or amido substituted straight-chain, cyclic or branched, saturated, unsaturated or aromatic, preferably saturated radicals, preferably derived from polyols, from dihydroxy carboxylic acids with up to 25 carbon atoms, preferably selected from the groups consisting of 2,2′-di-hydroxymethyl propanoic acid, 9,10-dihydroxy stearic acid, or from polyhydroxy carboxylic acids with up to 25 carbon atoms, preferably selected from the groups consisting of 3,5-dihydroxy-3-methylpentanoic acid, and sugar acids, such as gluconic acid, glyceric acid, xylonic acid, and ascorbic acid.

23. A hair care formulation according to any of the previous embodiments, wherein in the compound of formula (I), (III) or (IV):

R¹⁰ is selected from the group consisting of hydrogen, n-, iso-, or tert.-C₁-C₂₂-alkyl, more preferred hydrogen.

24. A hair care formulation according to any of the previous embodiments, wherein in the compound of the formulas (I), (III) or (IV):

R⁶ is derived from monohydroxy carboxylic acids with up to 25 carbon atoms, preferably independently selected from the group consisting of glycolic acid, lactic acid, 2-hydroxy butyric acid, 3-hydroxy-butyric acid, 4-hydroxy butyric acid, 14-hydroxy tetradecanoic acid, 10-hydroxy stearic acid, 12-hydroxy stearic acid, ricinoleic acid, and lesquerolic acid.

25. A hair care formulation according to any of the embodiments 1 to 13, 15 to 18, 20 and 22 to 24, wherein in the compound of formula (I) or (III) R⁷ is derived from carboxylic acids with up to 25 carbon atoms which do not have hydroxyl substituents, preferably independently selected from the group consisting of acetic acid, propanoic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, docosanoic acid, 2-ethyl hexanoic acid, 2,2-dimethyl propionic acid, 2,2-dimethyl heptanoic acid, 2,2-dimethyl octanoic acid, neodecanoic acid, undecyl-10-en-ic acid, oleic acid, linoleic acid, linolenic acid, and erucic acid.

26. A hair care formulation according to any of the embodiments 1-5, 7, 10, 14 to 17, 19, 21 to 24, wherein in the compound of formula (IV) R¹¹ is derived from linear or branched carboxylic acids, or linear or branched alcohols with up to 26 carbon atoms, preferably independently selected from the group consisting of methanol, ethanol, 2-propanol, 1-butanol, t-butanol, undec-10-en-ol, oleyl alcohol, stearyl alcohol, 1,2,-propanediol, 1,3-propanediol, 1,3-butanediol, 1,4-butanediol, 1,2 hexanediol, 1,6-hexanediol, glycerol, diglycerol, triglycerol, and linear or branched oligoglycerols formally comprising from 4 to 6 glycerol units, trimethylol propane, castor oil (ricinoleic acid triglyceride), lesquerella oil (lesquerolic acid triglyceride), pentaerythritol, sorbitol, polyalkylene oxides, such as ethylene oxide-, propylene oxide- and/or butylene oxide-based polyethers, 1,2,-propanediol, 1,3-propanediol, 1,3-butanediol, 1,4-butanediol, 1,2 hexanediol, and 1,6-hexanediol.

27. A hair care formulation according to any of the embodiments 1 to 13, 15 to 18, 20 and 22 to 25, wherein in the compound of formula (I) or (III) at least one, preferably both of R⁶ and R⁷ of the moieties of the general formula (II) are derived from unsaturated carboxylic acids.

28. A hair care formulation according to any of the previous embodiments, wherein R¹ in the compound of the formula (IV) or R² in the compound of the formula (III) are selected from the group consisting of:
residues derived from polyols, preferably derived from the group consisting of 1,2-propanediol, 1,3-propanediol, 1,3-butanediol, 1,4-butanediol, 1,2-hexanediol, 1,6-hexanediol, glycerol, diglycerol, triglycerol and linear or branched oligoglycerols such as oligoglycerols formally comprising from 4 to 6 glycerol units, trimethylol propane, pentaerythritol, sorbitol,
residues derived from polyalkylene oxides, such as ethylene oxide-, propylene oxide- and/or butylene oxide-based polyethers, in particular derived from polyethylene glycols, such as diethylene glycol, triethylene glycol, tetraethylene glycol, and pentaethylene glycol., or derived from polypropylene glycols, such as dipropylene glycol, (in particular derived from 2,2′-oxydi-1-propanol, 1,1′-oxydi-2-propanol, and 2-(2-hydroxypropoxy)-1-propanol), tripropylene glycol, tetrapropylene glycol, pentapropylene glycol, derived from mixed ethylene oxide and butylene oxide based copolyethers, derived from mixed propylene oxide and butylene oxide based copolyethers, and derived from mixed ethylene oxide and propylene oxide and butylene oxide based copolyethers, oligomeric or polymeric polyols, such as hydroxy-functional polyacrylates, hydroxy-functional polyesters, hydroxy-functional polyurethanes.
residues derived from dihydroxycarboxylic acids, preferably derived from the group consisting of 2,2′-dihydroxymethyl propanoic acid and 9,10-dihydroxy stearic acid,
residues derived from polyhydroxy carboxylic acids with up to 25 carbon atoms, preferably selected from the groups consisting of 3,5-dihydroxy-3-methylpentanoic acid, and sugar acids, such as gluconic acid, glyceric acid, xylonic acid, and ascorbic acid,
residues derived from epoxy compounds, preferably derived from ether epoxy compounds, in total having more than one, preferred more than two carbon atoms, preferred selected from ethylene oxide, propylene oxide, butylene oxide, glycidyl ethers such as prepared in particular from the reaction of epichlorhydrin with alcohols, such as methanol, ethanol, 2-propanol, 1-butanol, t-butanol, undec-10-en-ol, oleyl alcohol, stearyl alcohol, 1,2,-propanediol, 1,3-propanediol, 1,3-butanediol, 1,4-butanediol, 1,2 hexanediol, 1,6-hexanediol, glycerol, diglycerol, triglycerol, and linear or branched oligoglycerols, in particular formally comprising from 4 to 6 glycerol units, trimethylol propane, castor oil (ricinoleic acid triglyceride), lesquerella oil (lesquerolic acid triglyceride), pentaerythritol, sorbitol, polyalkylene oxides, such as ethylene oxide-, propylene oxide- and/or butylene oxide-based polyethers, in particular derived from polyethylene glycols, such as diethylene glycol, triethylene glycol, tetraethylene glycol, and pentaethylene glycol, or derived from polypropylene glycols, like dipropylene glycol (in particular derived from 2,2′-oxydi-1-propanol, 1,1′-oxydi-2-propanol, and 2-(2-hydroxypropoxy)-1-propanol), tripropylene glycol, tetrapropylene glycol, pentapropylene glycol, derived from mixed ethylene oxide and butylene oxide based copolyethers, derived from mixed propylene oxide and butylene oxide based copolyethers, and derived from mixed ethylene oxide and propylene oxide and butylene oxide based copolyethers, or preferred glycidyl esters, with acids, in particular neodecanoic acid, oligomeric or polymeric polyols, such as hydroxy-functional polyacrylates, hydroxy-functional polyesters, hydroxy-functional polyurethanes,
residues derived from ester compounds obtained from the reaction of polyols or epoxides with hydroxy carboxylic acids, ester compounds obtained from the reaction of alcohols, in particular alcohols selected from 1,2,-propanediol, 1,3-propanediol, 1,3-butanediol, 1,4-butanediol, 1,2 hexanediol, 1,6-hexanediol, glycerol, diglycerol, triglycerol and linear or branched oligoglycerols such as formally comprising from 4 to 6 glycerol units, trimethylol propane, castor oil (ricinoleic acid triglyceride), lesquerella oil pentaerythritol, sorbitol, polyalkylene oxides, such as ethylene oxide-, propylene oxide- and/or butylene oxide-based polyethers, in particular derived from polyethylene glycols, such as diethylene glycol, triethylene glycol, tetraethylene glycol, and pentaethylene glycol, or derived from polypropylene glycols, such as dipropylene glycol (in particular derived from 2,2′-oxydi-1-propanol, 1,1′-oxydi-2-propanol, and 2-(2-hydroxypropoxy)-1-propanol), tripropylene glycol, tetrapropylene glycol, pentapropylene glycol, derived from mixed ethylene oxide and butylene oxide based copolyethers, derived from mixed propylene oxide and butylene oxide based copolyethers, and derived from mixed ethylene oxide and propylene oxide and butylene oxide based copolyethers,

or epoxy compounds, in particular selected from ethylene oxide, propylene oxide, butylene oxide, glycidyl ethers, with alcohols, in particular. methanol, ethanol, 2-propanol, 1-butanol, t-butanol, undec-10-en-ol, oleyl alcohol, stearyl alcohol, 1,2,-propanediol, 1,3-propanediol, 1,3-butanediol, 1,4-butanediol, 1,2 hexanediol, 1,6-hexanediol, glycerol, diglycerol, triglycerol and higher linear or branched oligoglycerols, trimethylol propane, castor oil (ricinoleic acid triglyceride), lesquerella oil (lesquerolic acid triglyceride), pentaerythritol, sorbitol, polyalkylene oxides, such as ethylene oxide-, propylene oxide- and/or butylene oxide-based polyethers, in particular derived from polyethylene glycols, such as diethylene glycol, triethylene glycol, tetraethylene glycol, and pentaethylene glycol., or derived from polypropylene glycols, such as dipropylene glycol (in particular derived from 2,2′-oxydi-1-propanol, 1,1′-oxydi-2-propanol, and 2-(2-hydroxypropoxy)-1-propanol), tripropylene glycol, tetrapropylene glycol, pentapropylene glycol, derived from mixed ethylene oxide and butylene oxide based copolyethers, derived from mixed propylene oxide and butylene oxide based copolyethers, and derived from mixed ethylene oxide and propylene oxide and butylene oxide based copolyethers, or preferred glycidyl esters, with hydroxyl functionalized carboxylic acids, in particular lactic acid, 4-hydroxy butanoic acid, ricinoleic acid, lesquerolic acid, 2,2-bis(hydroxymethyl) propionic acid, malic acid, tartaric acid, gluconic acid, especially preferred are the esters of glycerol with ricinoleic acid, i.e. castor oil, and lesquerolic acid, i.e. lesquerella oil,
residues derived from ester compounds obtained from the reaction of alcohols or epoxides with hydroxyl-free carboxylic acids,

such as divalent to hexavalent, preferably divalent to tetravalent, more preferably divalent to trivalent, even more preferably divalent, trivalent and tetravalent optionally amino or amido substituted straight-chain, cyclic or branched, saturated, unsaturated or aromatic radicals, derived from the reaction of alcohols, i.e. 1,2,-propanediol, 1,3-propanediol, 1,3-butanediol, 1,4-butanediol, 1,2 hexanediol, 1,6-hexanediol, glycerol, diglycerol, triglycerol and higher linear or branched oligoglycerols, trimethylol propane, castor oil (ricinoleic acid triglyceride), lesquerella oil pentaerythritol, sorbitol, polyalkylene oxides, such as ethylene oxide-, propylene oxide- and/or butylene oxide-based polyethers, in particular derived from polyethylene glycols, like diethylene glycol, triethylene glycol, tetraethylene glycol, and pentaethylene glycol, or derived from polypropylene glycols, such as dipropylene glycol (in particular derived from 2,2′-oxydi-1-propanol, 1,1′-oxydi-2-propanol, and 2-(2-hydroxypropoxy)-1-propanol), tripropylene glycol, tetrapropylene glycol, pentapropylene glycol, derived from mixed ethylene oxide and butylene oxide based copolyethers, derived from mixed propylene oxide and butylene oxide based copolyethers, and derived from mixed ethylene oxide and propylene oxide and butylene oxide based copolyethers, or

epoxy compounds, in particular ethylene oxide, propylene oxide, butylene oxide, glycidyl ethers with alcohols, in particular methanol, ethanol, 2-propanol, 1-butanol, t-butanol, undec-10-en-ol, oleyl alcohol, stearyl alcohol, 1,2,-propanediol, 1,3-propanediol, 1,3-butanediol, 1,4-butanediol, 1,2-hexanediol, 1,6-hexanediol, glycerol, diglycerol, triglycerol and higher linear or branched oligoglycerols, trimethylol propane, castor oil (ricinoleic acid triglyceride), lesquerella oil (lesquerolic acid triglyceride), pentaerythritol, sorbitol, polyalkylene oxides, such as ethylene oxide-, propylene oxide- and/or butylene oxide-based polyethers, in particular derived from polyethylene glycols, such as diethylene glycol, triethylene glycol, tetraethylene glycol, and pentaethylene glycol, or derived from polypropylene glycols, such as dipropylene glycol (in particular derived from 2,2′-oxydi-1-propanol, 1,1′-oxydi-2-propanol, and 2-(2-hydroxypropoxy)-1-propanol), tripropylene glycol, tetrapropylene glycol, pentapropylene glycol, derived from mixed ethylene oxide and butylene oxide based copolyethers, derived from mixed propylene oxide and butylene oxide based copolyethers, and derived from mixed ethylene oxide and propylene oxide and butylene oxide based copolyethers, or

preferably glycidyl esters,

with acids, in particular neodecanoic acid, with carboxylic acids, in particular acetic acid, propionic acid, butyric acid, dodecanoic acid, stearic acid, oleic acid, oxalic acid, malonic acid, succinic acid, maleic acid, itaconic acid, phthalic acid, terephthalic acid, trimellitic acid, pyromellitic acid, dimer fatty acids, preferably derived from a reaction of anhydrides or acid chlorides with alcohols,
residues derived from monocarboxylic acids, as mentioned above, or polycarboxylic acids such as succinic acid, maleic acid, itaconic acid, phthalic acid, terephthalic acid, trimellitic acid, pyromellitic acid, dimer fatty acids, preferably their anhydrides or acid chlorides, and polyacrylic acids.

29. A hair care formulation according to any of the previous embodiments, wherein at least one of R² and R³ in the at least one compound of formula (III) or R¹ in the compound of the formula (IV) is a divalent hydrocarbon radical, derived from the polyalkylene oxy groups as mentioned above of the general formula

—[CH₂CH₂O]_{q1}—[CH₂CH(CH₃)O]ᵣ₁—[CH₂CH(C₂H₅)O]ₛ₁—{[CH₂CH₂]_{g2}—[CH₂CH(CH₃)]ᵣ₂—[CH₂CH(C₂H₅)]ₛ₂}—

with

q1=0 to 49, preferred 0 to 10, more preferred 1 to 10, even more preferred 1 to 5,

r1=0 to 32, preferred 0 to 10, more preferred 1 to 10, even more preferred 1 to 5,

s1=0 to 24, preferred 0 to 10, more preferred 1 to 10, even more preferred 1 to 5,

q2=0 or 1,

r2=0 or 1,

s2=0 or 1, and

Σ(q2+r2+s2)=1,

with the proviso that the sum of the carbon atoms in such polyalkylene oxide groups is 2 to 100, preferred 2 to 50, more preferred 2 to 30, even more preferred 2 to 20, specifically 2 to 15.

30. A hair care formulation according to any of the previous embodiments, wherein at least one of R² and R³ in the compound of formula (III) or R¹ in the compound of the formula (IV)

is

a divalent hydrocarbon radical derived from oligoglycerols of the general formula:

—[CH₂CH(R⁸)CH₂O]ₜ₁—[CH₂CH(R⁸)CH₂)]ₜ₂—

with

t1=0 to 32, preferred 0 to 10, more preferred 1 to 10, even more preferred 1 to 5, specifically 1 and 2,

t2=1,

R⁸═OH or —O—C(O)—R⁶—N⁺(R¹⁰)₃, wherein R¹⁰ and R⁶ are as defined above,

with the proviso that the sum of the carbon atoms of R⁸ is 2 to 100, preferred 2 to 50, more preferred 2 to 30, even more preferred 2 to 20, specifically 2 to 15.

31. A hair care formulation according to any of the previous embodiments, wherein at least one of R² and R³ in the compound of formula (III) or R¹ in the compound of the formula (IV):

is a divalent hydrocarbon radical comprising at least one ester group of the general formulas:

[CH₂CH₂O]_{q1}—R⁹—[CH₂CH₂O]_{g1}—[CH₂CH₂]_{g2}—

with

q1 can be the same or different and are as defined above and q2=1

and

—[CH₂CH(R⁸)CH₂O]ₜ₁—R⁹—[CH₂CH(R⁸)CH₂O]ₜ₁—[CH₂CH(R⁸)CH₂]ₜ₂—

with

t1=0 to 32, preferred 0 to 10, more preferred 1 to 10, even more preferred 1 to 5, specifically 1 and 2,

t2=1,

R⁸═OH or —O—C(O)—R⁶-N+(R¹⁰)₃, wherein R¹⁰ and R⁶ are as defined above,

with the proviso that the sum of the carbon atoms in R8 is 2 to 100, preferred 2 to 50, more preferred 2 to 30, even more preferred 2 to 20, specifically 2 to 15 and R⁹ is selected from —C(O)C(O)O—, —C(O)(CH₂)₁₋₈C(O)O—, such as derived from succinic acid, adipic acid, sebacic acid, or —C(O)(C₆H₄)C(O)O—, i.e. derived from phthalic and terephthalic acid, —C(O)CH═CHC(O)O—, —C(O)C(═CH₂)—CH₂C(O)O—, —C(O)CH(OH)CH(OH)C(O)O—,

with the proviso that the sum of the carbon atoms in R⁹ is 2 to 100, preferred 2 to 50, more preferred 2 to 30, even more preferred 2 to 20, specifically 2 to 15.

32. A hair care formulation according to any of the previous embodiments, wherein R² in the compound of formula (III) or R¹ in the compound of formula (IV) is a hydrocarbon residue which does not contain a heteroatom or contains one or more groups —O—, preferably one to five —O— groups, and wherein the groups —O— are preferably ether groups, but can also form an ester group together with a carbonyl group, and wherein the groups R² can be optionally substituted by one or more hydroxyl groups, but are preferably not substituted by hydroxyl groups.

33. A hair care formulation according to any of the embodiments 1-13, 15-18, 20, 22-25, and 27 to 32, wherein in the compound of formula (I) or (III):

in the one or more groups F in at least one moiety of the formula:

—R⁶(—X—C(O)—R⁶)ₘ—X—C(O)—R⁷ (II)

X and m are as defined above, and

R⁶ is independently derived from lactic acid, ricinoleic acid, lesquerolic acid, 10-hydroxy stearic acid, 12-hydroxy stearic acid, 14-hydroxy tetradecanoic acid, and

R⁷ is derived from octadecanoic acid, eicosanoic acid, docosanoic acid, 2-ethyl hexanoic acid, 2,2-dimethyl propionic acid, neodecanoic acid, or oleic acid.

34. A hair care formulation according to any of the previous embodiments, wherein in the compound of formula (I), (III) or (IV):

R¹⁰ is preferably selected from the group consisting of hydrogen, or a bond to R¹ or R², thereby forming a cyclic structure of the schematic formulas:

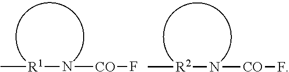

35. A hair care formulation according to any of the embodiments 1-13, 15-18, 20, 22-25, and 27 to 34, wherein in the compound of formula (I) or (III)

the groups F contain at least one moiety of the formula (II):

—R⁶(—X—C(O)—R⁶)ₘ—X—C(O)—R⁷ (II)

wherein R⁶, R⁷, X and m are as defined above, and wherein each moiety of the formula (II) comprises at least two different groups R⁶, wherein said groups R⁶ are arranged either random or blockwise.

36. A hair care formulation according to any of the embodiments 1-13, 15-18, 20, 22-25, and 27 to 35, wherein in the compound of formula (I) or (III)

the groups F contain at least one moiety of the formula (VI):

—R⁶(—X—C(O)—R⁶¹)ₘ₁(—X—C(O)—R⁶²)ₘ₂—X—C(O)—R⁷ (VI):

wherein R⁶ in formula (VI) is selected from R⁶¹ and R⁶², X and R⁷ are as defined above, and

R⁶¹ and R⁶² represent two different groups R⁶ as defined above,

and wherein

m1=0 to 20, preferred 0 to 10, more preferred 0 to 6, even more preferred 1 to 6, specifically 0, 1, 2, 3, 4, 5, or 6,

m2=0 to 20, preferred 0 to 10, more preferred 0 to 6, even more preferred 1 to 6, specifically 0, 1, 2, 3, 4, 5, or 6,

m=m1+m2, =0 to 20, preferred 0 to 10, more preferred 0 to 6, even more preferred 1 to 6, specifically 0, 1, 2, 3, 4, 5, or 6.

37. A hair care formulation according to any of the embodiments 1-13, 15-18, 20, 22-25, and 27 to 36, wherein in the compound of formula (I) or (III):

the groups F contain at least one moiety of the formula (VI):

—R⁶(—X—C(O)—R⁶¹)ₘ₁(—X—C(O)—R⁶²)ₘ₂—X—C(O)—R⁷ (VI)

wherein X, m1 and m2 are as defined above, and R⁶, R⁶¹, R⁶² and R⁷ are as defined in the following table:

| R⁶ derived from | R⁶¹ derived from | R⁶² derived from | R⁷ derived from |
|---|---|---|---|
| unsaturated acids, in | unsaturated acids, in | unsaturated acids, in | unsaturated acids, in |
| particular ricinoleic | particular ricinoleic | particular. ricinoleic | particular oleic acid |
| acid or lesquerolic | acid or lesquerolic | acid or lesquerolic | |
| acid | acid | acid | |
| unsaturated acids, in | unsaturated acids, in | unsaturated acids, in | saturated acids, in |
| particular ricinoleic | particular ricinoleic | particular ricinoleic | particular |
| acid or lesquerolic | acid or lesquerolic | acid or lesquerolic | octadecanoic acid or |
| acid | acid | acid | neodecanoic acid |
| unsaturated acids, in | unsaturated acids, in | saturated acids, in | saturated acids, in |
| particular ricinoleic | particular ricinoleic | particular 12- | particular |
| acid or lesquerolic | acid or lesquerolic | hydroxystearic acid | octadecanoic acid or |
| acid | acid | | neodecanoic acid |
| unsaturated acids, in | saturated acids, in | saturated acids, in | saturated acids, in |
| particular ricinoleic | particular 12- | particular 12- | particular |
| acid or lesquerolic | hydroxystearic acid | hydroxystearic acid | octadecanoic acid or |
| acid | | | neodecanoic acid |
| saturated acids, in | saturated acids, in | saturated acids, in | saturated acids, in |
| particular 12- | particular 12- | particular 12- | particular |
| hydroxystearic acid | hydroxystearic acid | hydroxystearic acid | octadecanoic acid or |
| | | | neodecanoic acid |
| saturated acids, in | saturated acids, in | saturated acids, in | unsaturated acids, in |
| particular 12- | particular 12- | particular 12- | particular oleic acid |
| hydroxystearic acid | hydroxystearic acid | hydroxystearic acid | |
| saturated acids, in | saturated acids, in | unsaturated acids, in | unsaturated acids, in |
| particular 12- | particular 12- | particular ricinoleic | particular oleic acid |
| hydroxystearic acid | hydroxystearic acid | acid or lesquerolic | |
| | | acid | |
| saturated acids, in | unsaturated acids, in | unsaturated acids, in | unsaturated acids, in |
| particular 12- | particular ricinoleic | particular ricinoleic | particular oleic acid |
| hydroxystearic acid | acid or lesquerolic | acid or lesquerolic | |
| | acid | acid | |
| saturated acids, in | unsaturated acids, in | saturated acids, in | unsaturated acids, in |
| particular 12- | particular ricinoleic | particular 12- | particular oleic acid |
| hydroxystearic acid | acid or lesquerolic | hydroxystearic acid | |
| | acid | | |
| unsaturated acids, in | saturated acids, in | unsaturated acids, in | saturated acids, in |
| particular ricinoleic | particular 12- | particular ricinoleic | particular |
| acid or lesquerolic | hydroxystearic acid | acid or lesquerolic | octadecanoic acid or |
| acid | | acid | neodecanoic acid |

38. A hair care formulation according to any of the embodiments 1-13, 15-18, 20, 22-25, and 27 to 37, wherein in the compound of formula (I) or (III)

the groups F contain at least one moiety of the formula (VI):

—R⁶(—X—C(O)—R⁶¹)ₘ₁(—X—C(O)—R⁶²)ₘ₂—X—C(O)—R⁷ (VI)

wherein X, m1, m2, R⁶, R⁶¹, R⁶² and R⁷ are each as defined above, and

the total number of carbon atoms in R⁶, R⁶¹, R⁶² and R⁷ (Σcarbon atoms R⁶, R⁶¹, R⁶² and R⁷) per group F is about 19 to 300, preferred 25 to 300, more preferred 30 to 300, specifically 30 to 200, more specifically 30 to 150.

39. A hair care formulation according to any of the previous embodiments, wherein in the compound of formula (I), (III) or (IV):

R⁶ is independently derived from mono- or poly-(such as di-, tri-, tetra-)hydroxy carboxylic acids selected from the group consisting of lactic acid, ricinoleic acid, lesquerolic acid, 10-hydroxy stearic acid, and 12-hydroxy stearic acid, 14-hydroxy tetradecanoic acid, 2,2′-di-hydroxymethyl propanoic acid, and 9,10-dihydroxy stearic acid, and gluconic acid, preferably at least one or all R⁶ are derived from ricinoleic acid or lesquerolic acid.

40. A hair care formulation according to any of the embodiments 1-13, 15-18, 20, 22-25, and 27 to 39, wherein in the compound of formula (I) or (III):

R⁷ is independently derived from octadecanoic acid, eicosanoic acid, docosanoic acid, 2-ethyl hexanoic acid, 2,2-dimethyl propionic acid, neodecanoic acid, oleic acid, preferably at least one or all R⁷ are derived from oleic acid.

41. A hair care formulation according to any of the previous embodiments, wherein in the compound of the formula (I), (III) or (IV) low melting and high melting fatty acids ≥C5 are specifically positioned within the R⁶ and R⁷ containing ester elements of the general formula (II) and the R⁶ and R¹¹ containing ester elements of the general formula (V):

—R⁶(—X—C(O)—R⁶)ₘ—X—C(O)—R⁷ (II) and

R⁶(—C(O)—X—R⁶)ₘ—C(O)—X—R¹¹ (V),

in particular in the moieties

—O—C(O)—R⁶—(O—C(O)—R⁶)ₘ—O—C(O)—R⁷ and

—NR¹⁰—C(O)—R⁶—(O—C(O)—R⁶)ₘ—O—C(O)—R⁷,

wherein X, R⁶, and R⁷ and R¹⁰ are as defined above,

and wherein

in one or more moieties of the formula (II) at least one, preferred more than one, more preferred one, two or three low melting fatty acids with 5 or more carbon atoms and a melting point of 40° C. or below each forming a group R⁶ are contained in the radical or the radicals R⁶ adjacent to R⁷, while at least one, preferred more than one, more preferred one, two or three high melting fatty acids with 5 or more carbon atoms and a melting point above 40° C. form the radical or radicals R⁶ at the opposite terminus of a R⁶- and R⁷-containing ester element of the formula (II), or in one or more moieties of the formula (II) at least one, preferred more than one, more preferred one, two or three high melting fatty acids ≥C5 each forming R⁶ form the radical or radicals R⁶ adjacent to R⁷, while at least one, preferred more than one, more preferred one, two or three low melting fatty acids with 5 or more carbon atoms and a melting point below 40° C. form the radical or radicals R⁶ at the opposite terminus of a R⁶- and R⁷-containing ester element of the formula (II), and t is preferable when at least 50% of all moieties of the formula (II) display such a positioning of R⁶-radicals derived from high-melting and low-melting fatty acids as described above, it is more preferred when more than 80% of all moieties of the formula (II) display such a positioning of R⁶-radicals derived from high-melting and low-melting fatty acids as described above, and it is most preferably when all moieties of the general formula (II) display such positioning of residues R⁶ and R⁷ as described above,

or wherein

in one or more moieties of the formula (V) at least one, preferred more than one, more preferred one, two or three low melting fatty acids ≥C5 each forming a group R⁶ are contained in the radical or the radicals R⁶ adjacent to R¹¹, while at least one, preferred more than one, more preferred one, two or three high melting fatty acids ≥C5 form the radical or radicals R⁶ at the opposite terminus of a R⁶- and R¹¹-containing ester element of the formula (V), or in one or more moieties of the formula (V) least one, preferred more than one, more preferred one, two or three high melting fatty acids ≥C5 each forming R⁶ form the radical or radicals R⁶ adjacent to R¹¹, while at least one, preferred more than one, more preferred one, two or three low melting fatty acids ≥C5 form the radical or radicals R⁶ at the opposite terminus of a R⁶- and R¹¹-containing ester element of the formula (V), and it is preferable when at least 50% of all moieties of the formula (V) display such a positioning of R⁶-radicals derived from high-melting and low-melting fatty acids as described above, it is more preferred when more than 80% of all moieties of the formula (V) display such a positioning of R⁶-radicals derived from high-melting and low-melting fatty acids as described above, and most preferably all moieties of the general formula (V) display such positioning of residues R⁶ and R¹¹ as described above.

42. A hair care formulation according to any of the embodiments 6-13, 15-18, 20, 22-25, and 27 to 41, wherein in the compound of formula (III):

R⁴ is selected from divalent to tetravalent, such as divalent, trivalent, tetravalent, preferably divalent, optionally substituted straight-chain, cyclic or branched, saturated, unsaturated or aromatic hydrocarbon radicals which have up to 200 carbon atoms, more preferred up to 150 carbon atoms, even more preferred up to 100 carbon atoms, specifically up to 80 carbon atoms, and preferably has at least 2, more preferred at least 10, more preferred as least 14 carbon atoms, which optionally contain one or more groups selected from —O—, —NH—, and —C(O)—, and wherein if a plurality of R⁴ is present in formula (III), they can be the same or different.

43. A hair care formulation according to any of the embodiments 6-13, 15-18, 20, 22-25, and 27 to 42, wherein in the compound of the general formula (III)

R⁴ comprises at least one ester group (—O—C(O)—, or —C(O)—O—, respectively).

44. A hair care formulation according to any of the embodiments 6-13, 15-18, 20, 22-25, and 27 to 43, wherein in the compound of formula (III):

R⁴ is derived from dicarboxylic acids, tricarboxylic acids or tetracarboxylic acids, in particular dicarboxylic acids, such as succinic acid, oxalic acid, malonic acid, malic acid, tartaric acid, maleic acid, itaconic acid, succinic acid, sebacic acid, dimer acids, amino-functional dicarboxylic acids, such as D-glutamic acid, and a dicarboxylic acid of the formula:

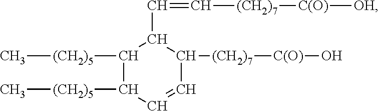

and condensation products of hydroxy carboxylic acids, in particular, from ricinoleic acid or lesquerolic acid, and dicarboxylic acids, such as succinic acid, oxalic acid, malonic acid, malic acid, tartaric acid, maleic acid, itaconic acid, succinic acid, sebacic acid, dimer acids, amino-functional dicarboxylic acids, such as D-glutamic acid, and a dicarboxylic acid of the formula:

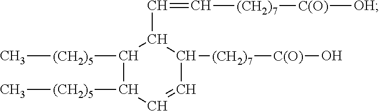

or

R⁴ is derived from amide condensation products of amino acids with maleic acid or succinic acid, such as N-maleoyl-β-alanine ((E)-4-(2-carboxyethylamino)-4-oxo-but-2-enoic acid), N-succinyl-μ-alanine (4-[(2-hydroxy-1-methyl-2-oxo-ethyl)amino]-4-oxo-butanoic acid), N-maleoyl-asparagine (4-amino-2-[[(E)-4-hydroxy-4-oxo-but-2-enoyl]amino]-4-oxo-butanoic acid); or R⁴ is derived from the ester condensation products of divalent alcohols, i.e. ethylene glycol, 1,2-propylene glycol, 1,3-propylene glycol, 1,3-butane diol, 1,4-butanediol, with dicarboxylic acid anhydrides, i.e. maleic anhydride, succinic anhydride, phthalic anhydride;

or

R⁴ is derived from tri- or tetracarboxylic acids, such as citric acid, isocitric acid, trimelletic acid, pyromellitic acid, cyclobutane tetracarboxylic acid; or

R⁴ is derived from the bis-amide condensation products of amino acids with maleic acid or succinic acid, i.e. bis-(N-maleoyl)-lysine; or

R⁴ is derived from the ester condensation products of trivalent alcohols, such as glycerol, trimethylolpropane with dicarboxylic acid anhydrides, such as maleic anhydride, succinic anhydride, phthalic anhydride; or

R⁴ is derived from the ester condensation products of tetravalent alcohols, such as diglycerol, pentaerythritol with dicarboxylic acid anhydrides, such as maleic anhydride, succinic anhydride, phthalic anhydride, and

most preferred R⁴ is derived from the condensation products of ricinoleic acid or lesquerolic acid and succinic acid.

45. A compound of the formula (Ia):

R¹(—X—C(O)—F)ₚ (Ia)

wherein

R¹ in formula (Ia) is selected from a p-valent, optionally substituted hydrocarbon radical and may contain optionally one or more groups selected from —O—, —NH—, —C(O)—, —C(S)—, tertiary amino groups

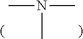

and quaternary ammonium groups, and can be optionally substituted by one or more selected from carboxyl groups or hydroxyl groups,

p≥2, more preferably 2-811,

X can be the same or different and is selected from —O—, or —NR¹⁰—, wherein R¹⁰ is selected from the group consisting of hydrogen, or optionally substituted straight-chain, cyclic or branched, saturated, unsaturated or aromatic hydrocarbon radicals which have up to 100 carbon atoms which optionally contain one or more groups selected from —O—, —NH—, —C(O)—, —C(S)—, tertiary amino groups

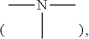

or in formula (I) R¹⁰ may form a bond to R¹ to form a cyclic structure,

F can be the same or different and is selected from optionally substituted straight-chain, cyclic or branched, saturated, unsaturated or aromatic hydrocarbon radicals which have up to 1005 carbon atoms, which optionally contain one or more groups selected from —O—, —NH—, —C(O)—, —C(S)—, and tertiary amino groups

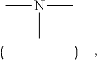

and can be optionally substituted by one or more selected from carboxyl, hydroxyl or halide groups, with the proviso that at least one of the radicals F contains at least one moiety of the formula (IIa):

—R⁶(—X—C(O)—R⁶)ₘ—X—C(O)—R⁷ (IIa)

wherein

X is as defined above,

m=1 to 20, preferably 2 to 20,

R⁶ is independently selected from a divalent optionally substituted straight-chain, cyclic or branched, saturated or unsaturated hydrocarbon radical which have up to 36 carbon atoms,

R⁷ is independently selected from optionally substituted straight-chain, cyclic or branched, saturated or unsaturated hydrocarbon radicals which have 1 to 1000 carbon atoms, optionally containing one or more groups selected from —O—, —NH—, —C(O)—, —C(S)—, tertiary amino groups

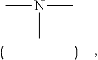

quaternary ammonium groups

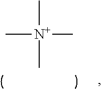

and which can be substituted with carboxyl, hydroxyl, or halide groups, wherein the radical R⁷ cannot contain an internal carboxy group or amide, i.e. R⁷ cannot contain a combination of a —C(O)— group and a —O— group or a combination of a —C(O)— group and a —NH— or tertiary amino group, with the proviso that in at least one moiety of the formula (IIa) R⁷ has at least 2, preferably at least 6 carbon atoms, and in the same moiety of the formula (IIa) at least one R⁶ has at least 6, preferably at least 8 carbon atoms,

or

containing at least one compound of the general formula (IVa)

R¹(—C(O)—X-G)_{q} (IVa),

wherein X is as defined above,

R¹ in formula (IVa) is selected from q-valent, optionally substituted hydrocarbon radicals which preferably have up to 1000 carbon atoms, and may contain optionally one or more groups selected from —O—, —NH—, —C(O)—, —C(S)—, tertiary amino groups

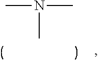

quaternary ammonium groups, and can be optionally substituted by one or more selected from carboxyl groups or hydroxyl groups,

q=2 to 55, preferably 2 to 40, more preferably 2 to 4, and

G can be the same or different and is selected from optionally substituted straight-chain, cyclic or branched, saturated, unsaturated or aromatic hydrocarbon radicals which have up to 1005 carbon atoms, which optionally contain one or more groups selected from —O—, —NH—, —C(O)—, —C(S)—, and tertiary amino groups

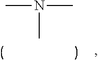

and can be substituted by one or more selected from carboxyl, hydroxyl or halide groups, with the proviso that at least one of the radicals G contains at least one moiety of the formula (Va):

—R⁶(—C(O)—X—R⁶)ₘ—C(O)—X—R¹¹ (Va)

wherein X is as defined above,

m=1 to 20, preferably 2 to 20,

R⁶ in formula (Va) is as defined above for formula (Ia),

R¹¹ is independently selected from optionally substituted straight-chain, cyclic or branched, saturated or unsaturated hydrocarbon radicals which have 1 to 1000 carbon atoms, optionally containing one or more groups selected from O—, —NH—, —C(O)—, —C(S)—, tertiary amino groups

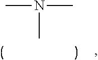

quaternary ammonium groups

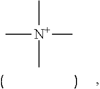

and which can be substituted with carboxyl, hydroxyl, or halide groups, wherein the radical R¹¹ cannot contain an internal carboxy group or amide, i.e. R¹¹ cannot contain a combination of a —C(O)— group and a —O— group or a combination of a —C(O)— group and a —NH— or tertiary amino group,

with the proviso that in at least one moiety of the formula (Va) R¹¹ has at least 2, preferably at least 6 carbon atoms, and in the same moiety of the formula (Va) at least one R⁶ has at least 6, preferably at least 8 carbon atoms.

46. A compound of the formula (Ia) or (IVa) according to embodiment 45, wherein

R¹ is as defined above having up to 10000 carbon atoms, preferably up to 1000, more preferably up to 300, even more preferably up to 100, most preferably up to 50 carbon atoms.

47. A compound of the formula (Ia) or (IVa) according to embodiments 45 and 46, wherein the number of carbon atoms in any R⁷ or R¹¹ of the compound is from 3 to 300, preferably 3 to 100, more preferably 3 to 50, even more preferably 3 to 36, further preferably 3 to 24, and most preferably 11 to 24.

48. A compound of the formula (Ia) or (IVa) according to any of embodiments 45 to 47, wherein the compound of the formula (Ia) or (IVa) has a molecular weight in the range of from 1500 to 200000 g/mol, preferably 1500 to 100000 g/mol, more preferably 1500 to 30000 g/mol, even more preferably 1500 to 10000 g/mol, further preferably 1500 to 5000 g/mol, and most preferably 1500 to 3000 g/mol.

49. A compound of the formula (Ia) or (IVa) according to any of embodiments 45 to 48, wherein the compound of the formula (Ia) contains 2 to 100 moieties of the formula (IIa), more preferably 2 to 50 moieties of the formula (IIa), even more preferably 2 to 20, further preferably 2 to 10, and even further preferably 2 to 6, and most preferably 2 to 4 moieties of the formula (IIa), or wherein the compound of the formula (IVa) contains 2 to 100 moieties of the formula (Va), more preferably 2 to 50 moieties of the formula (Va), even more preferably 2 to 20, further preferably 2 to 10, and even further preferably 2 to 6, and most preferably 2 to 4 moieties of the formula (Va).

50. A compound of the formula (Ia) according to any of embodiments 45 to 49, which is represented by the general formula (IIIa):

{[(F—C(O)—X—)₁₋₅R³(—X—C(O)—)]₁₋₃R⁴—C(O)—X—}ᵣ—R²(—X—C(O)—F)ₛ (IIIa)

wherein

X can be the same or different and is selected from —O—, or —NR¹⁰—, wherein R¹⁰ is selected from the group consisting of hydrogen, or optionally substituted straight-chain, cyclic or branched, saturated, unsaturated or aromatic hydrocarbon radicals which have up to 100 carbon atoms which optionally contain one or more groups selected from —O—, —NH—, —C(O)—, —C(S)—, tertiary amino groups

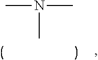

or in formula (IIIa) R¹⁰ may form a bond to R² to form a cyclic structure,

R² in formula (IIIa) is selected from (r+s)-valent, optionally substituted hydrocarbon radicals which have up to 1000 carbon atoms, and may contain optionally one or more groups selected from —O—, —NH—, —C(O)—, —C(S)—, tertiary amino groups

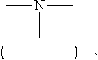

and quaternary ammonium groups, and can be optionally substituted by one or more carboxylic groups or hydroxyl groups, and optionally forms a bond to the nitrogen atom in the group —NR¹⁰—, in case R¹⁰ is a bond to R²,

R³ is selected from di- to hexavalent, optionally substituted hydrocarbon radicals which have up to 1000 carbon atoms, and may contain optionally one or more groups selected from —O—, —NH—, —C(O)—, —C(S)—, tertiary amino groups

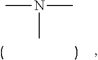

wherein if a plurality of R³ is present in formula (IIIa), they can be the same or different,

R⁴ is selected from divalent to tetravalent optionally substituted straight-chain, cyclic or branched, saturated, unsaturated or aromatic hydrocarbon radicals which have up to 300 carbon atoms, which optionally contain one or more groups selected from —O—, —NH—, —C(O)—, —C(S)—, tertiary amino groups

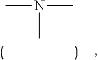

wherein if a plurality of R⁴ is present in formula (IIIa), they can be the same or different,

and wherein

r+s=2 to 55,

r=0 to 54,

s≥1,

and in formula (IIIa) F can be the same or different and is selected from optionally substituted straight-chain, cyclic or branched, saturated, unsaturated or aromatic hydrocarbon radicals which have up to 1005 carbon atoms, which optionally contain one or more groups selected from —NH—, —C(O)—, —C(S)—, and tertiary amino groups

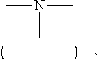

and can be optionally substituted by one or more selected from carboxyl, hydroxyl or halide groups, with the proviso that at least one of the radicals F contains at least one moiety of the formula (IIa):

—R⁶(—X—C(O)—R⁶)ₘ—X—C(O)—R⁷ (IIa)

wherein

X is as defined above,

with m=1 to 20, preferably m=2 to 20,

R⁶ is independently selected from a divalent optionally substituted straight-chain, cyclic or branched, saturated or unsaturated hydrocarbon radical which have up to 36 carbon atoms,

R⁷ is independently selected from optionally substituted straight-chain, cyclic or branched, saturated or unsaturated hydrocarbon radicals which have 1 to 1000 carbon atoms, optionally containing one or more groups selected from —O—, —NH—, —C(O)—, —C(S)—, tertiary amino groups

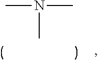

quaternary ammonium groups

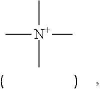

and which can be substituted with carboxyl, hydroxyl, or halide groups, wherein the radical R⁷ cannot contain an internal carboxy group or amide, i.e. R⁷ cannot contain a combination of a —C(O)— group and a —O— group or a combination of a —C(O)— group and a —NH— or tertiary amino group,

and with the proviso that in at least one moiety of the formula (IIa) R⁷ has at least 2, preferably at least 6 carbon atoms,

and in the same moiety of the formula (IIa) at least one R⁶ has at least 6, preferably at least 8 carbon atoms.

51. A compound of the formula (Ia), (IIIa) or (IVa) according to any of the embodiments 45 to 50, wherein R¹⁰ is selected from the group consisting of hydrogen, n-, iso-, or tert.-C₁-C₂₂-alkyl, C₂-C₂₂-alkoxyalkyl, C₅-C₃₀-cycloalkyl, C₆-C₃₀-aryl, C₆-C₃₀-aryl(C₁-C₆)alkyl, C₆-C₃₀-alkylaryl, C₂-C₂₂-alkenyl, C₂-C₂₂-alkenyloxyalkyl, which optionally can be each substituted by hydroxyl and halogen, and which optionally can contain one or more ether groups (—O—), preferably R¹⁰ is selected from hydrogen or n-, iso-, and tert.-01-C₂₂-alkyl.

52. A compound of the formula (IIIa) or (IVa) according to any of the embodiments 45 to 51, wherein:

R¹ in the compound of formula (IVa) or R² in the compound of formula (IIIa) is selected from optionally substituted hydrocarbon radicals which have up to 2 to 300 carbon atoms, more preferred 3 to 200 carbon atoms, even more preferred 3 to and 150 carbon atoms, specifically 3 to 50 carbon atoms, more specifically 3 to 20 carbon atoms may contain optionally one or more groups selected from —O—, —NH—, —C(O)—, —C(S)— and tertiary amino groups

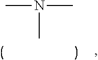

and can be optionally substituted with carboxy groups, hydroxyl groups, and quaternary ammonium groups.

53. A compound of the formula (IIIIa) or (IVa) according to any of the embodiments 45 to 52, wherein:

R¹ in the compound of formula (IVa) or R² in the compound of the formula (IIIa) is selected from divalent to hexavalent, preferred divalent to tetravalent, more preferred divalent to trivalent, in particular divalent, trivalent, tetravalent, pentavalent, or hexavalent optionally substituted hydrocarbon radicals.

54. A compound of the formula (IIIIa) according to any of the embodiments 50 to 53, wherein:

R³ is selected from di- to hexavalent residues.

55. A compound of the formula (IIIIa) according to any of the embodiments 50 to 54, wherein:

R³ is selected from optionally substituted straight-chain, cyclic or branched, saturated, unsaturated or aromatic hydrocarbon radicals which have up to 300 carbon atoms, preferred 1 to 200 carbon atoms, more preferred 1 to 150 carbon atoms, even more preferred 1 to 50 carbon atoms, specifically 1 to 20 carbon atoms, more specifically 1 to 10 carbon atoms which optionally contain one or more groups selected from —O—, —NH—, —C(O)—, —C(S)—, tertiary amino groups

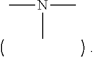

56. A compound of the formula (IIIa) according to any of the previous embodiments 50 to 55, wherein R⁴ is selected from divalent to tetravalent, preferred divalent, trivalent, tetravalent optionally substituted straight-chain, cyclic or branched, saturated, unsaturated or aromatic hydrocarbon radicals which have 2 to 300 carbon atoms, preferred 5 to 200 carbon atoms, more preferred 8 to 150 carbon atoms, even more preferred 10 to 120 carbon atoms, which optionally contain one or more groups selected from —O—, —NH—, —C(O)—, —C(S)—, tertiary amino groups

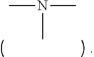

57. A compound of the formula (IIIa) according to any of the embodiments 50 to 56, wherein: r=0 to 50, preferred 0 to 20, more preferred 0 to 10, even more preferred 1 to 10, specifically 1 to 5, more specifically 0, 1, 2, 3, 4, 5, even more specifically r=2.

58. A compound of the formula (Ia) or (IIIa) according to any of the embodiments 45 to 57, wherein:

at least one of the radicals F contains at least one moiety

—R⁶(—O—C(O)—R⁶)ₘ—O—C(O)—R⁷,

—R⁶(—NR¹⁰—C(O)—R⁶)ₘ—O—C(O)—R⁷,

—R⁶(—NR¹⁰—C(O)—R⁶)ₘ—NR¹⁰—C(O)—R⁷,

preferably —R⁶(—O—C(O)—R⁶)ₘ—O—C(O)—R⁷, wherein R¹⁰, R⁶, R⁷, and m are as defined above.

59. A compound of the formula (IVa) according to any of the embodiments 45 to 49 and 51 to 53, wherein at least one of the radicals G contains at least one moiety

—R⁶(—C(O)—O—R⁶)ₘ—C(O)—O—R¹¹,

—R⁶(—C(O)—NR¹⁰—R⁶)ₘ—C(O)—O—R¹¹,

—R⁶(—C(O)—NR¹⁰—R⁶)ₘ—C(O)—NR¹⁰—R¹¹,

preferably —R⁶(—C(O)—O—R⁶)ₘ—C(O)—O—R¹¹, wherein R¹⁰, R⁶, R¹¹, and m are as defined above.

60. A compound of the formula (Ia), (IIIa) or (IVa) according to any of the previous embodiments 45 to 59, wherein low melting and high melting fatty acids ≥C5 are specifically positioned within the R⁶ and R⁷ containing ester elements of the general formula (IIa) and within the R⁶ and R¹¹ containing ester elements of the general formula (Va):

—R⁶(—X—C(O)—R⁶)ₘ—X—C(O)—R⁷ (IIa) and

—R⁶(—C(O)—X—R⁶)ₘ—C(O)—X—R¹¹ (Va),

in particular in the moieties

—O—C(O)—R⁶—(O—C(O)—R⁶)ₘ—O—C(O)—R⁷ and

—NR¹⁰—C(O)—R⁶—(O—C(O)—R⁶)ₘ—O—C(O)—R⁷,

wherein X, R⁶, R⁷, R¹¹ and R¹⁰ are as defined above,

and wherein

in one or more moieties of the formula (IIa) at least one, preferred more than one, more preferred one, two or three low melting fatty acids ≥C5 each forming a group R⁶ are contained in the radical or the radicals R⁶ adjacent to R⁷, while at least one, preferred more than one, more preferred one, two or three high melting fatty acids ≥C5 form the radical or radicals R⁶ at the opposite terminus of a R⁶- and R⁷-containing ester element of the formula (II), or in one or more moieties of the formula (IIa) at least one, preferred more than one, more preferred one, two or three high melting fatty acids ≥C5 each forming R⁶ form the radical or radicals R⁶ adjacent to R⁷, while at least one, preferred more than one, more preferred one, two or three low melting fatty acids ≥C5 form the radical or radicals R⁶ at the opposite terminus of a R⁶- and R⁷-containing ester element of the formula (IIa), and t is preferable when at least 50% of all moieties of the formula (IIa) display such a positioning of R⁶-radicals derived from high-melting and low-melting fatty acids as described above, it is more preferred when more than 80% of all moieties of the formula (IIa) display such a positioning of R⁶-radicals derived from high-melting and low-melting fatty acids as described above, and it is most preferably when all moieties of the general formula (IIa) display such positioning of residues R⁶ and R⁷ as described above,

or wherein

in one or more moieties of the formula (Va) at least one, preferred more than one, more preferred one, two or three low melting fatty acids ≥C5 each forming a group R⁶ are contained in the radical or the radicals R⁶ adjacent to R¹¹, while at least one, preferred more than one, more preferred one, two or three high melting fatty acids ≥C5 form the radical or radicals R⁶ at the opposite terminus of a R⁶- and R¹¹-containing ester element of the formula (Va), or in one or more moieties of the formula (Va) least one, preferred more than one, more preferred one, two or three high melting fatty acids ≥C5 each forming R⁶ form the radical or radicals R⁶ adjacent to R¹¹, while at least one, preferred more than one, more preferred one, two or three low melting fatty acids ≥C5 form the radical or radicals R⁶ at the opposite terminus of a R⁶- and R¹¹-containing ester element of the formula (Va), and it is preferable when at least 50% of all moieties of the formula (Va) display such a positioning of R⁶-radicals derived from high-melting and low-melting fatty acids as described above, it is more preferred when more than 80% of all moieties of the formula (Va) display such a positioning of R⁶-radicals derived from high-melting and low-melting fatty acids as described above, and most preferably all moieties of the general formula (Va) display such positioning of residues R⁶ and R¹¹ as described above.

61. A compound of the formula (Ia), (IIIa) or (IVa) according to any of the embodiments 45 to 60, wherein in the moieties of the formula (IIa)

m=1 to 10, more preferred 1 to 6, even more preferred 2 to 6, specifically 1, 2, 3, 4, 5, 6, more specifically 1 or 2.

62. A compound of the formula (Ia), (IIIa) or (IVa) according to any of the embodiments 45 to 61, wherein:

R⁶ is independently selected from optionally substituted straight-chain, cyclic or branched, saturated or unsaturated hydrocarbon radicals which have up to 24 carbon atoms, preferred 1 to 24, more preferred 2 to 20 carbon atoms, even more preferred 8 to 18 carbon atoms.

63. A compound of the formula (Ia) or (IIIa) according to any of the embodiments 45 to 58 and 60 to 62, wherein:

R⁷ is independently selected from optionally substituted straight-chain, cyclic or branched, saturated or unsaturated hydrocarbon radicals which have 1 to 36 carbon atoms, preferred 1 to 24 carbon atoms, more preferred 1 to 18 carbon atoms, even more preferred 8 to 18 carbon atoms.

64. A compound of the formula (Ia) or (IIIa) according to any of the embodiments 45 to 58 and 60 to 63, wherein the total number of carbon atoms in R⁶+R⁷ (Σcarbon atoms R⁶, R⁷) in each single moiety of the general formula (IIa) composed of R⁶ and R⁷ is 10 to 300, preferred 15 to 200, more preferred 20 to 150, even more preferred 30 to 100.

65. A compound of the formula (IVa) according to any of the embodiments 45 to 49, 51 to 53 and 59 to 62, wherein the total number of carbon atoms in R⁶+R¹¹ (Σcarbon atoms R⁶, R¹¹) in each single moiety of the general formula (a) composed of R⁶ and R⁷ is 10 to 300, preferred 15 to 200, more preferred 20 to 150, even more preferred 30 to 100.

66. A compound of the formula (Ia), (IIIa) or (IVa) according to any of the embodiments 45 to 65, wherein:

R⁶ is derived from monohydroxy carboxylic acids with up to 25 carbon atoms, preferably independently selected from the groups consisting of glycolic acid, lactic acid, 2-hydroxy butyric acid, 3-hydroxy-butyric acid, 4-hydroxy butyric acid, 14-hydroxy tetradecanoic acid, 10-hydroxy stearic acid, 12-hydroxy stearic acid, ricinoleic acid, and lesquerolic acid.

67. A compound of the formula (Ia) or (IIIa) according to any of the embodiments 45 to 58, 60 to 64, and 66, wherein:

R⁷ is derived from carboxylic acids with up to 25 carbon atoms which do not have hydroxyl substituent, preferably independently selected from the group consisting of acetic acid, propanoic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, docosanoic acid, 2-ethyl hexanoic acid, 2,2-dimethyl propionic acid, 2,2-dimethyl heptanoic acid, 2,2-dimethyl octanoic acid, neodecanoic acid, undecyl-10-en-ic acid, oleic acid, linoleic acid, linolenic acid, and erucic acid.

68. A compound of the formula (Ia) or (IIIa) according to any of the previous embodiments 45 to 58 and 60 to 64, and 66 to 67, wherein at least one, preferably both of R⁶ and R⁷ of the moieties of the general formula (IIa) are derived from unsaturated carboxylic acids.

69. A compound of formula (IIIa) according to any of the embodiments 50 to 58, 60 to 64, and 66 to 68, wherein R⁴ is selected from divalent to tetravalent, such as divalent, trivalent, tetravalent, preferably divalent, optionally substituted straight-chain, cyclic or branched, saturated, unsaturated or aromatic hydrocarbon radicals which have up to 200 carbon atoms, more preferred up to 150 carbon atoms, even more preferred up to 100 carbon atoms, specifically up to 80 carbon atoms, and preferably has at least 2, more preferred at least 10, more preferred as least 14 carbon atoms, which optionally contain one or more groups selected from —O—, —NH—, and —C(O)—, and wherein if a plurality of R⁴ is present in formula (111), they can be the same or different.

70. A compound of formula (IIIa) according to any of the embodiments 50 to 58, 60 to 64, and 66 to 69, wherein in the compound of the general formula (III) R⁴ comprises at least one ester group (—O—C(O)—, or —C(O)—O—, respectively).

71. A compound of formula (IIIa) according to any of the embodiments 50 to 58, 60 to 64, and 66 to 70, wherein in the compound of formula (III) R⁴ is derived from dicarboxylic acids, tricarboxylic acids or tetracarboxylic acids, in particular dicarboxylic acids, such as succinic acid, oxalic acid, malonic acid, malic acid, tartaric acid, maleic acid, itaconic acid, succinic acid, sebacic acid, dimer acids, amino-functional dicarboxylic acids, such as D-glutamic acid, and a dicarboxylic acid of the formula:

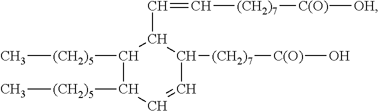

and condensation products of hydroxy carboxylic acids, in particular, from ricinoleic acid or lesquerolic acid, and dicarboxylic acids, such as succinic acid, oxalic acid, malonic acid, malic acid, tartaric acid, maleic acid, itaconic acid, succinic acid, sebacic acid, dimer acids, amino-functional dicarboxylic acids, such as D-glutamic acid, and a dicarboxylic acid of the formula:

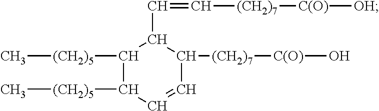

or

R⁴ is derived from amide condensation products of amino acids with maleic acid or succinic acid, such as N-maleoyl-β-alanine ((E)-4-(2-carboxyethylamino)-4-oxo-but-2-enoic acid), N-succinyl-β-alanine (4-[(2-hydroxy-1-methyl-2-oxo-ethyl)amino]-4-oxo-butanoic acid), N-maleoyl-asparagine (4-amino-2-[[(E)-4-hydroxy-4-oxo-but-2-enoyl]amino]-4-oxo-butanoic acid); or R⁴ is derived from the ester condensation products of divalent alcohols, i.e. ethylene glycol, 1,2-propylene glycol, 1,3-propylene glycol, 1,3-butane diol, 1,4-butanediol, with dicarboxylic acid anhydrides, i.e. maleic anhydride, succinic anhydride, phthalic anhydride;

or

R⁴ is derived from tri- or tetracarboxylic acids, such as citric acid, isocitric acid, trimelletic acid, pyromellitic acid, cyclobutane tetracarboxylic acid; or

R⁴ is derived from the bisamide condensation products of amino acids with maleic acid or succinic acid, i.e. bis-(N-maleoyl)-lysine; or

R⁴ is derived from the ester condensation products of trivalent alcohols, such as glycerol, trimethylolpropane with dicarboxylic acid anhydrides, such as maleic anhydride, succinic anhydride, phthalic anhydride; or

R⁴ is derived from the ester condensation products of tetravalent alcohols, such as diglycerol, pentaerythritol with dicarboxylic acid anhydrides, such as maleic anhydride, succinic anhydride, phthalic anhydride, and

most preferred R⁴ is derived from the condensation products of ricinoleic acid or lesquerolic acid and succinic acid.

72. Use of the compound of the formula (Ia), (IIIa) or (IVa) as defined in any of the embodiments 45 to 71 in cosmetic formulations for skin and/or hair care, such as conditioners and shampoos.

73. Use of the compound of the formula (Ia), (IIIa) or (IVa) as defined in any of the embodiments 45 to 71,

for the treatment of fibers, preferred amino acid based fibers, more preferred human hair, in particular being useful for hair treatment, such as for hair treatment with color retention, for hair shine enhancement, for hair color enhancement, for hair color protection, for hair conditioning, for hair smoothening or softening, for improving manageability of the hair, in particular for improving the combability of the hair, and for improving the anti-frizz and antistatic properties of hairs.

74. Compositions containing at least one compound of the formula (I), (III) and (IV) or (Ia), (IIIa) and (IVa) as defined in the embodiments 1-71 for the treatment of hair selected from the group consisting of hair shampoo compositions, hair conditioning compositions, hair strengthening compositions, hair coloration or dyeing compositions, hair combability improving compositions, anti-frizz compositions, and hair rinse-off and leave-on compositions.

### Composition Examples

Below, a number of typical examples of these types of compositions are provided, in which the polymeric fatty acid compounds of the invention, specifically the compounds of the formulas (I), (III), (IV), (Ia), (IIIa) and (IVa) as defined above, may be advantageously used: Typical adjuvants in these types of compositions are, e.g., those materials described in A. Domsch: Die kosmetischen Praeparate [Cosmetic Preparations] Vol. I and II, 4th Edition, Verl. fuer chem. Industrie [Publishers for the Chemical Industry], U. Ziolkowsky, K G, Augsburg, and the International Cosmetic Ingredient Dictionary and Handbook 7^{th} Ed. 1997 by J. A. Wenninger, G. N. McEwen Vol. 1-4 by The Cosmetic, Toiletry and Fragrance Association Washington D.C.

Anionic Shampoo

This formulation example is intended as a basic formulation. Anionic shampoos customarily contain, but are not limited to, the following components:
alkylsulfates, alkylether sulfates, sodium lauryl sulfate, sodium lauryl ether sulfate, ammonium lauryl sulfate, ammonium lauryl-ether sulfate, TEA-lauryl sulfate, TEA-lauryl-ether sulfate, alkylbenzene sulfonates, α-olefinsulfonates, paraffin sulfonates, sulfosuccinates, N-acyltaurides, sulfate-glycerides, sulfatized alkanolamides, carboxylate salts, N-acyl-amino acid salts, silicones, etc.

| Components | wt-% |
|---|---|
| Ammonium lauryl sulphate | 10.00-30.00 |
| Ammonium lauryl-ether sulphate | 5.00-20.00 |
| Cocamidopropyl betaine | 0.00-15.00 |
| Lauramide DEA | 0.00-5.00 |
| Cocamide MEA | 0.00-5.00 |
| Dimethicone copolyol | 0.00-5.00 |
| (dimethylsiloxane glycol copolymer) | |
| Cyclopentasiloxane | 0.00-5.00 |
| Polymeric fatty acid | 0.50-5.00 |
| compound of the invention | |
| Polyquaternium-10 | 0.00-2.00 |
| Preservatives | 0.00-0.50 |
| Fragrance | 0.00-5.00 |
| Deionized water | q.s. 100% |
| Sodium chloride | q.s. |

Non-Ionic Shampoo

This formulation example is intended as a basic formulation. Non-ionic shampoos customarily contain, but are not limited to, the following components: monoalkanolamides, monoethanolamides, monoisopropanolamides, polyhydroxy derivatives, sucrose monolaurate, polyglycerine ether, amine oxides, polyethoxylated derivatives, sorbitol derivatives, silicones, etc.

| Components | wt-% |
|---|---|
| Lauramide DEA | 10.00-30.00 |
| Lauramide oxide | 5.00-20.00 |
| Cocamide Mea | 0.00-5.00 |
| Dimethicone copolyol | 0.00-5.00 |
| Polymeric fatty acid | 0.50-5.00 |
| compound of the invention | |
| Preservatives | 0.00-0.50 |
| Fragrance | 0.00-5.00 |
| Deionized water | q.s. 100% |
| Sodium chloride | q.s. |

Amphoteric Shampoo

This formulation example is intended as a basic formulation. Formulations of this category customarily contain, but are not limited to, the following components: N-alkyl-iminodipropionates, N-alkyl-iminopropionates, amino acids, amino acid derivatives, amido betaine, imidazolinium derivatives, sulfobetaines, sultaines, betaines, silicones, etc.

| Components | wt-% |
|---|---|
| PEG-80-sorbitane laurate | 10.00-30.00 |
| Lauroamphoglycinate | 0.00-10.00 |
| Cocamidopropyl-hydroxysultain | 0.00-15.00 |
| PEG-150-distearate | 0.00-5.00 |
| Laurylether-13-carboxylate | 0.00-5.00 |
| Polymeric fatty acid | 0.50-5.00 |
| compound of the invention | |
| Fragrance | 0.00-5.00 |
| Deionized water | q.s. 100% |
| Sodium chloride | q.s. |

Cationic Shampoo

This formulation example is intended only as a basic formulation. Formulations of this category customarily contain, but are not limited to, the following components: Bis-quaternary ammonium compounds, bis-(trialkylammonium acetyl)diamines, amido amines, ammonium alkylesters, silicones, etc.

| Components | wt-% |
|---|---|
| Laurylether-13-carboxylate | 10.00-30.00 |
| Isopropylmyristate | 5.00-20.00 |
| Cocamidopropyl-betaine | 0.00-15.00 |
| Lauramide DEA | 0.00-5.00 |
| Cocamide MEA | 0.00-5.00 |
| Polymeric fatty acid | 0.50-5.00 |
| compound of the invention | |
| Preservatives | 0.00-0.50 |
| Fragrance | 0.00-5.00 |
| Deionized water | q.s. 100% |
| Sodium chloride | q.s. |

Setting Agents

This formulation example is intended only as a basic formulation. Formulations of this category customarily contain, but are not limited to, the following components: Fatty acids, fatty acid esters, ethoxylated fatty acids, ethoxylated fatty acid esters, fatty alcohols, ethoxylated fatty alcohols, glycols, glycol esters, glycerin, glycerin esters, lanolin, lanolin derivatives, mineral oil, petrolatum, lecithin, lecithin derivatives, waxes, wax derivatives, cationic polymers, proteins, protein derivatives, amino acids, amino acid derivatives, humectants, thickeners, silicones, etc.

| Components | wt-% |
|---|---|
| Ceteareth-20 | 0.10-10.00 |
| Steareth-20 | 0.10-10.00 |
| Stearyl alcohol | 0.10-10.00 |
| Stearamidopropyl-dimethylamine | 0.00-10.00 |
| Dicetyldimonium-chloride | 0.00-10.00 |
| Polymeric fatty acid | 0.50-5.00 |
| compound of the invention | |
| Cyclopentasiloxane | 0.00-5.00 |
| Dimethicone | 0.00-5.00 |
| Preservatives | 0.00-0.50 |
| Fragrance | 0.00-5.00 |
| Deionized water | q.s. 100% |

“Clear Rinse-Off” Setting Agents

This formulation example is intended as a basic formulation. Formulations of this category customarily contain, but are not limited to, the following components: Fatty acids, fatty acid esters, ethoxylated fatty acids, ethoxylated fatty acid esters, fatty alcohols, ethoxylated fatty alcohols, glycols, glycol esters, glycerin, glycerin esters, lanolin, lanolin derivatives, mineral oil, petrolatum, lecithin, lecithin derivatives, waxes, wax derivatives, cationic polymers, proteins, protein derivatives, amino acids, amino acid derivatives, humectants, thickening agents, silicones, etc.

| Components | wt-% |
|---|---|
| Glycerin | 0.10-10.00 |
| Cetrimonium chloride | 0.00-10.00 |
| Polymeric fatty acid | 0.50-5.00 |
| compound of the invention | |
| Hydroxyethyl cellulose | 0.00-5.00 |
| Preservatives | 0.00-0.50 |
| Fragrance | 0.00-5.00 |
| Deionized water | q.s. 100% |

Foam Setting Agents for Hair

This formulation example is intended as a basic formulation. Formulations of this category contain, but are not limited to, the following components: Fatty acids, fatty acid esters, ethoxylated fatty acids, ethoxylated fatty acid esters, fatty alcohols, ethoxylated fatty alcohols, glycols, glycol esters, glycerin, glycerin esters, lanolin, lanolin derivatives, mineral oil, petrolatum, lecithin, lecithin derivatives, waxes, wax derivatives, cationic polymers, proteins, protein derivatives, amino acids, amino acid derivatives, humectants, thickening agents, silicones, solvents, ethanol, isopropanol, isoparaffin solvents, butane, propane, isobutane, CFC's fluorinated aerosol propellants, dimethylether, compressed gases, etc.

| Components | wt-% |
|---|---|
| Polymeric fatty acid | 0.50-5.00 |
| compound of the invention | |
| Nonoxynol-15 | 0.00-2.00 |
| Nonoxynol-20 | 0.00-2.00 |
| Aerosol propellants | 0.00-20.00 |
| Preservatives | 0.00-0.50 |
| Fragrance | 0.00-5.00 |
| Deionized water | q.s. 100% |

Pump Spray (Setting Agents) for Hair

This formulation example is intended only as a basic formulation. Formulations of this category customarily contain, but are not limited to, the following components: Fatty acids, fatty acid esters, ethoxylated fatty acids, ethoxylated fatty acid esters, fatty alcohols, ethoxylated fatty alcohols, glycols, glycol esters, glycerin, glycerin esters, lanolin, lanolin derivatives, mineral oil, petrolatum, lecithin, lecithin derivatives, waxes, wax derivatives, cationic polymers, proteins, protein derivatives, amino acids, amino acid derivatives, humectants, thickening agents, silicones, solvents, ethanol, isopropanol, isoparaffin solvents, etc.

| Components | wt-% |
|---|---|
| Polymeric fatty acid | 0.50-5.00 |
| compound of the invention | |
| Cyclomethicone | 0.00-80.00 |
| Ethanol | 0.00-80.00 |
| Preservatives | 0.00-0.50 |
| Fragrance | 0.00-5.00 |
| Deionized water | q.s. 100% |

Setting Agent Spray for Hair

This formulation example is intended as a basic formulation. Formulations of this category customarily contain, but are not limited to, the following components: fatty acids, fatty acid esters, ethoxylated fatty acids, ethoxylated fatty acid esters, fatty alcohols, ethoxylated fatty alcohols, glycols, glycol esters, glycerin, glycerin esters, lanolin, lanolin derivatives, mineral oil, petrolatum, lecithin, lecithin derivatives, waxes, wax derivatives, cationic polymers, proteins, protein derivatives, amino acids, amino acid derivatives, humectants, thickening agents, silicones, solvents, ethanol, isopropanol, isoparaff in solvents, butane, propane, isobutane, CFC's fluorinated aerosol propellants, dimethylether, compressed gases, etc.

| Components | wt-% |
|---|---|
| Polymeric fatty acid | 0.50-5.00 |
| compound of the invention | |
| Cyclomethicone | 0.00-80.00 |
| Ethanol | 0.00-50.00 |
| Aerosol propellants | 0.00-50.00 |
| Preservatives | 0.00-0.50 |
| Fragrance | 0.00-5.00 |
| Deionized water | q.s. 100% |

Gel Setting Agents for Hair

This formulation example is intended as a basic formulation. Formulations of this category customarily contain, but are not limited to, the following components: thickening agents, cellulose derivatives, acrylic acid derivatives, fixative polymers, conditioning chemicals, glycols, glycol esters, glycerin, glycerin esters, lanolin, lanolin derivatives, mineral oil, petrolatum, lecithin, lecithin derivatives, waxes, wax derivatives, cationic polymers, proteins, protein derivatives, amino acids, amino acid derivatives, humectants, silicones, solvents, ethanol, isopropanol, isoparaffin solvents, etc.

| Components | wt-% |
|---|---|
| Polymeric fatty acid | 0.50-5.00 |
| compound of the invention | |
| Hydroxyethyl cellulose | 0.00-2.00 |
| Citric acid | 0.00-2.00 |
| Preservatives | 0.00-0.50 |
| Fragrance | 0.00-5.00 |
| Deionized water | q.s. 100% |

Rinse Off Conditioner

This formulation example is intended as a basic formulation. Formulations of this category customarily contain, but are not limited to, the following components: hydrocarbon based cationic conditioning agents, silicone based cationic conditioning agents, high melting fatty compounds, low melting oil like ester compounds, thickening agents, cellulose derivatives, fixative polymers, ethylene glycols, propylene glycols, glycol esters, glycerin, glycerin esters, monohydric alcohols, polyhydric alcohols, cationic polymers, nonionic and betaine co-emulsifiers, silicones, complexing agents, solvents, fragrances, vitamins, solvents, etc.

| Components | wt-% |
|---|---|
| Polymeric fatty acid | 0.50-5.00 |
| compound of the invention | |
| Cetyl Hydroxyethyl cellulose | 0.00-3.00 |
| Cetearyl alcohol | 0.00-3.00 |
| Citric acid | 0.00-2.00 |
| Glyceryl stearate and | 0.00-3.00 |
| PEG-100 Stearate | |
| Tetrasodium EDTA | 0.00-1.00 |
| Deionized water | q.s. 100% |

Styling Gel for Hair

This formulation example is intended as a basic formulation. Formulations of this category customarily contain, but are not limited to, the following components: Fixative polymers, lacquers, acrylic acid derivatives, cellulose derivatives, vinyl derivatives, conditioning chemicals, glycols, glycol esters, glycerin, glycerin esters, lanolin, lanolin derivatives, mineral oil, petrolatum, lecithin, lecithin derivatives, waxes, wax derivatives, cationic polymers, proteins, protein derivatives, amino acids, amino acid derivatives, humectants, thickening agents, silicones, solvents, ethanol, isopropanol, isoparaffin solvents, etc.

| Components | wt-% |
|---|---|
| Polymeric fatty acid | 0.50-5.00 |
| compound of the invention | |
| Fixing agents | 0.10-10.00 |
| Hydroxyethyl cellulose | 0.00-2.00 |
| Citric acid | 0.00-2.00 |
| Fragrance | 0.00-5.00 |
| Deionized water | q.s. 100% |

Styling Spray for Hair

This formulation example is intended as a basic formulation. Formulations of this category customarily contain, but are not limited to, the following components: Fixative polymers, lacquers, vinyl derivatives, fatty acids, fatty acid esters, ethoxylated fatty acids, ethoxylated fatty acid esters, fatty alcohols, ethoxylated fatty alcohols, glycols, glycol esters, glycerin, glycerin esters, lanolin, lanolin derivatives, mineral oil, petrolatum, lecithin, lecithin derivatives, waxes, wax derivatives, cationic polymers, proteins, protein derivatives, amino acids, amino acid derivatives, humectants, thickening agents, silicones, solvents, ethanol, isopropanol, isoparaffin solvents, butane, propane, isobutane, CFC's fluorinated aerosol propellants, dimethylether, compressed gases, etc.

| Components | wt-% |
|---|---|
| Polymeric fatty acid | 0.50-5.00 |
| compound of the invention | |
| Cyclomethicone | 0.00-80.00 |
| Fixing agents | 0.10-10.00 |
| Ethanol | 0.00-50.00 |
| Aerosol propellants | 0.00-50.00 |
| Preservatives | 0.00-0.50 |
| Fragrance | 0.00-5.00 |
| Deionized water | q.s. 1.00% |

Pump Spray (Styling) for Hair

This formulation example is intended as a basic formulation. Formulations of this category customarily contain, but are not limited to, the following components: vinyl derivatives, fixative polymers, lacquers, fatty acids, fatty acid esters, ethoxylated fatty acids, ethoxylated fatty acid esters, fatty alcohols, ethoxylated fatty alcohols, glycols, glycol esters, glycerin, glycerin esters, lanolin, lanolin derivatives, mineral oil, petrolatum, lecithin, lecithin derivatives, waxes, wax derivatives, cationic polymers, proteins, protein derivatives, amino acids, amino acid derivatives, humectants, thickening agents, silicones, solvents, ethanol, isopropanol, isoparaffin solvents, butane, propane, isobutane, CFC's fluorinated aerosol propellants, dimethylether, compressed gases, etc.

| Components | wt-% |
|---|---|
| Polymeric fatty acid | 0.50-5.00 |
| compound of the invention | |
| Cyclomethicone | 0.00-80.00 |
| Fixing agents | 0.10-10.00 |
| Ethanol | 0.00-50.00 |
| Preservatives | 0.00-0.50 |
| Fragrance | 0.00-5.00 |
| Deionized water | q.s. 100% |

The use of the polymeric fatty acid derivatives specified in the invention for applications in the hair care field produces favorable results with respect to strengthening, shine, fixing (hold), body, volume, moisture regulation, color retention, protection against environmental factors (UV, salt water, etc.), manageability, combability, anti-frizz, anti-static properties, ability to dye, etc.

### Further Formulation Examples

In the following formulation examples, all values given represent the amount in “wt-% of the total composition” unless otherwise noted.

Naturally Derived′ Crystal Clear, Betaine-Free Conditioning: Shampoo

Phase A

| | | |
|---|---|---|
| Aqua | | q.s. to 100 |
| Polyquta 400 KC (KCl Limited) | Anti-static cationic | 0.2 |
| (Polyquaternium-10) | polyquaternium | |
| | polymer | |

Phase B

| | | |
|---|---|---|
| Pureact WS Conc (Innospec) | mild anionic | 9.4 |
| (Sodium methyl cocoyl taurate) | surfactant | |
| Pureact Gluco L (Innospec) | Non ionic | 3.6 |
| (Lauryl glucoside) | surfactant foam | |
| | boosting/cleanser | |
| Pureact MS-CG (Innospec) | Mild anionic | 3.6 |
| (Sodium methyl oleoyl taurate) | surfactant | |
| Pureact LSR (Innospec) | Mild anionic | 1.35 |
| (Sodium lauroyl sarcosinate) | surfactant | |

Phase C

| | | |
|---|---|---|
| Polymeric fatty acid | Nonionic | 1 |
| compound of the invention | conditioning agent | |
| Surfac SB09 (Surfachem) | amphoteric | 9.4 |
| (Cocamidopropyl hydroxysultaine) | surfactants | |

Phase D

| | | |
|---|---|---|
| Sodium benzoate | Preservatives | 0.5 |

Phase E

| | |
|---|---|
| Pomette (Azur Fragrances) | 0.5 |
| (Fragrance) | |

Phase F

| | | |
|---|---|---|
| Citric acid (50% w/w) | pH adjuster | q.s. to pH |
| | | 4.2-4.7 |

Phase G

| | |
|---|---|
| Sodium chloride | qs to 4500- |
| | 8000 cps |
| | (max 0.8%) |

Procedure

To a vortex of aqua Polyquta 400 KC was added and mixed until it is fully dispersed and clear. Phase A was heated to 40-45° C. Pureact WS Conc, Pureact Gluco L and Pureact MS-CG were homogenized by heating to 40-45° C. and mixing the products before adding to the main vessel. Sequentially, the ingredients in phase B were added and mix until uniform and clear. Slowly, Surfac SB09 & the polymeric fatty acid compound of the invention (phase C) were added to the main vessel and mixed until uniform. The vessel was cooled to below 40° C. and then the preservative was added, it was mixed until the mixture was clear and uniform. Fragrance was added and mixed until it is fully emulsified and clear. It was adjust pH to 4.2-4.7 with citric acid solution (50% w/w) as required. Small aliquots of sodium chloride, (0.2% w/w) were added as required until the desired viscosity was obtained.

Fatal Attraction Hair Mist

Phase A

| | | |
|---|---|---|
| Water | | 85.03 |
| Panthenol | Active | 0.5 |
| Dissolvine GL-38(AkzoNobel speciality chemicals) | Chelating | 0.15 |
| (Water, tetrasodium glutamate diacetate, sodium | agent | |
| hydroxide) | | |
| Gluconolactone SB (MakingCosmetics) | Preservative | 2 |
| (Gluconolactone, sodium benzoate, calcium | | |
| gluconate) | | |
| Propanediol | Humectant | 2 |

Phase B

| | | |
|---|---|---|
| Keracyn (Centerchem) (Propanediol, Water, | Antioxidant | 1 |
| Glycerin, Cynara Scolymus (Artichoke) Leaf | | |
| Extract) | | |
| Sensfeel for Her (Centerchem) (Propanediol, | Fragrance | 5 |
| Jasminum Officinale Flower Extract, Ceratonia | | |
| Siliqua Fruit Extract, Phenethyl Alcohol) | | |

Phase C

| | | |
|---|---|---|
| Fragrance | | 0.25 |
| Polymeric fatty acid | Nonionic | 1 |
| compound of the invention | conditioning | |
| | agent | |
| Makigreen solve+ (Daito Kasei) (Caprylyl/capryl | natural | 2.5 |
| glycoside, polyglyceryl-4 caprate, polyglyceryl-6 | solubilizer | |
| laurate, pentylene glycol, sodium | | |
| dilauramidoglutaminde lysine) | | |

Phase D

| | |
|---|---|
| Sodium hydroxide, 20% | 0.57 |

Procedure

Phase A was added to a vessel with gentle agitation. It was mixed until transparent. With agitation at RT, phase B was added into phase A. It was mixed until homogeneous. With continued agitation phase C was slowly added into Phase A/B with fitting mixer. When the mixture was uniform, the pH value was adjusted to 5.00-5.50 with phase D.

Have A Peachy Day Jelly Shampoo

Phase A

| | | |
|---|---|---|
| Deionized water | | 39.95 |
| Endiquest GL-47S (Coast Southwest) | Chelating | 0.6 |
| (Tetrasodium glutamate diacetate) | agent | |
| Glycerin 99.7% USP Kosher (Coast Southwest, | Humectant | 3 |
| Inc.) (Glycerin) | | |
| Green Tea Concentrate (Coast Southwest, Tea | Active | 2 |
| Guys) (Water (and) camellia sinensis) | | |
| Synthalen W2000 (Coast Southwest, 3V | Anionic | 4 |
| Sigma-USA) (Acrylates/palmeth-25 acrylate | acrylic | |
| copolymer) | copolymer | |

Phase B

| | | |
|---|---|---|
| Endinol MILD B-SF65 (Coast Southwest) | Sulfate-free | 40 |
| (Sodium cocoyl isethionate (and) cocamidopropyl | surfactant | |
| hydroxysultaine (and) lauryl glucoside (and) | package | |
| cocamidopropylamine (and) | | |
| caprylyl/capryl glucoside) | | |
| NaOH 40% w/w solution (Sodium hydroxide) | | q.s. |

Phase C

| | | |
|---|---|---|
| Yangu Oil (Coast Southwest, International | Active | 3 |
| Cosmetic Science Centre) (Calodendrum capense | | |
| nut oil) | | |
| Polymeric fatty acid | Nonionic | 1 |
| compound of the invention | conditioning | |
| | agent | |
| Cosmosil 660 Shea Oil (Coast Southwest, | Active | 1.5 |
| International Cosmetic Science Centre) | | |
| (Butyrospermum parkii (shea) oil) | | |
| Olivatis 19 (Coast Southwest, Medolla Limited) | Active | 3 |
| (Olive oil polyglyceryl-6 esters (and) | | |
| phospholipids) | | |

Phase D

| | | |
|---|---|---|
| Sharomix CPC30 (Coast Southwest, Sharon- | Preservative | 0.5 |
| Laboratories)(Phenylpropanol (and) caprylyl | | |
| glycol (and) chlorphenesin) | | |
| Fragrance | | 1.45 |

In the main vessel, the phase A ingredients in formula order were combined with shear mixing and heated to 140° F. to 149° F. (60-65° C.). Phase B was added to phase A in formula order with continuous mixing. The solution thickened once neutralized to desired pH. In a separate vessel, phase C ingredients were combined and heated to 140° F. to 149° F. (60-65° C.). Once uniform, phase C was added to phase AB under propeller mixing. Phase D was combined in a separate vessel, then added to the main vessel under continuous mixing. The mixture was transfer to the final container.

PEG-Free Shampoo

Phase A

| | | |
|---|---|---|
| Standapol ES-2 (BASF) (Sodium laureth | Anionic | 25.2 |
| sulfate) | Surfactant | |
| Plantaren 2000 N UP (BASF) (Decyl | Nonionic | 15.1 |
| glucoside) | surfactant | |
| Lexaine C (Inolex) (Cocamidopropyl | Amphoteric | 10.2 |
| betaine) | surfactant | |
| Sulfochem AEG Surfactant Blend | Surfactant | 4 |
| (Lubrizol) (Ammonium lauryl sulfate | blend | |
| (and) ALES (and) CAPB (and) cocamide | | |
| DEA (and) lauramide DEA) | | |
| Glycerin, USP (Dow Chemical) | Humectant | 0.5 |
| (Glycerin) | | |

Phase B

| | | |
|---|---|---|
| Polymeric fatty acid | nonionic | 1 |
| compound of the invention | conditioning | |
| | agent | |
| Floraesters K-20W Jojoba (Floratech) | Oil | 5.3 |
| (Hydrolyzed jojoba esters (and) | | |
| Floratech water (Aqua)) | | |

Phase C

| | | |
|---|---|---|
| Deionized water | | q.s. 100 |
| Quatrisoft Polymer LM-200 (Dow | cationic | 0.1 |
| Chemical) (Polyquaternium-24) | polyquaternuim | |
| | polymer | |
| Tauranol I-78 (Innospec Performance | Isethionate | 4.7 |
| Chemicals) (Sodium cocoyl isethionate) | surfactant | |

Phase D

| | |
|---|---|
| Preservative | q.s. |
| Fragrance | q.s. |
| Color | q.s. |

The ingredients of phase A were mixed with moderate propeller agitation while heating to 70° C. until uniform. Phase B was added to phase A and mixed until uniform. Deionized water of phase C was heated to 65-70° C. and the Quatrisoft Polymer LM-200 was dissolved. Slowly Tauranol 1-78 was added once the Quatrisoft Polymer LM-200 had completely dissolved. Phase C was slowly added to phase AB. It was mix until uniform and cooled to 50° C. Phase D was added in the order listed to Phase ABC with moderate propeller agitation. The mixture was cooled to room temperature.

Repairing Shampoo Bar

Phase A

| | | |
|---|---|---|
| Sodium Cocoyl Isethionate, Stearic Acid | Anionic | 38 |
| | surfactant | |
| | with fatty | |
| | acid | |
| Water | | 8.7 |
| Citric Acid, 50% | | 0.5 |
| AMA-PROT (Centerchem) (Water, Glycerin, | Extract | 3 |
| Amaranthus Caudatus Seed Extract, Zea Mays | | |
| (Corn) Starch.) | | |
| KERACYN(Centerchem) (Propanediol, Water, | Extract | 3 |
| Glycerin, Cynara Scolymus (Artichoke) Leaf | | |
| Extract.) | | |
| BAICAPIL(Centerchem) (Propanediol, Water, | Active | 2 |
| Arginine, Lactic Acid, Glycine Soja (Soybean) | | |
| Germ Extract, Triticum Vulgare (Wheat) Germ | | |
| Extract, Scutellaria Baicalensis Root Extract.) | | |

Phase B

| | | |
|---|---|---|
| Sucrose Palmitate | Nonionic | 1 |
| | surfactant | |
| Sodium Methyl Cocoyl Taurate | Mild anionic | 27 |
| | solid | |
| | surfactant | |
| Erythritol | | 6 |

Phase C

| | | |
|---|---|---|
| Polymeric fatty acid | Nonionic | 1 |
| compound of the invention | conditioning | |
| | agent | |
| VITAOILS PLUS (Centerchem) (Helianthus Annuus | Oils | 8 |
| (Sunflower) Seed Oil, Cocos Nucifera (Coconut) | | |
| Oil, Linum Usitatissimum (Linseed) Seed Oil, | | |
| Persea Gratissima (Avocado) Oil, Argania Spinosa | | |
| Kernel Oil, Macadamia Ternifolia Seed Oil.) | | |
| Sucrose Tetrastearate Triacetate | Fatty acid | 1.5 |
| | modifed | |
| | sugar | |

Phase D

| | |
|---|---|
| Fragrance | 0.3 |

In the main vessel, the components of Phase A were added with gentle mixing, and it was heated to 70-75° C. Phase B was added into Phase A with continued mixing and maintaining a temperature of 70-75° C. Phase C was added into Phase AB with continued mixing and maintaining a temperature of 70-75° C. When the batch is uniform, it was cooled Phase D was added to the batch. When the batch was cooled to 45° C., the pre-heated sticks were filled. The sticks were placed in a freezer for 12-24 hrs. before the first use.

Frozen yogurt hair mask

Phase A

| | | |
|---|---|---|
| Deionized water (Water) | | 57.64 |
| Phytic Acid Extreme (Phytic acid, water) | Active | 0.5 |
| Liponic Bio EG-1 (Glycereth-26) | Humectant/ | 5 |
| | emulsifier | |

Phase B

| | | |
|---|---|---|
| Carbopol Ultrez 21 (Acrylates/C10-30 alkyl acrylate | polyacrylic | 0.35 |
| crosspolymer) | acid | |
| | derivative | |

Phase C

| | |
|---|---|
| Sodium hydroxide 10% solution (Water, sodium | 1.91 |
| hydroxide) | |

Phase D

| | | |
|---|---|---|
| Lipomulse Luxe MB (Vantage Personal Care ) | Non ionic | 3 |
| (Cetearyl alcohol, glyceryl stearate, PEG-40 | Surfactant/ | |
| stearate, ceteareth-20) | Emulsifier | |
| Lipovol C-76 (Cocos nucifera (coconut) oil) | conditioning | 6 |
| | Oil | |
| Polymeric fatty acid | Nonionic | 1 |
| compound of the invention | condioning | |
| | agent | |
| Avocado oil organic (Persea gratissima (avocado) | Conditioning | 6 |
| oil) | oil | |
| Iso Jojoba 35 (Simmondsia chinensis (jojoba) butter) | Conditioning | 3 |
| | oil | |

Phase E

| | |
|---|---|
| Preservative | 0.6 |

Phase F

| | | |
|---|---|---|
| Coconut Avocado Hair Milk (Water, cocos nucifera | Active | 15 |
| (coconut) oil, persea gratissima (avocado) oil, | | |
| propanediol, glyceryl stearate, phospholipids, cocos | | |
| nucifera (coconut) water, cocos nucifera (coconut) | | |
| fruit juice, polyglyceryl-10 oleate, polyglyceryl-10 | | |
| dioleate, cetearyl alcohol, sodium stearoyl lactylate, | | |
| glycerin) | | |

Procedure

In the main beaker, Phase A was weighed and heated to 75° C. Phase B was sprinkled on the aqueous phase and it was waited until carbopol was fully hydrated. It was homogenized, Phase C was added to neutralize, and it was homogenized again. In an annex container, Phase D was weighed and heated to 75° C. D was added slowly into the aqueous phase under high stirring. Then, the emulsion was cooled down with moderate stirring. At 35° C., Phase E and Phase F were added and homogenized.

Crystal Clear Healthy Hair Shampoo

Phase A

| | | |
|---|---|---|
| Deionized Water | | 59 |
| Hostapon SCI 85P (Clariant) (Sodium cocoyl | Mild anionic | 3.5 |
| isethionate) | Surfactant | |
| Glucotain Plus (Clariant) (Capryloyl/Caproyl | Mild nonionic | 10 |
| Methyl Glucamide (and) Lauroyl/Myristoyl | Surfactant | |
| Methyl Glucamide) | | |
| Genopal LT (Clariant) (PEG 150 PG-2) | PEG based | 1.5 |
| | Thickner | |
| Amphosol CS-50 (Stepan)(Cocamidopropyl | Amphoteric | 9 |
| Hydroxysultaine) | Surfactant/ | |
| | foam booster | |

Phase B

| | | |
|---|---|---|
| Glucquat 125 (Lubrizol) (Lauryl Methyl | Humenctant | 1.5 |
| Gluceth-10 HydroxypropylDimonium Chloride) | | |
| Celquat 240C (Polyquaternium-10 (Akzo | Cationic | 0.15 |
| Nobel) | polyquaternium | |
| | conditioner | |
| Deionized water | | 10 |

Phase C

| | | |
|---|---|---|
| Polymeric fatty acid | nonionic | 1 |
| compound of the invention | conditioning | |
| | agent | |
| Nipaguard SCP (Clariant) (Phenoxyethanol | Preservative | 1 |
| (and) Sorbitan Caprylate) | | |

Phase D

| | |
|---|---|
| Sodium Hydroxide/Citric Acid | q.s. |
| Deionized Water | q.s. |

Procedure

In an appropriate vessel the water was added. While the water was heated to 80-85″C, the Hostapon SCI, Glucotain Plus and Amphosol CS-50 were added. With the temperature at 80-85° C., it was mixed until uniform and removed from the heat. In a separate beaker, Celquat 240C, Glucquat 125 and Deionized water were mixed until uniform. Once homogenous, Celquat/Gluquat Blend were added to the Main Batch (Phase A). Phase C ingredients were added one by one and mixed well. The solution pH was adjusted with 20% citric acid or 20% NaOH to pH 6.0 to 6.5. The batch was filled to 100% with deionized water.

Hair Repairing Serum

Phase A

| | | |
|---|---|---|
| Deionized water | | 61 |
| Tetrasodium glutamate diacetate, | Stabilizer | 0.2 |
| sodium hydroxide, water | | |
| Hydroxyethyl cellulose | Thickner | 2 |
| Potassium sorbate | Stabilizer/ | 0.15 |
| | preservative | |
| Sodium benzoate | Preservatives | 0.15 |

Phase B

| | | |
|---|---|---|
| Water | | 19.92 |
| Polyquaternium-16 | Cationic | 0.5 |
| | polyquarternium | |
| | polymer | |

Phase C

| | | |
|---|---|---|
| Phenethyl alcohol | Masking agent | 0.8 |
| Polymeric fatty acid | nonionic | 1 |
| compound of the invention | condioning | |
| | agent | |
| PPG-26 butheth-26, PEG-40 | non-ionic | 5.4 |
| hydrogenated castor oil | surfactants | |
| Propanediol | humectant | 1.25 |

Phase D

| | | |
|---|---|---|
| Lactic acid, 50% | pH adjuster | 0.13 |

Phase E

| | | |
|---|---|---|
| Amber Extract MS (Provital | Active | 2.5 |
| S.A./Centerchem Inc.) | | |
| Keratrix (Provital | Active | 5 |
| S.A./Centerchem Inc.) | | |

Procedure:

In separate vessel, the components of phase A were added separately with mixing while heating to a temperature of 50° C. It was mixed until uniform and homogeneous. The mixture was cooled to a temperature <35° C. Each in separate vessel, the components of phase B and phase C were added separately with mixing and mixed until uniform. When the main vessel had cooled to 35° C., phases B and C were added to phase A. It was mixed until uniform. Phase D was added to the main vessel to adjust pH to 4.80-5.40. Phase E was added to the main vessel with gentle agitation, and it was mixed until uniform.

Hair Repairing Serum with Keratrix

Phase A

| | | |
|---|---|---|
| Deionized water | | 66 |
| Dissolvine GL-38(AkzoNobel speciality | Chelating | 0.2 |
| chemicals) (Water, tetrasodium glutamate | agent | |
| diacetate, sodium hydroxide) | | |
| Hydroxyethyl cellulose | Suger based | 2 |
| | thickner | |
| Potassium sorbate | | 0.15 |
| Sodium benzoate | | 0.15 |

Phase B

| | | |
|---|---|---|
| Water | | 19.92 |
| Polyquaternium-16 | Cationic | 0.5 |
| | Polyquarternium | |
| | polymer | |

Phase C

| | | |
|---|---|---|
| Phenethyl Alcohol | | 0.8 |
| Polymeric fatty acid | Nonionic | 1 |
| compound of the invention | conditioning | |
| | agent | |
| Solubilisant LRI (Sensient Cosmetic & | non-ionic | 0.4 |
| Fragrances) (PPG-26 Butheth-26, PEG-40 | surfactants | |
| hydrogenated castor oil) | mixture | |
| Propanediol | | 1.25 |

Phase D

| | |
|---|---|
| Lactic acid, 50% | 0.13 |

Phase E

| | |
|---|---|
| Amber Extract MS (Provital/Centerchem) | 2.5 |
| Keratrix (Provital/Centerchem) | 5 |

In separate vessel, the components of phase A were added separately with mixing while heating to a temperature of 50° C. It was mixed until uniform and homogeneous and the mixture was cooled to a temperature <35° C. Each in a separate vessel, the components of phase B and phase C were added separately with mixing, and mixed until uniform. When the main vessel had cooled to 35° C., phases B and C were added to phase A, and it was mixed until uniform. Phase D was added to the main vessel to adjust pH to 4.80-5.40. Phase E was added to the main vessel with gentle agitation, and it was mixed until uniform.

Put More Life In Your Hair Clay Mask

Phase A

| | | |
|---|---|---|
| Deionized water (Deionized water) | | 70.9 |
| Dissolvine NA2-S (Coast Southwest, Akzo Nobel | | 0.5 |
| Functional Chemicals) (Disodium EDTA) | | |
| Glycerin 99.7% USP Kosher (Coast Southwest) | | 2 |
| Conditioner P10 (Coast Southwest, 3V Sigma | cationic | 0.8 |
| USA)(Polyquaternium-10) | polyquaternium | |
| | polymer | |

Phase B

| | | |
|---|---|---|
| Polymeric fatty acid | nonionic | 1 |
| compound of the invention | conditioning | |
| | agent | |
| Olivatis 19 (Coast Southwest, Medolla Limited) | polyglyceryl | 3 |
| (Olive oil polyglyceryl-6 esters (and) | ester with | |
| phospholipids) | phospholipids | |
| Olivatis 18 (Coast Southwest, Medolla Limited) | polyglyceryl | 4 |
| (Olive oil polyglyceryl-6 esters (and) sodium | ester with | |
| stearyl Lactylate (and) cetearyl alcohol) | fatty alcohols | |
| Cosmodan 20 (Coast Southwest, International | | 4 |
| Cosmetic Science Centre) (Elaeis guineensis | | |
| (palm) oil (and) brassica campestris | | |
| (rapeseed) seed oil) | | |
| Tamanu Butter (Coast Southwest, Inc., | Butter | 2 |
| International Cosmetic Science Centre) | | |
| (Calophyllum inophyllum seed oil (and) | | |
| butyrospermum parkii nut extract) | | |
| Kpangnan Butter (Coast Southwest, International | | 2 |
| Cosmetic Science Centre) (Pentadesma | | |
| butyraceae seed butter) | | |
| Coconut Olein (Coast Southwest, | | 1 |
| International Cosmetics Science Centre) | | |
| (Cocos nucifera (coconut) oil) | | |
| Cosmosil B (Coast Southwest, Inc., | Oil | 0.5 |
| International Cosmetic Science Centre) | | |
| (Brassica campestris seed oil | | |
| (and) oryza sativa bran oil) | | |
| Endimate 33V (Coast Southwest) | vegetable- | 1 |
| (Caprylic/capric triglyceride) | origin, | |
| | medium chain | |
| | triglyceride | |
| Glossamer L6600 (Coast Southwest) (Brassica | | 2 |
| campestris/aleurites fordi oil copolymer) | | |

Phase C

| | | |
|---|---|---|
| Pelavie Yellow Clay (Coast Southwest, | Clay | 4 |
| Inc., The Innovation Company) (Bentonite) | | |

Phase D

| | |
|---|---|
| Sharomix 704 (Coast Southwest., Sharon | 0.8 |
| Laboratories) (Benzoic Acid (and) | |
| dehydroacetic acid (and) phenoxyethanol) | |

Procedure:

Phase A ingredients were combined in formula order into main vessel with propeller mixing and heated to 60-70° C. In a separate vessel, the phase B ingredients in formula order were combined under propeller mixing and it was heated to 60-70° C. Once both phase A and phase B were fully uniform, phase B was added to phase A with continuous mixing. Once fully dispersed, heat was discontinued. Once the temperature is at 35-40° C., phase C was added to phase AB with continuous mixing. Phase D was added to Phase ABC, mixing was discontinued and it was switched to a homogenizer. The mixture was homogenized for 10-30 seconds. Once complete, the mixture was transferred to a holding vessel.

Detox Hair Sleeping Pack

Phase A

| | | |
|---|---|---|
| Water | | 87.03 |
| Panthenol | Multifuctional | 2 |
| | benefits | |
| Ethylhexylglycerin | Preservative | 0.5 |
| Citric acid, 50% | pH adjuster | 0.6 |
| Sodium methylparaben | Preservative | 0.25 |
| Lexgard Natural MB (INOLEX | Emollient | 1 |
| Incorporated)Glyceryl | | |
| caprylate, glyceryl undecylenate | | |

Phase B

| | | |
|---|---|---|
| Polymeric fatty acid | nonionic | 1 |
| compound of the invention | conditioning | |
| | agent | |
| Acrylkates/C10-30 alkyl acrylate | polyacrylic | 1 |
| crosspolymer | acid | |
| | derivative | |

Phase C

| | | |
|---|---|---|
| Ama-leaf (Provital S.A./Centerchem) | Active | 1 |
| Kercyn (Provital S.A./Centerchem) | Active | 2 |
| Pronalen bio-protect znsn (Provital | Active | 2 |
| S.A./Centerchem) | | |
| Sodium hydroxide, 20% | | 1.62 |

Procedure

In main vessel, thecomponents of phase A were added separately with gentle mixing until the mixture was uniform. Phase B was dispersed into the vessel with high shear mixing. When the batch was uniform, the components of phase C were added individually, wherein it was mixed until uniform before thenext addition. The pH value was adjusted to 5.40-6.00 with sodium hydroxide, and it was mixed until the gel was uniform.

Clean Beauty Light & Clean Conditioner

Phase A

| | |
|---|---|
| Water, deionized (water) | 89.8 |

Phase B

| | | |
|---|---|---|
| Emulsense HC (Inolex) (Brassicyl | cationic | 1.5 |
| isoleucinate esylate, brassica alcohol) | anti-static | |
| | agent and | |
| | emulsifier | |
| Argan Oil (DSM Nutritional Products) | Conditioning | 0.5 |
| (Argania Spinosa kernel oil) | oil | |
| Cetyl alcohol | co-surfactant | 0.7 |
| Neossance Hemisqualane CN | Emollient | 1 |
| (Aprinnova/Centerchem) (C13-16 isoparaffin) | | |

Phase C

| | | |
|---|---|---|
| Polymeric fatty acid | nonionic | 1 |
| compound of the invention | conditioning | |
| | agent | |
| Ama-prot (Provital S.A./Centerchem) (Water, | Active | 1.5 |
| glycerin, amaranthus caudatus seed extract, | | |
| zea mays starch) | | |
| Baicapil (Provital S.A./Centerchem) | Active | 2 |
| (Propanediol, water, arginine, lactic acid, | | |
| glycine soja(soybean) germ extract, triticum | | |
| vulgare (wheat) germ extract, Scutellaria | | |
| baicalensis root extract) | | |
| Leucidal SF Max (Active Micro Technologies) | Active | 2 |
| (Lactobacillus ferment) | | |

Phase A was heated to 75° C. under agitation. In a separate vessel, the ingredients of phase B were heated to 75° C. under agitation. Phase B was added to phase A and mixing for 10 minutes was continued. The heat was removed and stirring was continued until the product reached 40° C. The ingredients of phase C were combined and mixed well under medium agitation. Phase C was added to phase A/B at 40° C. and stirring was continued until the product reached room temperature Heat phase A to 75° C. under agitation. In a separate vessel, heat ingredients of phase B to 75° C. under agitation. Add phase B to phase A and continue mixing for 10 minutes. Remove heat and continue stirring until product reaches 40° C. Combine ingredient of phase C and mix well, under medium agitation. Add phase C to phase A/B at 40° C. and continue stirring until product reaches room temperature

Gently Bubbles It′ Mild Shampoo

Phase A

| | |
|---|---|
| Water (Aqua) | q.s. to 100 |

Phase B

| | | |
|---|---|---|
| Polyquta 400 KC (KCl Limited) | cationic | 0.2 |
| (Polyquaternium-10) | polyquaternium | |
| | polymer | |

Phase C

| | | |
|---|---|---|
| Iselux Ultra Mild (Innospec) (Aqua, sodium | Mild anionic, | 32 |
| lauroyl methyl isethionate, cocamidopropyl | amphoteric & | |
| betaine, sodium methyl oleoyl taurate, | nonionic | |
| lauryl glucoside, coco-glucoside) | surfactants | |

Phase D

| | | |
|---|---|---|
| Polymeric fatty acid | Nonionic | 2 |
| compound of the invention | conditioning | |
| | agent | |
| Emulsil S-393 (Innospec) (PEG-12 | hydrophilic | 0.75 |
| dimethicone) | silicones | |

Phase E

| | |
|---|---|
| Odersynthesis fragrance (Intarome) | 0.25 |
| (Fragrance) | |

Phase F

| | | |
|---|---|---|
| Euxyl K100 (Schülke) | preservative | 0.05 |
| (Methylchloroisothiazolinone, | | |
| methylisothiazolinone, benzyl alcohol) | | |

Phase G

| | | |
|---|---|---|
| Citric acid (50% solution) | pH adjuster | q.s. to pH |
| (Citric acid, water) | | 5.5-6.0 |

Water was charged into a mixing vessel and Polyquaternium-10 (B) was sprinkled into the water, and it was mixed until clear. With moderate mixing Iselux Ultra Mild (C) was poured into the main vessel. Phase (D) was mixed to the batch. It was mix until clear, then the desired fragrance and preservative was added and the pH was adjusted using citric acid (50% w/w solution) (G).

Coconut Dream Conditioner

Phase A

| Water | | to 100.00 |
|---|---|---|
| Dehyquart A-CA (BASF) (Cetrimonium | cationic | 0.5 |
| chloride) | Surfactant | |
| Sodium EDTA | | 0.15 |

Phase B

| | | |
|---|---|---|
| Crodacol C90 (Croda) (Cetyl alcohol) | Emulsion | 1.5 |
| | stabiliser | |
| Crodacol S95 (Croda) (Stearyl alcohol) | Emulsion | 2.5 |
| | stabiliser | |
| Polymeric fatty acid | nonionic | 1 |
| compound of the invention | conditioning | |
| | agent | |
| Coconut Oil | conditioning | 0.3 |
| | oil | |
| Arquat 2HT-75 PG (Akzo Nobel) | cationic | 0.8 |
| (Quaternium-18, propylenglycol) | quaternary | |
| | conditioning | |
| | agent | |
| Eumulgin B2 (BASF) (Ceteareth-20) | non-ionic | 0.5 |
| | emulsifier | |

Phase C

| | | |
|---|---|---|
| Tocopherolacetate | active | 0.2 |
| BeauSil AMO 8950 EM (CHT) | Amodimethicone | 3.5 |
| (Amodimethicone, cetrimonium | | |
| chloride, trideceth-12) | | |

Phase D

| | | |
|---|---|---|
| Euxyl K320 (schülke) (Preservative | Preservative | 0.5 |
| agent) | | |
| Fragrance | | q.s. |

Procedure

The ingredients of Phase A were mixed and heat to 80° C. The ingredients of phase B were blended at 80° C. Phase B was added to phase A. It is cooled down to 40° C. and phase C is added. It is cooled further, phase D is added, and the mixture is adjusted to pH 4.3-4.7.

Glycolic Acid Shampoo

Phase A

| | | |
|---|---|---|
| Deionized water | | 51.3 |
| Glycerin (Coast Southwest) | Humenctant | 4 |
| Endiquest GLDA (Coast | Stabilizer | 0.2 |
| Southwest) (Tetrasodium | | |
| glutamate diacetate) | | |
| Synthalen W2000 (Coast | anionic | 5 |
| Southwest, 3V-Sigma USA) | acrylic | |
| (Acrylates/palmeth-25 acrylate | copolymer | |
| copolymer) | | |

Phase B

| | | |
|---|---|---|
| Deionized water | | 5 |
| GlyAcid 70 (Coast Southwest, | active | 4 |
| CrossChem) (Glycolic acid) | | |
| NaOH (30% aq.) (Sodium | | q.s. |
| hydroxide) | | |

Phase C

| | | |
|---|---|---|
| Endinol Mild SF-65 (Coast | Mild anionic, | 20 |
| Southwest) (Sodium cocoyl | nonanionic & | |
| isethionate, cocamidopropyl | amphoteric | |
| hydroxysultaine, lauryl glucoside, | Surfactant | |
| cocamidopropylamine oxide, | blend | |
| caprylyl/capryl glucoside) | | |
| Polymeric fatty acid | nonionic | 1 |
| compound of the invention | conditioning | |
| | agent | |
| GlucoTain Clear (Coast | Nonionic | 5 |
| Southwest, Clariant) | Surfactants | |
| (Capryloyl/caproyl methyl | | |
| glucamide) | | |
| Enditeric COAB (Coast | Amphoteric | 5 |
| Southwest) (Cocamidopropyl | surfactant | |
| betaine) | | |

Phase D

| | |
|---|---|
| NaOH (30% aq.) (Sodium | q.s. |
| hydroxide) | |

Procedure

In the main vessel, phase A was added and mixed until uniform. Phase B is added to phase

A. In a side vessel, phase C is combined, then it was add slowly to phase AB, wherein the pH is required to be >4. The preservative was added to phase ABC. While the batch was initially discontinuous, mixing was continued. Slowly add the surfactant to phase ABCD. The batch will become uniform and increase in viscosity. Finally fragrance was added.

2-Phase Super Hydration Hair Treatment

Phase A

| | |
|---|---|
| Water (aqua) | to 100.00 |

Phase B

| | | |
|---|---|---|
| Jaguar C-162 (Solvay) | | 0.9 |
| (hydroxypropyl guar, | | |
| hydroxypropyltrimonium | | |
| chloride) | | |
| Glycerin (Merck) | humectant | 0.9 |

Phase C

| | | |
|---|---|---|
| Lactic Acid 80 (Lactic acid) | pH adjuster | 0.15 |
| BeauSil AMO 918 (CHT) | Amodimethicone | 1.1 |
| (Gluconamido | | |
| amodimethicone, trideceth-7, | | |
| trideceth-8) | | |
| Panthenol | multifictional | 0.2 |
| | active | |
| Sodium benzoate | Preservative | 0.1 |

Phase D

| | | |
|---|---|---|
| Polymeric fatty acid | nonionic | 1 |
| compound of the invention | conditioning | |
| | agent | |
| BeauSil Fluid 8301 (CHT) | emolient | 7 |
| (C13-15 alkane, | | |
| Isododecane, caprylyl | | |
| methicone) | | |
| BeauSil PEG 010 (CHT) | ambiphilic | 1 |
| (PEG/PPG-15/5 | surfactant | |
| dimethicone) | | |
| BeauSil Gum 8501 (CHT) | conditioning | 5 |
| (C13-15 alkane, | agent | |
| isododecane, caprylyl | | |
| methicone, dimethiconol) | | |

Phase E

| | |
|---|---|
| Dye and fragrance | q.s. |

Procedure

The ingredients of phase B were blended and added to phase A while mixing. Then the ingredients of phase C were added to phase AB. The ingredients of phase D were blended and added with high-shear to phase ABC. Then phase E was added.

Cleanse & Nourish Oil Shampoo

Phase A

| | | |
|---|---|---|
| Water deionized (Aqua) | | to 100 |
| Crodateric CAS 50 (Croda) (Cocamidopropyl | Amphoteric | 8 |
| hydroxysultaine (and) water (aqua)) | surfactant | |
| Crodateric CAB 30 (Croda) (Cocamidopropyl | Amphoteric | 15 |
| betaine (and) water (aqua)) | surfactant | |
| Jeelate ES-3 (Jeen) Sodium laureth sulfate 30%) | Anionic | 35 |
| | surfactant | |

Phase B

| | | |
|---|---|---|
| Versathix (Croda) (PEG-150 pentaerythrityl | | 1.5 |
| tetrastearate (and) PPG-2 hydroxyethyl | | |
| cocamide (and) water (aqua)) | | |
| Crovol A70 (Croda) (PEG-60 almond glycerides) | Nonionic | 2 |
| | emollient | |
| Cromollient SCE (Croda) (Di-PPG-2 myreth-10 | | 0.5 |
| adipate) | | |
| Polymeric fatty acid | nonionic | 1 |
| compound of the invention | conditioning | |
| | agent | |
| Arlasilk EFA (Croda) (Linoleamidopropyl | cationic | 1.5 |
| PG-dimonium chloride phosphate (and) propylene | surfactant | |
| glycol (and) water (aqua)) | | |
| Cropure Almond (Croda) (Prunus amygdalus | | 0.25 |
| dulcis (sweet almond) oil) | | |
| Procetyl AWS (Croda) (PPG-5-ceteth-20) | | 3 |

Phase C

| | |
|---|---|
| Phytessence French Oak (Crodarom) (Water | 0.5 |
| (aqua) (and) glycerin (and) quercus petraea | |
| fruit extract) | |
| Neolone 950 (Dow) | 0.1 |
| (Methylchloroisothiazolinone) | |
| Citric acid (25% solution) | 0.07 |
| Sodium chloride | 0.5 |

Phase A was heated to 75-80° C. Phase B was premixed and added to part A with medium speed mixing. It was cooled to 40° C. and Phase C was added. The pH was checked and adjusted if necessary using citric acid solution.

Hair Wax Formulation

Phase A

| Water (aqua) (deionized) | to 100 |
|---|---|
| Propylene glycol | 10 |
| Sorbitol | 7 |

Phase B

| | | |
|---|---|---|
| Mineral oil (Paraffinum liquidum) | | 12 |
| Sensolene Care DD (Hallstar Italia) | fatty | 3 |
| (Lauryl olivate) | acid/esters | |
| Steareth-20 | Nonionic | 20 |
| | surfactant | |
| Steareth-2 | Nonionic | 3 |
| | surfactant | |
| Polymeric fatty acid | Nonionic | 1 |
| compound of the invention | conditioning | |
| | agent | |
| SALCARE SC 96 (BASF) (Polyquaternium-37, | cationic | 2.5 |
| propylene glycol dicaprylate/dicaprate, PPG-1 | polyquaternium | |
| trideceth-6) | polymer | |
| BHT | | 0.1 |

Phase C

| | |
|---|---|
| Silica, titanium dioxide, tin oxide | 0.5 |

Phase D

| | |
|---|---|
| Preservative | a.n. |
| Fragrance (Parfum) | a.n. |

Phase A was prepared and heated to 75-80° C. Phase B was prepared and heated to 70-75° C. Phase B was added to phase A and homogenized for a few minutes using a suitable dispersion unit (e.g. Silverson, Ultra Turrax, etc.). It was cooled to 40° C., and the phases. C and D were added and mixed for a few minutes. The mixture was cooled to room temperature.

On-the-Go Hair Sherbet

Phase A

| | | |
|---|---|---|
| Water | | 81.25 |
| Trisodium ethylenediamide disuccinate | | 0.15 |
| Glyceryl caprylate, glyceryl undecylenate | Non ionic | 0.5 |
| | surfactant | |
| Panthenol | | 0.3 |
| Pentylene glycol | | 3 |

Phase B

| | | |
|---|---|---|
| Dehydroxanthan gum | | 0.6 |
| Sodium polyacrylate starch | polyacrylate | 0.5 |
| | derivative | |

Phase C

| | | |
|---|---|---|
| Polymeric fatty acid | nonionic | 1 |
| compound of the invention | conditioning | |
| | agent | |
| SME 253 PF (Momentive performance | Amodimethicone | 4 |
| materials) Amodimethicone, C11-15 pareth- | | |
| 7, laureth-9, glycerin, trideceth-12 | | |
| Lauryl methyl glyceth-10 | cationic | 0.5 |
| hydroxypropyldimonium chloride | polymer | |
| Isopentyldiol | | 4 |

Phase D

| | |
|---|---|
| Keracyn (Provital S.A./Centerchem Inc.) | 2 |
| Keranutri (Provital S.A./Centerchem Inc.) | 2 |
| Lactic Acid, 50% | 0.07 |
| Bismuth Oxychloride, mica, chromium oxide | 0.03 |
| green (CI 77299) | |
| Fragrance | 0.1 |

Phase A was added to a vessel with gentle agitation while heating to 45-50° C. It was mixed until uniform. With agitation, phase B was added into phase A. It was mixed until homogeneous. With continued agitation, phase C was added into phase A/B. When uniform, phase D ingredients were added individually to phase A/B/C with gentle agitation Between each addition it was mixed.

Dreamy Curls 24-Hr Weightless Foam

Phase A

| | | |
|---|---|---|
| Deionized water (Aqua) | | 92.66 |
| Styleze ES-1 polymer (Ashland) (Guar | | 1 |
| hydroxypropyltrimonium chloride) | | |
| Citric Acid (20% aq. Solution) (Local) (Citric | | 0.24 |
| acid) | | |
| Benecel E4M HPMC (Ashland) (Hydroxypropyl | | 0.1 |
| methylcellulose) | | |
| Amphosol CA (Stepan) (Cocamidopropyl | Amphoteric | 3 |
| betaine) | surfactant | |
| Polymeric fatty acid compound of the invention | nonionic | 1 |
| | conditioning | |
| | agent | |
| Glycerin USP (Jeen International) (Glycerin) | | 1 |
| Optiphen BSB-W preservative (Benzyl alcohol, | | 1 |
| aqua (water), sodium benzoate, potassium | | |
| sorbate) | | |

Procedure:

Water was added to the main container and mixed with propeller agitation. Styleze ES-1 was added into the vortex to disperse. Citric acid was added and mixed for approximately 10-15 min. Benecel E4M was added and mixed until no particles were seen. The polymeric fatty acid compound of the invention, Amphosol CA, glycerin and Optiphen BSB-W were added one by one and mixed until uniform.

Sea Salt 2-in-1 Scalp Treatment Shampoo

Phase A

| | | |
|---|---|---|
| Water | | q.s. to 100 |
| Glycerin | | 7 |
| Polyquta 400 KC (KCl) (Polyquaternium-10) | Cationic | 0.1 |
| | polyquaternium | |
| | polymer | |

Phase B

| | | |
|---|---|---|
| Empigen BB (Innospec) (Lauryl betaine) | amphoteric | 3 |
| | surfactant | |
| Pureact Gluco C (Innospec) (Coco- | Nonionic | 3 |
| glucoside) | surfactant | |
| Pureact WS Conc (Innospec) (Sodium | Mild anionic | 17 |
| methyl cocoyl taurate) | surfactant | |

Phase C

| | | |
|---|---|---|
| Empilan EGDS/A (Innospec) (Glycol | Nonionic | 2.5 |
| distearate) | surfactant | |
| Iselux (Innospec) (Sodium lauroyl methyl | Mild anionic | 8 |
| isethionate) | isethionate | |

Phase D

| | |
|---|---|
| Citric acid (50% w/w solution) (Water, citric | q.s. to pH |
| acid) | 5.5-6.0 |

Phase E

| | | |
|---|---|---|
| Polymeric fatty acid | nonionic | 1 |
| compound of the invention | conditioning | |
| | agent | |
| Macadamia Nut Oil (Macadamia integrifolia | | 7 |
| (macadamia) seed oil) | | |
| Jojoba Seed Oil (Vantage) (Simmondsia | | 7 |
| chinesis (jojoba) seed oil) | | |

Phase F

| | |
|---|---|
| Cetearyl Alcohol (Naturally Thinking) | 3 |

Phase G

| | |
|---|---|
| Coarse sea salt crystals (Sodium chloride) | 40 |
| Orchid 107745 (Sozio) (Fragrance) | 0.5 |

Procedure:

Polyquaternium-10 was slowly added to water. Next, glycerin was added in the main vessel and mixed until fully dispersed and clear. Sequentially, Phase (B) ingredients were added and it was heated to 65-70° C. and mixed until homogenous. Phase (C) ingredients were added, the temperature was maintained at 65-70° C. and it was mixed until homogenous. The pH was adjusted with citric acid (50% w/w solution) to 5.5-6.0. Phase (E) ingredients were added and mixed until homogenous. Phase (F) was added while keeping the temperature at −65° C. and it was mixed until structure was obtained. Heating was stopped and cooled to 30° C., Phase (G) ingredients were added with mixing.

Anti-Humidity Sparkling Hair Serum

Phase A

| | | |
|---|---|---|
| Avocado Oil (Provital/Centerchem) | | 3 |
| AMA-Oil (Provital/Centerchem) | | 0.5 |
| Cyclopentasiloxane | cyclomethicone | 49 |
| Polymeric fatty acid | Nonionic | 1 |
| compound of the invention | conditioning | |
| | agent | |
| Vegelight 1214LC (Grant Industries, Inc.) | | 30.05 |
| (Coconut Alkanes, Coco-Caprylate/Caprate) | | |
| Tocopherol | | 0.2 |
| Dicaprylyl Ether | | 16 |

Phase B

| | |
|---|---|
| Fragrance | 0.15 |

Phase C

| | |
|---|---|
| KTZ SM INTERVAL BLUE (Kobo Products, | 0.1 |
| Inc) (Synthetic Fluorphlogopite, | |
| Titanium Dioxide) | |

Procedure:

In the main vessel, the components of Phase A were added separately with gentle mixing until the mixture was uniform and transparent. The remaining Phases were added individually, wherein it was mixed until uniform before the next addition.

D5 Free Primer with Argan Oil

Phase A

| | | |
|---|---|---|
| BeauSil Fluid 8301 (CHT) (C13-15 | Dimethicone | to 100.00 |
| alkane, isododecane and caprylyl | | |
| methicone) | | |
| Polymeric fatty acid | nonionic | 1 |
| compound of the invention | conditioning | |
| | agent | |

Phase B

| | | |
|---|---|---|
| BeauSil Gel 8017 (CHT) (C13-15 alkane, | dimethicone/ | 70 |
| isododecane and | vinyldimethicone | |
| dimethicone/vinyldimethicone | crosspolyme | |
| crosspolymer and caprylyl methicone) | | |

Phase C

| | |
|---|---|
| BeauSI Wax 070 (CHT)(Cetyl | 0.6 |
| dimethicone) | |
| Argan Oil (Argania spinose kernel oil) | 0.2 |
| Vitamin E (Tocopherol) | 0.05 |
| Fragrance | q.s. |

Procedure

Ingredients of phase B were added to phase A and mixed with low to medium shear. The ingredients of phase C were blended and added to phase AB.

### EXAMPLES

(The percentages refer to weight-% unless otherwise indicated.

As used herein, the term “castor oil” generally refers to ricinoleic acid triglyceride).

The term “DI water” refers to deionized water, i.e. water which has almost all of its mineral ions removed, for example by distillation, or, as applied most frequently, deionization using specially manufactured ion-exchange resins.

Remarks on the Nomenclature Used Herein for Estolide Moieties and Estolide Compounds

In the nomenclature for denoting the structure of the estolide groups as used in the following examples, which refers to the compounds from which the estolide moieties are at least formally obtained by esterification, the carboxylic acids from which the estolide moieties are at least formally derived are given in a sequential manner in parentheses. In case there are several subunits derived from the same acid in a row present in the estolide moiety and these are indicated in parentheses, wherein a subscript integer indicates the number of repeating units, the carboxylic acids are given in brackets.

It is noted that the specific carboxylic acids given in parentheses or brackets are not combined in a random structure, but they have exactly the sequence of hydroxyl-carboxylic acid-derived residues and carboxylic acid-derived residues, respectively, as indicated in the term used. Therein, the last carboxylic acid given in the term in parentheses or brackets, respectively, is the terminal carboxylic acid of the estolide moiety. Going from the beginning of the term in parentheses or brackets to the end of the term, the order of carboxylic acid residues linked by ester groups is displayed in the correct order and number of residues contained.

For example, the term “(12-hydroxy stearic acid-ricinoleic acid-oleic acid)” refers to an estolide moiety in which formally 12-hydroxy stearic acid molecule is linked via its OH group to the carboxylic acid group of a ricinoleic acid molecule by forming an ester group. The hydroxyl group of the said ricinoleic acid group is linked to an oleic acid molecule by forming an ester group with the carboxylic acid group of the oleic acid molecule. The oleic acid is in this example considered to be the terminal group of this specific estolide moiety, as, if the estolide moiety is a substituent of a higher-level structure (i.e. a more complex molecule), in general the estolide moiety is linked to the overall structure via linkage to the carboxylic acid group of the first mentioned residue of the term used for the estolide moiety. In this case, this is the first mentioned 12-hydroxy stearic acid residue, and the oleic acid residue is the terminal group of the estolide moiety.

Accordingly, in case the term used refers to a carboxylic acid chloride of an estolide structure, the acyl chloride group is necessarily formed from the carboxylic acid group of the first-mentioned carboxylic acid residue in parentheses, i.e. the most remote one from the terminal group.

In case terms as “dimer” or “trimer” and so on are used, this refers to the number of carboxylic acid-derived subunits of the estolide moieties.

In the same manner, the term “[(ricinoleic acid)₂-oleic acid] estolide” refers to an estolide moiety or compound in which formally a ricinoleic acid molecule or residue is linked via its OH group to the carboxylic acid group of a further ricinoleic acid molecule by forming an ester group. The hydroxyl group of the latter ricinoleic acid group mentioned is linked to an oleic acid molecule by forming an ester group with the carboxylic acid group of the oleic acid molecule. The oleic acid is considered to be the terminal group of this specific estolide moiety, as, if the estolide moiety is a substituent of a higher-level structure (i.e. a more complex molecule), the estolide moiety is linked to the overall structure via linkage to the carboxylic acid group of the first mentioned ricinoleic acid residue, and the oleic acid residue is the terminal group of the estolide moiety.

It is further noted that the exact structure of the estolides is primarily clarified by the structural formulas, which are thoroughly provided for the example compounds, and that the structures of the example compounds can also be clearly derived by the skilled artisan from the detailed experimental procedures provided.

### Synthesis Example 1

Synthesis of a (Ricinoleic Acid-Oleic Acid) Dimer Chloride (Estolide Dimer Chloride)

In a 100 ml three-necked bottle, equipped with refluxing condenser, thermometer and magnetic stirrer, dropping funnel and gas outlet tube, 34.91 g (0.117 mol) ricinoleic acid were placed at room temperature under a nitrogen atmosphere. The material was heated to 50° C. Upon stirring, 35.2 g (0.117 mol) of oleic acid chloride were added slowly for 0.5 h. During the addition process the temperature was increased to 80° C. The temperature was maintained for 1 h. The complete conversion of the OH groups was confirmed by means of ¹H NMR spectroscopy.

The bottle content was cooled to 50° C. 27.83 g (0.234 mol) SOCl₂ were added during 0.5 h. The evolution of gas bubbles indicated the progress of the reaction. The temperature was increased to 80° C. and maintained for 2 h. Afterwards, volatiles were removed under reduced pressure (80° C./0.5 h/20 mmHg). The complete conversion of the C(O)OH groups was confirmed by means of ¹H NMR spectroscopy.

A brownish, transparent oil essentially having the following structure was obtained:

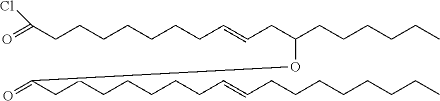 [SMILES: CCCCCCCC/C=C/CCCCCCCC(=O)OC(C/C=C/CCCCCCCC(=O)Cl)CCCCCC]

### Synthesis Example 2 (not According to the Invention)

Synthesis of the Acetyl Derivative of Castor Oil

In a 100 ml three-necked bottle, equipped with refluxing condenser, thermometer and magnetic stirrer, dropping funnel and gas outlet tube, 64 g (0.0686 mol) castor oil were placed at room temperature. 17.76 g (0.2263 mol) acetic acid chloride were added within 0.5 h. The temperature increased to 38° C. during the addition. The temperature was increased to 80° C. and maintained at this temperature for 4 h. Afterwards, volatiles were removed under reduced pressure (70° C./4 h/20 mmHg). The product was washed 3 times with 10 g deionized water and volatiles were finally removed under reduced pressure (70° C./2 h/20 mmHg). The complete conversion of the OH groups was confirmed by means of ¹H NMR spectroscopy.

A slightly yellowish transparent oil essentially having the following structure was obtained:

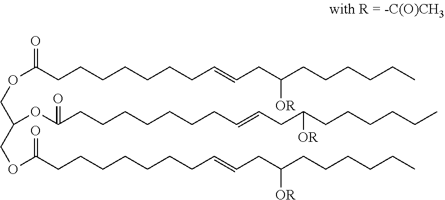

### Example 1

Synthesis of the Pivaloyl Derivative of Castor Oil

In a 100 ml three-necked bottle, equipped with refluxing condenser, thermometer and magnetic stirrer, dropping funnel and gas outlet tube 58 g (0.0621 mol) of castor oil were placed and heated to 80° C. 24.72 g (0.2051 mol) pivalic acid chloride were added within 1 h. The reaction was continued at 80° C. for 12 h. Afterwards, volatiles were removed under reduced pressure (70° C./2 h/20 mmHg). The product was washed 3 times with 10 g deionized water and volatiles were finally removed under reduced pressure (70° C./3 h/20 mmHg). The complete conversion of the OH groups was confirmed by means of ¹H NMR spectroscopy.

A yellowish transparent oil essentially having the following structure was obtained:

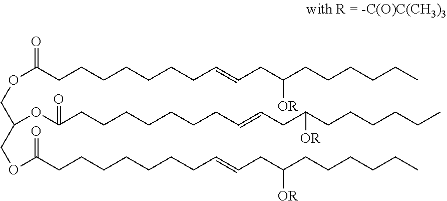

### Example 2

Synthesis of the Oleyl Derivative of Castor Oil

In a 100 ml three-necked bottle, equipped with refluxing condenser, thermometer and magnetic stirrer, dropping funnel and gas outlet tube, 42 g (0.0450 mol) castor oil were placed at room temperature. 40.62 g (0.135 mol) oleic acid chloride were added within 1 h. The reaction temperature increased during the addition to 34° C. The temperature was increased to 80° C. and was then maintained for 4 h. Afterwards, volatiles were removed under reduced pressure (70° C./2 h/20 mmHg). The product was washed 3 times with 10 g DI water and volatiles were finally removed under reduced pressure (70° C./3 h/20 mmHg). The complete conversion of the OH groups was confirmed by means of ¹H NMR spectroscopy.

A brownish transparent oil essentially having the following structure was obtained:

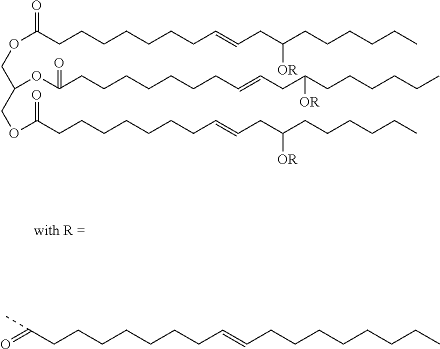

### Example 3

Synthesis of the (Ricinoleic Acid-Oleic Acid) Dimer Derivative of Castor Oil

In a 100 ml three-necked bottle, equipped with refluxing condenser, thermometer and magnetic stirrer, dropping funnel and gas outlet tube, 28 g (0.03 mol) castor oil were placed at room temperature. 52.32 g (0.09 mol) of the (ricinoleic acid-oleic acid) dimer chloride from synthesis example 1 were added within 1 h. The reaction temperature increased during the addition to 28° C. The temperature was increased to 100° C. and then maintained for 16 h. Afterwards, volatiles were removed under reduced pressure (70° C./2 h/20 mmHg). The product was washed 3 times with 10 g deionized water and volatiles were finally removed under reduced pressure (70° C./3 h/20 mmHg). The complete conversion of the OH groups was confirmed by means of ¹H NMR spectroscopy.

A brownish transparent oil essentially having the following structure was obtained:

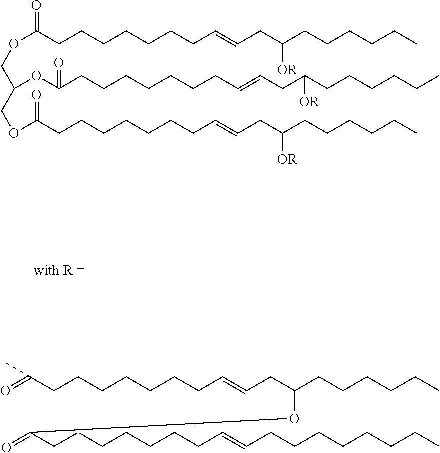

### Example 4

Synthesis of an Oligomerized (Ricinoleic Acid-Oleic Acid) Dimer Derivative of Castor Oil Having Succinyl Bridges

In a 100 ml three-necked bottle, equipped with refluxing condenser, thermometer and magnetic stirrer, dropping funnel and gas outlet tube, 28.9 g (0.0302 mol) castor oil were placed at room temperature. 3.19 g (0.0206 mol) succinic acid dichloride were added within 0.5 h. The reaction temperature was increased during the addition to 80° C. Afterwards, the temperature was maintained at 80° C. for 1 h. The partial conversion of the OH groups of castor oil was confirmed by means of ¹H NMR spectroscopy.

30 g (0.0516 mol) of the ricinoleic acid-oleic acid dimer chloride from synthesis example 1 were added at 80° C. within 0.5 h. The reaction was continued at 80° C. for 6 h. The product was washed with 20 g deionized water and volatiles were finally removed under reduced pressure (40° C./4 h/20 mmHg). The complete conversion of the OH groups of castor oil was confirmed by means of ¹H NMR spectroscopy.

A brown transparent oil essentially having the following structure was obtained:

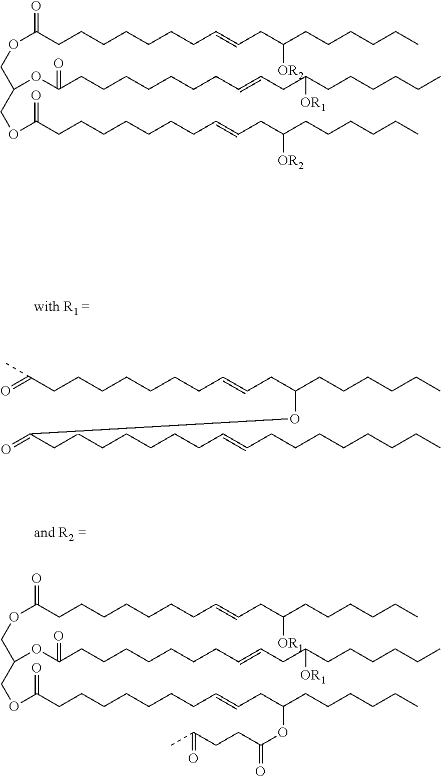

### Example 5

Synthesis of an Oligomerized (Ricinoleic Acid-Oleic Acid) Dimer Derivative of Castor Oil Having Estolide Bridges

In a 100 ml three-necked bottle, equipped with refluxing condenser, thermometer and magnetic stirrer, dropping funnel and gas outlet tube, 10.26 g (0.0344 mol) ricinoleic acid were placed at room temperature. 2.66 g (0.0172 mol) succinic acid dichloride were added and then heated to 80° C. for 0.5 h. The complete conversion of the OH groups was confirmed by means of ¹H NMR spectroscopy. 12.27 g (0.103 mol) SOCl₂ were added at 40° C. and the temperature was increased to 80° C. for 0.5 h. The excess of SOCl₂ was removed under reduced pressure (70° C./0.5 h/20 mmHg). Afterwards, 24.08 g (0.0258 mol) castor oil were added at 80′C and the reaction was continued for 1 h. The temperature increased during the addition to 80° C. Afterwards, the temperature was maintained at 80° C. for 1 h. The partial conversion of the OH groups was confirmed by means of ¹H NMR spectroscopy. 25 g (0.0430 mol) of the (ricinoleic acid-oleic acid) dimer chloride from synthesis example 1 were added at 80° C. and the temperature was increased to 100° C. for 2 h. The product was mixed with 10 g deionized water and volatiles were finally removed under reduced pressure (85° C./1 h/20 mmHg). The complete conversion of the OH groups was confirmed by means of ¹H NMR spectroscopy.

A brown transparent oil essentially having the following structure was obtained:

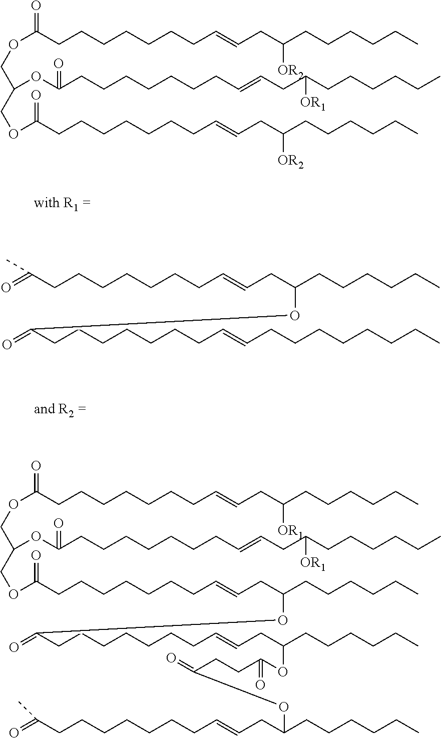

### Example 6

Synthesis of a (Ricinoleic Acid-Oleic Acid) Estolide Trimer Based Branched Derivative of Castor Oil

### Example 6a

Synthesis of a [(Ricinoleic Acid)₂-Oleic Acid] Estolide Trimer

In a 250 ml four-necked bottle, equipped with refluxing condenser, thermometer and magnetic stirrer, dropping funnel and gas outlet tube, 34.87 g (0.293 mol) SOCl₂ were placed at room temperature under a nitrogen atmosphere. Upon stirring, 110 g (0.195 mol) of the estolide dimer acid intermediate of synthesis example 1 were added slowly during 1 h. After the end of the addition the temperature was increased to 80° C. The temperature was maintained at 80° C. for 1 h. Volatiles were removed under reduced pressure (80° C./2 h/20 mmHg). Nitrogen was used to break the vacuum and 57.75 g (0.195 mol) ricinoleic acid were added to the carboxylic acid chloride intermediate at 80° C. during 45 minutes. The temperature was maintained for 2 h. Volatiles were removed under reduced pressure (40° C./2 h/20 mmHg). The conversion of the OH groups was determined by means of ¹H NMR spectroscopy. The conversion of the OH groups was 100%.

A brownish, transparent oil essentially having the following structure was obtained:

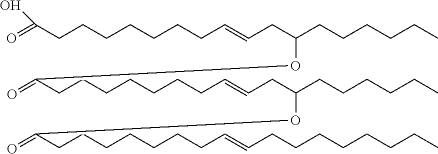 [SMILES: CCCCCCCC/C=C/CCCCCCCC(=O)OC(C/C=C/CCCCCCCC(=O)OC(C/C=C/CCCCCCCC(=O)O)CCCCCC)CCCCCC]

### Example 6b

Synthesis of a [(Ricinoleic Acid)₅Oleic Acid] Estolide Hexamer and the Corresponding [(Ricinoleic Acid)₅Oleic Acid] Chloride Hexamer

Two 100 ml three-necked bottles A and B, equipped with refluxing condenser, thermometer and magnetic stirrer, dropping funnel and gas outlet tube were flushed with nitrogen.

Bottle A was used to react fatty acid chlorides with ricinoleic acid yielding a chain extended fatty ester acid. Subsequent addition of SOCl₂ yielded the corresponding fatty ester acid chloride.

Bottle B was used to react the formed fatty ester acid chloride with ricinoleic acid yielding a chain extended fatty ester acid. Subsequent addition of SOCl₂ yielded the corresponding fatty ester acid chloride. This fatty acid chloride was transferred back to bottle A and was reacted with ricinoleic acid. The above described cycle was repeated until the hexamer estolide [(ricinoleic acid)₅-oleic acid] was prepared.

General Procedure for the Synthesis of Chain Extended Fatty Ester Acids:

The calculated amount on ricinoleic acid was placed in a bottle. An equimolar amount on fatty ester acid chloride was added slowly at room temperature. In order to complete the reaction, the temperature was increased to 80° C. for 3 h. The complete conversion of the OH groups was determined by means of ¹H NMR spectroscopy.

General Procedure for the Synthesis of Fatty Ester Acid Chlorides:

The calculated amount of fatty ester acid was placed in a bottle. SOCl₂ (threefold molar excess) was added slowly at room temperature. Afterwards, the mixture was heated to 80° C. The temperature was maintained for 3 h. Afterwards, the excess of SOCl₂ was removed under reduced pressure (80° C./2 h/20 mmHg). The complete conversion of the C(O)OH groups towards C(O)Cl groups was determined by means of ¹H NMR spectroscopy.

The following table summarizes the materials and the quantities used.

| | | fatty ester | ricinoleic | | |
|---|---|---|---|---|---|
| | | derivative | acid | SOCl₂ | |
| bottle | fatty ester derivative | amount [g] | amount [g] | amount [g] | target product |
| A | oleoyl chloride | 30.00 | 29.76 | | [(rici)₁-oleic acid] |
| A | [(rici)₁-oleic acid] | 59.76 | | 37.89 | [(rici)₁-oleic acid] |
| | | | | | chloride |
| B | [(rici)₁-oleic acid] | 46.26 | 23.75 | | [(rici)₂-oleic acid] |
| | chloride | | | | |
| B | [(rici)₂-oleic acid] | 70.01 | | 29.63 | [(rici)₂-oleic acid] |
| | | | | | chloride |
| A | [(rici)₂-oleic acid] | 52.00 | 18.01 | | [(rici)₃-oleic acid] |
| | chloride | | | | |
| A | [(rici)₃-oleic acid] | 70.01 | | 22.23 | [(rici)₃-oleic acid] |
| | | | | | chloride |
| B | [(rici)₃-oleic acid] | 55.50 | 14.50 | | [(rici)₄-oleic acid] |
| | chloride | | | | |
| B | [(rici)₄-oleic acid] | 70.00 | | 17.79 | [(rici)₄-oleic acid] |
| | | | | | chloride |
| A | [(rici)₄-oleic acid] | 57.87 | 12.14 | | [(rici)₅-oleic acid] |
| | chloride | | | | |
| A | [(rici)₅-oleic acid] | 50 | | 10.60 | [(rici)₅-oleic acid] |
| | | | | | chloride |

Note:
The term “rici” replaces the term “ricinoleic acid” in denoting a ricinoleyl radical.

A brownish, transparent oil essentially having the following structure [(rici)₅-oleic acid] was obtained:

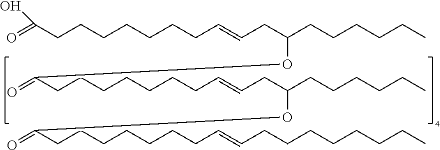

The corresponding [(rici)₅-oleic acid] chloride has the structure:

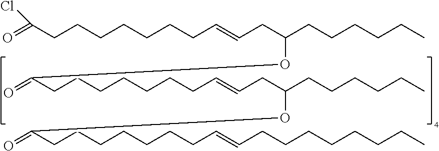

### Example 6c

Synthesis of an Alpha Branched Bis-[(Ricinoleic Acid)₂-Oleic Acid] Estolide Based on 2,2-Hydroxymethyl Propionic Acid

In a 100 ml three-necked bottle, equipped with refluxing condenser, thermometer and magnetic stirrer, dropping funnel and gas outlet tube 48.88 g (0.0567 mol) of the [(ricinoleic acid)₂-oleic acid] chloride of example 6b were mixed with 3.80 g (0.0284 mol) bis 2,2-hydroxymethyl propionic acid. The mixture was heated to 100° C. for 8 h. Volatiles were removed under reduced pressure (80° C./1 h/20 mmHg). The complete conversion of the OH groups was determined by means of ¹H NMR spectroscopy.

A brownish, transparent oil essentially having the following structure was obtained:

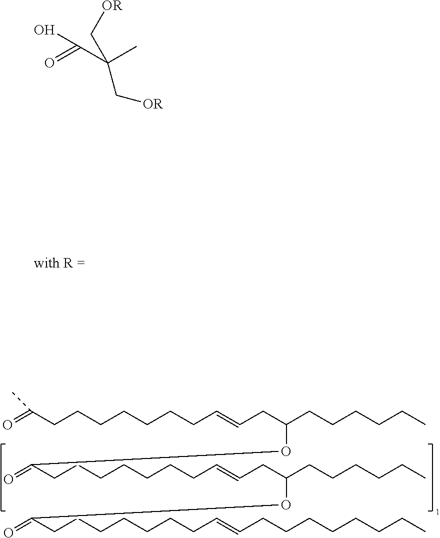

### Example 6d

Synthesis of a [(ricinoleic acid)₂-oleic acid] estolide Trimer-Based Branched Derivative of Castor Oil

In a 100 ml three-necked bottle, equipped with refluxing condenser, thermometer and magnetic stirrer, dropping funnel and gas outlet tube, 15.60 g (0.00874 mol) of the alpha branched bis-[(ricinoleic acid)₂-oleic acid] from example 6c were placed and heated to 60° C. 3.12 g (0.0262 mol) SOCl₂ were added within 10 minutes. Gas bubbles appeared. The temperature was increased to 80° C. and maintained for 3 h. Afterwards, volatiles were removed under reduced pressure (70 to 80° C./0.5 h/20 mmHg). The complete conversion of the OH groups was confirmed by means of ¹H NMR spectroscopy. 2.72 g (2.91 mol) castor oil was added and the mixture was maintained at 80° C. for 9 h. The complete conversion of the OH groups was confirmed by means of ¹H NMR spectroscopy.

A brownish transparent oil essentially having the following structure was obtained:

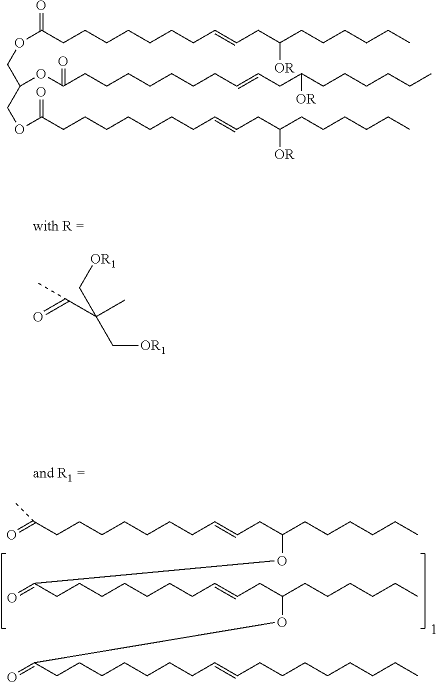

### Example 7a

Synthesis of a (12-hydroxy stearic acid-ricinoleic acid-oleic acid) estolide Trimer

Two 250 ml three-necked bottles A and B, equipped with refluxing condenser, thermometer and magnetic stirrer, dropping funnel and gas outlet tube were flushed with nitrogen. Bottle A was used to react oleic acid chloride with ricinoleic acid yielding a chain extended fatty ester acid. Subsequent addition of SOCl₂ yielded the corresponding fatty ester acid chloride. Bottle B was used to react the formed fatty ester acid chloride with 12-hydroxy stearic acid yielding a chain extended fatty ester acid.

General Procedure for the Synthesis of Chain Extended Fatty Ester Acids:

The calculated amount of 12-hydroxy stearic acid was placed in a bottle. An equimolar amount on fatty ester acid chloride was added slowly at room temperature. In order to complete the reaction, the temperature was increased to 80° C. for 3 h. The complete conversion of the OH groups was determined by means of ¹H NMR spectroscopy.

General Procedure for the Synthesis of Fatty Ester Acid Chlorides:

The calculated amount fatty ester acid was placed in a bottle. SOCl₂ (threefold molar excess) was added slowly at room temperature. Afterwards, the mixture was heated to 80° C. The temperature was maintained for 3 h. Afterwards, the excess of SOCl₂ was removed under reduced pressure (80° C./2 h/20 mmHg). The complete conversion of the C(O)OH groups towards C(O)Cl groups was determined by means of ¹H NMR spectroscopy.

The following table summarizes the materials and the quantities used.

| | | fatty ester | | SOCl₂ | |
|---|---|---|---|---|---|
| | fatty ester | derivative | fatty acid, | amount | |
| bottle | derivative | amount [g] | amount [g] | [g] | target product |
| A | oleoyl | 55.00 | ricinoleic acid | | (rici-oleic acid) |
| | chloride | | acid 55.3 | | |
| A | (rici-oleic | 102.91 | | 38.5 | (rici-oleic acid) |
| | acid) | | | | chloride |
| B | (rici-oleic | 100.00 | 12 hydroxy | | (12-hydroxy |
| | acid) | | stearic | | stea-rici-oleic |
| | chloride | | acid 51.68 | | acid) |

Note:
The term “rici” replaces the term “ricinoleic acid” in denoting a ricinoleyl radical, the term “hydroxyl-stea” replaces the term “12-hydroxy stearic acid” in denoting a 12-hydroxyl stearyl radical.

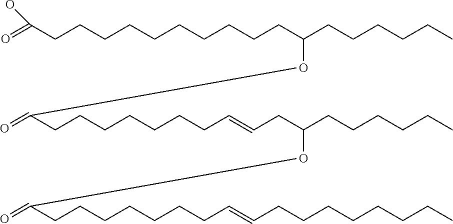 [SMILES: CCCCCCCC/C=C/CCCCCCCC(=O)OC(C/C=C/CCCCCCCC(=O)OC(CCCCCC)CCCCCCCCCCC(=O)O)CCCCCC]

### Example 7b

Synthesis of a (12-hydroxy stearic acid-ricinoleic acid-oleic acid) estolide Trimer Derivative of Castor Oil

In a 100 ml three-necked bottle, equipped with refluxing condenser, thermometer and magnetic stirrer, dropping funnel and gas outlet tube, 17.00 g (0.0201 mol) of the (12-hydroxy stearic acid-ricinoleic acid-oleic acid) trimer from example 7a were placed and heated to 60° C. 7.18 g (0.0603 mol) SOCl₂ were added within 10 minutes. Gas bubbles appeared. The temperature was increased to 80° C. and maintained for 3 h. Afterwards, volatiles were removed under reduced pressure (70 to 80° C./0.5 h/20 mmHg). The complete conversion of the OH groups was confirmed by means of ¹H NMR spectroscopy. 6.25 g (0.0067 mol) castor oil were added and the mixture was maintained at 80° C. for 13 h. The complete conversion of the OH groups was confirmed by means of ¹H NMR spectroscopy.

A brownish transparent oil essentially having the following structure was obtained:

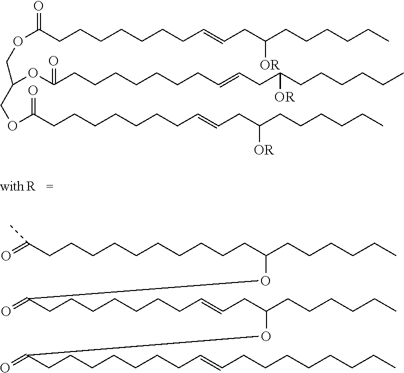

Table 1 summarizes the viscosities of the castor oil derivatives described in the examples 1 to 7b. The viscosity of the compounds of the examples 1, 2, 3, 4, 5, 6d and 7b was determined using a Brookfield DV2T viscosimeter, spindle 4, 200RPM, 23° C.:

| example | 1 | 2 | 3 | 4 | 5 | 6d | 7b | castor oil |
|---|---|---|---|---|---|---|---|---|
| viscosity | 352 | 224 | 608 | 1728 | 3616 | 3040 | 2784 | 928 |
| mPa · s | | | | | | | | |

### Example 8

Synthesis of an oligomerized (ricinoleic acid-oleic acid) Dimer Derivative of Castor Oil Having Estolide Bridges

In a 250 ml three-necked bottle, equipped with refluxing condenser, thermometer and magnetic stirrer, dropping funnel and gas outlet tube, 7.65 g (0.0493 mol) succinic acid dichloride were placed at room temperature. 29.45 g (0.0987 mol) ricinoleic acid were added within five minutes and the mixture was heated to 70° C. for 1 h. The complete conversion of the OH groups was confirmed by means of ¹H NMR spectroscopy. 35.23 g (0.296 mol) SOCl₂ were added at 70° C. within 10 minutes and the temperature afterwards increased to 80° C. for 1 h. The excess of SOCl₂ was removed under reduced pressure (70° C./0.5 h/20 mmHg). Afterwards, 55.27 g (0.0592 mol) castor oil were added at 45° C., the temperature increased to 80° C. and the reaction continued for 1 h. The partial conversion of the OH groups was confirmed by means of ¹H NMR spectroscopy. 45.89 g (0.0789 mol) of the (ricinoleic acid-oleic acid) dimer chloride from synthesis example 1 were added at 80° C. within 15 minutes and the temperature afterwards increased to 90° C. for 1.5 h. The product was mixed with 10 g deionized water and volatiles were finally removed under reduced pressure (70° C./1 h/20 mmHg). The complete conversion of the OH groups was confirmed by means of ¹H NMR spectroscopy.

A brown transparent oil is obtained essentially being a derivatized castor oil hexamer consisting of (on average) a tetramer of the core element being terminated with one R₂ and one R₃ element:

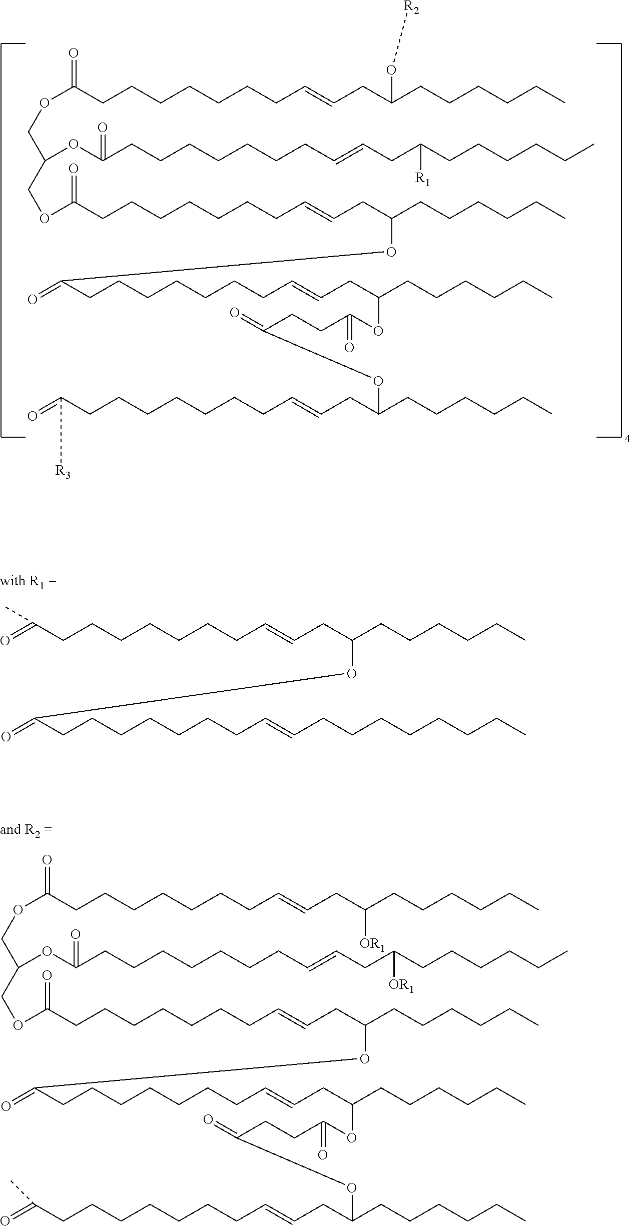

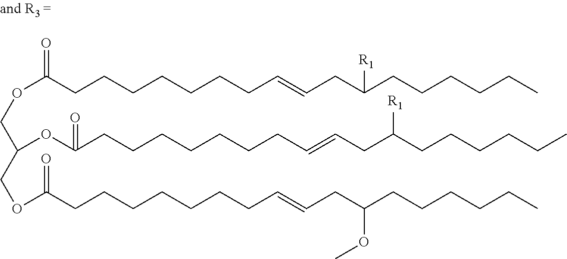

### Example 9

Synthesis of an Oligomerized Oleic Acid Derivative of Castor Oil Having Succinic Acid Ester Bridges

In a 250 ml three-necked bottle, equipped with refluxing condenser, thermometer and magnetic stirrer, dropping funnel and gas outlet tube, 85.75 g (0.0919 mol) castor oil were placed at room temperature. 11.86 g (0.0768 mol) succinic acid dichloride were added within five minutes and the mixture heated to 80° C. for 2 hrs. The complete conversion of the OH groups was confirmed by means of ¹H NMR spectroscopy. 36.86 g (0.122 mol) oleic acid chloride were added at 80° C. within 10 minutes, the temperature afterwards increased to 90° C. and maintained for 2.5 hrs. The product was mixed with 10 g deionized water and volatiles were finally removed under reduced pressure (70° C./1 h/20 mmHg). The complete conversion of the OH groups was confirmed by means of ¹H NMR spectroscopy.

A brown transparent oil is obtained essentially being a derivatized castor oil hexamer consisting of (on average) a tetramer of the core element being terminated with one R₂ and one R₃ element:

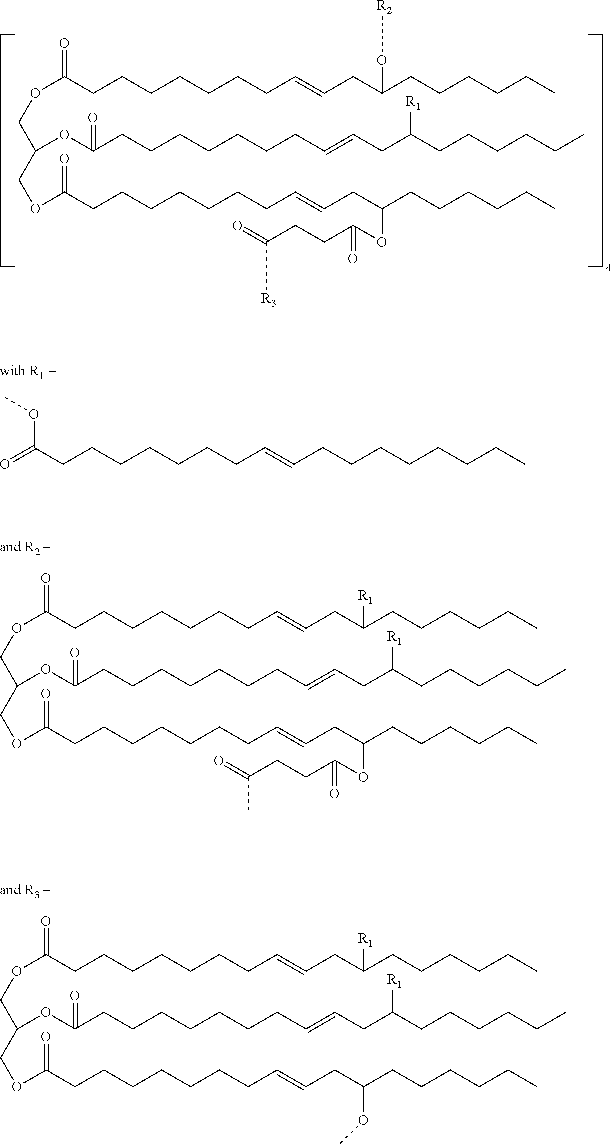

### Example 10

Synthesis of an Oligomerized Acetic Acid Derivative of Castor Oil Having Succinic Acid Ester Bridges

In a 250 ml three-necked bottle, equipped with refluxing condenser, thermometer and magnetic stirrer, dropping funnel and gas outlet tube, 110 g (0.1178 mol) castor oil were placed at room temperature. 15.22 g (0.0982 mol) succinic acid dichloride were added within five minutes and the mixture heated to 80° C. for 1.5 hrs. The complete conversion of the OH groups was confirmed by means of ¹H NMR spectroscopy. The mixture is cooled to 45° C. 12.33 g (0.157 mol) acetic acid chloride were added within 30 minutes, the temperature afterwards increased to 80° C. and maintained for 3 hrs. The product was mixed with 10 g deionized water and volatiles were finally removed under reduced pressure (85° C./1 h/20 mmHg). The complete conversion of the OH groups was confirmed by means of ¹H NMR spectroscopy.

A brown transparent oil is obtained essentially being a derivatized castor oil hexamer consisting of (on average) a tetramer of the core element being terminated with one R₂ and one R₃ element:

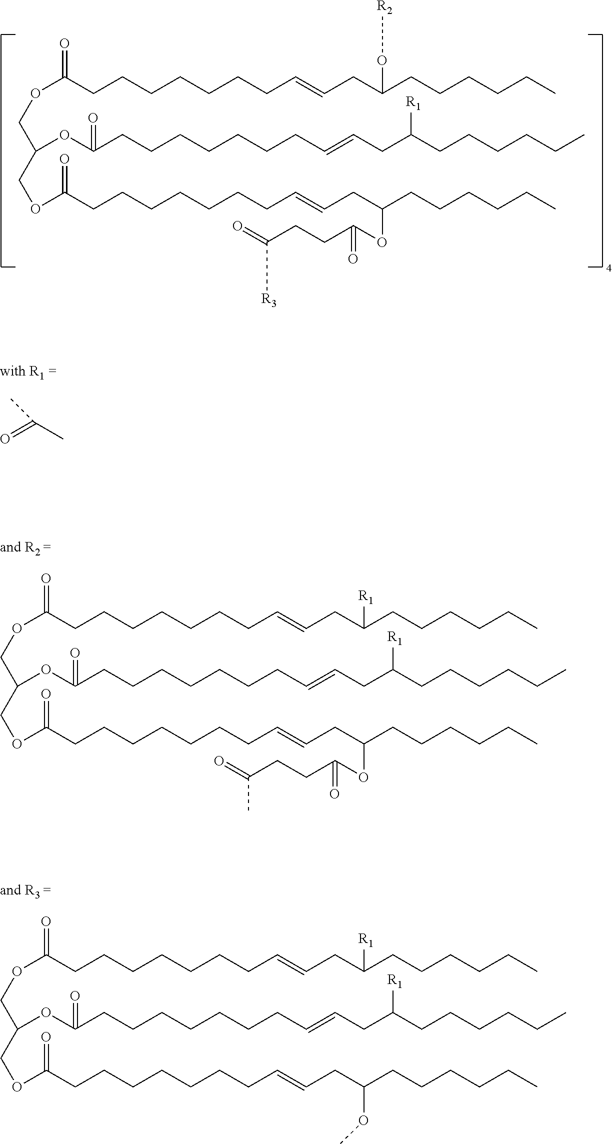

### Example 11

Synthesis of an Oligomerized (Ricinoleic Acid-Stearic Acid) Dimer Derivative of Castor Oil Having Estolide Bridges

In a 250 ml three-necked bottle, equipped with refluxing condenser, thermometer and magnetic stirrer, dropping funnel and gas outlet tube, 5.85 g (0.0377 mol) succinic acid dichloride were placed at room temperature. 22.52 g (0.0754 mol) ricinoleic acid were added within 20 minutes and the mixture heated to 70° C. for 1 h. The complete conversion of the OH groups was confirmed by means of ¹H NMR spectroscopy. 26.91 g (0.2262 mol) SOCl₂ were added and the temperature was increased to 80° C. for 1 h. The excess of SOCl₂ was removed under reduced pressure (70° C./0.5 h/20 mmHg). Afterwards, 52.81 g (0.0258 mol) castor oil were added and the reaction was continued at 80° C. for 1 h. The partial conversion of the OH groups was confirmed by means of ¹H NMR spectroscopy. 55.06 g (0.0944 mol) of the ricinoleic acid-stearic acid dimer chloride intermediate from synthesis example 1a were added at 80° C. and the temperature was increased to 90° C. for 2 h. The product was mixed with 10 g deionized water and volatiles were finally removed under reduced pressure (85° C./1 h/20 mmHg). The complete conversion of the OH groups was confirmed by means of ¹H NMR spectroscopy.

A brown transparent oil essentially having the following structure was obtained:

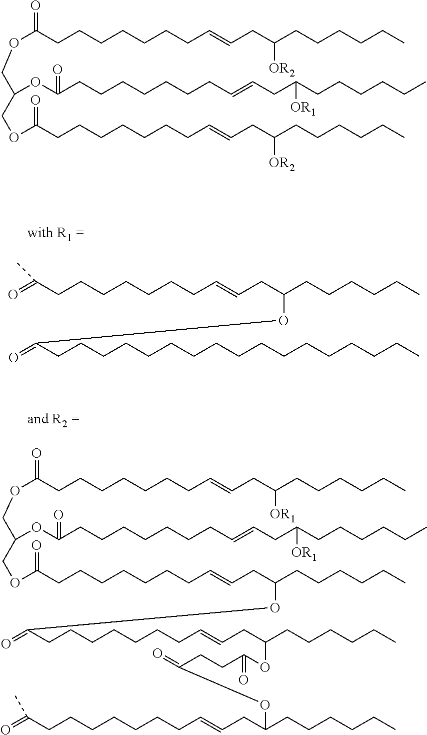

### Example 12

Synthesis of an Oligomerized Mixed (ricinoleic acid-oleic acid)+(12-hydroxy stearic acid-oiec acid) Dimer Derivative of Castor Oil Having Estolide Bridges

In a 250 ml three-necked bottle, equipped with refluxing condenser, thermometer and magnetic stirrer, dropping funnel and gas outlet tube, 5.85 g (0.0377 mol) succinic acid dichloride were placed at room temperature. 22.52 g (0.0754 mol) ricinoleic acid were added within 20 minutes and the mixture heated to 70° C. for 1 h. The complete conversion of the OH groups was confirmed by means of ¹H NMR spectroscopy. 26.91 g (0.2262 mol) SOCl₂ were added and the temperature was increased to 80° C. for 1 h. The excess of SOCl₂ was removed under reduced pressure (70° C./0.5 h/20 mmHg). Afterwards, 52.81 g (0.0258 mol) castor oil were added and the reaction was continued at 80° C. for 1 h. The partial conversion of the OH groups was confirmed by means of ¹H NMR spectroscopy. A mixture consisting of 41.13 g (0.0708 mol) of the ricinoleic acid-oleic acid dimer chloride intermediate from synthesis example 1 and 13.76 g (0.0236 mol) of the 12-hydroxy stearic acid-oleic acid dimer chloride intermediate from synthesis example 1b were added at 80° C. and the temperature was increased to 90° C. for 2 h. The product was mixed with 10 g deionized water and volatiles were finally removed under reduced pressure (85° C./1 h/20 mmHg). The complete conversion of the OH groups was confirmed by means of ¹H NMR spectroscopy.

A brown transparent oil essentially having the following structure was obtained:

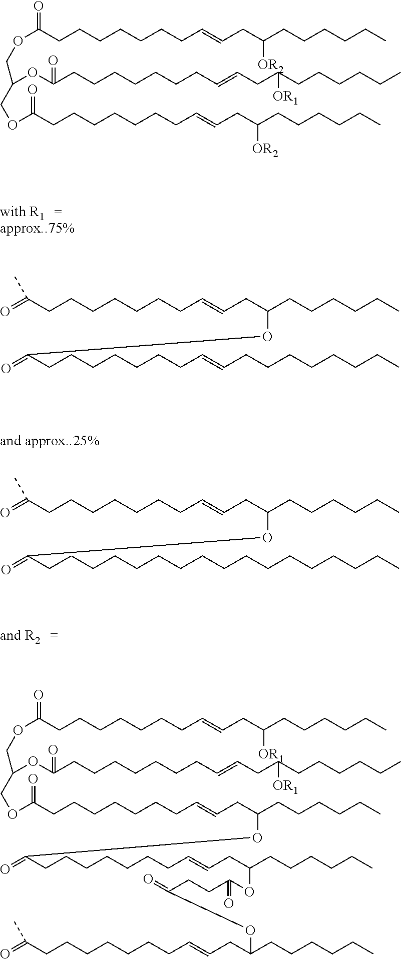

### Example 13

Synthesis of an Oligomerized Oleic Acid Derivative of Castor Oil Having Estolide Bridges

In a 250 ml three-necked bottle, equipped with refluxing condenser, thermometer and magnetic stirrer, dropping funnel and gas outlet tube, 7.35 g (0.0474 mol) succinic acid dichloride were placed at room temperature. 28.32 g (0.0948 mol) ricinoleic acid were added within 20 minutes and the mixture heated to 70° C. for 1 h. The complete conversion of the OH groups was confirmed by means of ¹H NMR spectroscopy. 33.84 g (0.2844 mol) SOCl₂ were added and the temperature was increased to 80° C. for 1 h. The excess of SOCl₂ was removed under reduced pressure (70° C./0.5 h/20 mmHg). Afterwards, 66.43 g (0.0712 mol) castor oil were added and the reaction was continued at 80° C. for 1 h. The partial conversion of the OH groups was confirmed by means of ¹H NMR spectroscopy. 35.69 g (0.1186 mol) of oleic acid chloride were added at 80° C. and the temperature was increased to 90° C. for 2 h. The product was mixed with 10 g deionized water and volatiles were finally removed under reduced pressure (70° C./1 h/20 mmHg). The complete conversion of the OH groups was confirmed by means of ¹H NMR spectroscopy.

A brown transparent oil essentially having the following structure was obtained:

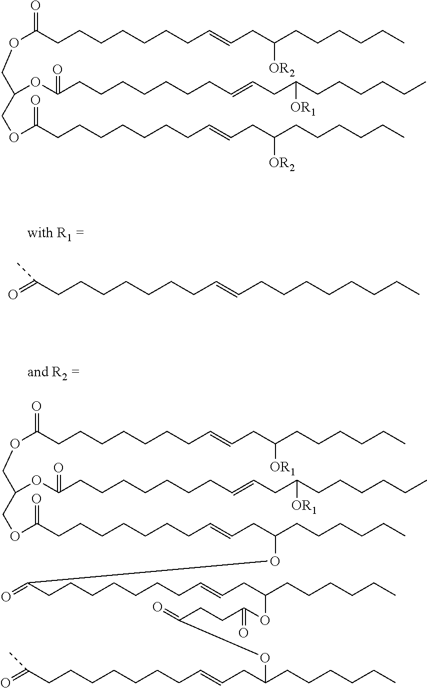

### Example 14

Synthesis of an Oligomerized (Ricinoleic Acid-Ricinoleic Acd-Oleic Acid) Trimer Derivative of Castor Oil Having Estolide Bridges

In a 250 ml three-necked bottle, equipped with refluxing condenser, thermometer and magnetic stirrer, dropping funnel and gas outlet tube, 4.86 g (0.0314 mol) succinic acid dichloride were placed at room temperature. 18.73 g (0.0628 mol) ricinoleic acid were added within 20 minutes and the mixture heated to 70° C. for 1 h. The complete conversion of the OH groups was confirmed by means of ¹H NMR spectroscopy. 22.41 g (0.1884 mol) SOCl₂ were added and the temperature was increased to 80° C. for 1 h. The excess of SOCl₂ was removed under reduced pressure (80° C./0.5 h/20 mmHg). Afterwards, 43.94 g (0.0471 mol) castor oil were added and the reaction was continued at 80° C. for 1.5 h. The partial conversion of the OH groups was confirmed by means of¹H NMR spectroscopy. 67.62 g (0.0785 mol) of the ricinoleic acid-ricinoleic acid-oleic acid trimer chloride intermediate from synthesis example 6b were added at 80° C. and the temperature was increased to 90° C. for 2 h. The product was mixed with 10 g deionized water and volatiles were finally removed under reduced pressure (70° C./1 h/20 mmHg). The complete conversion of the OH groups was confirmed by means of ¹H NMR spectroscopy.

A brown transparent oil essentially having the following structure was obtained:

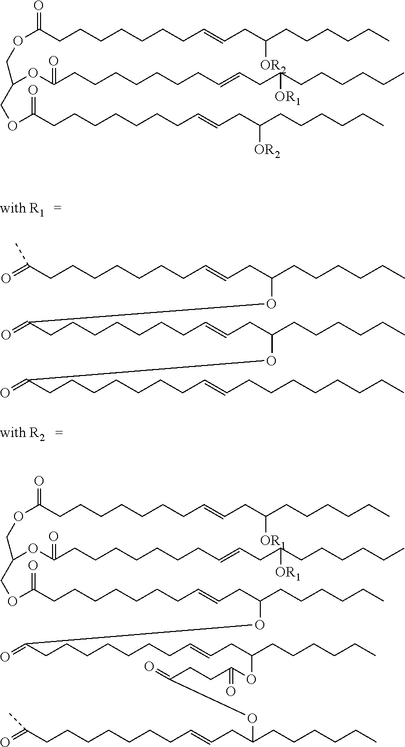

### Example 15

Synthesis of an Oligomerized (Ricinoleic Acd-Oleic Acid) Dimer Derivative of Castor Oil Having Succinic Acid Ester Bridges

In a 250 ml three-necked bottle, equipped with refluxing condenser, thermometer and magnetic stirrer, dropping funnel and gas outlet tube, 57.8 g (0.062 mol) castor oil were placed at room temperature. 6.38 g (0.0412 mol) succinic acid dichloride were added within 10 minutes. The mixture is heated to 80° C. for 1.5 h. 60 g (0.1032 mol) of the ricinoleic acid-oleic acid dimer chloride intermediate from synthesis example 1 were added during 1 h and the reaction continued at 80° C. for 3 hrs. The product was mixed with 10 g deionized water and volatiles were finally removed under reduced pressure (70° C./1.5 h/15 mmHg). The complete conversion of the OH groups was confirmed by means of ¹H NMR spectroscopy.

A brown transparent oil essentially having the following structure was obtained:

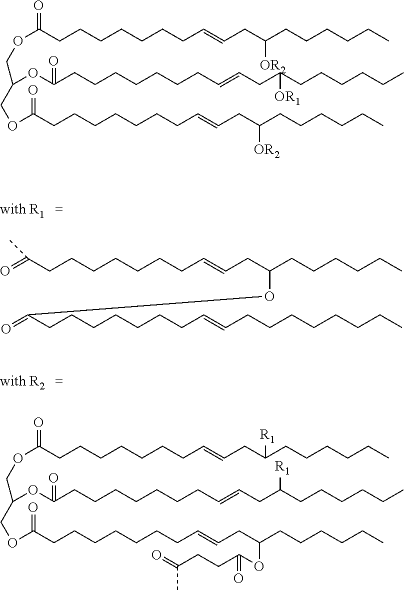

Table 2 summarizes the viscosities of the castor oil derivatives described in the examples 8 to 15. The viscosity of the compounds of the examples 8, 9, 10, 11, 12, 13, 14 and 15 was determined using a Brookfield DV2T viscosimeter, spindle 4, 200 RPM, 23° C.:

| example | 8 | 9 | 10 | 11 | 12 | 13 | 14 | 15 |
|---|---|---|---|---|---|---|---|---|
| viscosity | 7168 | 5312 | 12380 | 3744 | 2848 | 2368 | 5472 | 2176 |
| mPa · s | | | | | | | | |

Application Tests

Combing Force Measurements

Combing force measurements were carried out l order to quantify the effect of the compounds according to the invention. A Miniature Tensile Tester 175 (Dia-Stron Limited) was used.

Two different types of hair (Kerling International) were selected for these measurements:

| Hair type | Hair finishing method |
|---|---|
| White buffalo belly hair, 20 cm long | 1 |
| “buffalo hair” | |
| Euro-Hair, bleached heavily, 20 cm long | 2 |
| “damaged human hair” | |

Hair Finishing Method 1 (Buffalo Hair)

The weight of the portion of the hair tresses to be finished is determined and the calculated total amount on active substance (based on the target mg active/1 g buffalo hair) dissolved in 2-propanol. The amount on 2-propanol used was calculated by the following formula:

m₂₋ₚᵣₒₚₐₙₒₗ (g)=1.94×m_{hair finished}

The 2-propanol solutions are evenly distributed over the hair tresses. The tresses are air dried for 2 h and further processed as outlined in the general protocol.

Hair Finishing Method 2 (Damaged Human Hair)

The weight of the portion of the hair tresses to be finished is determined and the calculated total amount on active substance (based on the target mg active/1 g buffalo hair) dissolved in 2-propanol. The amount on 2-propanol used was calculated by the following formula:

m₂₋ₚᵣₒₚₐₙₒₗ (g)=0.64×m_{hair finished}

The 2-propanol solutions are evenly distributed over the hair tresses. The tresses are air dried for 2 h and further processed as outlined in the general protocol.

General Protocol for the Pretreatment and Handling of Hair Tresses

Individual hair tresses (2.5 cm) were cut off from the respective stock tress and equilibrated in a humidity chamber at 50% rel. hum. for 12 h. Afterwards, the dry tear off force and the wet average force (tresses rinsed with 38° C. tap water for 30 seconds) were determined for the untreated tresses (baseline measurements). Three strokes were carried out. The force data of the third stroke were used for the calculations.

The tresses were air dried and equilibrated in the climate chamber for additional 15 h. Afterwards, they were finished with the 2-propanol solutions as outlined for the hair finishing methods 1 and 2, air dried for two hours and equilibrated in the climate chamber for additional 15 h. Finally, the dry tear off force and the wet average force (tresses rinsed with 38° C. tap water for 30 seconds) were determined for the finished tresses (measurements finished hair). Three strokes were carried out. The force data of the third stroke were used for the calculations.

The ratio between the required combing force before finishing (baseline measurements) and the combing force after finishing (measurements finished hair) describes the effectiveness of a conditioning agent.

The following formula was used to calculate the relative combing force reduction:

Force reduction (%)=(Force_{baseline}−Force_{finished})×100/Force_{baseline}

Results Combing Force Measurements

Buffalo Hair

| | | concentration active | dry tear | wet average |
|---|---|---|---|---|
| | compound | (mg active/1 g | off force | force |
| run | example | buffalo hair) | (reduction %) | (reduction %) |
| 1 | synthesis | 5 | 25.2 | 14.4 |
| | example 2 | | | |
| 2 | 1 | 5 | 38.4 | 0.5 |
| 3 | 3 | 5 | 27.7 | 65.9 |
| 4 | 2 | 5 | 31.1 | 48.2 |
| 5 | 2 | 10 | 46.4 | 44.2 |
| 6 | 2 | 50 | 67.9 | 38.3 |
| 7 | 2 | 100 | 58.8 | 22.0 |

Damaged Human Hair

| | | concentration active | dry tear | wet average |
|---|---|---|---|---|
| | | (mg active/1 g | off force | force |
| run | compound | damaged human hair) | (reduction %) | (reduction %) |
| 8 | 3 | 5 | 72.9 | 72.3 |
| 9 | 4 | 5 | 88.5 | 58.9 |
| 10 | 5 | 5 | 84.8 | 87.0 |
| 11 | 6d | 5 | 96.0 | 81.0 |
| 12 | 7b | 5 | 93.3 | 81.3 |

The data in the above two tables on buffalo hair and damaged human hair show that the compounds according to the invention are able to reduce the combing forces on different keratinous substrates. A comparison of the data for compound 3 on buffalo hair and damaged human hair highlights the specific effectiveness of the inventive compounds on human hair. The data for compounds 4 and 5 on damaged human hair highlight the effectiveness of inventive materials having higher molecular weights. The data on compound 6d specifically highlight the value of branched structures when applied on damaged human hair.

The data on compound 7b specifically highlight the value of structures containing high melting fatty acid ester moieties when applied on damaged human hair.

Further Test on Damaged Human Hair

Damaged Human Hair

| | | concentration active | dry tear | wet average |
|---|---|---|---|---|
| | compound | (mg active/1 g | off force | force |
| run | example | damaged human hair) | (reduction %) | (reduction %) |
| 13 | 8 | 5 | 70.3 | 77.7 |
| 14 | 9 | 5 | 55.9 | 75.1 |
| 15 | 11 | 5 | 23.8 | 74.2 |
| 16 | 12 | 5 | 37.4 | 68.5 |
| 17 | 13 | 5 | 56.5 | 85.0 |
| 18 | 14 | 5 | 62.2 | 60.2 |

The data in the above two table on damaged human hair show that the compounds according to the invention are able to reduce the combing forces on different keratinous substrates. The data for compounds 8 and 9 on damaged human hair highlight the effectiveness of inventive materials having higher molecular weights. The data on compounds 11 and 12 specifically highlight the value of structures bearing saturated, higher melting fatty acid moieties when applied on damaged human hair. The data on compound 14 highlight the value of structures bearing long poly fatty acid moieties whereas the data on compound 13 demonstrate that relatively short chains when incorporated in carefully selected structures according to the invention are also beneficial on damaged human hair.

### Further Examples

### Synthesis Example 16

Synthesis of a (Ricinoleic Acid-Oleic Acid) Dimer Acid Containing Mixture

In a 100 ml three-necked bottle, equipped with refluxing condenser, thermometer and magnetic stirrer, dropping funnel, N₂ inlet tube and vacuum outlet, 30 g (0.1062 mol) oleic acid and 15 g (0.0354 OH groups) of a ricinoleic acid containing 15% oleic acid and having a degree on free OH groups of 70% of the theoretical value were placed at room temperature. The mixture was heated to 160° C. at 20 mbar. Additional 30.28 g (0.0707 mol OH) of the ricinoleic acid were added during 1 hr at 160° C./20 mbar. The esterification was continued at 160° C./20 mbar for 29 hrs.

Afterwards, a conversion of 96.9% of the OH groups was confirmed by means of ¹H NMR spectroscopy.

A brownish, transparent oil containing the following compounds was obtained:

Ricinoleic acid-oleic acid dimer (main product approx. 80%)

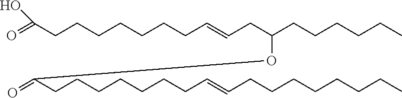 [SMILES: CCCCCCCC/C=C/CCCCCCCC(=O)OC(C/C=C/CCCCCCCC(=O)O)CCCCCC]

Ricinoleic acid-ricinoleic acid-oleic acid trimer (approx. 10%)

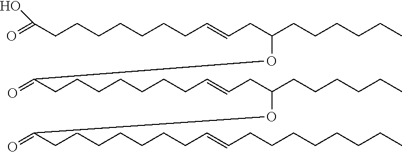 [SMILES: CCCCCCCC/C=C/CCCCCCCC(=O)OC(C/C=C/CCCCCCCC(=O)OC(C/C=C/CCCCCCCC(=O)O)CCCCCC)CCCCCC]

Additionally in total approx. 10% of acids of the types (ricinoleic acid)₃₋₆-oleic acid, ricinoleic acid dimer, ricinoleic acid, oleic acid.

### Synthesis Example 17

Synthesis of a (Ricinoleic Acid-Oleic Acid) Dimer Acid Containing Mixture

In a 100 ml three-necked bottle, equipped with refluxing condenser, thermometer and magnetic stirrer, dropping funnel, N₂ inlet tube and vacuum outlet, 30 g (0.1062 mol) oleic acid and 15 g (0.0354 OH groups) of a ricinoleic acid containing 15% oleic acid and having a degree on free OH groups of 70% of the theoretical value were placed at room temperature. The mixture was heated to 200° C. at 20 mbar. Additional 30.28 g (0.0707 mol OH) of the ricinoleic acid were added during 1 hr at 200° C./20 mbar. The esterification was continued at 200° C./20 mbar for 13.5 hrs. Afterwards, a conversion of 98% of the OH groups was confirmed by means of ¹H NMR spectroscopy.

A brownish, transparent oil containing the following compounds was obtained:

Ricinoleic acid-oleic acid dimer (main product approx. 80%)

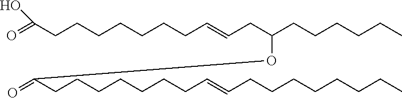 [SMILES: CCCCCCCC/C=C/CCCCCCCC(=O)OC(C/C=C/CCCCCCCC(=O)O)CCCCCC]

Ricinoleic acid-ricinoleic acid-oleic acid trimer (approx. 10%)

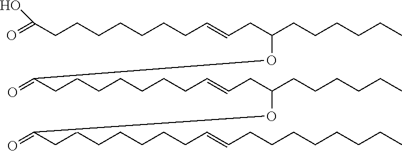 [SMILES: CCCCCCCC/C=C/CCCCCCCC(=O)OC(C/C=C/CCCCCCCC(=O)OC(C/C=C/CCCCCCCC(=O)O)CCCCCC)CCCCCC]

Additionally in total approx. 10% of acids of the type (ricinoleic acid)₃₋₆-oleic acid, ricinoleic acid dimer, ricinoleic acid, oleic acid were obtained.

### Synthesis Example 18

Synthesis of a (ricinoleic acid-oleic acid) dimer acid containing mixture

In a 100 ml three-necked bottle, equipped with refluxing condenser, thermometer and magnetic stirrer, dropping funnel, N2 inlet tube and vacuum outlet, 35 g (0.1239 mol) oleic acid and 12.32 g (0.0412 OH groups) of a ricinoleic acid containing 15% oleic acid and having a degree on free OH groups of 70% of the theoretical value were placed at room temperature. The mixture was heated to 200° C. at 20 mbar. Additional 24.66 g (0.0826 mol OH) of the ricinoleic acid were added during 0.5 hr at 200° C./20 mbar. The esterification wass continued at 200° C./20 mbar for 13.5 hrs. Afterwards, a conversion of 99% of the OH groups was confirmed by means of ¹H NMR spectroscopy.

A brownish, transparent oil containing the following compounds was obtained:

Ricinoleic acid-oleic acid dimer (main product approx. 70%)

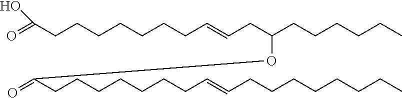 [SMILES: CCCCCCCC/C=C/CCCCCCCC(=O)OC(C/C=C/CCCCCCCC(=O)O)CCCCCC]

Ricinoleic acid-ricinoleic acid-oleic acid trimer (approx. 10%)

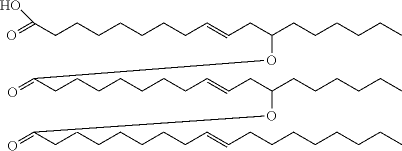 [SMILES: CCCCCCCC/C=C/CCCCCCCC(=O)OC(C/C=C/CCCCCCCC(=O)OC(C/C=C/CCCCCCCC(=O)O)CCCCCC)CCCCCC]

Additionally in total approx. 20% of acids of the type (ricinoleic acid)₃₋₆-oleic acid, ricinoleic acid dimer, ricinoleic acid, oleic acid.

### Synthesis Example 19

Synthesis of a (Ricinoleic Acid-Succinic Acid-Ricinoleic Acid) Trimer Di-Acid Containing Mixture

In a 100 ml three-necked bottle, equipped with refluxing condenser, thermometer and magnetic stirrer, dropping funnel, N₂ inlet tube and gas/vacuum outlet, 9.87 g (0.0836 mol) succinic acid, 9.87 g DI water and 24.1 g (0.0565 mol OH) of a ricinoleic acid containing 15% oleic acid and having a degree on free OH groups of 70% of the theoretical value were placed at room temperature. A gentle N₂ stream passes through the gas volume above mixure's surface. The mixture was slowly heated to 160° C. within 3 hrs. At 100° C., 110° C., 120° C. and 140° C. additional quantities on ricinoleic acid (13.53 g, 11.57 g, 12.39 g, 9.77 g; total amount ricinoleic acid in mixture 71.36 g) were added to the mixture. The esterification reaction was continued at 160° C. for additional 13 hrs while the N2 stream removed the water from the mixture. Afterwards, a conversion of 98.9% of the OH groups was confirmed by means of ¹H NMR spectroscopy.

A brownish, transparent oil containing the following compounds was obtained:

ricinoleic acid-succinic acid-ricinoleic acid trimer (main product approx. 80%)

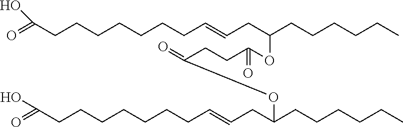 [SMILES: CCCCCCC(C/C=C/CCCCCCCC(=O)O)OC(=O)CCC(=O)OC(C/C=C/CCCCCCCC(=O)O)CCCCCC]

ricinoleic acid-succinic acid-ricinoleic acid-oleic acid tetramers and pentamers (in total approx. 10%)

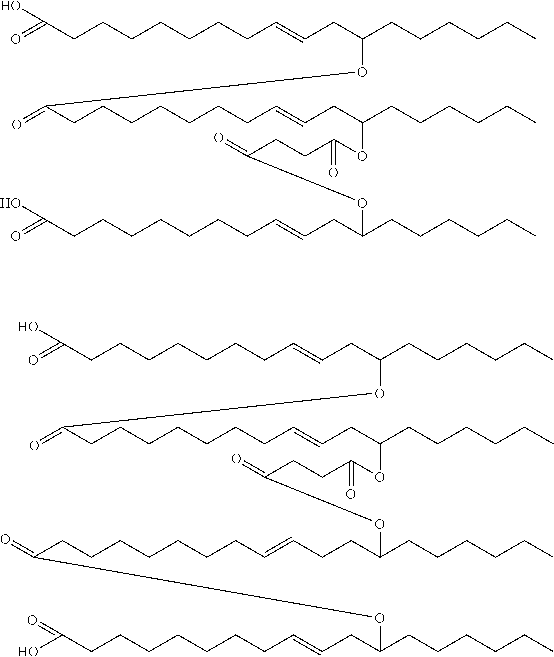 [SMILES: CCCCCCC(C/C=C/CCCCCCCC(=O)O)OC(=O)CCCCCCC/C=C/CC(CCCCCC)OC(=O)CCC(=O)OC(C/C=C/CCCCCCCC(=O)O)CCCCCC ; CCCCCCC(C/C=C/CCCCCCCC(=O)O)OC(=O)CCCCCCC/C=C/CCC(CCCCCC)OC(=O)CCC(=O)OC(C/C=C/CCCCCCCC(=O)OC(C/C=C/CCCCCCCC(=O)O)CCCCCC)CCCCCC]

and additionally in total approx. 10% of acids of the types ricinoleic acid-succinic acid dimer

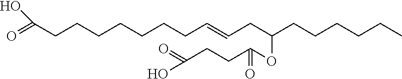 [SMILES: CCCCCCC(C/C=C/CCCCCCCC(=O)O)OC(=O)CCC(=O)O]

ricinoleic acid-ricinoleic acid-succinic acid trimer

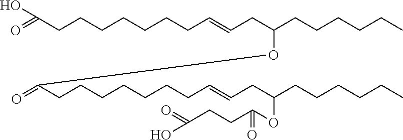 [SMILES: CCCCCCC(C/C=C/CCCCCCCC(=O)O)OC(=O)CCCCCCC/C=C/CC(CCCCCC)OC(=O)CCC(=O)O]

ricinoleic acid-oleic acid dimer, ricinoleic acid-ricinoleic acid-oleic acid trimer, higher oligomers of these compounds, succinic acid and oleic acid.

### Synthesis Example 20

Synthesis of a (Ricinoleic Acid-Succinic Acid-Ricinoleic Acid) Trimer Di-Acid Containing Mixture

In a 100 ml three-necked bottle, equipped with refluxing condenser, thermometer and magnetic stirrer, dropping funnel, N₂ inlet tube and gas/vacuum outlet, 12.86 g (0.1089 mol) succinic acid, 12.86 g DI water and 21 g (0.0704 mol) of a ricinoleic acid containing 15% oleic acid and having a degree on free OH groups of 70% of the theoretical value were placed at room temperature. A gentle N₂ stream was passed through the gas volume above mixure's surface. The mixture was slowly heated to 160° C. within 2 hrs. At 110° C., 120° C., 130° C., 150° C. additional quantities of ricinoleic acid (12.2 g, 11.61 g, 11.08 g, 9.11 g; total amount ricinoleic acid in mixture 65 g) were added to the mixture. The esterification reaction was continued at 160° C. for additional 28 hrs while the N₂ stream removed the water from the mixture. Afterwards, a conversion of 99.4% of the OH groups was confirmed by means of ¹H NMR spectroscopy.

A brownish, transparent oil containing the following compounds was obtained:

ricinoleic acid-succinic acid-ricinoleic acid trimer (main product approx. 70%)

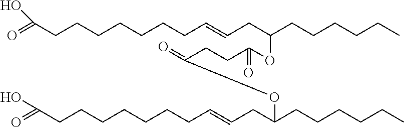 [SMILES: CCCCCCC(C/C=C/CCCCCCCC(=O)O)OC(=O)CCC(=O)OC(C/C=C/CCCCCCCC(=O)O)CCCCCC]

ricinoleic acid-succinic acid-ricinoleic acid-oleic acid tetramers and pentamers (in total approx. 10%)

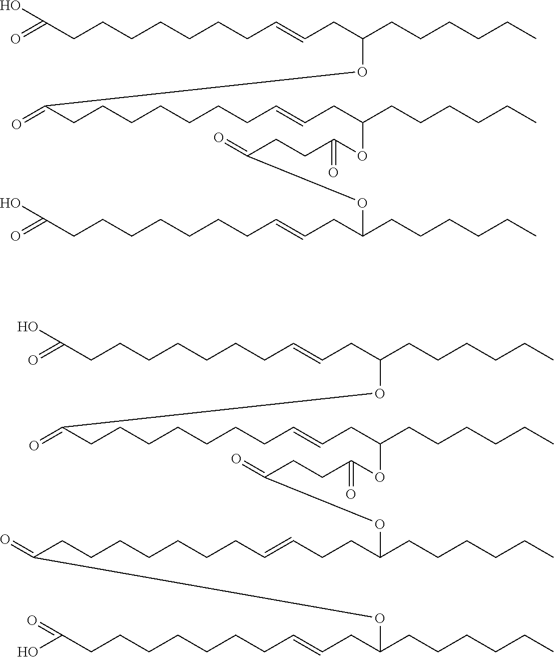 [SMILES: CCCCCCC(C/C=C/CCCCCCCC(=O)O)OC(=O)CCCCCCC/C=C/CC(CCCCCC)OC(=O)CCC(=O)OC(C/C=C/CCCCCCCC(=O)O)CCCCCC ; CCCCCCC(C/C=C/CCCCCCCC(=O)O)OC(=O)CCCCCCC/C=C/CCC(CCCCCC)OC(=O)CCC(=O)OC(C/C=C/CCCCCCCC(=O)OC(C/C=C/CCCCCCCC(=O)O)CCCCCC)CCCCCC]

Additionally in total approx. 20% of acids of the types ricinoleic acid-succinic acid dimer

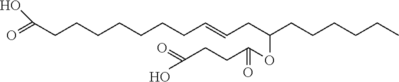 [SMILES: CCCCCCC(C/C=C/CCCCCCCC(=O)O)OC(=O)CCC(=O)O]

ricinoleic acid-ricinoleic acid-succinic acid trimer

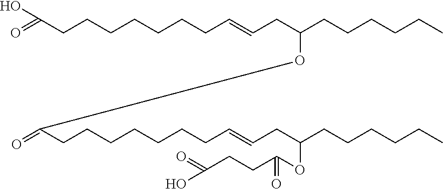 [SMILES: CCCCCCC(C/C=C/CCCCCCCC(=O)O)OC(=O)CCCCCCC/C=C/CC(CCCCCC)OC(=O)CCC(=O)O]

ricinoleic acid-oleic acid dimer, ricinoleic acid-ricinoleic acid-oleic acid trimer, higher oligomers of these compounds, succinic acid and oleic acid.

### Example 21

Synthesis of an Oligomerized (Ricinoleic Acid-Oleic Acid) Dimer Derivative of Castor Oil Having Estolide Bridges—Sequential Addition

In a 100 ml three-necked bottle, equipped with refluxing condenser, thermometer and magnetic stirrer, dropping funnel and N₂/vacuum inlet/outlet, 30 g (0.0321 mol) castor oil and 14.35 g (0.0214 mol) of the (ricinoleic acid-succinic acid-ricinoleic acid) trimer di-acid from example 19 were placed at room temperature and heated to 180° C. at a pressure of 25 mbar for 20 hrs. Afterwards, 31.66 g (0.0562 mol) of the (ricinoleic acid-oleic acid) dimer acid from example 17 were added and the reaction continued at 180° C. at a pressure of 25 mbar for 16 hrs.

The conversion of OH groups was 83.1% (1H-NMR).

A transparent yellow to brownish liquid having the following approximate structure was obtained:

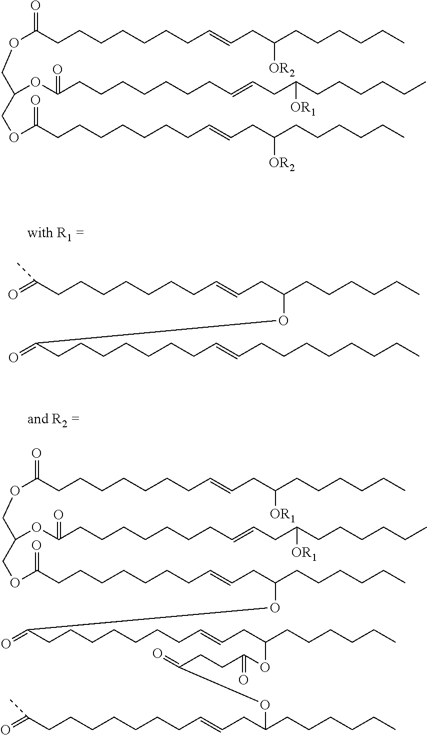

Additionally, the material contains minor portions of higher Mw oligomers of the shown structure, the di-acid intermediate from example 19, the mono-acid intermediate from example 17 as well as the side products outlined in the examples 19 an 17.

### Example 22

Synthesis of an Oligomerized (Ricinoleic Acid-Oleic Acid) Dimer Derivative of Castor Oil Having Estolide Bridges—all in Mix

In a 100 ml three-necked bottle, equipped with refluxing condenser, thermometer and magnetic stirrer, dropping funnel and N₂/vacuum inlet/outlet 30 g (0.0321 mol) castor oil, 14.35 g (0.0214 mol) of the (ricinoleic acid-succinic acid-ricinoleic acid) trimer di-acid from example 19 and 31.66 g (0.0562 mol) of the (ricinoleic acid-oleic acid) dimer acid from example 17 were mixed at room temperature, the temperature increased to 180° C. at a pressure of 25 mbar and the conditions maintained for 16 hrs.

The conversion of OH groups was 84.7% (¹H-NMR).

A transparent yellow to brownish liquid having the following approximate structure was obtained:

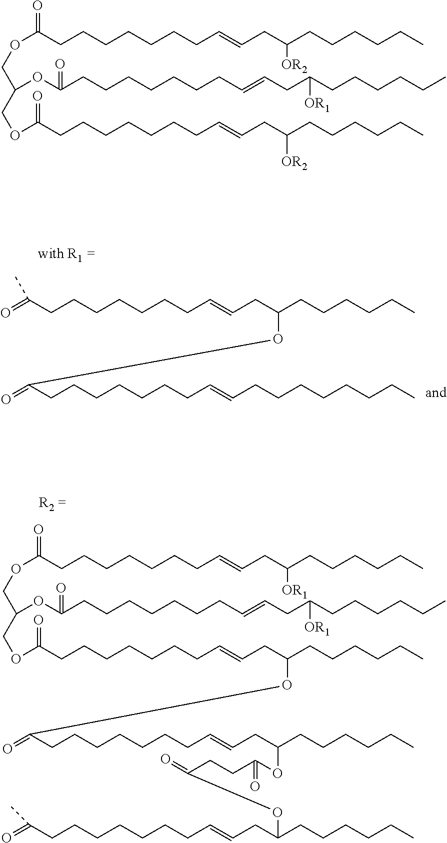

Additionally, the material contains minor portions of higher Mw oligomers of the shown structure, the di-acid intermediate from example 19, the mono-acid intermediate from example 17 as well as the side products outlined in the examples 19 and 17.

### Example 23

Synthesis of an Oligomerized (Ricinoleic Acid-Oleic Acid) Dimer Derivative of Castor Oil Having Estolide Bridges—Sequential Addition

In a 100 ml three-necked bottle, equipped with refluxing condenser, thermometer and magnetic stirrer, dropping funnel and N₂/vacuum inlet/outlet, 10 g (0.0107 mol) castor oil and 13.83 g (0.0204 mol) of the (ricinoleic acid-succinic acid-ricinoleic acid) trimer di-acid from example 19, 0.72 g (0.0061 mol) succinic acid and 0.72 g DI water were mixed at room temperature. There was a gentle N2 stream through the bottle. The mixture was heated to 160° C. within 15 minutes and the temperature maintained for 6 hrs. Afterwards, 20 g (0.0214 mol) castor oil were added and the reaction was continued at 160° C. for 9 hrs. 30.15 g (0.0536 mol) of the (ricinoleic acid-oleic acid) dimer acid from example 18 were added and the reaction continued at 200° C. at a pressure of 23 mbar for 17 hrs.

The conversion of OH groups was 91.5% (1H-NMR).

A transparent brownish liquid having the following approximate structure was obtained:

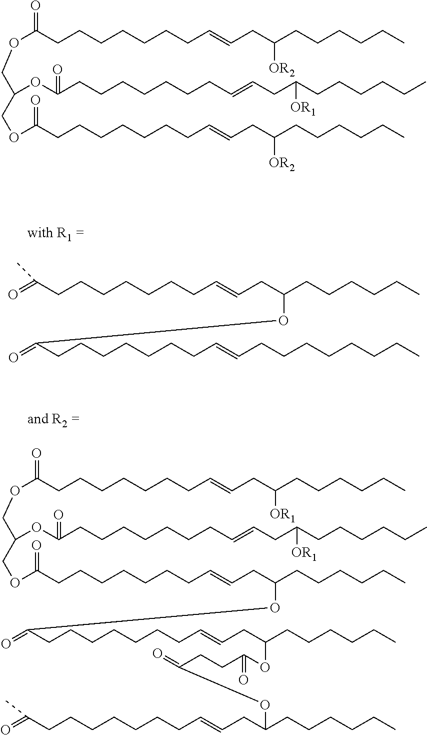

Additionally, the material contains minor portions of higher Mw oligomers of the shown structure, the di-acid intermediate from example 19, the mono-acid intermediate from example 17 as well as the side products outlined in the examples 19 and 18.

### Example 24

Synthesis of an Oligomerized (Ricinoleic Acid-Oleic Acid) Dimer Derivative of Castor Oil Having Estolide Bridges—Partial all in Mixture

In a 100 ml three-necked bottle, equipped with refluxing condenser, thermometer and magnetic stirrer, dropping funnel and N₂/vacuum inlet/outlet, 10 g (0.0107 mol) castor oil and 13.83 g (0.0204 mol) of the (ricinoleic acid-succinic acid-ricinoleic acid) trimer di-acid from example 19, 0.72 g (0.0061 mol) succinic acid and 0.72 g DI water were mixed at room temperature. There was a gentle N₂ stream through the bottle. The mixture was heated to 160° C. within 15 minutes and the temperature maintained for 6 hrs. Afterwards, 20 g (0.0214 mol) castor oil and 30.15 g (0.0536 mol) of the (ricinoleic acid-oleic acid) dimer acid from example 18 were added and the reaction continued at 160° C. at a pressure of 23 mbar for 8 hrs. Afterwards, the temperature was increased to 200° C. and the reaction continued under vacuum for 16 hrs. The conversion of OH groups was 91.6% (′H-NMR).

A transparent brownish liquid having the following approximate structure was obtained:

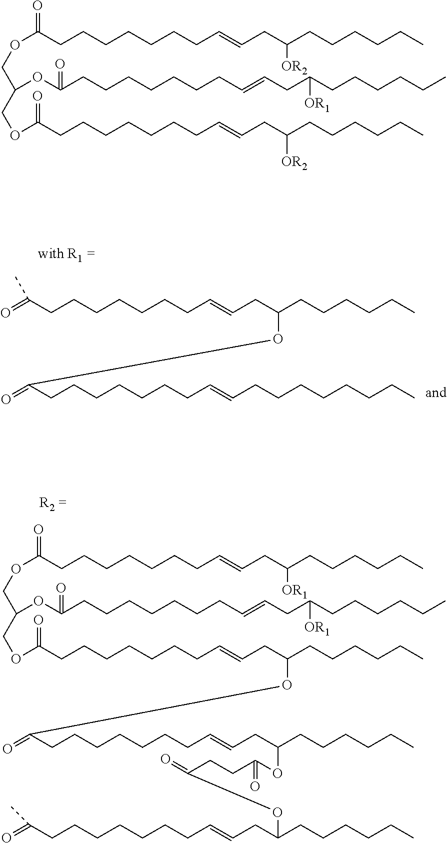

Additionally, the material contains minor portions of higher Mw oligomers of the shown structure, the di-acid intermediate from example 19, the mono-acid intermediate from example 17 as well as the side products outlined in the examples 19 and 18.

### Example 25

Synthesis of a (Ricinoleic Acid-Stearic Acid) Dimer Acid Containing Mixture

In a 500 ml three-necked bottle, equipped with refluxing condenser, thermometer and magnetic stirrer, dropping funnel, N₂ inlet tube and vacuum outlet, 163.45 g (0.5746 mol) stearic acid were heated to 70° C. A gentle N₂ stream was flushed through the bottle during the course of the reaction. 81.66 g (0.2736 mol) of a ricinoleic acid containing 15% oleic acid and having a degree on free OH groups of 70% of the theoretical value were added. The mixture was heated to 160° C. for 1 h. Additional 163.34 g (0.5417 mol) of the ricinoleic acid were added during 1 hr at 160° C. and the temperature maintained for 5 hrs. Afterwards, the temperature was increased to 200° C. and maintained for 17 hrs.

The complete conversion of the OH groups was confirmed by means of ¹H NMR spectroscopy. A grey-brownish wax containing as main component the ricinoleic acid-stearic acid dimer was obtained.

Ricinoleic acid-stearic acid dimer (main product approx. 80%)

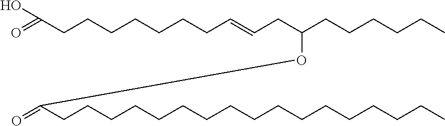 [SMILES: CCCCCCCCCCCCCCCCCC(=O)OC(C/C=C/CCCCCCCC(=O)O)CCCCCC]

Ricinoleic acid-ricinoleic acid-stearic acid trimer (approx. 10%)

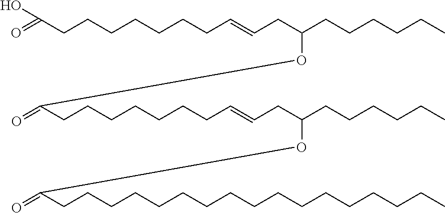 [SMILES: CCCCCCCCCCCCCCCCCC(=O)OC(C/C=C/CCCCCCCC(=O)OC(C/C=C/CCCCCCCC(=O)O)CCCCCC)CCCCCC]

Additionally in total approx. 10% of acids of the type (ricinoleic acid)₃₋₆-stearic acid, ricinoleic acid dimer, ricinoleic acid, stearic acid were obtained.

### Example 25a

Synthesis of an Oligomerized (Ricinoleic Acid-Stearic Acid) Dimer Derivative of Castor Oil Having Estolide Bridges

In a 100 ml three-necked bottle, equipped with refluxing condenser, thermometer and magnetic stirrer, dropping funnel, N₂ inlet and gas outlet tube, 2.4 g (0.00257 mol) castor oil, 11.03 g (0.0171 mol) of the ricinoleic acid-succinic acid-ricinoleic acid derivative from synthesis example 19, 0.61 g (0.0052 mol) succinic acid and 0.61 g DI water were placed. A gentle stream of N² flushed through the bottle during the course of the reaction. The mixture was heated to 160° C. for 5 hrs. 21.6 g (0.0231 mol) ricinoleic acid were added and the reaction was continued at 160° C. for 9 hrs. Afterwards, 20.55 g (0.0363 mol) of the ricinoleic acid-stearic acid dimer from example 25 and 3.66 g (0.0129 mol) stearic acid were added and the reaction continued at 160° C. for 8 hrs. Finally, the reaction temperature was increased to 200° C. for 8 hrs.

The complete conversion of the OH groups was confirmed by means of ¹H NMR spectroscopy.

A grey-brownish wax essentially having the following structure was obtained:

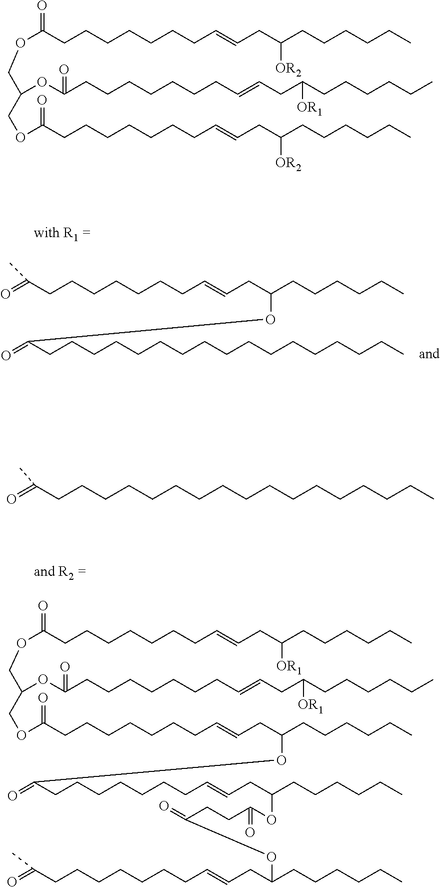

### Example 25b

Synthesis of an Oligomerized (Ricinoleic Acid-Stearic Acid) Dimer Derivative of Castor Oil Having Estolide Bridges

In a 100 ml three-necked bottle, equipped with refluxing condenser, thermometer and magnetic stirrer, dropping funnel, N₂ inlet and gas outlet tube, 2.4 g (0.00257 mol) castor oil, 11.03 g (0.0171 mol) of the ricinoleic acid-succinic acid-ricinoleic acid derivative from synthesis example 19, 0.61 g (0.0052 mol) succinic acid and 0.61 g DI water were placed. A gentle stream of N₂ flushed through the bottle during the course of the reaction. The mixture was heated to 160° C. for 5 hrs. 21.6 g (0.0231 mol) ricinoleic acid were added and the reaction continued at 160° C. for 9 hrs. Afterwards, 24.21 g (0.0428 mol) of the ricinoleic acid-stearic acid dimer from example 25 were added and the reaction continued at 160° C. for 8 hrs. Finally, the reaction temperature was increased to 200° C. for 8 hrs.

The complete conversion of the OH groups was confirmed by means of ¹H NMR spectroscopy. A grey-brownish oil-wax mixture essentially having the following structure was obtained:

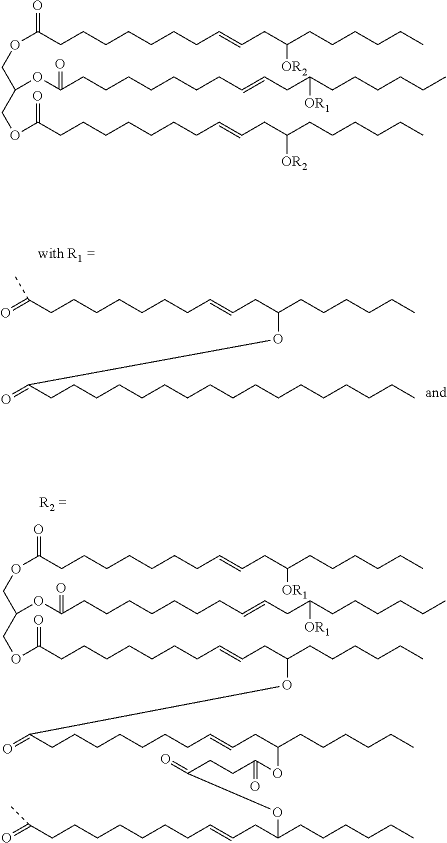

### Example 26

Synthesis of a Stearyl Alcohol Ester of a (Ricinoleic Acid₂-Succinic Acid-Ricinoleic Acid₂) Pentamer Di-Acid Containing Mixture

In a 100 ml three-necked bottle, equipped with refluxing condenser, thermometer and magnetic stirrer, dropping funnel, N₂ inlet tube and gas/vacuum outlet, 25 g (0.037 mol) of the ricinoleic acid-succinic acid-ricinoleic acid trimer from example 19 were heated to 200° C. A gentle N2 stream was passed through the gas volume above the liquid's surface. 21.98 g (0.074 mol) of a ricinoleic acid containing 15% oleic acid and having a degree on free OH groups of 70% of the theoretical value were added within 20 minutes. The temperature was maintained for 12 hrs. Afterwards, 19.91 g (0.074 mol) stearyl alcohol were added and the reaction was continued for 15 hrs. A conversion of 83% of the OH groups was determined by means of ¹H NMR spectroscopy.

A yellow-brownish wax containing the following averaged structure as main component was obtained.

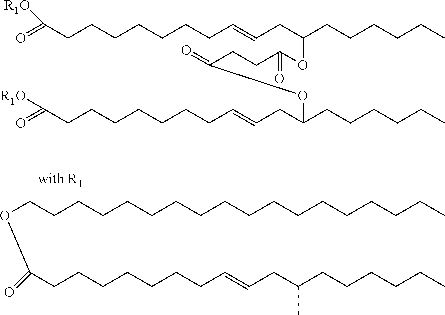

### Example 27

### Synthesis Example 27a

Synthesis of a (Ricinoleic Acid₂-Butanediol-Ricinoleic Acid₂) Pentamer Diol

In a 100 ml three-necked bottle, equipped with refluxing condenser, thermometer and magnetic stirrer, dropping funnel, N₂ inlet tube and gas/vacuum outlet, 7.22 g (0.080 mol) 1,4-butane diol and 47.82 g (0.16 mol) ricinoleic acid were mixed and heated to 160° C. for 17 hrs. A gentle N₂ stream was passed through the gas volume above liquid's surface.

42.16 g (0.065 mol) of the received intermediate of the approximate structure

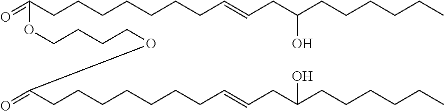 [SMILES: CCCCCCC(O)C/C=C/CCCCCCCC(=O)OCCCCOC(=O)CCCCCCC/C=C/CC(O)CCCCCC]

and 38.66 g (0.13 mol) ricinoleic acid were mixed in a separate bottle and heated to 160° C. for 10 hrs. Afterwards, the temperature was increased to 200° C. for 6 hrs. A gentle N₂ stream was passed through the gas volume above the liquid's surface in the course of this reaction.

A yellow-brownish liquid containing the following averaged structure as main component was obtained.

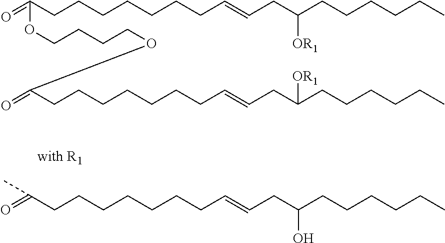

### Example 27b

Synthesis of a Stearic Acid Ester of a (Ricinoleic Acid₂-Butanediol-Ricinoleic Acid₂) pentamer diol

In a 100 ml three-necked bottle, equipped with refluxing condenser, thermometer and magnetic stirrer, dropping funnel, N₂ inlet tube and gas/vacuum outlet, 39.07 g (0.0322 mol) of the pentamer diol from example 27a and 18.34 g (0.0664 mol) stearic acid were mixed and heated to 200° C. for 23 hrs. A gentle N₂ stream was passed through the gas volume above the liquid's surface in the course of this reaction. A conversion of 98.4% of the OH groups was determined by means of ¹H NMR spectroscopy.

A yellow-brownish wax containing the following averaged structure as main component was obtained.

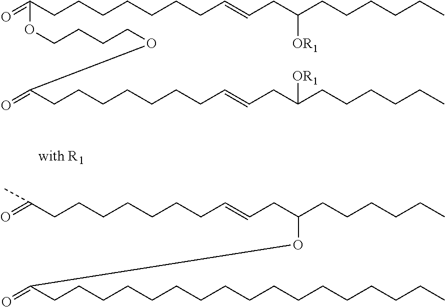

### Synthesis Example 28

Synthesis of a Stearyl Alcohol Ester of a (Succinic Acid-Ricinoleic Acid₂-Butanediol-Ricinoleic Acid₂-Succinic Acid) Heptamer Di-Acid

In a 100 ml three-necked bottle, equipped with refluxing condenser, thermometer and magnetic stirrer, dropping funnel, N₂ inlet tube and gas/vacuum outlet, 36 g (0.0297 mol) of the pentamer diol from example 27a and 5.94 g (0.0594 mol) succinic anhydride were mixed and heated to 200° C. for 17 hrs.

Afterwards, 16.07 g (0.0594 mol) stearyl alcohol were added and the reaction continued for 17 hrs. A gentle N₂ stream passes through the gas volume above liquid's surface in the course of the esterification of the stearyl alcohol. A conversion of 97.6% of the OH groups was determined by means of ¹H NMR spectroscopy.

A yellow-brownish wax containing the following averaged structure as main component was obtained.

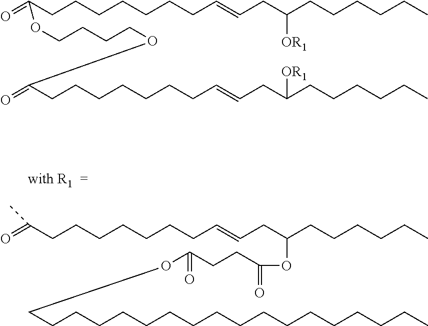

### Example 29

### Synthesis Example 29a

Synthesis of a Branched Bis-Stearic Acid Estolide Based on 2,2-Hydroxymethyl Propionic Acid

In a 100 ml three-necked bottle, equipped with refluxing condenser, thermometer and magnetic stirrer, dropping funnel and gas outlet tube 50 g (0.165 mol) stearic acid chloride were mixed with 11.07 g (0.083 mol) bis-2,2-hydroxymethyl propionic acid. The mixture was heated to 100° C. for 6 hrs. Volatiles were removed under reduced pressure (80° C./1 h/20 mmHg). The complete conversion of the OH groups was determined by means of ¹H NMR spectroscopy.

A greyish wax having the following structure was obtained:

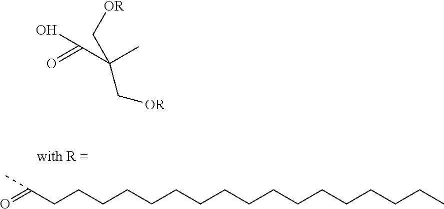

### Example 29b

Synthesis of a Branched Stearic Acid Ester of a (Ricinoleic Acid₂-Butanediol-Ricinoleic Acid₂) Pentamer Diol

In a 100 ml three-necked bottle, equipped with refluxing condenser, thermometer and magnetic stirrer, dropping funnel, N₂ inlet tube and gas/vacuum outlet, 10 g (0.015 mol) of the trimer diol intermediate from example 27a

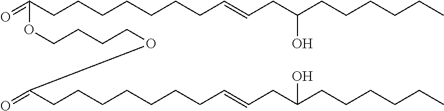 [SMILES: CCCCCCC(O)C/C=C/CCCCCCCC(=O)OCCCCOC(=O)CCCCCCC/C=C/CC(O)CCCCCC]

and 27.5 g (0.092 mol) ricinoleic acid were mixed and heated to 160° C. for 16 hrs. A gentle N₂ stream was passed through the gas volume above the liquid's surface.

An intermediate of the following averaged structure was obtained

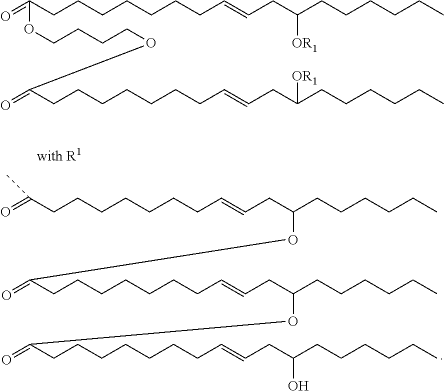

20.5 g (0.031 mol) of the bis-stearic acid estolide from example 29a were added and the reaction was continued at 200° C. for 18 hrs.

A conversion of 98.4% of the OH groups was determined by means of ¹H NMR spectroscopy.

A yellow-brownish wax containing the following averaged structure as main component was obtained.

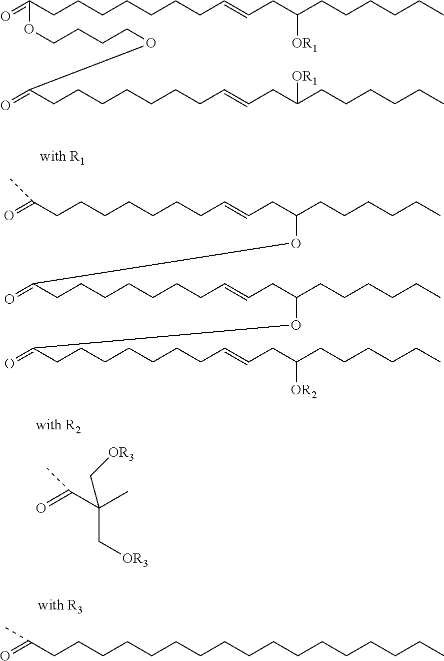

Hair Conditioner Application Tests

Hair conditioner formulation

| | | | |
|---|---|---|---|
| Chemical Name/ | | | |
| INCI Name | Conditioner base* | Example 11* | Example 8* |

Phase A

| | | | |
|---|---|---|---|
| Cetearyl alcohol | 2 | 2 | 2 |
| Stearyl Alcohol | 3.6 | 3.6 | 3.6 |
| Stearamidopropyl | 1.9 | 1.9 | 1.9 |
| Dimethylamine | | | |

Phase B

| | | | |
|---|---|---|---|
| Aqua | q.s. to 100 | q.s. to 100 | q.s. to 100 |
| Lactic acid | 0.5 | 0.5 | 0.5 |

Phase C

| | | | |
|---|---|---|---|
| Example 11 | 0 | 2 | 0 |
| Example 8 | 0 | 0 | 2 |

Phase D

| | | | |
|---|---|---|---|
| DMDM hydantoin | 0.5 | 0.5 | 0.5 |

*All values of the amounts of the components given indicate “parts by weight based on 100 parts by weight of the total composition”

Procedure:

Phase A and phase B are heated separately at 80° C. & 60° C., respectively. Phase A is mixed in Phase B.

After addition, the mixture is stirred for 30 min at 60° C. Phase C is added and the reaction is brought to atemperature of 25° C. Phase D is added and stirred for 15 min. The composition is stored in a suitable container.

Combing Analysis:

Each conditioner is evaluated in duplicates and the average as shown in the following data is considered for conclusion.

The combing force measurements were carried out using a Dia-Stron MTT 175 (Dia-Stron Limited) as described for above combing force measurements.

Dry and wet combing force measurement Procedure:
1. Asian hair tresses (2.5 gm) were prewashed with 2% NaOH followed by 10% SLES wash
2. The total work done in wet & dry combing was measured.
   Therein “total work done” is defined as work done during the movement of the comb across the hair tress and measured by Dia-Stron MTT175.
3. The hair tresses were washed with 350 mg of conditioner followed by washing with warm water thoroughly
4. The total work done in wet & dry combing was measured
5. The % of work done reduction was calculated.

Dry and Wet Combing Measurements: Total Work Done

| | Dry combing | Wet combing |
|---|---|---|
| | % Reduction in | % Reduction in |
| | total work done | total work done |
| | Treated vs. untreated | Treated vs. untreated |
| | (average two runs) | (average two runs) |
| cond. Base alone | 8 | 36 |
| cond. Base + ex. 11 | 40 | 61 |
| cond. Base + ex. 8 | 38 | 61 |

The data show that the addition of the compounds according to the invention of examples 11 and 8 to a conditioner formulation provides a dry and wet combing total work reduction which goes significantly beyond the reduction caused by the conditioner base alone.

Hair Dry Friction Measurement

Procedure:
1. Asian hair tresses (2.5 gm) were prewashed with 2% NaOH followed by 10% SLES wash
2. Hair tresses were dried thoroughly, equilibrated at 50% humidity, and the dry friction was measured
   (The dry friction was measured by means of Tribometer instrument from CSM)
3. Hair tresses were washed with 350 mg of conditioner followed by washing with warm water thoroughly
4. Hair tresses were dried thoroughly and equilibrated at 50% humidity and the dry friction measured
5. % Friction reduction calculated

% dry friction reduction:

| | Dry friction |
|---|---|
| | % Reduction in coefficient of friction (CoF) |
| | Treated vs. untreated |
| | (average of two runs) |
| cond. Base alone | 4 |
| cond. Base + 11 | 14 |
| cond. Base + 8 | 16 |

The data show that the addition of the compounds according to the invention of examples 11 and 8 to a conditioner formulation provides a dry coefficient of friction reduction which goes significantly beyond the reduction caused by the conditioner base alone.

1.-106. (canceled)

107. A hair care formulation containing at least one compound of the formula (I):
R¹(—X—C(O)—F)ₚ (I)
wherein
R¹ in formula (I) is selected from a p-valent, optionally substituted hydrocarbon radical and may contain optionally one or more groups selected from —O—, —NH—, —
C(O)—, —C(S)—, tertiary amino groups 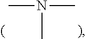
and can be optionally substituted by one or more selected from carboxyl groups or hydroxyl groups,
p≥2,
X can be the same or different and is selected from —O—, or —NR¹⁰—, wherein R¹⁰ is selected from the group consisting of hydrogen, or optionally substituted straight-chain, cyclic or branched, saturated, unsaturated or aromatic hydrocarbon radicals which have up to 100 carbon atoms which optionally contain one or more groups selected from —O—, —NH—, —C(O)—, —C(S)—, tertiary amino groups 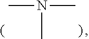
or in formula (I) R¹⁰ may form a bond to R¹ to form a cyclic structure,
F can be the same or different and is selected from optionally substituted straight-chain, cyclic or branched, saturated, unsaturated or aromatic hydrocarbon radicals which have up to 1005 carbon atoms, which optionally contain one or more groups selected from —O—, —NH—, —C(O)—, —C(S)—, and tertiary amino groups 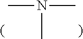
and can be optionally substituted by one or more selected from carboxyl, hydroxyl or halide groups,
with the proviso that at least one of the radicals F contains at least one moiety of the formula (II) or formula (II*):
—R⁶(—X—C(O)—R⁶)ₘ—X—C(O)—R⁷ (II)
—R⁶(—X—C(O)—R⁶)ₘ—X—C(O)—R⁷* (II*)
wherein
X is as defined,
m=0 to 20,
R⁶ is independently selected from a divalent optionally substituted straight-chain, cyclic or branched, saturated or unsaturated hydrocarbon radical which have up to 36 carbon atoms,
R⁷ is independently selected from optionally substituted straight-chain, cyclic or branched, saturated or unsaturated hydrocarbon radicals which have 1 to 1000 carbon atoms, optionally containing one or more groups selected from —O—, —NH—, —C(O)—, —C(S)—, tertiary amino groups 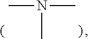
and which can be substituted with carboxyl, hydroxyl, or halide groups, wherein the radical R⁷ cannot contain an internal carboxy group or amide,
with the proviso that in at least one moiety of the formula (II) R⁷ has at least 2, carbon atoms, and in the same moiety of the formula (II) at least one R⁶ has at least 6 carbon atoms,
R⁷* is independently selected from optionally substituted branched or dendrimeric hydrocarbon radicals which have 1 to 1000 carbon atoms, optionally containing one or more groups selected from —O—, —NH—, —C(O)—, —C(S)—, tertiary amino groups 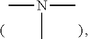
and which can be substituted with carboxyl, hydroxyl, or halide groups, wherein the radical R⁷* is terminated by two or more groups of the general structure
—X—C(O)-T
wherein X is as defined, and
T is a monovalent straight-chain, cyclic or branched, saturated or unsaturated hydrocarbon radical optionally substituted with carboxyl, hydroxyl, or halide groups with up to 36 carbon atoms,
with the proviso that in at least one moiety of the formula (II*) R⁷* is terminated by one or more groups T having at least 2 carbon atoms, and in the same moiety of the formula (II*) at least one R⁶ has at least 6 carbon atoms,
or
containing at least one compound of the general formula (IV)
R¹(—C(O)—X-G)_{q} (IV),
wherein
X is as defined,
R¹ in formula (IV) is selected from q-valent, optionally substituted hydrocarbon radicals which preferably have up to 1000 carbon atoms, and may contain optionally one or more groups selected from —O—, —NH—, —C(O)—, —C(S)—, tertiary amino groups 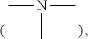
and can be optionally substituted by one or more selected from carboxyl groups or hydroxyl groups,
q=2 to 55, and
G can be the same or different and is selected from optionally substituted straight-chain, cyclic or branched, saturated, unsaturated or aromatic hydrocarbon radicals which have up to 1005 carbon atoms, which optionally contain one or more groups selected from —O—, —NH—, —C(O)—, —C(S)—, and tertiary amino groups 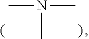
and can be substituted by one or more selected from carboxyl, hydroxyl or halide groups, with the proviso that at least one of the radicals G contains at least one moiety of the formula (V):
—R⁶(—C(O)—X—R⁶)ₘ—C(O)—X—R¹¹ (V)
wherein
X is as defined
m=0 to 20,
R⁶ in formula (V) is as defined for formula (I),
R¹¹ is independently selected from optionally substituted straight-chain, cyclic or branched, saturated or unsaturated hydrocarbon radicals which have 1 to 1000 carbon atoms, optionally containing one or more groups selected from —O—, —NH—, —C(O)—, —C(S)—, tertiary amino groups 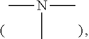
and which can be substituted with carboxyl, hydroxyl, or halide groups, wherein the radical R¹¹ cannot contain an internal carboxy group or amide,
with the proviso that in at least one moiety of the formula (V) R¹¹ has at least 2 carbon atoms, and in the same moiety of the formula (V) at least one R⁶ has at least 6 carbon atoms,
with the general proviso that the compound of the formula (I) is not exclusively composed of glycerol and ricinoleic acid moieties.

108. The hair care formulation according to claim 107, wherein at least one compound of the general formula (I) is represented by the general formula (III)
{[(F—C(O)—X—)₁₋₅R³(—X—C(O)—)]₁₋₃R⁴—C(O)—X—}ᵣ—R²(—X—C(O)—F)ₛ (III)
wherein X is as defined,
R² in formula (III) is selected from (r+s)-valent, optionally substituted hydrocarbon radicals which have up to 1000 carbon atoms, and may contain optionally one or more groups selected from —O—, —NH—, —C(O)—, —C(S)—, tertiary amino groups 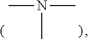
and can be optionally substituted by one or more carboxylic groups or hydroxyl groups, and optionally forms a bond to the nitrogen atom in the group —NR¹⁰—, in case R¹⁰ is a bond to R²,
R³ is selected from di- to hexavalent, optionally substituted hydrocarbon radicals which have up to 1000 carbon atoms, and may contain optionally one or more groups selected from —O—, —NH—, —C(O)—, —C(S)—, tertiary amino groups 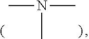
wherein if a plurality of R³ is present in formula (III), they can be the same or different,
R⁴ is selected from divalent to tetravalent optionally substituted straight-chain, cyclic or branched, saturated, unsaturated or aromatic hydrocarbon radicals which have up to 300 carbon atoms, which optionally contain one or more groups selected from —O—, —NH—, —C(O)—, —C(S)—, tertiary amino groups 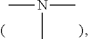
wherein if a plurality of R⁴ is present in formula (III), they can be the same or different,
and wherein
r+s=2 to 55,
r=0 to 54,
s≥1 and
in formula (III) F is as defined with the proviso that at least one of the radicals F contains at least one moiety of the formula (II) or formula (II*):
—R⁶(—X—C(O)—R⁶)ₘ—X—C(O)—R⁷ (II)
—R⁶(—X—C(O)—R⁶)ₘ—X—C(O)—R⁷* (II*)
wherein
X, R⁶, R⁷ and R⁷* are as defined,
m=0 to 20,
and with the proviso that in at least one moiety of the formula (II) R⁷ has at least 2 carbon atoms, and in the same moiety of the formula (II) at least one R⁶ has at least 6 carbon atoms, or that in at least one moiety of the formula (II*) R⁷* is terminated by one or more groups T having at least 2 carbon atoms, and in the same moiety of the formula (II*) at least one R⁶ has at least 6 carbon atoms.

109. The hair care formulation according to claim 108 wherein in the compound of formula (III):
R³ is selected from optionally substituted hydrocarbon radicals which have up to 300 carbon atoms, and may contain optionally one or more groups selected from —O—, —NH—, —C(O)—, —C(S)—, tertiary amino groups

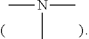

110. The hair care formulation according to claim 108, wherein in the compound of formula (III):
R⁴ is selected from a divalent to tetravalent, optionally substituted straight-chain, cyclic or branched, saturated, unsaturated or aromatic hydrocarbon radicals which have 2 to 300 carbon atoms and which optionally contain one or more groups selected from —O—, —NH—, —C(O)—, —C(S)—, tertiary amino groups

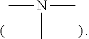

111. The hair care formulation according to claim 108, wherein in the compound of formula (III):
r=1 to 50.

112. The hair care formulation according to claim 107, wherein in the compound of the formulas (I) or (IV):
R⁶ is derived from monohydroxy carboxylic acids with up to 25 carbon atoms.

113. A hair care formulation according to claim 107, wherein in the compound of formula (I) R⁷ is derived from carboxylic acids with up to 25 carbon atoms which do not have hydroxyl substituents.

114. The hair care formulation according to claim 107, wherein in the compound of formula (I) at least one of R⁶ and R⁷ of the moieties of the general formula (II) are derived from unsaturated carboxylic acids.

115. A hair care formulation according to claim 108 wherein at least one of R² and R³ in the compound of formula (III):
is a divalent hydrocarbon radical comprising at least one ester group of the general formulas:
—[CH₂CH₂O]_{q1}—R⁹—[CH₂CH₂O]_{q1}—[CH₂CH₂]_{q2}—
with
q1 can be the same or different and are as defined and q2=1
and
—[CH₂CH(R⁸)CH₂O]ₜ₁—R⁹—[CH₂CH(R⁸)CH₂O]ₜ₁—[CH₂CH(R⁸)CH₂)]ₜ₂—
with
t1=0 to 32, t2=1,
R⁸═OH or —O—C(O)—R⁶—N⁺(R¹⁰)₃, wherein R¹⁰ and R⁶ are as defined,
with the proviso that the sum of the carbon atoms in R8 is 2 to 100, and
R⁹ is selected from —C(O)C(O)O—, —C(O)(CH₂)₁₋₈C(O)O—, or —C(O)(C₆H₄)C(O)O—, —C(O)CH═CHC(O)O—, —C(O)C(═CH₂)—CH₂C(O)O—, and —C(O)CH(OH)CH(OH)C(O)O—, with the proviso that the sum of the carbon atoms in R⁹ is 2 to 100.

116. A hair care formulation according to claim 107 wherein in the compound of formula (I):
in the one or more groups F in at least one moiety of the formula:
—R⁶(—X—C(O)—R⁶)ₘ—X—C(O)—R⁷ (II)
X and m are as defined, and
R⁶ is independently derived from lactic acid, ricinoleic acid, lesquerolic acid, 10-hydroxy stearic acid, 12-hydroxy stearic acid, or 14-hydroxy tetradecanoic acid, and
R⁷ is derived from octadecanoic acid, eicosanoic acid, docosanoic acid, 2-ethyl hexanoic acid, 2,2-dimethyl propionic acid, neodecanoic acid, or oleic acid.

117. The hair care formulation according to claim 107, wherein in the compound of formula (I)
the groups F contain at least one moiety of the formula (VI):
—R⁶(—X—C(O)—R⁶¹)_{M1}(—X—C(O)—R⁶²)ₘ₂—X—C(O)—R⁷ (VI):
wherein R⁶ in formula (VI) is selected from R⁶¹ and R⁶², X and R⁷ are as defined, and R⁶¹ and R⁶² represent two different groups R⁶ as defined,
and wherein
m1=0 to 20, m2=0 to 20, m=m1−m2, =0 to 20.

118. The hair care formulation according claim 107, wherein in the compound of the formula (I), or (IV) low melting and high melting fatty acids ≥C5 are specifically positioned within the R⁶ and R⁷ containing ester elements of the general formula (II) and the R⁶ and R¹¹ containing ester elements of the general formula (V):
—R⁶(—X—C(O)—R⁶)ₘ—X—C(O)—R⁷ (II) and
—R⁶(—C(O)—X—R⁶)ₘ—C(O)—X—R¹¹ (V),
wherein X, R⁶, and R⁷ and R¹⁰ are as defined,
and wherein
in one or more moieties of the formula (II) low melting fatty acids with 5 or more carbon atoms and a melting point of 40° C. or below each forming a group R⁶ are contained in the radical or the radicals R⁶ adjacent to R⁷, while at least one two or three high melting fatty acids with 5 or more carbon atoms and a melting point above 40° C. form the radical or radicals R⁶ at the opposite terminus of a R⁶- and R⁷-containing ester element of the formula (II), or in one or more moieties of the formula (II) at least one high melting fatty acids ≥C5 each forming R⁶ form the radical or radicals R⁶ adjacent to R⁷, while at least one two or three low melting fatty acids with 5 or more carbon atoms and a melting point below 40° C. form the radical or radicals R⁶ at the opposite terminus of a R⁶- and R⁷-containing ester element of the formula (II) or wherein
in one or more moieties of the formula (V) at least one low melting fatty acids ≥C5 each forming a group R⁶ are contained in the radical or the radicals R⁶ adjacent to R¹¹, while at least one two or three high melting fatty acids ≥C⁵ form the radical or radicals R⁶ at the opposite terminus of a R⁶- and R¹¹-containing ester element of the formula (V), or in one or more moieties of the formula (V) least onehigh melting fatty acids ≥C5 each forming R⁶ form the radical or radicals R⁶ adjacent to R¹¹, while at least one low melting fatty acids ≥C5 form the radical or radicals R⁶ at the opposite terminus of a R⁶- and R¹¹-containing ester element of the formula (V), or
wherein in the compound of the formula (I), (III) or (IV) low melting and high melting fatty acids ≥C5 are specifically positioned within the R⁶ and R⁷* containing ester elements of the general formula (II*)
—R⁶(—X—C(O)—R⁶)ₘ—X—C(O)—R⁷* (II*)
wherein X, R⁶, and R⁷* are as defined,
and wherein
in one or more moieties of the formula (II*) at least one low melting fatty acids with 5 or more carbon atoms and a melting point of 40° C. or below each forming a group R⁶ are contained in the radical or the radicals R⁶ adjacent to R⁷*, while at least one, high melting fatty acids with 5 or more carbon atoms and a melting point above 40° C. form the radical or radicals R⁶ at the opposite terminus of a R⁶- and R⁷*-containing ester element of the formula (II*), or in one or more moieties of the formula (II*) at least one high melting fatty acids ≥C5 each forming R⁶ form the radical or radicals R⁶ adjacent to R⁷*, while at least one low melting fatty acids with 5 or more carbon atoms and a melting point below 40° C. form the radical or radicals R⁶ at the opposite terminus of a R⁶- and R⁷*-containing ester element of the formula (II*).

119. The hair care formulation according to claim 107, wherein in at least one of the moieties of the formula (II), (II*) or (V) of the at least one compound of the formula (I), or (IV) the groups R⁶ and R⁷, R⁶ and R⁷*, or R⁶ and R¹¹ are not based on the same carboxylic acid structure.

120. The hair care composition according to claim 107, wherein in at least one compound of the general formula (I)
p is 2-6, R¹ is selected from di- to hexavalent linear, branched or cyclic alkylene groups, linear, branched or cyclic alkenylene groups, linear, branched or cyclic alkynylene groups, linear, branched or cyclic alkarylene groups, linear, branched or cyclic aralkylene groups and linear, branched or cyclic arylene groups,
and at least one group F contains one or more moieties of the general formula (II*)
—R⁶(—X—C(O)—R⁶)ₘ—X—C(O)—R⁷* (II*)
wherein R⁶, R⁷* and m are as defined.

121. The hair care composition according to claim 107, wherein in at least one compound of the general formula (I) one or more groups R⁷* are each terminated by three or more groups —O—C(O)-T, wherein T is as defined.

122. The hair care composition according to claim 112, wherein in at least one compound of the general formula (I) one or more groups R⁷* each contain at least two branching structures of the general formula
—C(O)—B(—O—)_{b},
wherein B is a linear or branched hydrocarbon group having 2-20 carbon atoms, and b
is 2 or more, and wherein the b groups (—O—) linked to the group B on the one side are linked to a C atom which may be the C atom of a CH² group or of a carbonyl group on the other side.

123. The hair care formulation according to claim 107, wherein the at least one compound of the general formula (I) is represented by one of the following schematic ester structures:
i) (fatty acid)-C(O)—O-(mono or oligo C8-C24 hydroxy fatty acid)-C(O)—O—(C2-C10 hydrocarbon)-O—C(O)-(mono or oligo C8-C24 hydroxy fatty acid)-O—C(O)-(fatty acid) or
ii) (branched fatty acid)-C(O)—O-(mono or oligo C8-C24 hydroxy fatty acid)-C(O)—O—(C2-C10 hydrocarbon)-O—C(O)-(mono or oligo C8-C24 hydroxy fatty acid)-O—C(O)-(branched fatty acid)
or
iii) (dendrimeric fatty acid)-C(O)—O-(mono or oligo C8-C24 hydroxy fatty acid)-C(O)—O—(C2-C10 hydrocarbon)-O—C(O)-(mono or oligo C8-C24 hydroxy fatty acid)-O—C(O)-(dendrimeric fatty acid).

124. The hair care formulation according to claim 107, wherein the compound of the formula (I), or (IV) comprises at least one moiety of the general formula
—([—O—C(O)—R⁶(—O—C(O)—R⁶)ₗ—O—C(O)-L-C(O)—O—(R⁶—C(O)—O)ₗ—R⁶—C(O)O])—
wherein R⁶ is as defined,
l is an integer independently selected from 0-20, and
L is a divalent hydrocarbon radical which may have 1 to 30 carbon atoms and may contain optionally one or more groups selected from —O—, —S—, —NH—, —C(O)—, —C(S)—, and tertiary amino groups

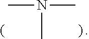

125. The hair care formulation according to claim 107, wherein the compound of the formula (I) or (IV) comprises at least one moiety of the following structure:
(fatty alcohol)-O—C(O)-(mono or oligo C8-C24 hydroxy fatty acid)-O—C(O)—(C1-C12 hydrocarbon)-C(O)—O-(mono or oligo C8-C24 hydroxy fatty acid)-C(O)—O-(fatty alcohol)

126. The hair care formulation according to claim 107, wherein the compound of the formula (I), or (IV) comprises at least one moiety of the structure
—([—O—C(O)—R⁶(—O—C(O)—R⁶)l-O—C(O)-L-C(O)—O—(R6-C(O)—O)l-R⁶—C(O)OD-R¹¹,
wherein L, l, R⁶ and R¹¹ are as defined.

127. A compound of the formula (Ia):
R¹(—X—C(O)—F)ₚ (Ia)
wherein
R¹ in formula (Ia) is selected from a p-valent, optionally substituted hydrocarbon radical and may contain optionally one or more groups selected from —O—, —NH—, —C(O)—, —C(S)—, tertiary amino groups 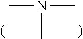
and can be optionally substituted by one or more selected from carboxyl groups or hydroxyl groups,
p≥2,
X can be the same or different and is selected from —O—, or —NR¹⁰—, wherein R¹⁰ is selected from the group consisting of hydrogen, or optionally substituted straight-chain, cyclic or branched, saturated, unsaturated or aromatic hydrocarbon radicals which have up to 100 carbon atoms which optionally contain one or more groups selected from —O—, —NH—, —C(O)—, —C(S)—, tertiary amino groups 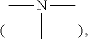
or in formula (I) R¹⁰ may form a bond to R¹ to form a cyclic structure,
F can be the same or different and is selected from optionally substituted straight-chain, cyclic or branched, saturated, unsaturated or aromatic hydrocarbon radicals which have up to 1005 carbon atoms, which optionally contain one or more groups selected from —O—, —NH—, —C(O)—, —C(S)—, and tertiary amino groups 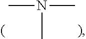
and can be optionally substituted by one or more selected from carboxyl, hydroxyl or halide groups,
with the proviso that at least one of the radicals F contains at least one moiety of the formula (IIa) or formula (IIa*):
—R⁶(—X—C(O)—R⁶)ₘ—X—C(O)—R⁷ (IIa)
—R⁶(—X—C(O)—R⁶)ₘ—X—C(O)—R⁷* (IIa*)
wherein
X is as defined,
m=1 to 20
R⁶ is independently selected from a divalent optionally substituted straight-chain, cyclic or branched, saturated or unsaturated hydrocarbon radical which have up to 36 carbon atoms,
R⁷ is independently selected from optionally substituted straight-chain, cyclic or branched, saturated or unsaturated hydrocarbon radicals which have 1 to 1000 carbon atoms, optionally containing one or more groups selected from —O—, —NH—, —C(O)—, —C(S)—, tertiary amino groups 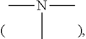
and which can be substituted with carboxyl, hydroxyl, or halide groups, wherein the radical R⁷ cannot contain an internal carboxy group or amide,
with the proviso that in at least one moiety of the formula (IIa) R⁷ has at least 2, carbon atoms, and in the same moiety of the formula (IIa) at least one R⁶ has at least 6 carbon atoms,
R⁷* is independently selected from optionally substituted branched or dendrimeric hydrocarbon radicals which have 1 to 1000 carbon atoms, optionally containing one or more groups selected from —O—, —NH—, —C(O)—, —C(S)—, tertiary amino groups 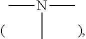
and which can be substituted with carboxyl, hydroxyl, or halide groups, wherein the radical R⁷* is terminated by two or more groups of the general structure
—X—C(O)-T
wherein X is as defined, and
T is a monovalent straight-chain, cyclic or branched, saturated or unsaturated hydrocarbon radical optionally substituted with carboxyl, hydroxyl, or halide groups with up to 36 carbon atoms,
with the proviso that in at least one moiety of the formula (II*) R⁷* is terminated by one or more groups T having at least 2 carbon atoms, and in the same moiety of the formula (IIa*) at least one R⁶ has at least 6 carbon atoms,
or
containing at least one compound of the general formula (IVa)
R¹(—C(O)—X-G)_{q} (IVa),
wherein X is as defined,
R¹ in formula (IVa) is selected from q-valent, optionally substituted hydrocarbon radicals, and may contain optionally one or more groups selected from —O—, —NH—, — C(O)—, —C(S)—, tertiary amino groups 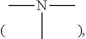
and can be optionally substituted by one or more selected from carboxyl groups or hydroxyl groups,
q=2 to 55, and
G can be the same or different and is selected from optionally substituted straight-chain, cyclic or branched, saturated, unsaturated or aromatic hydrocarbon radicals which have up to 1005 carbon atoms, which optionally contain one or more groups selected from —O—, —NH—, —C(O)—, —C(S)—, and tertiary amino groups 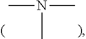
and can be substituted by one or more selected from carboxyl, hydroxyl or halide groups, with the proviso that at least one of the radicals G contains at least one moiety of the formula (Va):
—R⁶(—C(O)—X—R⁶)ₘ—C(O)—X—R¹¹ (Va)
wherein X is as defined,
m=1 to 20,
R⁶ in formula (Va) is as defined for formula (Ia),
R¹¹ is independently selected from optionally substituted straight-chain, cyclic or branched, saturated or unsaturated hydrocarbon radicals which have 1 to 1000 carbon atoms, optionally containing one or more groups selected from O—, —NH—, —C(O)—, — C(S)—, tertiary amino groups 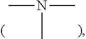
and which can be substituted with carboxyl, hydroxyl, or halide groups, wherein the radical R¹¹ cannot contain an internal carboxy group or amide, with the proviso that in at least one moiety of the formula (Va) R¹¹ has at least 2 carbon atoms, and in the same moiety of the formula (Va) at least one R⁶ has at least 6 carbon atoms,
with the general proviso that the compound of the formula (Ia) is not exclusively composed of glycerol and ricinoleic acid moieties.

128. The compound of the formula (Ia) according to claim 127, which is represented by the general formula (IIIa):
{[(F—C(O)—X—)₁₋₅R³(—X—C(O)—)]₁₋₃R⁴—C(O)—X—}ᵣ—R²(—X—C(O)—F)ₛ (IIIa)
wherein
X can be the same or different and is selected from —O—, or —NR¹⁰—, wherein R¹⁰ is selected from the group consisting of hydrogen, or optionally substituted straight-chain, cyclic or branched, saturated, unsaturated or aromatic hydrocarbon radicals which have up to 100 carbon atoms which optionally contain one or more groups selected from —O—, —NH—, —C(O)—, —C(S)—, tertiary amino groups 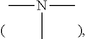
or in formula (IIIa) R¹⁰ may form a bond to R² to form a cyclic structure,
R² in formula (IIIa) is selected from (r+s)-valent, optionally substituted hydrocarbon radicals which have up to 1000 carbon atoms, and may contain optionally one or more groups selected from —O—, —NH—, —C(O)—, —C(S)—, tertiary amino groups 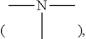
and can be optionally substituted by one or more carboxylic groups or hydroxyl groups, and optionally forms a bond to the nitrogen atom in the group —NR¹⁰—, in case R¹⁰ is a bond to R²,
R³ is selected from di- to hexavalent, optionally substituted hydrocarbon radicals which have up to 1000 carbon atoms, and may contain optionally one or more groups selected from —O—, —NH—, —C(O)—, —C(S)—, tertiary amino groups 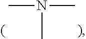
wherein if a plurality of R³ is present in formula (IIIa), they can be the same or different,
R⁴ is selected from divalent to tetravalent optionally substituted straight-chain, cyclic or branched, saturated, unsaturated or aromatic hydrocarbon radicals which have up to 300 carbon atoms, which optionally contain one or more groups selected from —O—, —NH—,
—C(O)—, —C(S)—, tertiary amino groups 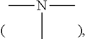
wherein if a plurality of R⁴ is present in formula (IIIa), they can be the same or different,
and wherein
r+s=2 to 55,
r=0 to 54,
s≥1,
and in formula (IIIa) F can be the same or different and is selected from optionally substituted straight-chain, cyclic or branched, saturated, unsaturated or aromatic hydrocarbon radicals which have up to 1005 carbon atoms, which optionally contain one or more groups selected from —O—, —NH—, —C(O)—, —C(S)—, and tertiary amino groups 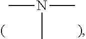
and can be optionally substituted by one or more selected from carboxyl, hydroxyl or halide groups, with the proviso that at least one of the radicals F contains at least one moiety of the formula (IIa) or (IIa*):
—R⁶(—X—C(O)—R⁶)ₘ—X—C(O)—R⁷ (IIa)
—R⁶(—X—C(O)—R⁶)ₘ—X—C(O)—R⁷* (IIa*)
wherein
X is as defined,
with m=1 to 20,
R⁶ is independently selected from a divalent optionally substituted straight-chain, cyclic or branched, saturated or unsaturated hydrocarbon radical which have up to 36 carbon atoms,
R⁷ is independently selected from optionally substituted straight-chain, cyclic or branched, saturated or unsaturated hydrocarbon radicals which have 1 to 1000 carbon atoms, optionally containing one or more groups selected from —O—, —NH—, —C(O)—, — C(S)—, tertiary amino groups 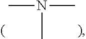
and which can be substituted with carboxyl, hydroxyl, or halide groups, wherein the radical R⁷ cannot contain an internal carboxy group or amide,
and with the proviso that in at least one moiety of the formula (IIa) R⁷ has at least 2 carbon atoms,
and in the same moiety of the formula (IIa) at least one R⁶ has at least 6 carbon atoms,
R⁷* is independently selected from optionally substituted branched or dendrimeric hydrocarbon radicals which have 1 to 1000 carbon atoms, optionally containing one or more groups selected from —O—, —NH—, —C(O)—, —C(S)—, tertiary amino groups 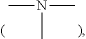
and which can be substituted with carboxyl, hydroxyl, or halide groups, wherein the radical R⁷* is terminated by two or more groups of the general structure
—X—C(O)-T
wherein X is as defined, and
T is a monovalent straight-chain, cyclic or branched, saturated or unsaturated hydrocarbon radical optionally substituted with carboxyl, hydroxyl, or halide groups with up to 36 carbon atoms,
with the proviso that in at least one moiety of the formula (IIa*) R⁷* is terminated by one or more groups T having at least 2 carbon atoms, and in the same moiety of the formula (IIa*) at least one R⁶ has at least 6 carbon atoms.

129. The compound of the formula (IIIIa) according to claim 128,
wherein:
R² in the compound of the formula (IIIa) is selected from divalent to hexavalent, optionally substituted hydrocarbon radicals.

130. The compound of the formula (IIIIa) according to claim 128, wherein:
R³ is selected from di- to hexavalent residues.

131. The compound of the formula (IIIa) according to claim 127,
wherein R⁴ is selected from divalent to tetravalent, optionally substituted straight-chain, cyclic or branched, saturated, unsaturated or aromatic hydrocarbon radicals which have 2 to 300 carbon atoms, which optionally contain one or more groups selected from —O—, —NH—, —C(O)—, —C(S)—, tertiary amino groups

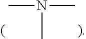

132. The compound of the formula (IIIa) according to claim 127, wherein:
r=1 to 10.

133. The compound of the formula (Ia), or (IVa) according to claim 127, wherein low melting and high melting fatty acids ≥C5 are specifically positioned within the R⁶ and R⁷ containing ester elements of the general formula (IIa) and within the R⁶ and R¹¹ containing ester elements of the general formula (Va):
—R⁶(—X—C(O)—R⁶)ₘ—X—C(O)—R⁷ (IIa) and
—R⁶(—C(O)—X—R⁶)ₘ—C(O)—X—R¹¹ (Va),
wherein X, R⁶, R⁷, and R¹¹ are as defined,
or within the R⁶ and R⁷* containing ester elements of the general formula (IIa*)
—R⁶(—X—C(O)—R⁶)ₘ—X—C(O)—R⁷* (IIa*),
wherein X, R⁶, R⁷* are as defined,
and wherein
in one or more moieties of the formula (IIa) at least one low melting fatty acids ≥C5 each forming a group R⁶ are contained in the radical or the radicals R⁶ adjacent to R⁷, while at least one high melting fatty acids ≥C5 form the radical or radicals R⁶ at the opposite terminus of a R⁶- and R⁷-containing ester element of the formula (II), or in one or more moieties of the formula (IIa) at least one high melting fatty acids ≥C5 each forming R⁶ form the radical or radicals R⁶ adjacent to R⁷, while at least one low melting fatty acids ≥C5 form the radical or radicals R⁶ at the opposite terminus of a R⁶- and R⁷-containing ester element of the formula (IIa),
or wherein in one or more moieties of the formula (IIa*) at least one low melting fatty acids ≥C5 each forming a group R⁶ are contained in the radical or the radicals R⁶ adjacent to R⁷*, while at least one high melting fatty acids ≥C5 form the radical or radicals R⁶ at the opposite terminus of a R⁶- and R⁷*-containing ester element of the formula (IIa*), or in one or more moieties of the formula (IIa*) at least one high melting fatty acids ≥C5 each forming R⁶ form the radical or radicals R⁶ adjacent to R⁷*, while at least one low melting fatty acids ≥C5 form the radical or radicals R⁶ at the opposite terminus of a R⁶- and R⁷*-containing ester element of the formula (IIa*),
or wherein
in one or more moieties of the formula (Va) at least one, low melting fatty acids ≥C5 each forming a group R⁶ are contained in the radical or the radicals R⁶ adjacent to R¹¹, while at least one, high melting fatty acids ≥C5 form the radical or radicals R⁶ at the opposite terminus of a R⁶- and R¹¹-containing ester element of the formula (Va), or in one or more moieties of the formula (Va) at least one, high melting fatty acids ≥C5 each forming R⁶ form the radical or radicals R⁶ adjacent to R¹¹, while at least one, low melting fatty acids ≥C5 form the radical or radicals R⁶ at the opposite terminus of a R⁶- and R¹¹-containing ester element of the formula (Va).

134. The compound of the formula (Ia) or (IVa) according to claim 127, wherein:
R⁶ is independently selected from optionally substituted straight-chain, cyclic or branched, saturated or unsaturated hydrocarbon radicals which have up to 24 carbon atoms.

135. The compound of the formula (Ia) according to claim 127, wherein:
R⁷ is independently selected from optionally substituted straight-chain, cyclic or branched, saturated or unsaturated hydrocarbon radicals which have 1 to 36 carbon atoms.

136. The compound of the formula (Ia) according to claim 127, wherein at least one of R⁶ and R⁷ of the moieties of the general formula (IIa) are derived from unsaturated carboxylic acids.

137. The compound of formula (IIIa) according to claim 128, wherein in the compound of formula (III) R⁴ is derived from dicarboxylic acids, tricarboxylic acids or tetracarboxylic acids,
and condensation products of hydroxy carboxylic acids;
or
R⁴ is derived from amide condensation products of amino acids with maleic acid or succinic acid; or R⁴ is derived from the ester condensation products of divalent alcohols, with dicarboxylic acid anhydrides; or
R⁴ is derived from tri- or tetracarboxylic acids; or
R⁴ is derived from the bisamide condensation products of amino acids with maleic acid or succinic acid; or
R⁴ is derived from the ester condensation products of trivalent alcohols, with dicarboxylic acid anhydrides; or
R⁴ is derived from the ester condensation products of tetravalent alcohols with dicarboxylic acid anhydrides.

138. The compound of the formula (Ia) or (IVa) according to claim 127, wherein in at least one of the moieties of the formula (IIa), (IIa*) or (Va) at least two different R⁶ groups are present.

139. The compound of the formula (Ia) according to claim 127, wherein the compound of the formula (Ia) or (IIIa) contains one or more groups R⁷* each terminated by three or more groups —O—C(O)-T, wherein T is as defined.

140. The compound of the formula (Ia) according to claim 127, wherein one or more groups R⁷* each contain at least two branching structures of the general formula
—C(O)—B(—O—)_{b},
wherein B is a linear or branched hydrocarbon group having 2-20 carbon atoms, and b is 2 or more, and wherein the b groups (—O—) linked to the group B on the one side are linked to a C atom which may be the C atom of a CH₂ group or of a carbonyl group on the other side.

141. The compound of the formula (Ia) according to claim 127, wherein one or more groups R⁷* are terminated by two or more groups of the structure
—R⁶(—X—C(O)—R⁶)ₜ—X—C(O)-T,
wherein R⁶, and T are as defined, and
X═O, t is independently 0-12.

142. The compound of the general formula (Ia) according to claim 127, wherein the compound is represented by one of the following schematic ester structures:
i) (fatty acid)-C(O)—O-(mono or oligo C8-C24 hydroxy fatty acid)-C(O)—O—(C2-C10 hydrocarbon)-O—C(O)-(mono or oligo C8-C24 hydroxy fatty acid)-O—C(O)-(fatty acid)
or
ii) (branched fatty acid)-C(O)—O-(mono or oligo C8-C24 hydroxy fatty acid)-C(O)—O—(C2-C10 hydrocarbon)-O—C(O)-(mono or oligo C8-C24 hydroxy fatty acid)-O—C(O)-(branched fatty acid)
or
iii) (dendrimeric fatty acid)-C(O)—O-(mono or oligo C8-C24 hydroxy fatty acid)-C(O)-0-(C2-C10 hydrocarbon)-O—C(O)-(mono or oligo C8-C24 hydroxy fatty acid)-O—C(O)-(dendrimeric fatty acid).

143. The compound of the general formula (Ia) or (IVa) according to claim 127, wherein the compound comprises at least one moiety of the general formula
—([—O—C(O)—R⁶(—O—C(O)—R⁶)ₗ—O—C(O)-L-C(O)—O—(R⁶—C(O)—O)ₗ—R⁶—C(O)O])—
wherein R⁶ is as defined,
l is an integer independently selected from 0-20, and
L is a divalent hydrocarbon radical which may have 1 to 30 carbon atoms and may contain optionally one or more groups selected from —O—, —S—, —NH—, —C(O)—, —C(S)—, and tertiary amino groups

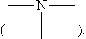

144. The compound of the general formula (Ia) or (IVa) according to claim 127, wherein the compound comprises at least one moiety of the following structure:
(fatty alcohol)-O—C(O)-(mono or oligo C8-C24 hydroxy fatty acid)-O—C(O)—(C1-C12 hydrocarbon)-C(O)—O-(mono or oligo C8-C24 hydroxy fatty acid)-C(O)—O-(fatty alcohol).

145. The compound of the general formula (Ia) or (IVa) according to claim 127, wherein the compound comprises at least one moiety of the structure
—([—O—C(O)—R⁶(—O—C(O)—R⁶)l—O—C(O)-L-C(O)—O—(R⁶—C(O)—O)l-R⁶—C(O)OD-R¹¹,
wherein L, l, R⁶ and R¹¹ are as defined.

146. A cosmetic formulation for skin and/or hair care comprising the compound of the formula (Ia), or (IVa) as defined in claim 127.

147. A method of treating fibers comprising employing the compound of the formula (Ia) or (IVa) as defined in claim 127.
